(19) United States
(12) Patent Application Publication
(10) Pub. No.: US 2024/0122709 A1
(43) Pub. Date: Apr. 18, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Lauren R. Freschauf, Mission Viejo, CA (US); Eric Michael Oberwise, Newport Beach, CA (US); Grant Matthew Stearns, Costa Mesa, CA (US); Chris J. Okos, Huntington Beach, CA (US); Eran Hoffer, Yahud (IL); Rupesh Gajanan Nawalakhe, Trabuco Canyon, CA (US); Sandip Vasant Pawar, Irvine, CA (US); Sergio Delgado, Irvine, CA (US); Munir Hicham Eltal, Irvine, CA (US)

(21) Appl. No.: 18/395,235

(22) Filed: Dec. 22, 2023

Related U.S. Application Data
(63) Continuation of application No. PCT/US2022/035672, filed on Jun. 30, 2022.
(60) Provisional application No. 63/308,940, filed on Feb. 10, 2022.
(60) Provisional application No. 63/278,037, filed on Nov. 10, 2021.
(60) Provisional application No. 63/217,622, filed on Jul. 1, 2021.

(51) Int. Cl. A61F 2/24

(52) U.S. Cl. CPC A61F 2/2451; A61F 2220/0008; A61F 2220/0041; A61F 2250/0007

(57) ABSTRACT

An implantable device or implant is configured to be positioned within a native heart valve to allow the native heart valve to form a more effective seal. The implantable device or implant can include a pair of paddles frame a coaptation element. The coaptation element defines a first area when viewed from above. The paddle frames define when viewed from above. A ratio of the second area to the first area is greater than or equal to 2 to 1.

### RELATED APPLICATIONS

The present application is a continuation of Patent Cooperation Treaty application no. PCT/US2022/035672, filed on Jun. 30, 2022, which claims the benefit of U.S. Provisional Patent Application No. 63/308,940, filed on Feb. 10, 2022, U.S. Provisional Patent Application No. 63/278,037, filed on Nov. 10, 2021, and U.S. Provisional Patent Application No. 63/217,622, filed on Jul. 1, 2021, which are all incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

In some implementations, an implantable device or implant is configured to be positioned within a native heart valve to allow the native heart valve to form a more effective seal. The implantable device or implant can include a paddle frame that includes an inner frame portion and an outer frame portion. A cover is configured to prevent or inhibit regurgitant blood flow between the inner frame portion and the outer frame portion.

In some implementations, the implantable device or implant includes a first anchor and a second anchor, where each of the first and second anchors have a paddle frame that includes an inner frame portion and an outer frame portion. The anchors are configured to be move to a closed position in which the inner frame portions and the outer frame portions compress one or more leaflets (e.g., leaflets of a heart valve, such as the mitral valve, tricuspid valve, etc.) within inner and outer pinch points (e.g., pinch regions or regions where the frame members press the leaflet(s)) to secure the implantable device to a native valve of a patient. The implantable device or implant further includes a cover that is attached to the paddle frames of the anchors. The cover is configured block or inhibit blood flow between the anchors.

In some implementations, the implantable device or implant includes anchors and/or paddle frames that are adjustable in width to a variety of different widths. In some implementations, the cover is configured to block blood flow between the anchors at any width of the anchors and/or paddle frames.

In some implementations, the paddle frames of the implantable device include an outer frame portion that is movable between a narrowed position and an expanded position. The proximal width of a proximal end of the outer frame portion is greater than a distal width of a distal end of the outer frame portion when the outer frame portion is in the expanded position.

In some implementations, the paddle frames of the implantable device include an outer frame portion that is movable between a narrowed position and an expanded position. The distal width of a distal end of the outer frame portion is greater than a proximal width of a proximal end of the outer frame portion when the outer frame portion is in the expanded position.

In some implementations, there is provided an implantable device or implant comprising a first anchor and a second anchor, wherein the first anchor and the second anchor are configured to be moved to a closed position in which the first anchor and the second anchor compress one or more leaflets of a native valve within a first pinch point and a second pinch point such that the implantable device is secured to the native valve. The first pinch point and the second pinch point can be between portions of the first anchor and the second anchor. A cover is attached to the first anchor and the second anchor and extends between the first anchor and the second anchor.

In some implementations, the first pinch point is formed between first frame members of the first and second anchors. In some implementations, the second pinch point is formed between second frame portions (e.g., outer frame portions) of the first and second anchors.

In some implementations, the first frame members are separate and distinct from the second frame members. In some implementations, the first frame members are integrally formed with the second frame members for each of the first and second anchor.

In some implementations, the first frame members are protrusions or protruding portions of the first and second anchors, e.g., protruding or extending from a surface of the first and second anchors. In some implementations, the second frame members are protrusions or protruding portions of the first and second anchors, e.g., protruding or extending from a surface of the first and second anchors.

In some implementations, the first anchor comprises an inner frame portion that is rigid and an outer frame portion member that is flexible.

In some implementations, the cover extends across an inner surface of the first anchor. In some implementations, the cover extends across an outer surface of the first anchor.

In some implementations, the cover is positioned between at least a portion of an area defined by an interior surface of the first anchor. In some implementations, the cover extends across an entirety of an area defined by an interior surface of the first anchor.

In some implementations, the cover encapsulates at least a portion of an outer paddle of each of the first and second anchors. In some implementations, the cover encapsulates an entirety of an outer paddle of each of the first and second anchors.

In some implementations, the cover comprises a first membrane that attaches to a paddle frame of the first anchor and a second membrane that attaches to a paddle frame of the second anchor.

In some implementations, the first and second membranes are connected such together to block blood flow between the first and second anchors.

In some implementations, the cover comprises a single membrane that attaches to paddle frames of both of the first and second anchors.

In some implementations, the cover (e.g., the single membrane, multiple membranes, etc.) is configured to form a canopy that extends between the first and second anchors.

In some implementations, the cover is made of a porous material that becomes impermeable to blood flow over time.

In some implementations, the cover is configured to provide a compressive force against the one or more leaflets in an area between the first pinch point and the second pinch point.

In some implementations, the first anchor and the second anchor are configured to be moved between a narrowed position having a first width and expanded position having a second width greater than the first width.

In some implementations, the cover is configured to be in a taut state when the outer frame portion is in the narrowed position, and wherein the cover is configured to stretch when the outer frame portion is in the expanded position.

In some implementations, the cover is configured to be in a taut state when the outer frame portion is in the narrowed position, and the cover includes one or more stretchable portions that allow the cover to stretch when the outer frame is are in the expanded position.

In some implementations, there is provided an implantable device or implant comprising a first anchor and a second anchor, wherein the first anchor and the second anchor are configured to be moved between a narrowed configuration having a first width and an expanded configuration having a second width greater than the first width, and wherein the first anchor and the second anchor are configured to be moved to a closed position in which the first anchor and the second anchor compress one or more leaflets of a native valve between portions thereof. The device or implant comprises a flexible cover attached to the first anchor and the second anchor.

In some implementations, the cover is configured to provide a compressive force against the one or more leaflets in an area between the portions compressing the one or more leaflets when the implantable device is secured to the native valve.

In some implementations, the first anchor and the second anchor are configured to be moved to a closed position in which the first anchor and the second anchor compress one or more leaflets of a native valve between a first pinch point and a second pinch point such that the implantable device is secured to the native valve. In some implementations, the cover is configured to provide a compressive force against the one or more leaflets in an area between the first pinch point and the second pinch point when the implantable device is secured to the native valve.

In some implementations, the cover is configured to be in a taut state when the first anchor and the second anchor are in the narrowed configuration, and the cover is configured to stretch when the first anchor and the second anchor are in the expanded configuration (e.g., remain in a taut state in the narrowed configuration, in the expanded configuration, and in positions between the narrowed configuration and the expanded configuration).

In some implementations, the implantable device is secured to the native valve first anchor comprises an inner frame portion that is rigid and an outer frame portion that is flexible.

In some implementations, the cover is connected to the first anchor by one or more stitches.

In some implementations, the cover extends across an inner surface of the first anchor. In some implementations, the cover extends across an outer surface of the first anchor.

In some implementations, the cover is positioned between at least a portion of an area defined by an interior surface of the first anchor. In some implementations, the cover extends across an entirety of an area defined by an interior surface of the first anchor.

In some implementations, the cover encapsulates at least a portion of an outer paddle of each of the first and second anchors. In some implementations, the cover encapsulates an entirety of an outer paddle of each of the first anchor and the second anchors.

In some implementations, the cover comprises a first membrane that attaches to the first anchor and a second membrane that attaches to the second anchor.

In some implementations, the first membrane and the second membrane are not connected such that a gap exists between bottom edges of the first membrane and the second membrane.

In some implementations, the cover comprises a single membrane that attaches to both of the first anchor and the second anchor.

In some implementations, the single membrane creates or forms a canopy that extends between the first anchor and the second anchor.

In some implementations, the cover is made of a porous material. In some implementations, the cover is made of an impermeable material.

In some implementations, an implantable device or implant includes a coaptation element (e.g., spacer, plug, filler, foam, sheet, membrane, coaption element, wedge, barrier, balloon, etc.) and one or more anchors. The coaptation element defines a first area when viewed from above. The one or more anchors are coupled to the coaptation element. The anchors are movable between an open position and a closed position. The one or more anchors are configured to attach to one or more leaflets of a native heart valve. Each of the anchors includes a paddle frame.

In some implementations, the paddle frame defines an outer portion of the implantable device when viewed from above and the anchors are in the closed position. In some implementations, the outer portion of the device has a second area when viewed from above. In some implementations, a ratio of the second area to the first area is greater than or equal to 2 to 1.

In some implementations, the ratio of the second area to the first area can be greater than or equal to 3 to 1, 4 to 1, 5 to 1, or 6 to 1.

In some implementations, the coaptation element can comprise one or more planar side surface and/or tapered side surfaces. The coaptation element can include a first portion that is rectangular and a second portion that is rounded.

In some implementations, the coaptation element can be injection molded. The coaptation element can comprise a polymer material.

In some implementations, the implantable device can comprise an attachment portion having a collar that is configured to attach to a delivery device. The collar of the attachment portion can be integral to the coaptation element.

In some implementations, the coaptation element comprises one or more attachment openings for aligning with one or more openings of a component of the anchors such that the component of the anchors can be attached to the coaptation element.

In some implementations, the anchors can include an inner paddle and an outer paddle and/or clasp. The paddle frame of the each of the anchors can comprise an inner paddle frame and an outer paddle frame.

In some implementations, the outer paddle frame defines the outer portion of the implantable device when viewed from above and the anchors are in the closed position.

In some implementations, the implantable devices or implants described above are incorporated into a system, e.g., a valve repair system comprising, for example, a delivery system and the implantable device or implant.

In some implementations, a valve repair system for repairing a native valve of a heart includes a delivery device, an implantable device, and a coupler. The delivery device has a width adjustment element (e.g., a width adjustment control, line, shaft, wire, tether, etc.) that includes an external threaded portion. The implantable device is configured to be implanted on the native valve. The anchor portion has one or more anchors. The one or more anchors are configured to attach to one or more leaflets of a native valve.

In some implementations, each of the anchors can have a paddle frame that includes an inner end.

In some implementations, the coupler removably connects the width adjustment element of the delivery device to the inner end of the anchors. In some implementations, the coupler comprises one or more attachment projections that extend inward from a body of the coupler. In some implementations, the attachment projections are configured to removably attach to the external threaded portion of the width adjustment element. The width adjustment element is configured to move the inner end of the anchors to move the paddle frame between a narrowed position and an expanded position.

In some implementations, the one or more attachment projections can include a first attachment projection and a second attachment projection. The first attachment projection can be offset from the second attachment projection along a height of the body of the coupler.

In some implementations, the coupler can have arms that are movable between a normal position and an engaged position. When the arms are in the normal position when the coupler is disconnected from the implantable device, and wherein the arms are in the engaged position when the coupler is connected to the width adjustment element.

In some implementations, a valve repair system for repairing a native valve of a patient includes a delivery device, an implantable device, and a coupler. The delivery device can have a width adjustment element (e.g., a width adjustment control, line, shaft, wire, tether, etc.). The implantable device can be configured to be implanted on the native valve of the patient. The implantable device includes an anchor portion having one or more anchors. The one or more anchors are configured to attach to one or more leaflets of a native valve.

In some implementations, the implantable device has a receiver (e.g., an internally threaded element, a column, a conduit, a hollow member, a notched receiving portion, a tube, a shaft, a sleeve, a post, a housing, tracks, a cylinder. etc.) defining a lumen that includes internal threads.

In some implementations, the receiver has an unattachable portion that prevents a coupler from connecting to the receiver when the coupler is disposed within the unattachable portion.

In some implementations, each of the anchors have a paddle frame that includes an inner end.

In some implementations, the coupler removably connects the width adjustment element of the delivery device to the inner end of the anchors.

In some implementations, the coupler comprises at least two arms that are movable between a normal position and an engaged position. In some implementations, the arms are in the normal position when the coupler is disconnected from the width adjustment element, and the arms are in the engaged position when the coupler is connected to the width adjustment element.

In some implementations, the arms are configured to attach to internal threads of the lumen of the receiver when the arms are in the normal position.

In some implementations, the arms can include one or more tabs that are configured to be inserted into one or more slots of the body of the coupler when the arms are in the normal position.

In some implementations, the arms are configured to allow the coupler to move within the lumen of the receiver when in the engaged position.

In some implementations, a first arm of the arms is offset from a second arm along a height of a body of the coupler.

In some implementations, a first portion of each arm extends into a lumen of the coupler when the arms are in the normal position and a second portion of each arm extends away from an exterior of a body of the coupler when the arms are in the normal position.

In some implementations, the arms can extend away from an exterior surface of a body of the coupler by between about 20 degrees and about 45 degrees when the arms are in the normal position.

In some implementations, the coupler comprises an upper body, a lower body, and a plurality of struts connected to the upper and lower bodies. The coupler is movable between a first position in which the plurality of struts are in a straight configuration and a second position in which the plurality of struts are in a spiraled configuration.

In some implementations, the coupler is configured to attach to the internal threads of the receiver when the plurality of struts are in the spiraled configuration.

In some implementations, the plurality of struts are normally in the spiraled configuration.

In some implementations, the coupler comprises at least two arms that are movable between a normal position and an engaged position. Each of the arms have a central portion, a first connection member that connects the central portion to a proximal end of the coupler, and a second connection member that connects the central portion to a distal end of the coupler.

In some implementations, the arms are configured to attach to the internal threads of the receiver when the arms are in the normal position.

In some implementations, the first and second connection members are normally in torsion such that a first portion of the central portion is disposed within a lumen of the coupler and a second portion of the central portion extends away from an exterior of the coupler.

In some implementations, the arms of the coupler have a “T” shape.

In some implementations, an implantable device or implant includes a coaptation element (e.g., spacer, plug, filler, foam, sheet, membrane, coaption element, wedge, barrier, balloon, etc.), an anchor portion, a cap, and a distal cover element (e.g., a cover, covering, fabric, polymer, weave, braid, a portion thereof, etc.). The coaptation element has a lumen for receiving one or more actuation elements of the delivery device. Each anchor is movable between an open position and a closed position.

In some implementations, the cap is operatively connected to the anchors such that movement of the cap relative to the coaptation element by the one or more actuation elements of the delivery device causes the anchors to move between the open and closed positions. In some implementations, the cap has a distal opening that is in communication with the lumen of the coaptation element.

In some implementations, the distal cover element is positioned to prevent or inhibit blood from moving through the distal opening of the cap and into an interior of the implantable device.

In some implementations, each anchor has a paddle frame having an inner end that is movable relative to the cap such that at least a portion of the paddle frame is capable of moving through the distal opening of the cap.

In some implementations, there is provided an implantable device having an anchor portion comprising a first anchor and a second anchor (e.g., which can be similar to or the same as any anchor shown or described anywhere herein), each of the first and second anchors comprising a paddle frame, wherein the paddle frame is configured to be moved between a narrowed configuration and expanded configuration, wherein the first and second anchors are configured to be moved to a closed position in which the first and second anchors compress one or more native leaflets such that the implantable device is secured to the native valve.

In some implementations, the implantable device further comprises a flexible cover (e.g., which can be similar to or the same as any cover shown or described anywhere herein) attached to the paddle frame, wherein the flexible cover is configured to be in a taut state when the paddle frame is in the narrowed configuration, and wherein the flexible cover is configured to stretch when the paddle frame is in the expanded configuration.

In some implementations, the paddle frame comprises an inner frame portion and an outer frame portion, and wherein the inner frame portion is rigid, and the outer frame portion is flexible.

In some implementations, the flexible cover comprises a first membrane that attaches to the paddle frame of the first anchor and a second membrane that attaches to the paddle frame of the second anchor.

In some implementations, the flexible cover comprises a single membrane that attaches to the paddle frame of both of the first and second anchors.

In some implementations, the paddle frame of each of the first and second anchors includes an inner frame portion and an outer frame portion, and wherein the paddle frame is configured such that the outer frame portion of the paddle frame changes shape as the paddle frame is moved between the narrowed configuration and the expanded configuration.

In some implementations, the paddle frame of each of the first and second anchors includes an inner frame portion and an outer frame portion, and wherein the first and second anchors are configured such that, in the closed position, the inner frame portion of each of the first and second anchors can compress the one or more native leaflets between an inner pinch point and the outer frame portion of each of the first and second anchors can compress the one or more native leaflets between an outer pinch point such that the implantable device is secured to the native valve.

In some implementations, there is provided an implantable device having an anchor portion comprising an anchor (e.g., which can be similar to or the same as any anchor shown or described anywhere herein), the anchor comprising a paddle frame, wherein the paddle frame is configured to be moved between a narrowed configuration and expanded configuration, and wherein the anchor is configured to be moved to a closed position in which the anchor compresses at least one native leaflet such that the implantable device is secured to a native valve.

In some implementations, the implantable device further comprises a cover (e.g., which can be similar to or the same as any cover shown or described anywhere herein) attached to the paddle frame, wherein the cover is configured to be in a taut state when the paddle frame is in the narrowed configuration, and wherein the cover is configured to stretch when the paddle frame is in the expanded configuration.

In some implementations, the paddle frame includes an inner frame portion and an outer frame portion, and wherein the inner frame portion is rigid, and the outer frame portion is flexible.

In some implementations, the cover extends across at least a portion of an inner surface of the paddle frame.

In some implementations, the cover extends across an entirety of an area defined by an inner surface of the paddle frame.

In some implementations, the anchor is a first anchor and the implantable device also includes a second anchor, and wherein the cover comprises a single membrane that attaches to the paddle frame of the first anchor and the second anchor. In some implementations, the single membrane creates or forms a canopy that extends between the first anchor and the second anchor.

In some implementations, the anchor is a first anchor and the implantable device includes a second anchor that also comprises a paddle frame, wherein the paddle frame of both the first anchor and the second anchor includes an inner frame portion and an outer frame portion, and wherein the cover is attached to the inner frame portion and the outer frame portion of the first and second anchors, and wherein the cover extends between the first anchor and the second anchor.

In some implementations, the paddle frame includes an inner frame portion and an outer frame portion, and wherein the paddle frame is configured such that when the paddle frame is moved between the narrowed configuration and the expanded configuration, the outer frame portion of the paddle frame changes shape between the narrowed configuration and the expanded configuration.

In some implementations, the paddle frame includes an inner frame portion and an outer frame portion, and wherein the anchor is configured such that, in the closed position, the inner frame portion of the anchor can compress the at least one native leaflet between an inner pinch point and the outer frame portion of each of the anchor can compress the at least one native leaflet between an outer pinch point such that the implantable device is secured to the native valve.

In some implementations, there is provided a valve repair system for repairing a native valve of a patient, the valve repair system comprising (A) a delivery device having a width adjustment element (e.g., which can be similar to or the same as any width adjustment element shown or described anywhere herein), and (B) an implantable device configured to be implanted at the native valve of the patient, the implantable device having: (i) an anchor portion having one or more anchors (e.g., which can be similar to or the same as any anchor shown or described anywhere herein), each of the one or more anchors having a paddle frame, wherein the one or more anchors are configured to attach to one or more leaflets of a native valve; and (ii) a coupler (e.g., which can be similar to or the same as any coupler shown or described anywhere herein) for removably connecting the width adjustment element of the delivery device to an end of the one or more anchors, wherein the coupler is configured such that it can secure the end of the one or more anchors in one of multiple potential positions within the implantable device such that the anchors are held in one of multiple potential configurations selected from the group comprising a narrowed configuration, an extended configuration, and an intermediate configuration between the narrowed configuration and the extended configuration.

In some implementations, the coupler comprises at least two arms that are movable between a normal position and an engaged position, wherein the arms are in the normal position when the coupler is disconnected from the width adjustment element, and wherein the arms are in the engaged position when the coupler is connected to the width adjustment element.

In some implementations, the coupler comprises an upper body, a lower body, and a plurality of struts connected to the upper and lower bodies.

In some implementations, a coaptation element defines a first area when viewed from above, wherein the paddle frames of the one or more anchors define an outer region of the implantable device when viewed from above and the one or more anchors are in a closed position, wherein the outer region of the implantable device has a second area when viewed from above, and wherein a ratio of the second area to the first area is greater than or equal to about 2 to 1.

In some implementations, the paddle frame comprises a connector that includes the end.

In some implementations, the implantable device further comprises a cover attached to the paddle frame.

In some implementations, the cover is configured to be in a taut state when the paddle frame is in the narrowed configuration, and wherein the cover is configured to stretch as the paddle frame transitions from the narrowed configuration to the expanded configuration.

In some implementations, the paddle frame includes an inner frame portion and an outer frame portion, and wherein the paddle frame is configured such that when the paddle frame is moved between the narrowed configuration and the expanded configuration, the outer frame portion of the paddle frame changes shape between the narrowed configuration and the expanded configuration.

In some implementations, the paddle frame includes an inner frame portion and an outer frame portion, and wherein the anchor is configured such that the anchor can be moved to a closed position in which the inner frame portion of the anchor can compress the one or more native leaflets between an inner pinch point and the outer frame portion of each of the anchor compresses the one or more native leaflets between an outer pinch point.

In some implementations, the width adjustment element includes an external threaded portion.

In some implementations, the coupler comprises one or more attachment projections that extend inward from a body of the coupler. In some implementations, the one or more attachment projections are configured to removably attach to the external threaded portion of the width adjustment element.

In some implementations, the system (e.g., a portion of the delivery device and/or the implantable device) includes a receiver (e.g., which can be similar to or the same as any receiver shown or described anywhere herein). In some implementations, the receiver defines a lumen that includes internal threads.

In some implementations, the coupler comprises at least two arms that are movable between a normal position and an engaged position, wherein the arms are in the normal position when the coupler is disconnected from the width adjustment element, wherein the arms are in the engaged position when the coupler is connected to the width adjustment element, and wherein the arms are configured to attach to the internal threads of the lumen of the receiver when the arms are in the normal position.

In some implementations, the receiver comprises an unattachable portion. In some implementations, the coupler is configured to be removably coupled to the receiver (e.g., to internal threads of the receiver, etc.) to secure the inner end of the one or more anchors in a desired position relative to the receiver. In some implementations, the unattachable portion of the receiver prevents the coupler from connecting to the receiver when the coupler is disposed within the unattachable portion.

In some implementations, the implantable device further comprises a coaptation element.

In some implementations, the implantable device further comprises a cap operatively connected to the one or more anchors such that movement of the cap relative to another portion of the implantable device (e.g., relative to a coaptation element, collar, proximal end, distal end, etc.) by one or more actuation elements of the delivery device causes the one or more anchors to move between an open position and a closed position.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIGS. 25A and 25B illustrate an example valve repair device that is similar to the valve repair device of FIG. 24, but includes a spacer;

FIG. 26 illustrates a perspective view of an example of an implantable device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane shown in FIG. 27;

FIG. 29 is a schematic illustration of an example implant catheter assembly coupled to an implantable device;

FIG. 30 is an illustration of the assembly of FIG. 29 with the implantable device rotated 90 degrees;

FIG. 31 shows a perspective view of an example implantable device or implant that includes an example paddle frame where the implantable device or implant is in an open position;

FIG. 32 shows a bottom view of the implantable device or implant of FIG. 31;

FIG. 33 shows a front view of the implantable device or implant of FIG. 31 where the implantable device or implant is in a closed position;

FIG. 34 shows a side view of the implantable device or implant of FIG. 31 attached to a native valve of a heart;

FIG. 35 shows a bottom view of the implantable device or implant of FIG. 31 attached to a native valve of a heart;

FIG. 36 shows a perspective view of an example implantable device or implant that includes an example paddle frame where the device includes an example means of moving the paddle frame from a normal position to a narrowed position;

FIG. 37 shows the paddle frame of FIG. 37 in the narrowed position;

FIG. 38 shows a front view of an example paddle frame for an implantable device or implant;

FIG. 39 shows a pair of the example paddle frames of FIG. 38 positioned adjacent to each other;

FIG. 40 shows a side view of an example of an implantable device or implant that includes the paddle frame of FIG. 38, where the paddle frame is in a narrowed position;

FIG. 41 shows a side view of the implantable device or implant of FIG. 40 where the paddle frame is in an expanded position;

FIG. 42 shows a partial side view of the implantable device or implant of FIG. 40 where the paddle frame is in the narrowed position;

FIG. 43 shows a partial side view of the implantable device or implant of FIG. 40 where the paddle frame is in the expanded position;

FIG. 44 shows a perspective view of the implantable device or implant of FIG. 40 with the paddle frame of FIG. 38;

FIG. 45 shows a front view of the implantable device or implant of FIG. 40 with the paddle frame of FIG. 38;

FIG. 46 show a perspective view of an example of inner and outer paddles for the implantable device or implant of FIG. 40;

FIG. 47 shows a side view of the inner and outer paddles of FIG. 46;

FIG. 48 shows a top view of the inner and outer paddles of FIG. 46;

FIG. 49 shows a perspective view of an example connection between the paddles of FIG. 47 and the paddle frame of FIG. 38;

FIG. 50 shows a front view of an example paddle frame for an implantable device or implant;

FIG. 51 shows a left side view of the paddle frame of FIG. 50;

FIG. 52 shows a top view of the paddle frame of FIG. 50;

FIG. 53 shows a perspective view of an example of an implantable device or implant that includes the paddle frame of FIG. 50;

FIG. 54 shows a front view of the implantable device or implant of FIG. 53 that includes the paddle frame of FIG. 50;

FIGS. 55-59 shows the implantable device or implant of FIG. 53 having an example means for moving the paddle frame of FIG. 50 between an expanded position and narrowed positions;

FIG. 60 shows a perspective view of a pair of example paddle frames for a pair of anchors of an implantable device or implant;

FIG. 61 shows a front view of the paddle frames of FIG. 60;

FIG. 62 shows a top view of the paddle frames of FIG. 60;

FIG. 63 shows a side view of the paddle frames of FIG. 60;

FIG. 64 illustrates an example of a width adjustment device or control device;

FIG. 65 illustrates an example of an adjustable paddle frame assembly;

FIG. 66 illustrates an example of an adjustable paddle frame assembly;

FIG. 67 illustrates an example of an adjustable paddle frame assembly;

FIG. 68 illustrates an example of an adjustment member of the adjustable paddle frame assemblies of FIGS. 66 and 67;

FIG. 69 illustrates an example of an adjustable paddle frame assembly;

FIG. 70 shows a front cross-section view of an implantable device or implant;

FIG. 71 shows a perspective cross section view of the device/implant of FIG. 70;

FIG. 72 shows a perspective view of the device/implant of FIG. 70;

FIG. 73 shows a side view of the device/implant of FIG. 70;

FIG. 74 shows a top view of the device/implant of FIG. 70;

FIGS. 75-80 show a partial view of the device/implant of FIG. 70 in various stages of assembly;

FIG. 81 shows a front view of the device/implant of 70 in an expanded position;

FIG. 82 shows a side view of the device/implant of 70 in an expanded position;

FIG. 83 shows a top view of the device/implant of 70 in an expanded position;

FIG. 84 shows a front view of the device/implant of 70 in a narrowed position;

FIG. 85 shows a side view of the device/implant of 70 in a narrowed position;

FIG. 86 shows a top view of the device/implant of 70 in a narrowed position;

FIG. 87 shows a front cross-section view of an example of an implantable device or implant;

FIG. 88 shows a side view of the device/implant of FIG. 87;

FIGS. 89-92 shows front views of the device/implant of FIG. 87 at various positions moving from an expanded position to a narrowed position;

FIG. 93 shows a front sectional view of an example paddle frame for an implantable device or implant;

FIG. 94 shows a top view of the frame of FIG. 93;

FIG. 95 is a front view of an example of a connection mechanism between a rigid inner portion and a flexible outer portion of a paddle frame;

FIG. 96 is a perspective view of the paddle frame assembly of FIG. 95;

FIG. 97 is a top view of the paddle frame assembly of FIG. 95;

FIG. 98 is a side view of the paddle frame assembly of FIG. 95;

FIG. 99 is a rear view of the paddle frame assembly of FIG. 95;

FIG. 100 is a front view of an example of a connection mechanism between a rigid inner portion and a flexible outer portion of a paddle frame;

FIG. 101 is a side view of the paddle frame assembly of FIG. 100;

FIG. 102 is a rear view of the paddle frame assembly of FIG. 100;

FIG. 103 is a front view of an example of a connection between a rigid inner portion and a flexible outer portion of a paddle frame;

FIG. 104 is a side view of the paddle frame of FIG. 103;

FIG. 105 is a perspective view of the paddle frame of FIG. 103;

FIG. 106 is a front view of an example of a connection between a rigid inner portion and a flexible outer portion of a paddle frame;

FIG. 107 is a perspective view of the paddle frame assembly of FIG. 106;

FIG. 108A shows an example of a cap engaged with a paddle;

FIG. 108B shows a close-up of the cap of FIG. 108A without the paddle;

FIG. 108C is a perspective view of the cap and the paddle shown in FIG. 108A;

FIG. 108D is a cross-sectional view that shows deflection of the paddle caused by various degrees of retraction of the paddle into the cap;

FIG. 108E is a perspective view that shows the degrees of deflection of FIG. 108D;

FIG. 108F is a schematic illustration that shows a configuration where the paddles are simultaneously deflected by coupled retraction into a cap;

FIG. 108G is a perspective view of a cap and the paddle assembly;

FIG. 109A is a partial cross-sectional view of an adjustable the paddle assembly;

FIG. 109B is a perspective view of the adjustable the paddle assembly of FIG. 109A;

FIG. 109C is a sectional view of the adjustable the paddle assembly of FIG. 109B;

FIG. 109D is a sectional view of the adjustable the paddle assembly of FIG. 109B;

FIG. 109E is a side view of the adjustable the paddle assembly of FIG. 109B;

FIG. 109F is a side view of an adjustable the paddle assembly showing the paddles in a first actuation position.

FIG. 109G is a side view of an adjustable the paddle assembly showing the paddles in a second actuation position.

FIG. 109H is a side view of an adjustable the paddle assembly showing the paddles in a third actuation position.

FIG. 110A is a side perspective view of an adjustable the paddle assembly;

FIG. 110B is a side view of the adjustable the paddle assembly of FIG. 110A;

FIG. 110C is a front view of the adjustable the paddle assembly of FIG. 110A;

FIGS. 110D and 110E show use of the adjustable the paddle assembly of FIG. 110A in a valve repair device or implant;

FIG. 111A shows an example of a paddle structure made from sheet material;

FIG. 111B is a side view of the paddle structure of FIG. 111A;

FIG. 111C is a top view the paddle structure of FIG. 111A;

FIG. 111D is a bottom the paddle structure of FIG. 111A;

FIG. 111E is another side view the paddle structure of FIG. 111A;

FIG. 111F shows detail of an example of eyelets of the structure the paddle structure of FIG. 111A;

FIG. 111G is a top view of the flat material used to make the paddle structure of FIG. 111A;

FIG. 111H shows an example of a valve repair device or implant that includes the paddle structure of FIG. 111A in a fully retracted position.

FIG. 111I shows the valve repair device or implant of FIG. 111H with the paddle structure in a partially open position;

FIG. 111J shows the valve repair device or implant of FIG. 111H with the paddle structure in a laterally extended or open position;

FIG. 112A is a perspective view example of a valve repair device or implant with compressible outer the paddle portions;

FIG. 112B is a perspective view showing a paddle of the valve repair device or implant illustrated by FIG. 112A;

FIG. 113 shows a perspective view of an example of a paddle frame connector and a width adjustment device for an implantable device;

FIG. 114 shows a perspective cross-sectional view of the paddle frame connector and the width adjustment device of FIG. 113;

FIG. 115 shows a front cross-sectional view of the paddle frame connector and the width adjustment device of FIG. 113;

FIG. 116 shows a bottom view of the paddle frame connector and the width adjustment device of FIG. 113;

FIG. 117 shows a front cross-sectional view of an example of a paddle frame connector and a width adjustment device for an implantable device;

FIG. 118 shows a front cross-sectional view of the paddle frame connector and the width adjustment device of FIG. 117 with an actuation element of a delivery device attached to a receiver of the width adjustment device and a width adjustment element attached to the paddle frame coupler;

FIG. 119 shows a perspective cross-sectional view of the paddle frame connector of FIG. 117;

FIG. 120 shows a top view of a receiver of the width adjustment device of FIG. 117;

FIG. 121 shows a front cross-sectional view of the paddle frame connector and the width adjustment device of FIG. 117 without the width adjustment element and actuation element of FIG. 118;

FIG. 122 shows a front cross-sectional view of the paddle frame connector and the width adjustment device of FIG. 117 with the width adjustment element and the actuation element of FIG. 118;

FIGS. 123-125 show various views of an example of a coupling between an actuation element of an implantable device and a component of an implantable device;

FIGS. 126-128 show various views of the coupling between the actuation element and the component of the implantable device of FIGS. 123-125, where the actuation element is moved in a proximal direction relative to the implantable device;

FIGS. 129-131 show various views of the coupling between the actuation element and the component of the implantable device of FIGS. 123-125, where the actuation element is disconnected from the implantable device;

FIG. 132 shows a front view of an example coupling between an actuation element of an implantable device and a component of an implantable device with a width adjustment element extending through the actuation element and into the implantable device;

FIG. 133 shows the coupling between the actuation element and the component of the implantable device of FIG. 132, where the width adjustment element is moved in a proximal direction relative to the actuation element;

FIG. 134 shows the coupling between the actuation element and the component of the implantable device of FIG. 132. is moved in a proximal direction relative to the implantable device;

FIG. 135 shows the coupling between the actuation element and the component of the implantable device of FIG. 132, where the actuation element is disconnected from the implantable device;

FIG. 136 shows a perspective view of an example of a coupler between a paddle frame connector of an implantable device and a width adjustment element;

FIG. 137 shows a front sectional view of an example of the coupler of FIG. 136;

FIG. 138 shows a side view of the coupler of FIG. 136;

FIG. 139 shows a partial front view of the paddle frame connector of FIG. 136;

FIG. 140 shows a front cross-sectional view of an example of a paddle frame connector and a width adjustment device for an implantable device;

FIG. 141 shows an example of a coupler for connection with an example width adjustment element for the width adjustment device of FIG. 140;

FIG. 142 illustrates a cross-sectional view of an example of a receiver for the width adjustment device of FIG. 140 with the distal portion of the actuation shaft of FIG. 141 moving through the receiver;

FIG. 143 shows a front perspective view of an example of an implantable device having a cover;

FIG. 144 shows a bottom perspective view of the implantable device of FIG. 143;

FIG. 145 is a partial schematic cross-sectional view of the implantable device of FIG. 143 taken along the plane indicated by lines 145A-145A shown in FIG. 143, showing an example paddle frame and cover of the implantable device;

FIG. 146 is a partial schematic cross-sectional view of the implantable device of FIG. 143 taken along the plane indicated by lines 146B-146B shown in FIG. 143, showing an example paddle frame and cover of the implantable device;

FIG. 147 shows a front view of an example of an implantable device having a cover;

FIG. 148 shows a side view of the implantable device of FIG. 147;

FIG. 149 is a partial cross-sectional view of the implantable device of FIG. 147 taken along the plane indicated by lines 149A-149A shown in FIG. 147, showing an example paddle frame and cover of the implantable device;

FIG. 150 is a partial schematic cross-sectional view of the implantable device of FIG. 147 taken along the plane indicated by lines 150B-150B shown in FIG. 147, showing an example paddle frame and cover of the implantable device;

FIG. 151 shows a front view of an example of an implantable device having a cover;

FIG. 152 is a partial schematic cross-sectional view of the implantable device of FIG. 151 taken along the plane indicated by lines 152A-152A shown in FIG. 151, showing an example paddle frame and cover of the implantable device;

FIG. 153 is a partial schematic cross-sectional view of the implantable device of FIG. 151 taken along the plane indicated by lines 153B-153B shown in FIG. 151, showing an example paddle frame and cover of the implantable device;

FIG. 154 shows a front perspective view of an example of an implantable device having a cover;

FIG. 155 is a partial schematic cross-sectional view of the implantable device of FIG. 154 taken along the plane indicated by lines 155A-155A shown in FIG. 154, showing an example paddle frame and cover of the implantable device;

FIG. 156 is a partial schematic cross-sectional view of the implantable device of FIG. 154 taken along the plane indicated by lines 156B-156B shown in FIG. 154, showing an example paddle frame and cover of the implantable device;

FIG. 157 shows a front view of an example of an implantable device having a cover;

FIG. 158 shows a side view of the implantable device of FIG. 157;

FIG. 159 shows a bottom view of the implantable device of FIG. 157;

FIG. 160 is a partial schematic cross-sectional view of the implantable device of FIG. 157 taken along the plane indicated by lines 160A-160A shown in FIG. 157, showing an example paddle frame and cover of the implantable device;

FIG. 161 is a partial schematic cross-sectional view of the implantable device of FIG. 157 taken along the plane indicated by lines 161B-161B shown in FIG. 157, showing an example paddle frame and cover of the implantable device;

FIG. 162 shows a front view of an example of an implantable device having a cover;

FIG. 163 shows a side perspective view of the implantable device of FIG. 162;

FIG. 164 is a partial schematic cross-sectional view of the implantable device of FIG. 162 taken along the plane indicated by lines 164A-164A shown in FIG. 162, showing an example paddle frame and cover of the implantable device;

FIG. 165 is a partial schematic cross-sectional view of the implantable device of FIG. 162 taken along the plane indicated by lines 165B-165B shown in FIG. 162, showing an example paddle frame and cover of the implantable device;

FIG. 166 shows a front view of an example of an implantable device having a cover;

FIG. 167 shows a side perspective view of the implantable device of FIG. 166;

FIG. 168 is a partial cross-sectional view of the implantable device of FIG. 166 taken along the plane indicated by lines 168A-168A shown in FIG. 166, showing an example paddle frame and cover of the implantable device; and

FIG. 169 is a partial schematic cross-sectional view of the implantable device of FIG. 166 taken along the plane indicated by lines 169B-169B shown in FIG. 166, showing an example paddle frame and cover of the implantable device;

FIG. 170 shows a perspective view of an example implantable device;

FIG. 171 shows a top view of the implantable device of FIG. 170;

FIG. 172 shows a side view of the implantable device of FIG. 170;

FIG. 173 shows a perspective view of a coaptation element of the implantable device of FIG. 170;

FIG. 174 shows a front view of the coaptation element of FIG. 173;

FIG. 175 shows a bottom view of the coaptation element of FIG. 173;

FIG. 176 shows a side view of the coaptation element of FIG. 173;

FIG. 177 shows a front cross-sectional view of the coaptation element of FIG. 173 taken along the plane indicated by lines 177-177 shown in FIG. 176;

FIG. 178 shows a perspective view of example paddles connected to the coaptation element of FIG. 173;

FIG. 179 shows a front cross-sectional view of an example implantable device having an example coupler for connecting an example width adjustment element to adjustable width paddles of the implantable device;

FIG. 180 shows a partial perspective view of the coupler of FIG. 179 connecting the width adjustment element to the adjustable width paddles of the implantable device;

FIG. 181 shows a partial cross-sectional view of a connection between the width adjustment member and the coupler of the implantable device of FIG. 179;

FIG. 182 shows a partial cross-sectional view of the connection between the coupler and the width adjustment member of FIG. 179;

FIG. 183 shows a front perspective view of the connection between the coupler and the width adjustment member of FIG. 179;

FIG. 184 shows a side perspective view of the connection between the coupler and the width adjustment member of FIG. 179;

FIG. 185 shows a front cross-sectional view of an example implantable device having an example coupler for connecting an example width adjustment member to the adjustable width paddles of the implantable device;

FIG. 186 shows a perspective cross-sectional view of the implantable device and the width adjustment member of FIG. 185;

FIG. 187 shows a front cross-sectional view of the implantable device of FIG. 185 with the connection between the coupler and the width adjustment member;

FIG. 188 shows a cutaway front view of the coupler of FIG. 185 connected with a width adjustment member;

FIG. 189 shows a cutaway perspective view of the coupler of FIG. 185 connected with the width adjustment member;

FIG. 190 shows a perspective cross-sectional view of the coupler of FIG. 185 connected with the width adjustment member;

FIG. 191 shows a cutaway front view of the coupler of FIG. 185 disconnected from the width adjustment member;

FIG. 192 shows a cutaway perspective view of the coupler of FIG. 185 disconnected from the width adjustment member;

FIG. 193 shows a perspective cross-sectional view of the coupler of FIG. 185 disconnected from the width adjustment member;

FIG. 194 is a front sectional view showing engagement between the coupler and a receiver of the implantable device of FIG. 185 when the width adjustment member is disconnected from the coupler;

FIG. 195 shows a perspective view of the engagement between the coupler and the receiver of the implantable device shown in FIG. 194;

FIG. 196 shows a perspective view of another example coupler, where the coupler is in a locking position;

FIG. 197 shows a perspective view of the coupler of FIG. 196, where the coupler is in an unlocked position;

FIG. 198 shows a left side view of the coupler of FIG. 196, where the coupler is in an unlocked position;

FIG. 199 shows a right-side view of the coupler of FIG. 196, where the coupler feature is in an unlocked position;

FIG. 200 shows a top view of the coupler of FIG. 196, where the coupler is in the locked position;

FIG. 201 shows front view of the coupler of FIG. 196, where the coupler is in the locked position;

FIG. 202 shows an example sheet material for manufacturing the coupler of FIG. 185;

FIG. 203 shows a front cross-sectional view of an example implantable device having an example coupler for connecting an example width adjustment element to adjustable width paddles of the implantable device;

FIG. 204 is a front sectional view showing engagement between the coupler and a receiver of the implantable device of FIG. 203 when the width adjustment element is disconnected from the coupler;

FIG. 205 shows a front cross-sectional view of an example implementation of the implantable device of FIG. 203 where the receiver has a non-threaded portion that prevents or inhibits the coupler from attaching to the receiver when within the non-threaded portion;

FIG. 206 shows a front cross-sectional view of another example implementation of the implantable device of FIG. 203 where the receiver has a window or opening that prevents or inhibits the coupler from attaching to the receiver when within the window or opening;

FIG. 207 shows a cutaway front view of a portion of the implantable device of FIG. 205 where the coupler is connected with the width adjustment element and disposed within the non-threaded portion of the receiver;

FIG. 208 shows a cutaway front view of a portion of the implantable device of FIG. 205 where the coupler is disconnected from the width adjustment element and disposed within the non-threaded portion of the receiver;

FIGS. 209-210 show an example coupler, where example struts of the coupler are in a substantially straight configuration and the coupler is in an unlocked position;

FIGS. 209A-210A show the coupler of FIGS. 209-210, where the struts of the coupler are in an entirely straight configuration and the coupler is in an unlocked position;

FIGS. 211-212 show the coupler of FIGS. 209-210, where the struts are in a spiraled configuration or rotationally offset configuration and the coupler is in a locking position;

FIG. 213 shows a perspective view of an example coupler, where the coupler is in an unlocked position;

FIG. 214 shows a top view of the coupler of FIG. 213, where the coupler is in a locking position;

FIG. 215 shows a perspective view of an example implantable device;

FIG. 216 shows a bottom view of the implantable device of FIG. 215;

FIG. 217 shows a perspective view of the implantable device of FIG. 215 with a cover element attached to a distal portion of the implantable device;

FIG. 218 shows a front view of the implantable device with the cover element of FIG. 217;

FIG. 219 shows a bottom view of the implantable device with the cover element of FIG. 217;

FIG. 220 shows a plan view of an example cover for attaching to an implantable device, where the example cover includes stretchable portions;

FIG. 221 shows a plan view of a portion of a stretchable portion for the cover of FIG. 220;

FIG. 222 shows a plan view of an example plain weave for the cover of FIG. 220;

FIG. 223 shows a plan view of a portion of the cover of FIG. 220, where the stretchable portions are in a pre-heated state;

FIG. 224 shows a plan view of a portion of the cover of FIG. 220, where the stretchable portions are in a post-heated state;

FIG. 225 shows a plan view of an example cover having a stretchable portion that is attached to a paddle frame of an implantable device, where the paddle frame is in an expanded position and the cover is in a stretched position;

FIG. 226 shows the cover attached to the paddle frame of FIG. 225, where the paddle frame is in a narrowed position and the cover is in a normal position;

FIG. 227 shows an example cover attached to a paddle frame of an implantable device, where the paddle frame is in a narrowed position;

FIG. 228 shows an example cover attached to a paddle frame of an implantable device, where the paddle frame is in a narrowed position;

FIG. 229 shows a front view of an example implantable device, where a paddle frame of the implantable device is in a fully expanded position;

FIG. 230 shows a front view of the implantable device of FIG. 229, where the paddle frame is in a first position between the fully expanded position and a fully narrowed position;

FIG. 231 shows a front view of the implantable device of FIG. 229, where the paddle frame is in a second position between the fully expanded position and the fully narrowed position;

FIG. 232 shows a front view of the implantable device of FIG. 229, where the paddle frame is in the fully narrowed position;

FIG. 233 shows a top view of a native valve with the implantable device of FIG. 229 attached thereto, where the paddle frame is in the fully expanded position shown in FIG. 229;

FIG. 234 shows a front view of an example implantable device, where the paddle frame is in a fully expanded position;

FIG. 235 shows a front view of the implantable device of FIG. 234, where the paddle frame is in a fully narrowed position;

FIG. 236 shows a top view of a native valve with the implantable device of FIG. 234 attached thereto, where the paddle frame is in the fully expanded position shown in FIG. 234;

FIG. 237 shows a perspective view of an example paddle frame for the implantable device of FIG. 234; and

FIG. 238 shows a top view of the paddle frame of FIG. 237.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, some implementations of implantable devices, valve repair devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the treatment techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

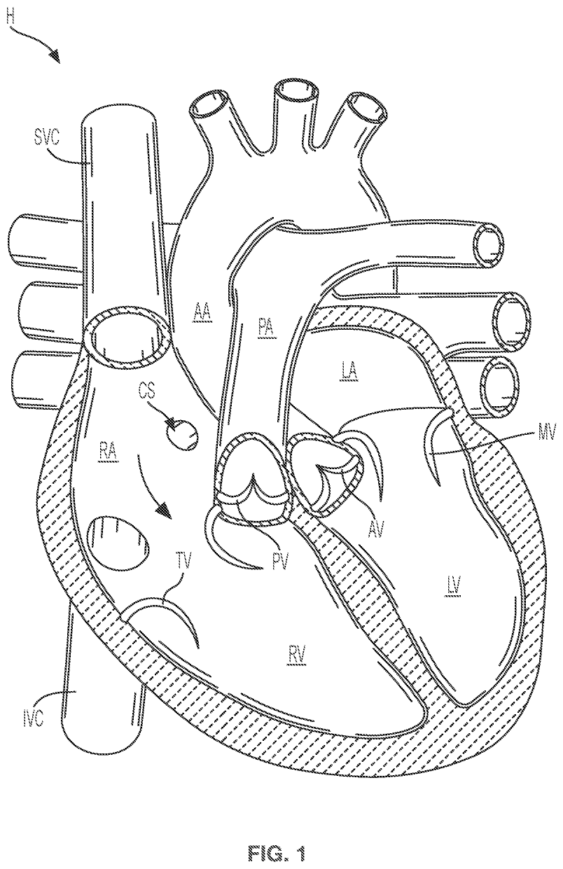

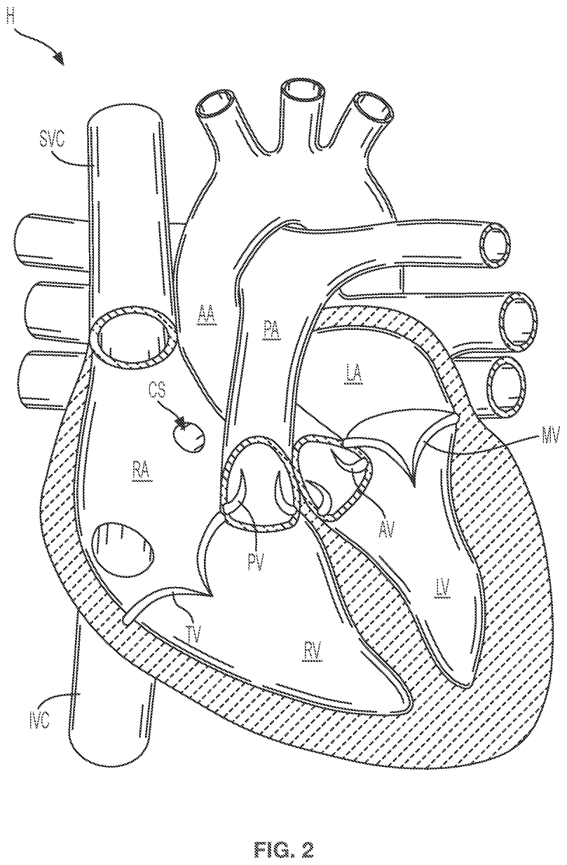

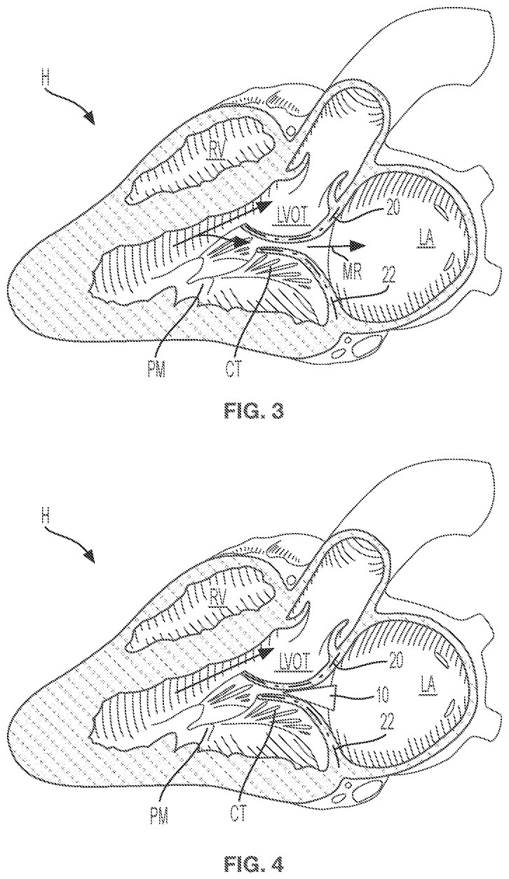

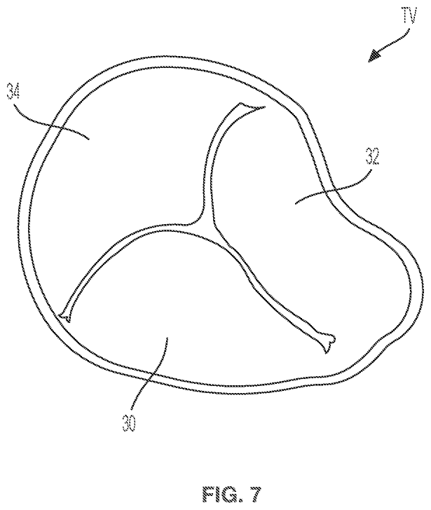

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent or inhibit the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit, or reduce blood from regurgitating from the left ventricle LV and back into the left atrium LA. Many of the devices described in the present application are designed to easily grasp and secure the native leaflets around a coaptation element (e.g., spacer, plug, filler, foam, sheet, membrane, coaption element, wedge, barrier, balloon, etc.) that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant— or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

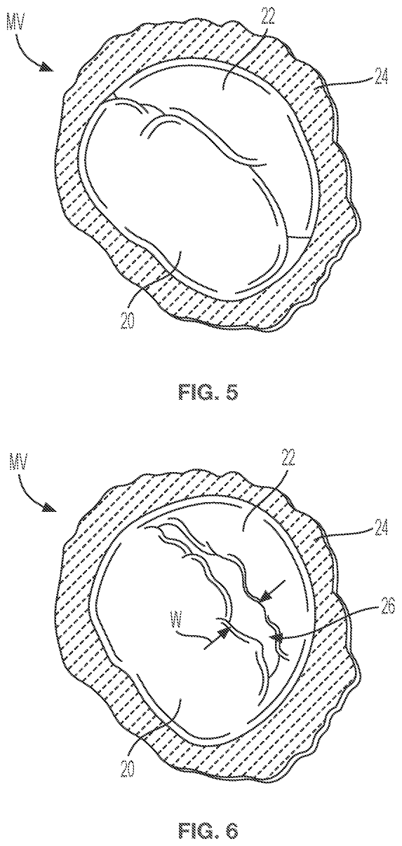

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, plug, filler, foam, sheet, membrane, coaption element, wedge, barrier, balloon, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, plug, filler, foam, sheet, membrane, coaption element, wedge, barrier, balloon, etc.) and at least one anchor (e.g., one, two, three, or more). In some implementations, an implantable device or implant can have any combination or sub-combination of the features disclosed herein without a coaptation element. When included, the coaptation element is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaption element, gap filler, plug, wedge, balloon, barrier, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element, e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

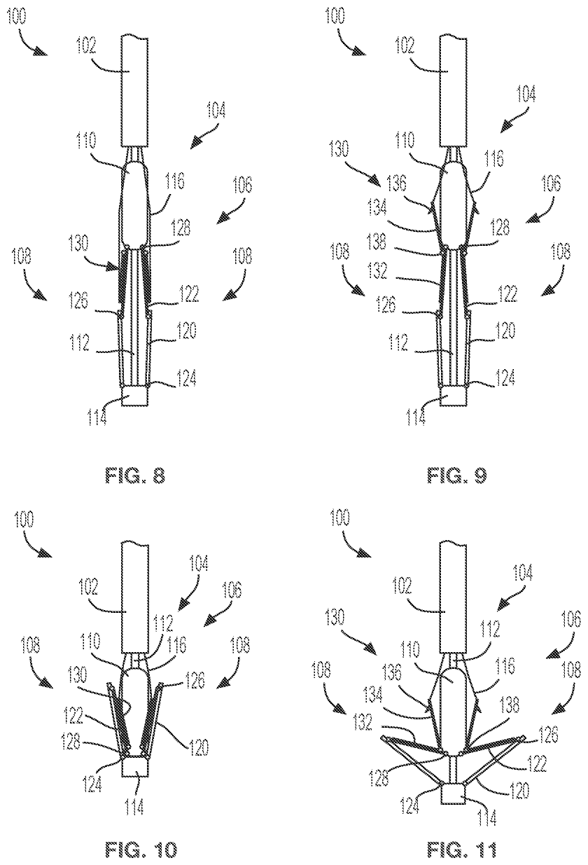

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion/coaptation region 104 and an anchor portion/anchor region 106.

In some implementations, the coaptation portion 104 of the device or implant 100 includes a coaptation element or means for coapting 110 (e.g., spacer, plug, filler, foam, sheet, membrane, coaption element, wedge, barrier, etc.) that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. In some implementations, as illustrated, gripping members can comprise clasps 130 that include a base or fixed arm 132, a moveable arm 134, optional friction-enhancing elements or other means for securing 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., spacer, plug, filler, foam, sheet, membrane, coaption element, wedge, barrier, etc.). The clasps 130 can be used to grasp and/or further secure the native leaflets by engaging the leaflets with barbs or other friction-enhancing elements 136 and pinching the leaflets between the moveable and fixed arms 134, 132. The barbs or other friction-enhancing elements 136 (e.g., protrusions, ridges, grooves, textured surfaces, adhesive, etc.) of the clasps or barbed clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110 (e.g., towards a spacer). The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

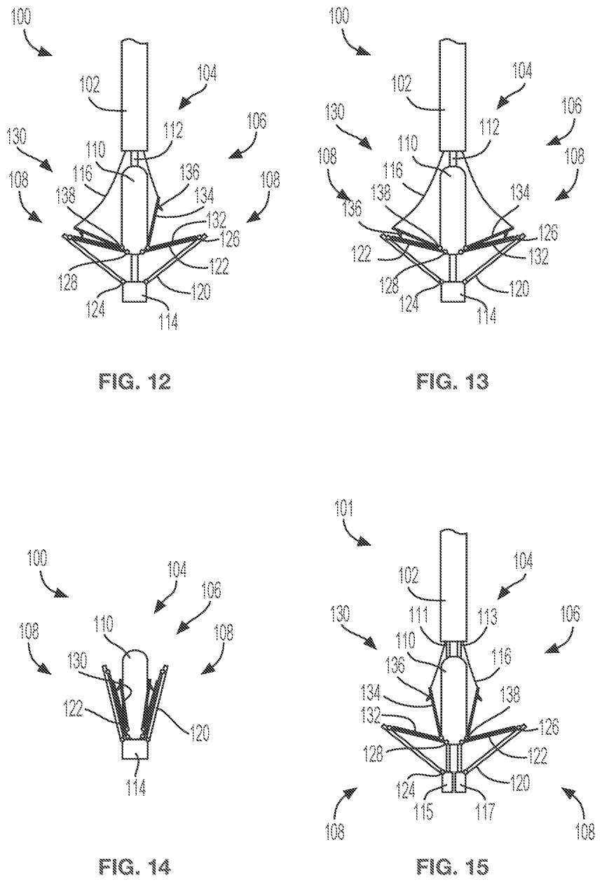

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements or actuation wires 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

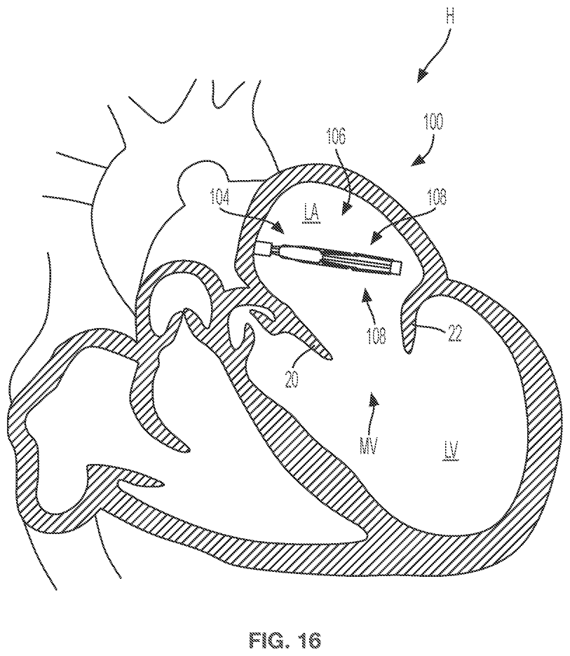

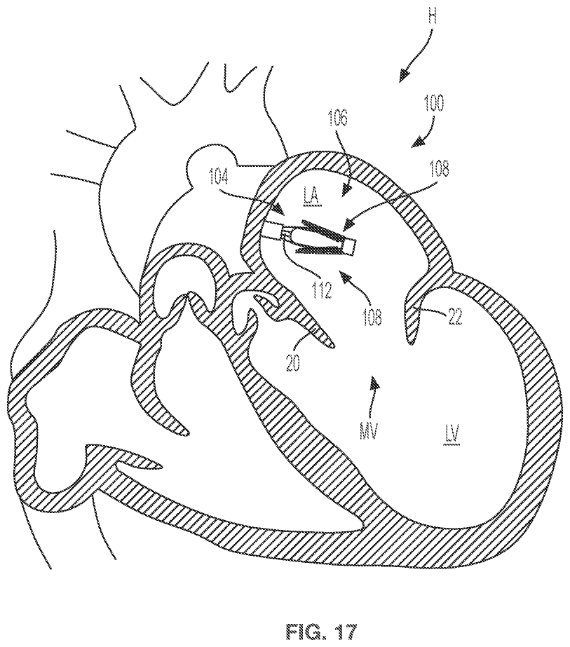

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

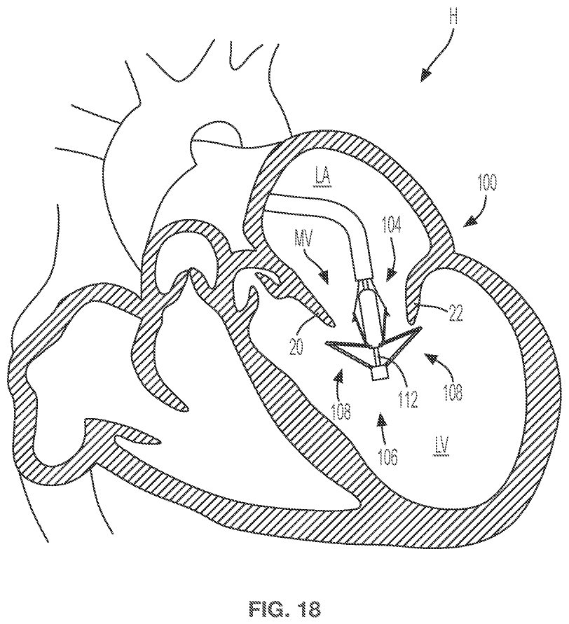

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

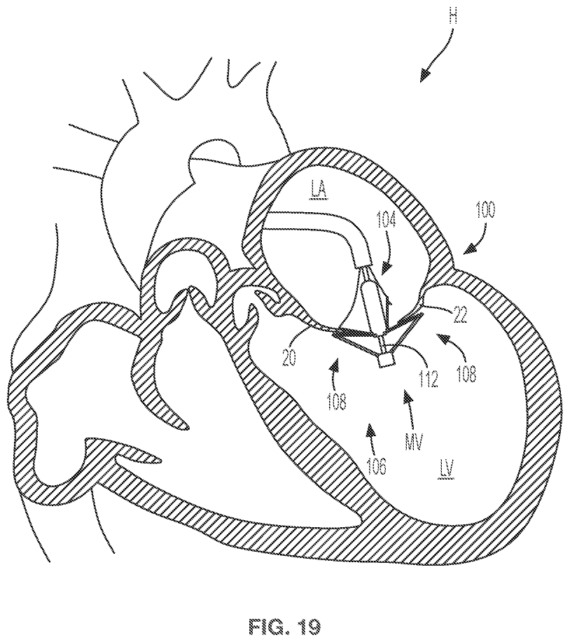

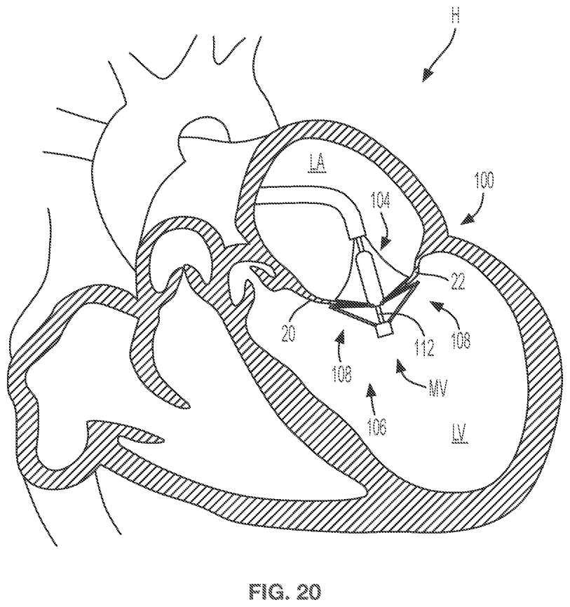

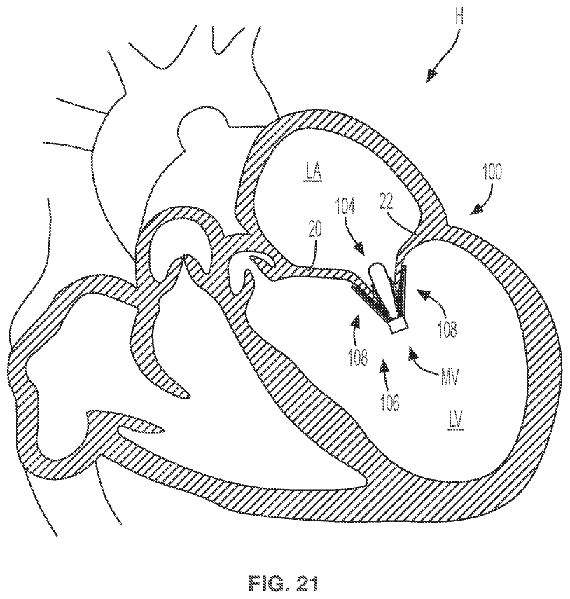

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion/region 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, gap filler, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and U.S. Provisional Patent App. No. 63/217,622, filed on Jul. 1, 2021. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or U.S. Provisional Patent App. No. 63/217,622. Patent Cooperation Treaty International Application No. PCT/US2018/028189 (International Publication No. WO 2018/195215) and U.S. Provisional Patent App. No. 63/217,622 are incorporated herein by reference in their entirety for all purposes.

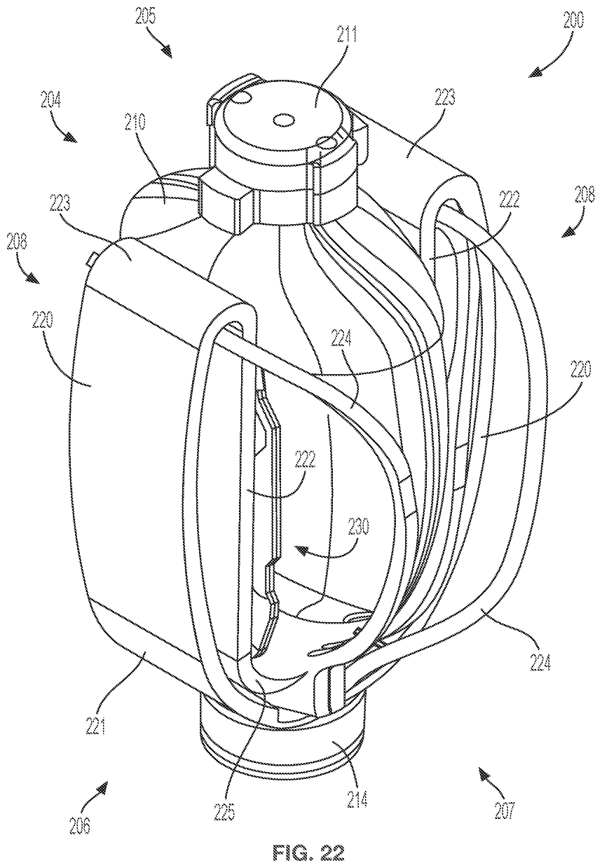

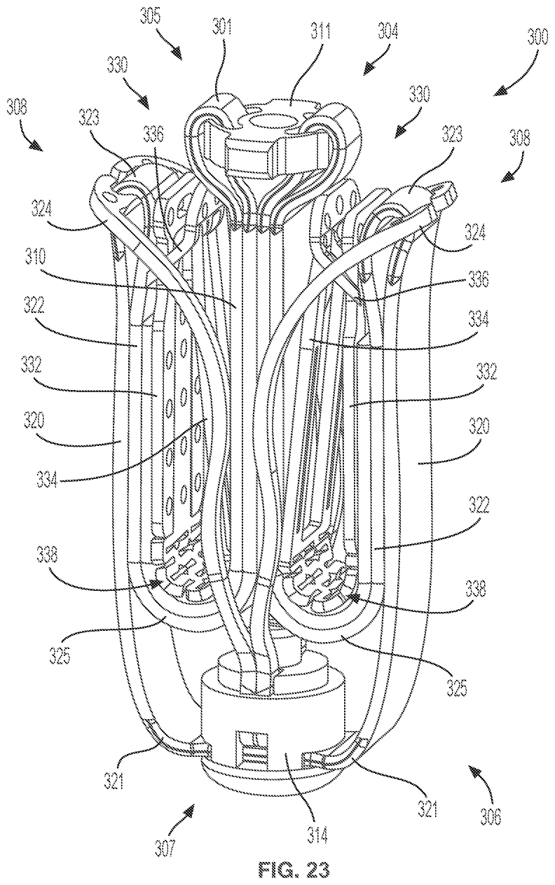

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion/region 304, and the coaptation portion/region 304 can optionally include a coaptation element 310 (e.g., spacer, plug, membrane, sheet, gap filler, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

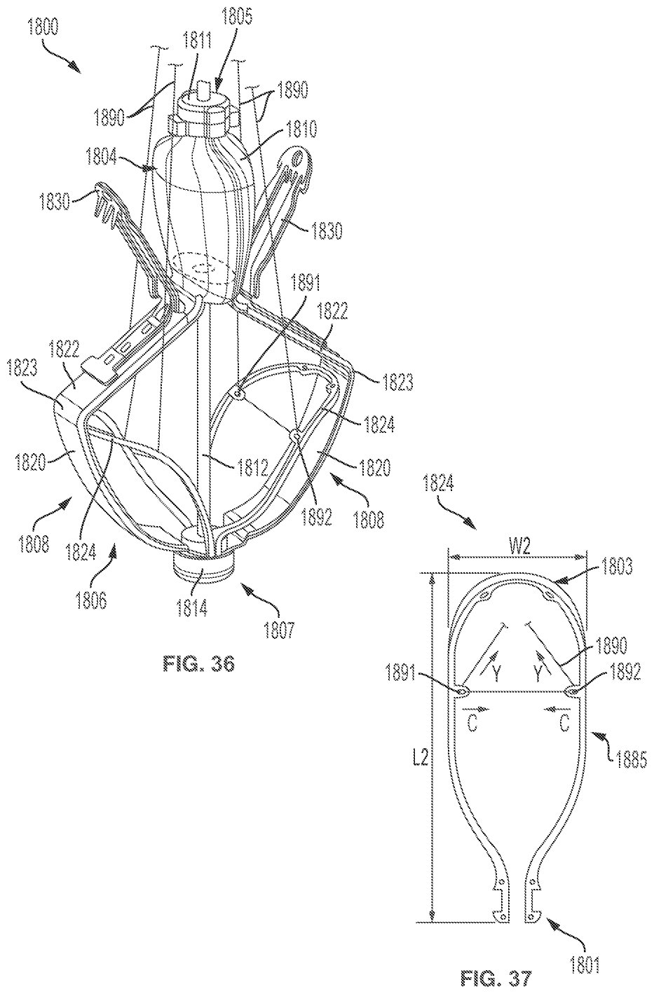

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the barbs 336. The clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or U.S. Provisional Patent App. No. 63/217,622. Patent Cooperation Treaty International Application No. PCT/US2019/055320 (International Publication No. WO 2020/076898) and U.S. Provisional Patent App. No. 63/217,622 are incorporated herein by reference in their entirety for all purposes.

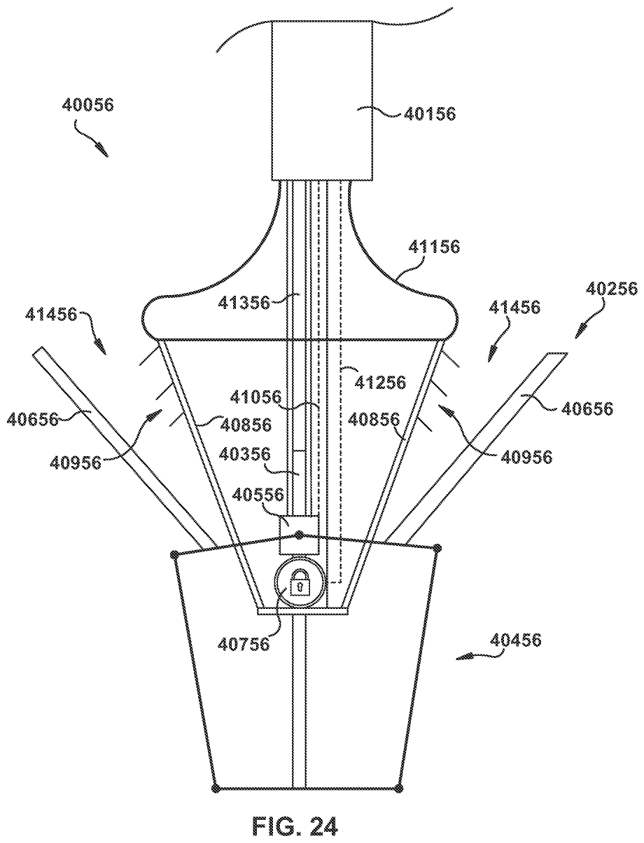

FIG. 24 illustrates another example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456, a pair of paddles 40656, and a pair of gripping members 40856. In one example, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include a barbed portion 40956 for attaching the gripping members to valve tissue when the valve repair device 40256 is attached to the valve tissue. When the paddles 40656 are in the closed position, the paddles engage the gripping members 40856, such that, when valve tissue is attached to the barbed portion 40956 of the gripping members, the paddles secure the valve repair device 40256 to the valve tissue. In some implementations, the gripping members 40856 are configured to engage the paddles 40656 such that the barbed portion 40956 engages the valve tissue member and the paddles 40656 to secure the valve repair device 40256 to the valve tissue member. For example, in certain situations, it can be advantageous to have the paddles 40656 maintain an open position and have the gripping members 40856 move outward toward the paddles 40656 to engage valve tissue and the paddles 40656.

While the example shown in FIG. 24 illustrates a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056, a gripper control mechanism 41156, and a lock control mechanism 41256. In some implementations, the paddle control mechanism 41056 is mechanically attached to the coupler 40556 to move the coupler along the shaft, which causes the paddles 40656 to move between the open and closed positions. The paddle control mechanism 41056 can take any suitable form, such as, for example, a shaft, wire, tube, rod, line, etc. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture or wire, a rod, a catheter, etc.

The lock control mechanism 41256 is configured to lock and unlock the lock. The lock 40756 locks the coupler 40556 in a stationary position with respect to the shaft 40356 and can take a wide variety of different forms and the type of lock control mechanism 41256 can be dictated by the type of lock used. In examples in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position.

The valve repair device 40256 is movable from an open position to a closed position. The base assembly 40456 includes links that are moved by the coupler 40556. The coupler 40556 is movably attached to the shaft 40356. In order to move the valve repair device from the open position to the closed position, the coupler 40556 is moved along the shaft 40356, which moves the links.

The gripper control mechanism 41156 is moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripper members 40856. When the line(s) is pulled, the gripping members 40856 move inward, which causes the opening 41456 between the gripping members and the paddles 40656 to become wider.

In order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

After the paddles 40656 are moved to the closed position, the lock 40756 is moved to the locked condition by the locking control mechanism 41256 to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 is disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or U.S. Provisional Patent App. No. 63/217,622. Patent Cooperation Treaty International Application No. PCT/US2019/012707 (International Publication No. WO 2019139904) and U.S. Provisional Patent App. No. 63/217,622 are incorporated herein by reference in their entirety for all purposes.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

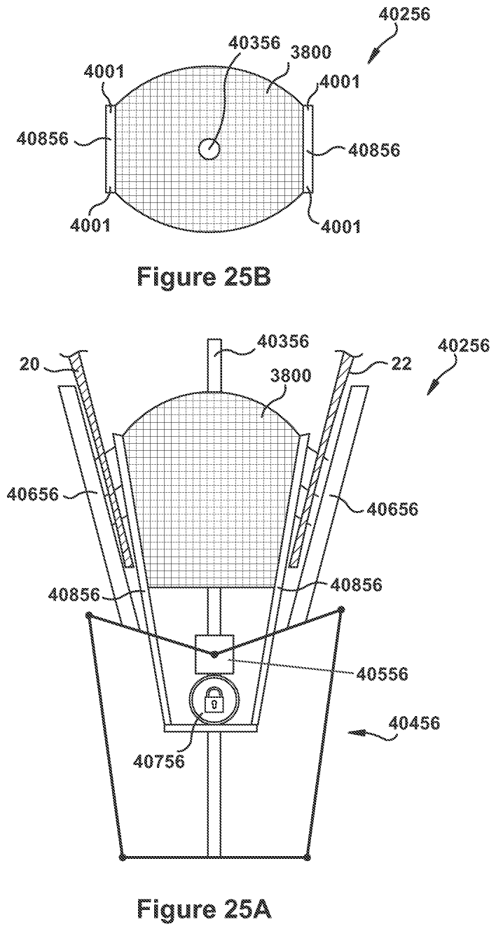

Referring to FIGS. 25A-25B, an example implementation of a valve repair device 40256 has a spacer or coaptation element 3800. The valve repair device 40256 can have the same configuration as the valve repair device illustrated by FIG. 24 with the addition of the spacer or coaptation element. The spacer or coaptation element 3800 can take a wide variety of different forms. The spacer or coaptation element 3800 can be compressible and/or expandable. For example, the spacer can be compressed to fit inside one or more catheters of a delivery system, can expand when moved out of the one or more catheters, and/or can be compressed by the paddles 40656 to adjust the size of the spacer or coaptation element. In the example illustrated by FIGS. 25A and 25B, the size of the spacer or coaptation element 3800 can be reduced by squeezing the spacer or coaptation element with the paddles 40656 and can be increased by moving the paddles 40656 away from one another. The spacer element 3800 can extend past outer edges 4001 of the gripping members or clasps 40856 as illustrated for providing additional surface area for closing the gap of a mitral valve.

The spacer or coaptation element 3800 can be coupled to the valve repair device 40256 in a variety of different ways. For example, the spacer or coaptation element 3800 can be fixed to the shaft 40356, can be slidably disposed around the shaft, can be connected to the coupler 40556, can be connected to the lock 40756, and/or can be connected to a central portion of the clasps or gripping members 40856. In some implementations, the coupler 40556 can take the form of the spacer element 3800. That is, a single element can be used as the coupler 40556 that causes the paddles 40656 to move between the open and closed positions and the spacer element 3800 that closes the gap between the leaflets 20, 22 when the valve repair device 40256 is attached to the leaflets.

The spacer or coaptation element 3800 can be disposed around one or more of the shafts or other control elements of the valve repair system 40056. For example, the spacer or coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

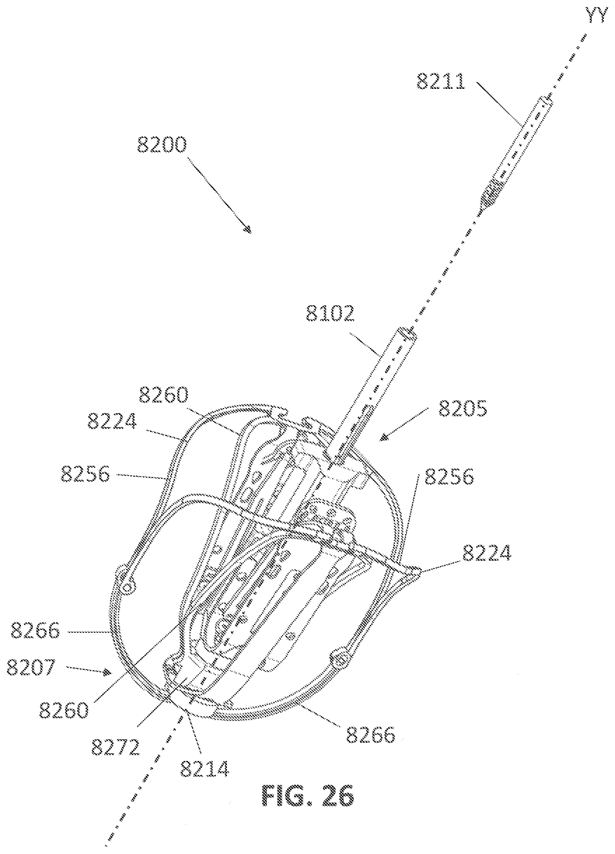

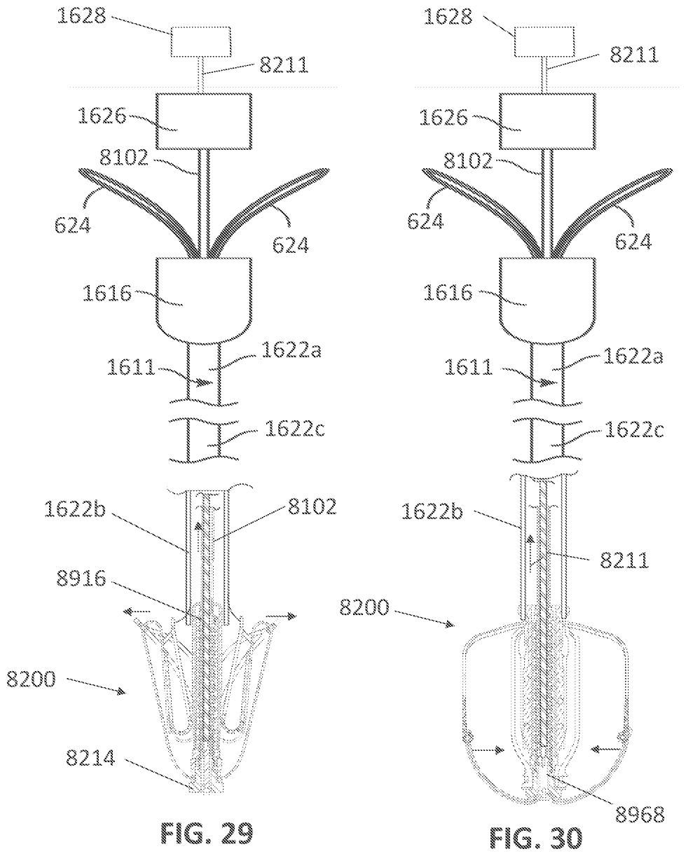

FIGS. 26-30 illustrate another example of one of the many valve repair systems for repairing a native valve of a patient that the concepts of the present application can be applied to. Referring to FIGS. 29 and 30, the valve repair system includes a delivery device 1611 and an implantable valve repair device 8200. Referring to FIGS. 26-28, the implantable device 8200 includes a proximal or attachment portion 8205, outer paddle portions 8120, inner paddle portions 8122, paddle frames 8224, and a distal portion 8207. The proximal portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 can include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the proximal portion 8205, the outer frame portions 8256 form a curved shape. For example, in the illustrated example, the shape of the outer frame portions 8256 resembles an apple shape in which the outer frame portions 8256 are wider toward the proximal portion 8205 and narrower toward the distal portion 8207. In some implementations, however, the outer frame portions 8256 can be otherwise shaped.

The inner frame portions 8260 extend from the proximal portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

A width adjustment element 8211 (e.g., width adjustment control, width adjustment wire, width adjustment shaft, width adjustment tube, width adjustment line, width adjustment cord, width adjustment suture, width adjustment screw or bolt, width adjustment tether, etc.) is configured to move the outer frame portions 8256 from the expanded position to the narrowed position by pulling the inner end 8968 (FIG. 28) and portions of the connector 8266 into the actuation cap 8214. The actuation element 8102 (e.g., actuation wire, actuation shaft, actuation tube, etc.). is configured to move the inner paddle frame portions 8260 to open and close the paddles in accordance with some implementations disclosed herein.

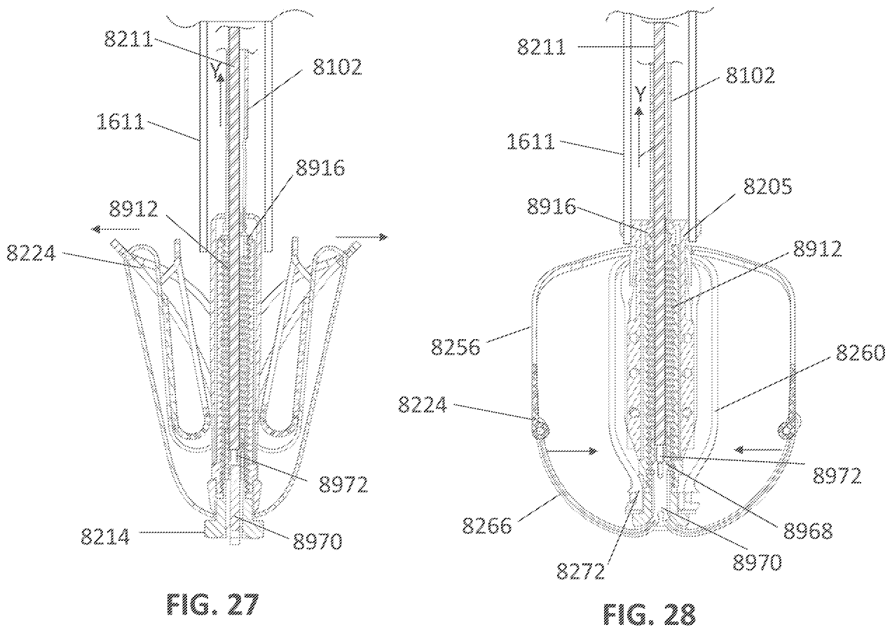

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, tracks, a cylinder. etc.) to move the outer frame portions 8256 between a narrowed position and an expanded position. In the illustrated example, the inner end 8968 comprises a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the tube. When the coupler 8972 is detached from the width adjustment element 8211, the coupler is secured to the tube. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some implementations disclosed above.

In the illustrated example, a driver head 8916 is disposed at a proximal end of the actuation element 8102. The driver head 8916 releasably couples the opening/closing actuation element 8102 to the receiver 8912. In the illustrated example, the width adjustment element 8211 extends through the actuation element 8102. The actuation tube is axially advanced in the direction opposite to direction Y to move the distal cap 8214. Movement of the distal cap 8214 relative to the attachment portion 8205 is effective to open and close the paddles, as indicated by the arrows in FIG. 27. That is movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the implantable device 8200. The actuation element 8102 can extend distally from the paddle actuation control 1626, through the handle 1616, through the delivery shaft or catheter of the delivery device 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the coupler 8972. The width adjustment element 8211 can be axially movable relative to the actuation element 8102, the outer shaft of the catheter assembly 1611, and the handle 1616. The clasp actuation lines 624 can extend through and be axially movable relative to the handle 1616 and the outer shaft of the catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the control 1628 widens and narrows the paddles. Advancing and retracting the actuation element 8102 with the control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

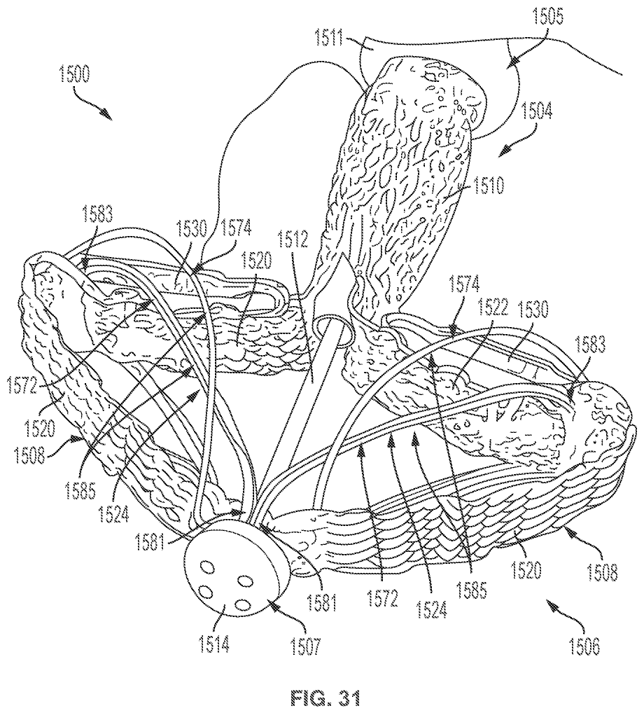

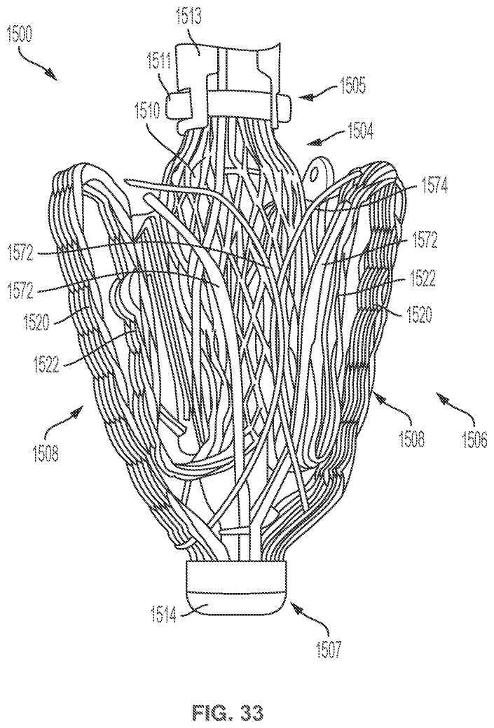

A Referring to FIGS. 31-35, an example of an implantable device or implant 1500 includes an anchor portion 1506 having one or more paddle frames 1524. The paddle frames 1524 are configured to allow the device 1500 to maneuver more easily into position for implantation in the heart by reducing the contact and/or friction between the native structures of the heart—e.g., chordae—and the device 1500. That is, the paddle frames 1524 are configured to move between an expanded position (when the device 1500 is in a closed position) and a narrowed position (when the device 1500 is in an open position) and/or the paddle frames can include a flexible outer portion that flexes inward to reduce the width of the paddles when the flexible outer portion contacts a native heart structure—e.g., chordae.

When the paddle frames 1524 are in the narrowed position, the friction between the native structures of the heart and the device 1500 is reduced. The device 1500 can include any other features for an implantable device or implant discussed in the present application or in the applications and patents incorporated by reference herein, and the device 1500 can be positioned to engage valve tissue 20, 22 as part of any suitable valve repair system (e.g., any valve repair system disclosed in the present application). In addition, any of the devices described herein can incorporate the features of the device 1500.

The implantable device or implant 1500 includes a coaptation portion 1504, a proximal or attachment portion 1505, an anchor portion 1506, and a distal portion 1507. The coaptation portion 1504, attachment portion 1505, and distal portion 1507 can take any suitable form, such as, for example, the form for these portions of the device 200 shown in FIG. 22, or any other form described in the present application. In some implementations, the coaptation portion 1504 optionally includes a coaptation element 1510 (e.g., a spacer, coaption element, gap filler, etc.) that can be used, for example, for implantation between the leaflets 20, 22 of the native mitral valve MV. The coaptation element, etc. 1510 can take any suitable form, such as, for example, any form described in the present application. In the illustrated example, the coaptation element is made from woven wires.

The attachment portion 1505 includes a first or proximal collar 1511 for engaging with a capture mechanism of a delivery system. The proximal collar 1511 can take any suitable form, such as, for example, any form described in the present application. The capture mechanism 1513 can take any suitable form, such as, for example, any form described in the present application.

The distal portion 1507 includes a cap 1514 that is attached to anchors 1508 of the anchor portion 1506 such that movement of the cap 1514 causes the anchors 1508 to move between open and closed positions. The cap 1514 can take any suitable form, such as, for example, any form described in the present application. In the illustrated example, an actuation element 1512 (e.g., an actuation wire, actuation shaft, etc.) extends from a delivery system (e.g., any delivery system described in the present application) and engages the cap 1514 to move the cap 1514 relative to the coaptation element or spacer 1510 to enable actuations of the device 1500. The actuation element 1512 can engage and move the cap by any suitable means, such as, for example, any means provided in the present application.

The anchor portion 1506 of the device 1500 can take any suitable form, such as, for example, the form of the anchor portion 206 of the device 200 shown in FIG. 22 (except that the paddle frame 224 is replaced with the paddle frame 1524 shown in FIGS. 91-95 and described in more detail below), or any other form described in the present application that can incorporate paddle frame 1524. The anchor portion 1506 can include a plurality of anchors 1508, each anchor 1508 including outer paddles 1520, inner paddles 1522, paddle extension members or paddle frames 1524, and clasps 1530.

The paddle frame 1524 includes a main support section 1585, first connection members for attaching to a cap of the implantable device or implant, and second connection members for attaching to anchors of the device. The connection members can be the same as or similar to other connection members described elsewhere herein. The paddle frame 1524 can attach to the connection portion of the anchors and the cap by any suitable means, such as, for example, any means described in the present application. The thickness and width of the paddle frame can take any suitable form, such as, for example, the thickness can be substantially identical to the width, the thickness can be greater than the width (as shown in FIGS. 91-95), or the width can be greater than the thickness.

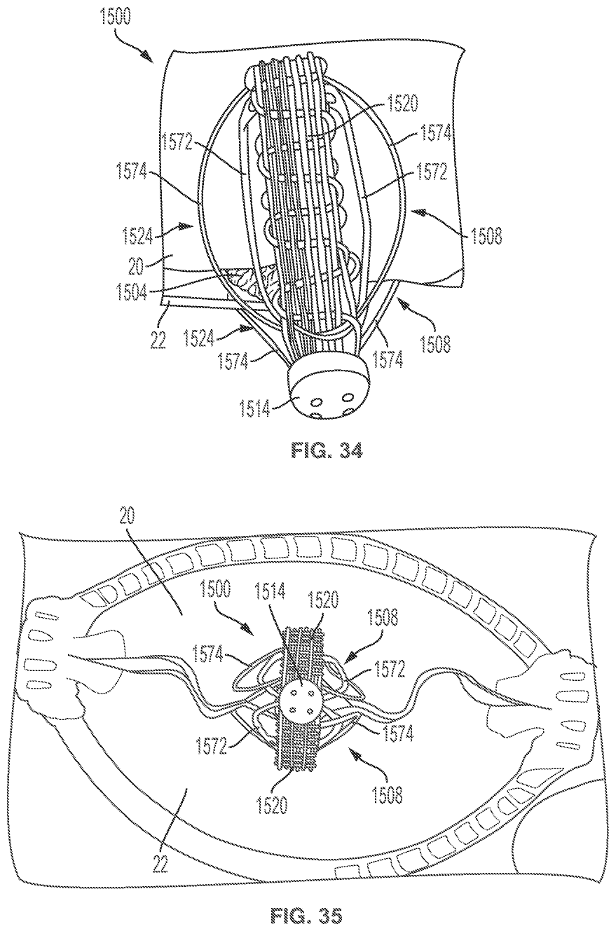

The main support section 1585 includes a rigid inner portion 1572 and a flexible outer portion 1574. The rigid inner portion 1572 has a first end 1581 that connects to the cap 1514 and a second end 1583 that connects to the anchors 1508. Referring to FIGS. 34 and 35, the rigid inner portion is configured to support the paddles 1520, 1522 of the anchors and provide a sufficient force to facilitate coaptation of the native leaflets 20, 22 against the coaptation element 1510 when the anchors 1508 are in the closed position. The rigid inner portion 1572 can be made of, for example, metals, plastics, etc.

Referring again to FIGS. 31-35, the flexible outer portion 1574 is connected to the rigid inner portion and defines the total width of the paddle frame 1524. That is, the flexible outer portion 1574 has a greater total width than the rigid inner portion 1572. The flexible outer portion 1574 is configured such that forces (e.g., forces from the flexible outer portion 1574 contacting the chordae during implantation of the device 1500) cause the flexible outer portion 1574 to flex and allow the device 1500 to maneuver more easily into position for implantation in the heart. Referring to FIGS. 34 and 35, when the anchors 1508 are in the closed position and causing leaflets to coapt against the coaptation element 1510, the flexible outer portion 1574 maintains its normal total width to provide for a larger surface area (relative to the rigid inner portion 1572) contacting the leaflets to hold the leaflets against the coaptation element 1510. The flexible outer portion 1574 can be made of, for example, metals, and plastics.

The total width of the flexible outer portion 1574 can be 5 mm and 15 mm, such as between 7 mm and 12 mm, such as between 9 mm and 11 mm, such as about 10 mm. The width of the inner portion 1572 can be between 2 mm and 8 mm, such as between 4 mm and 6 mm, such as about 5 mm.

In some implementations, the flexible outer portion 1574 are shaped set inward such that the total width of the outer portion 1574 narrows when the anchors 1508 are in the open position, and such that the outer portion moves back to its normal total width when the anchors 1508 are moved to the closed position.

While the illustrated example, shows rigid inner portion 1572 and the flexible inner portion 1574 having rounded shapes, it should be understood that the inner and outer portions 1572, 1574 can take any form that allows the device 1500 to more easily maneuver into position for implantation in the heart while providing sufficient support for facilitating coaptation of the leaflets of a native heart valve against the coaptation element 1510.

Referring to FIGS. 36-37, an example implementation of an implantable device or implant 1800 includes an anchor portion 1806 having one or more paddle frames 1824 that are movable to a narrowed position to allow the device 1800 to maneuver more easily into position for implantation in the heart by reducing the contact and/or friction between the native structures of the heart—e.g., chordae—and the device 1800. That is, width adjustment lines 1890 are controlled by a user to create a compression force C (FIG. 37) on the paddle frames 1824 to move the paddle frames 1824 to a narrowed position as the device 1800 is being positioned for implantation on the native leaflets of a native valve such that the contact and/or friction between the native structures of the heart and the device 1800 is reduced. The device 1800 can include any other features for an implantable device or implant discussed in the present application or in the applications and patents incorporated by reference herein, and the device 1800 can be positioned to engage valve tissue 20, 22 as part of any suitable valve repair system (e.g., any valve repair system disclosed in the present application). In addition, any of the devices described herein can incorporate the features of the device 1800.

The implantable device or implant 1800 includes a coaptation portion 1804, a proximal or attachment portion 1805, an anchor portion 1806, and a distal portion 1807. The coaptation portion 1804, attachment portion 1805, and distal portion 1807 can take any suitable form, such as, for example, the form for these portions of the device 200 shown in FIG. 22, or any other form described in the present application. In some implementations, the coaptation portion 1804 includes coaptation element 1810 (e.g., a spacer, coaption element, gap filler, etc.) that can be used, for example, for implantation between the leaflets 20, 22 of the native mitral valve MV. The coaptation element 1810 can take any suitable form, such as, for example, any form described in the present application.

The attachment portion 1805 includes a first or proximal collar 1811 for engaging with a capture mechanism of a delivery system. The capture mechanism and delivery system can be the same as or similar to other capture mechanisms and delivery systems described elsewhere herein. The proximal collar 1811 can take any suitable form, such as, for example, any form described in the present application.

The distal portion 1807 includes a cap 1814 that is attached to anchors 1808 of the anchor portion 1806 such that movement of the cap 1814 causes the anchors 1508 to move between open and closed positions. The cap 1814 can take any suitable form, such as, for example, any form described in the present application. In the illustrated example, an actuation element 1812 (e.g., an actuation wire, an actuation shaft, etc.) extends from a delivery system (e.g., any delivery system described in the present application) and engages the cap 1814 to move the cap 1814 relative to the coaptation element or spacer 1810 to enable actuations of the device 1800. The actuation element 1812 can engage and move the cap by any suitable means, such as, for example, any means provided in the present application.

The anchor portion 1806 can take any suitable form, such as, for example, the form of the anchor portion 206 of the device 200 shown in FIG. 22 or any other form described in the present application. The anchor portion 1806 can include a plurality of anchors 1808, each anchor 1808 including outer paddles 1820, inner paddles 1822, paddle extension members or paddle frames 1824, and clasps 1830. The paddle frames 1824 can include a main support section 1885, first connection members for attaching to the cap 1814, and second connection members for attaching to a connection portion 1823 of the anchors 1808. The paddle frame 1824 can attach to the connection portion of the anchors and the cap by any suitable means, such as, for example, any means described in the present application. The thickness and width of the paddle frame 1824 can take any suitable form, such as, for example, the thickness can be substantially identical to the width, the thickness can be greater than the width (as shown in FIGS. 91-95), or the width can be greater than the thickness.

The paddle frame 1824 includes an end 1801 that is configured to be attached to the cap 1814 and a free end 1803. The paddle frame 1824 includes a first opening 1891 and a second opening 1892 for receiving one or more width adjustment lines 1890 of the delivery system. Referring to FIGS. 36-37, in some examples, a single width adjustment line 1890 extends through the first and second openings 1891, 1892 of each paddle frame 1824 and into the delivery system such that a user can pull the width adjustment lines 1890 to cause the paddle frame 1824 to move to the narrowed position. In some implementations, the width adjustment lines 1890 can also extend through an opening of the clasp 1830 of each paddle before extending into the delivery system. Referring to FIG. 37, when a user pulls the width adjustment line 1890, a force is created on each end of the width adjustment line 1890 in the direction Y, which causes a compression force C on the paddle frame 1824 due to the width adjustment line extending through the openings 1891, 1892. The compression force C causes the paddle frame 1824 to move to the narrowed position.

Referring to FIG. 37, the paddle frames 1824 have a length L2 and a total width W2 when in the narrowed position. The width of the paddle frame 1824 when in the normal, expanded position can be between 5 mm and 15 mm, such as between 7 mm and 12 mm, such as between 9 mm and 11 mm, such as about 10 mm. The narrowed width W2 of the paddle frame 1824 can be between 3 mm and 12 mm, such as between 5 mm and 10 mm, such as between 7 mm and 9 mm, such as about 8 mm A ratio of the normal width to the narrowed width W2 can be between 10/9 and 3/1, such as between 5/4 and 2/1, such as between 4/3 and 3/2.

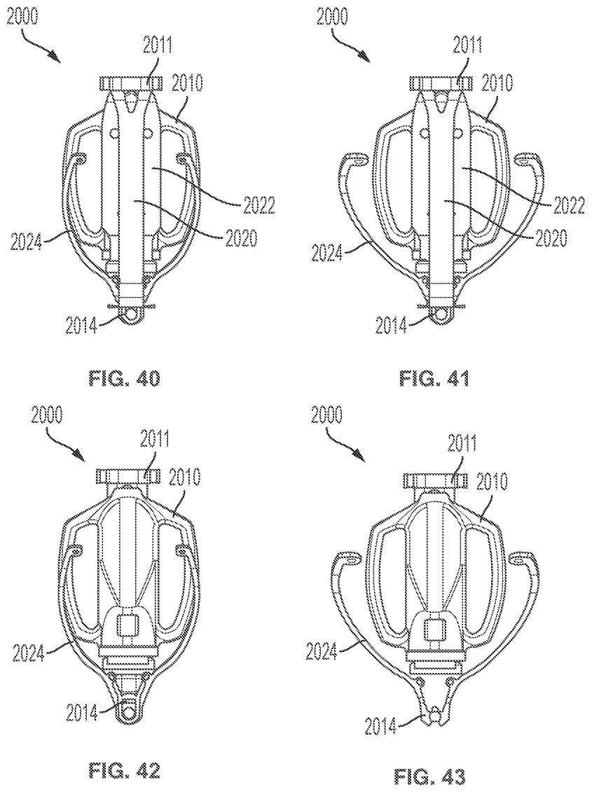

Referring to FIGS. 40-49, an example implementation of an implantable device or implant 2000 (FIGS. 40-45) includes an anchor portion 2006 having one or more paddle frames 2024. The paddle frames 2024 are configured to allow the device 2000 to maneuver more easily into position for implantation in the heart by reducing the contact and/or friction between the native structures of the heart—e.g., chordae—and the device 2000. For example, width adjustment lines are controlled by a user to create a compression force (e.g., compression force C shown in FIG. 37) on the paddle frames 2024 to move the paddle frames 2024 from a normal, expanded position (FIGS. 41 and 43) to a narrowed position (FIGS. 40 and 42) as the device 2000 is being positioned for implantation on the leaflets of a native valve such that the contact between the native structures of the heart and the device 2000 is reduced. The device 2000 can include any other features for an implantable device or implant discussed in the present application or in the applications and patents incorporated by reference herein, and the device 2000 can be positioned to engage valve tissue 20, 22 as part of any suitable valve repair system (e.g., any valve repair system disclosed in the present application). In addition, any of the devices described herein can incorporate the features of the device 2000.

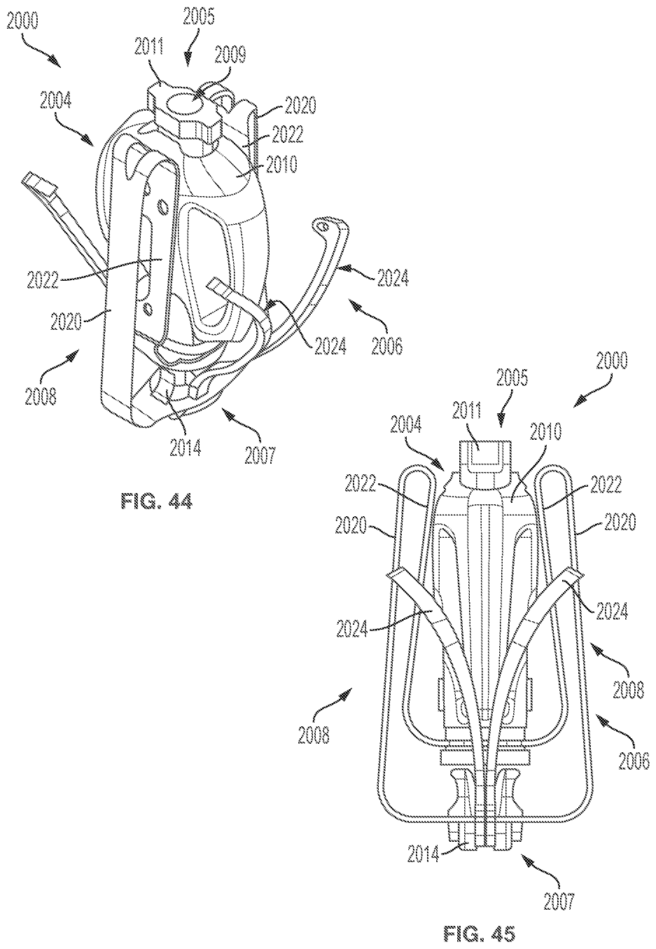

Referring to FIGS. 44-45, the implantable device or implant 2000 includes a coaptation portion 2004, a proximal or attachment portion 2005, an anchor portion 2006, and a distal portion 2007. The coaptation portion 2004, attachment portion 2005, and distal portion 2007 can take any suitable form, such as, for example, the form for these portions of the device 200 shown in FIG. 22, or any other form described in the present application. In some implementations, the coaptation portion 2004 optionally includes a coaptation element 2010 (e.g., a spacer, coaption element, gap filler, etc.) that can be used, for example, for implantation between the leaflets 20, 22 of the native mitral valve MV. The coaptation element, etc. 2010 can take any suitable form, such as, for example, any form described in the present application.

The attachment portion 2005 includes a first or proximal collar 2011 for engaging with a capture mechanism of a delivery system. The capture mechanism and delivery system can be the same as or similar to other capture mechanisms and delivery systems described elsewhere herein. The proximal collar 2011 can take any suitable form, such as, for example, any form described in the present application.

The distal portion 2007 includes a cap 2014 that is attached to anchors 2008 of the anchor portion 2006 such that movement of the cap 2014 causes the anchors 2008 to move between open and closed positions. The cap 2014 can take any suitable form, such as, for example, any form described in the present application. An actuation element (e.g., the same as or similar to actuation element 112 shown in FIGS. 8-20 or actuation element 8102 shown in FIGS. 26-30) extends from a delivery system (e.g., any delivery system described in the present application), through the coaptation element 2010 via opening 2009 (FIG. 44), and engages the cap 2014 to move the cap 2014 relative to the coaptation element 2010 to enable actuations of the device 2000. The actuation element can engage and move the cap by any suitable means, such as, for example, any means provided in the present application.

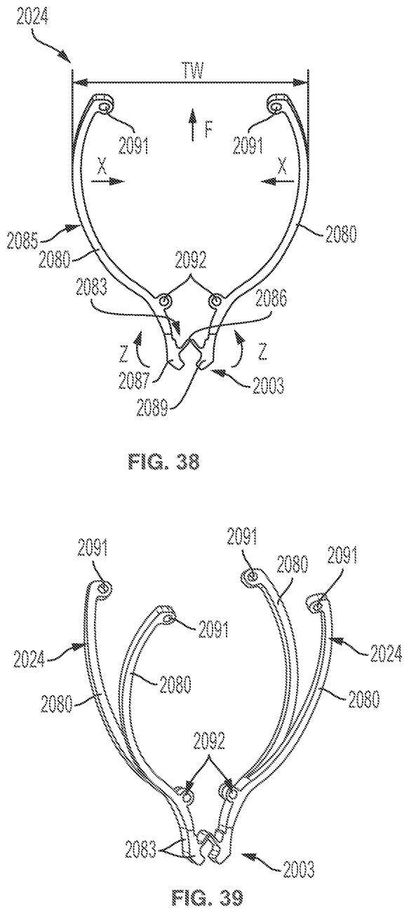

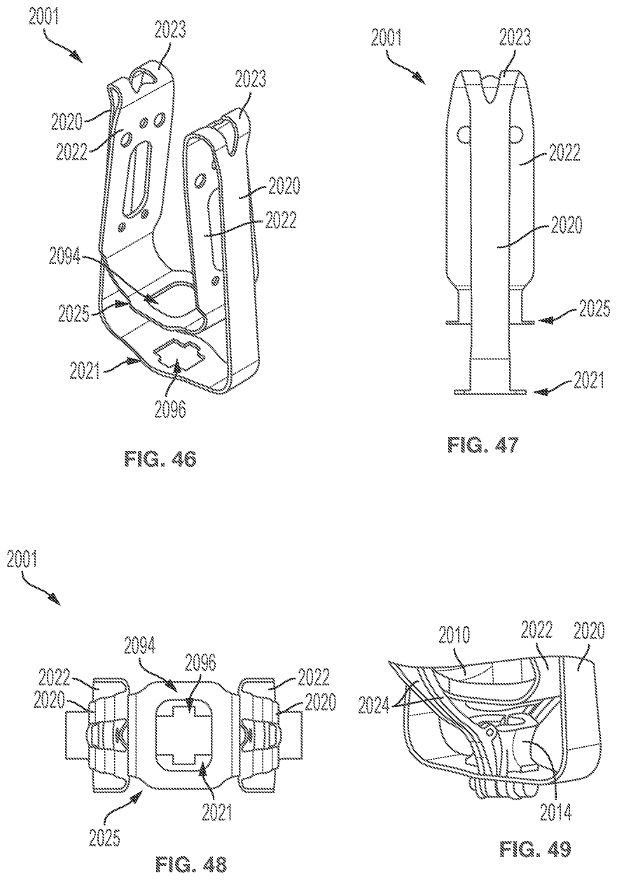

The anchor portion 2006 can take any suitable form, such as, for example, the form of the anchor portion 206 of the device 200 shown in FIG. 22 or any other form described in the present application. The anchor portion 2006 can include a plurality of anchors 2008, each anchor 2008 including outer paddles 2020, inner paddles 2022, paddle extension members or paddle frames 2024, and clasps (e.g., clasps 230 shown in FIG. 22). Referring to FIGS. 38 and 39, the paddle frames 2024 can include a main support section 2085 and connection members 2003 for attaching to the cap 2014. The paddle frame 2024 can attach to the cap 2014 by any suitable means, such as, for example, any means described in the present application. Referring to FIGS. 46-49, in the illustrated example both of the anchors 2008 include are defined by a paddle ribbon 2001 that includes the inner paddle 2022 and the outer paddle 2020 of each anchor 2008. The inner paddles 2022 of each anchor 2008 are attached by a connection portion 2025 that is configured to connect the inner paddles 2022 to the coaptation element 2010 (as shown in FIG. 49). In the illustrated example, the connection portion 2025 includes an opening 2094 for receiving a distal portion of the coaptation element 2010. The outer paddles 2020 of each anchor 2008 are attached by a connection portion 2021 that is configured to connect the outer paddles 2020 to the cap 214 (as shown in FIG. 49). In the illustrated example, the connection portion 2021 includes an opening 2096 for receiving a portion of the cap 2014. Each inner paddle 2022 is attached to the corresponding outer paddle 2020 by connection portion 2023.

Referring to FIGS. 38 and 39, the paddle frame 2024 includes two or more arms 2080 that define the total width TW of the anchors 2008, in which the at least some of the arms 2080 are connected at a distal portion of the paddle frame 2024 (e.g., a portion of the paddle frame 2024 proximate the connection members 2003). Each of the arms 2080 includes one or more openings 2091, 2092 for receiving one or more width adjustment lines (e.g., width adjustment lines 1890 shown in FIGS. 36-37) such that a user can pull on the width adjustment lines to cause the paddle frame 2024 to move to the narrowed position. The illustrated example includes two arms 2080 that each include a proximal opening 2091 and a distal opening 2092. In some implementations, a single width adjustment line can extend through each opening 2091, 2092 such that the single width adjustment line can cause the paddle frame 2024 to move to the narrowed position. It should be understood, however, that any suitable number of width adjustment lines can extend through the openings 2091, 2092 to cause the paddle frame 2024 to move to the narrowed position.

Referring to FIG. 38, the arms 2080 are connected to each other at the distal portion of the paddle frame 2024 by a connection link 2083. This connection between the two arms 2080 causes the arms 2080 to pivot, flex, and/or articulate about the connection link 2083 in an inward direction Z when a user causes a tensioning force F on the paddle frame 2024 by pulling the one or more width adjustment lines that extend through the openings 2091, 2092. This pivoting, flexing, and/or articulating of the arms 2080 causes the main support section 2085 of the arms 2080 to move in the inward direction X such that the paddle frame 2024 is in the narrowed position. In the illustrated example, the connection link 2083 has a first member 2087 attached to one arm 2080, a second member 2089 attached to the other arm 2080, and a thin arched member 2086 that connects the first member 2087 to the second member 2089. The connection link 2083 can, however, take any suitable form that allows the arms to pivot, flex, and/or articulate in the inward direction Z when a tensioning force F is applied to the paddle frame 2024. In some implementations, the connection link 2083 is integral to the arms 2080 of the paddle frame 2024.

Still referring to FIGS. 38, the total width TW of the paddle frame 1824 when in the normal, expanded position can be between 5 mm and 15 mm, such as between 7 mm and 12 mm, such as between 9 mm and 11 mm, such as about 10 mm. The narrowed width of the paddle frame 1824 can be between 3 mm and 12 mm, such as between 5 mm and 10 mm, such as between 7 mm and 9 mm, such as about 8 mm A ratio of the normal width to the narrowed width W2 can be between 10/9 and 3/1, such as between 5/4 and 2/1, such as between 4/3 and 3/2.

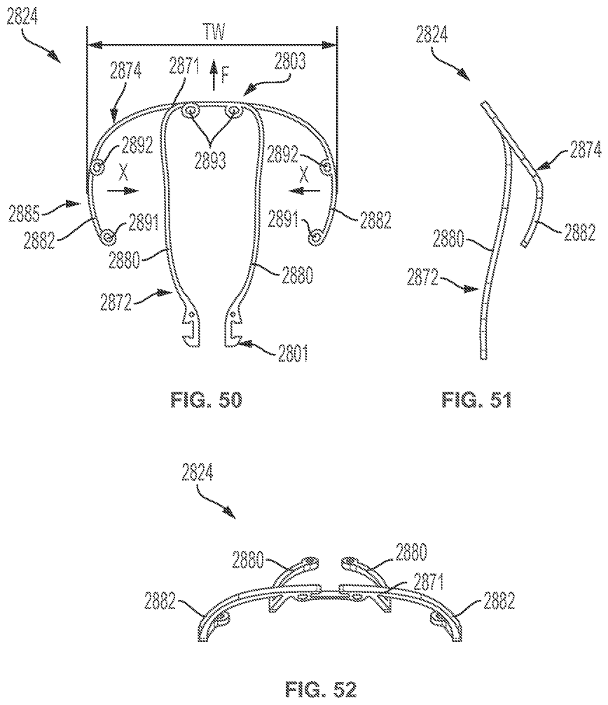

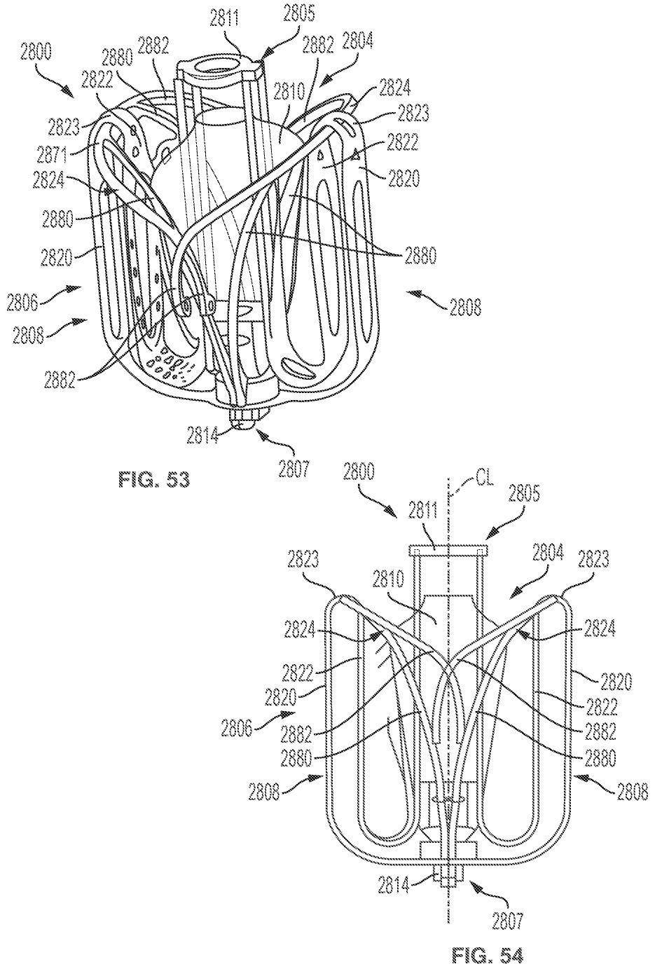

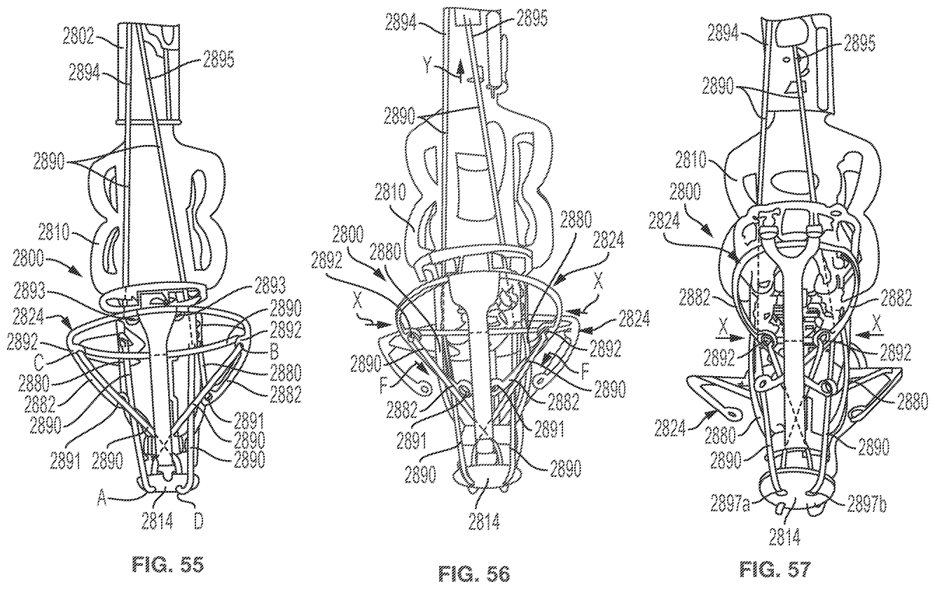

Referring to FIGS. 50-59, an example implementation of an implantable device or implant 2800 (FIGS. 53-59) includes an anchor portion 2806 having one or more paddle frames 2824 that are movable to a narrowed position to allow the device 2800 to maneuver more easily into position for implantation in the heart by reducing the contact and/or friction between the native structures of the heart—e.g., chordae—and the device 2800. That is, one or more width adjustment elements, such as the illustrated width adjustment lines 2890 (FIGS. 55-59) are controlled by a user to create a compression force on the paddle frames 2824 to move the paddle frames 2824 to a narrowed position as the device 2800 is being positioned for implantation on the leaflets of a native valve such that the contact between the native structures of the heart and the device 1800 is reduced. In some implementations, the width adjustment elements can be width adjustment wires, width adjustment cords, width adjustment sutures, other width adjustment elements described herein, etc.) The device 2800 can include any other features for an implantable device or implant discussed in the present application or in the applications and patents incorporated by reference herein, and the device 2800 can be positioned to engage valve tissue 20, 22 as part of any suitable valve repair system (e.g., any valve repair system disclosed in the present application). In addition, any of the devices described herein can incorporate the features of the device 2800.

Referring to FIGS. 53-54, the implantable device or implant 2800 includes a coaptation portion 2804, a proximal or attachment portion 2805, an anchor portion 2806, and a distal portion 2807. The coaptation portion 2804, attachment portion 2805, and distal portion 2807 can take any suitable form, such as, for example, the form for these portions of the device 200 shown in FIG. 22, or any other form described in the present application. In some implementations, the coaptation portion 2804 optionally includes a coaptation element 2810 (e.g., a spacer, coaption element, gap filler, etc.) that can be used, for example, for implantation between the leaflets 20, 22 of the native mitral valve MV. The coaptation element, etc. 2810 can take any suitable form, such as, for example, any form described in the present application.

The attachment portion 2805 includes a first or proximal collar 2811 for engaging with a capture mechanism of a delivery s system 2802. The capture mechanism and the delivery system 2802 can take any suitable form, such as, for example, any form described in the present application. The delivery system 2802 can be the same as or similar to other delivery systems herein, e.g., 102, 402, 502, etc. and can comprise one or more of a catheter, a sheath, a guide catheter/sheath, a delivery catheter/sheath, a steerable catheter, an implant catheter, a tube, a channel, a pathway, combinations of these, etc. The proximal collar 2811 can take any suitable form, such as, for example, any form described in the present application.

The distal portion 2807 includes a cap 2814 that is attached to anchors 2808 of the anchor portion 1806 such that movement of the cap 2814 causes the anchors 2808 to move between open and closed positions. The cap 2814 can take any suitable form, such as, for example, any form described in the present application. In the illustrated example, an actuation element (e.g., the same as or similar to actuation element 112 shown in FIGS. 8-20 or actuation element 8102 shown in FIGS. 26-30) extends from a delivery system (e.g., any delivery system described in the present application) and engages the cap 2814 to move the cap 2814 relative to the coaptation element 2810 to enable actuations of the device 2800. The actuation element can engage and move the cap by any suitable means, such as, for example, any means provided in the present application.

The anchor portion 2806 can take any suitable form, such as, for example, the form of the anchor portion 206 of the device 200 shown in FIG. 22 or any other form described in the present application. The anchor portion 2806 can include a plurality of anchors 2808, each anchor 2808 including outer paddles 2820, inner paddles 2822, paddle extension members or paddle frames 2824, and clasps (e.g., clasps 230 shown in FIG. 22). Referring to FIGS. 50-52, the paddle frames 2824 can include a main support section 2885, first connection members 2801 for attaching to the cap 1814, and second connection members 2803 for attaching to a connection portion 2823 of the anchors 2808. The paddle frame 2824 can attach to the connection portion of the anchors and the cap by any suitable means, such as, for example, any means described in the present application. The thickness and width of the paddle frame 2824 can take any suitable form, such as, for example, the thickness can be substantially identical to the width, the thickness can be greater than the width (as shown in FIGS. 91-95), or the width can be greater than the thickness.

The paddle frame 2824 includes an inner portion 2872 and an outer portion 2874. The inner portion 2872 has arms 2880 that extend from the connection members 2801 to a proximal portion of the paddle frame 2824. The outer portion 2874 includes arms 2882 that are connected to arms 2880 at connection point 2871 and extend outward from arms 2880. The arms 2882 define a total width TW of the anchors 2808. The arms 2882 can have one or more openings for receiving one or more width adjustment lines 2890 such that the width adjustment lines 2890 can be engaged by a user to move the paddle frame 2824 to the narrowed position by moving the arms 2882 in the inward direction X. In the illustrated example, each of the arms 2882 have a first opening 2892 and a second opening 2891 that is positioned distally from the first opening 2892. The inner portion 2872 can include one or more openings 2893 that can be used for connecting to the connection portion 2823 of the anchors 2808 and/or for receiving one or more width adjustment lines 2890.

Referring to FIGS. 51-55, the arms 2882 of the outer portion 2874 can be biased in the direction X (FIGS. 51-52) such that the arms 2882 are configured to extend beyond a center line CL (FIG. 54) of the device 2800 when the anchors 2808 are in the closed position. Referring to FIGS. 53-54, for illustrative purposes, the arms 2882 of the paddle frames 2824 are shown crossing each other to show that the arms 2882 are configured to extend beyond the center line CL of the device 2800. It should be understood, however, that the arms 2882 can be positioned to engage the arms 2882 of the other paddle frame 2824 (rather than cross each other) to create a pinching force between the two anchors 2808. In these examples, when the anchors 2808 have captured the leaflets 20, 22 of the mitral valve MV the biased arms 2882 of each paddle frame 2824 pinch the leaflet tissue between them to better secure the device 2800 to the mitral valve MV.

Referring to FIG. 52, the paddle frame 2824 can have a rounded shape that corresponds to the shape of the coaptation element 2810 such that the anchors 2808 conform around the coaptation element to better secure the leaflet tissue between the anchors 2808 and the coaptation element 2810. The paddle frames 2824 can be formed by shape setting a material such that the arms 2882 are biased away from the arms 2880. For example, the paddle frames 2824 can be made of metals, such as steel, nitinol, etc., plastics, etc.

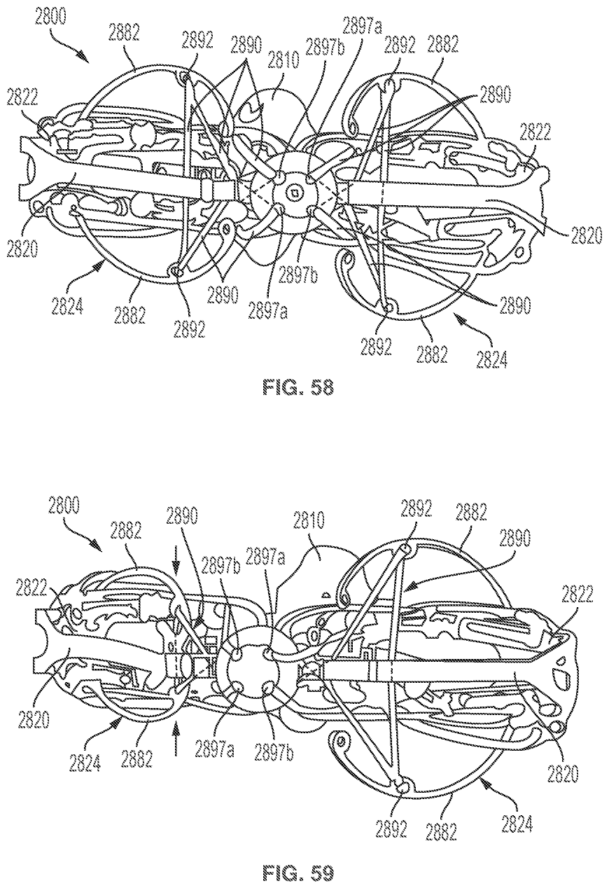

Referring to FIGS. 55-59, in some implementations, each paddle frame 2824 has a corresponding width adjustment line 2890 that is used to move the paddle frame 2824 from the normal, expanded position (FIGS. 55 and 58) to a narrowed position (FIGS. 56-57 and 59). Each width adjustment line 2890 can include two ends 2894, 2895 that extend from the delivery system 2802 such that a user can engage the ends 2894, 2895 to cause the paddle frame 2824 to move to the narrowed position. The width adjustment line 2890 can extend through the cap 2814 before extending through one or more openings (e.g., openings 2891, 2892, 2893) of the paddle frame 2824 and then extending back into the delivery system 2802.

Referring to FIG. 55, in the illustrated example, a first end 2894 of the width adjustment line 2890 extends from the delivery system 2802 and through an opening 2897a (FIGS. 57-59) of the cap 2814 at point A. Then the width adjustment line 2890 extends through the opening 2892 of one arm 2882 at point B and then through the opening 2892 of the other arm 2882 at point C. The width adjustment line 2890 extends back through an opening 2897b (FIGS. 57-59) the cap at point D, and then the second end 2895 of the width adjustment line 2890 extends back through the delivery system 2802.

Referring to FIG. 56, when a user pulls the ends 2894, 2895, of the width adjustment line 2890 in the direction Y, the width adjustment line 2890 causes a tensioning force F on the arms 2882 due to the width adjustment line extending through the openings 2892. This tensioning force F then causes the arms 2882 to move in the inward direction X such that the paddle frame 2824 is in a narrowed position. Referring to FIG. 57, if the user provides additional force to the ends 2894, 2895 of the width adjustment line 2890 in the direction Y, the tensioning force F (FIG. 56) continues on the arms 2882 such that the arms 2882 continue to move in the direction X, which can cause the arms 2882 to cross each other (as shown in FIG. 57) such that the paddle frame 2824 is in a more narrowed position. Referring to FIGS. 58 and 59, the paddle frames 2824 can be independently controllable between the normal and narrowed positions. For example, FIG. 58 shows both paddle frames 2824 in the normal position, and FIG. 59 shows one paddle frame 2824 moved to the narrowed position and the other paddle frame 2824 in the normal position.

Referring to FIG. 50, the total width TW of the paddle frame 2824 when in the normal, expanded position can be between 5 mm and 15 mm, such as between 7 mm and 12 mm, such as between 9 mm and 11 mm, such as about 10 mm. The narrowed width of the paddle frame 2824 can be between 3 mm and 12 mm, such as between 5 mm and 10 mm, such as between 7 mm and 9 mm, such as about 8 mm A ratio of the normal width TW to the narrowed width can be between 10/9 and 3/1, such as between 5/4 and 2/1, such as between 4/3 and 3/2.

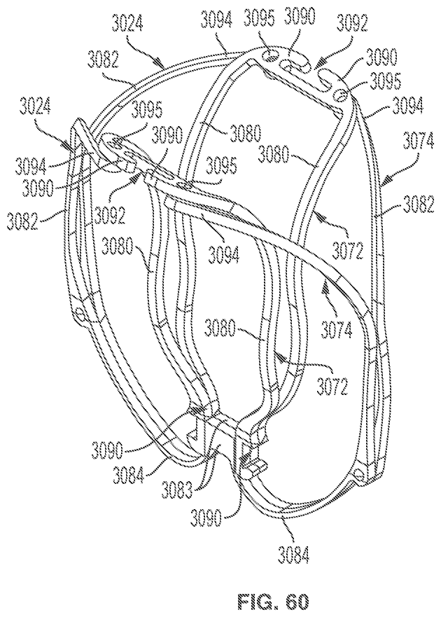

FIGS. 60-63 illustrate an example implementation of paddle frames 3024 for an implantable device or implant, such as any of the implantable device or implants disclosed herein. The paddle frames 3024 are configured to allow the device to maneuver more easily into position for implantation in the heart by reducing the contact and/or friction between the native structures of the heart—e.g., chordae—and the device.

Referring to FIGS. 60-63, each of the paddle frames 3024 can include an inner portion 3072 and an outer portion 3074. The inner portion 3072 includes one or more arms 3080 having a proximal end 3090 and a distal end 3091. The proximal ends 3090 can be connected and have an opening 3092 for receiving the paddles (e.g., the inner and outer paddles) of the anchors. The distal end 3091 can include connection members for attaching to a cap that is used to open and close the paddles. The illustrated example shows that the inner portion 3072 have two arms 3080, but it should be understood that the inner portion 3072 can have any suitable number of arms.

The outer portion 3074 of each of the paddle frames 3024 has a pair of arms 3082 having a proximal end 3093 and a distal end 3094. The proximal ends 3093 can be configured to attach to the proximal ends 3090 of the inner portion 3072. For example, the proximal ends 3090, 3093 of both the inner and outer portions 3072, 3074 can include openings 3095, 3096 for receiving a fastener that connects the inner and outer portions 3072, 3074 together. The distal ends 3094 are connected together at connection point or inner end 3083. The arms 3082 can be curved such that the distal ends 3094 extend above at least a portion of the remainder of the arms 3082. For example, in the illustrated example, the arms 3082 include curved portions 3084. In some implementations, the connection point or inner end 3083 of the distal ends of the arms 3082 is connected to the distal ends 3091 of the arms 3080 of the inner portion 3072 such that the distal ends 3091, 3094 can move together in the proximal direction PD or the distal direction DD with the connection point or inner end 3083 as it is moved in the proximal direction PD or the distal direction DD.

In some implementations, the arms 3082 are more flexible than the arms 3080. This increased flexibility allows the arms 3082 to flex when the connection portion or inner end 3083 is pulled into the arms 3080. This flexing allows the arms 3082 to narrow. The stiffer arms 3080 allow the paddles of the device to open and closed in the same or a similar manner to that shown in FIGS. 23, 27, and 30-37.

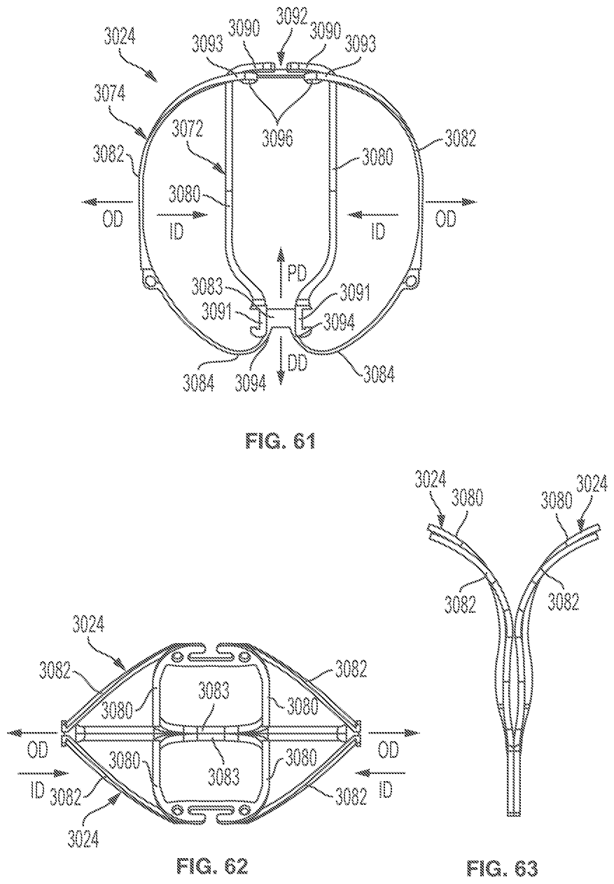

Referring to FIG. 61, movement of the connection point or inner end 3083 that connects the distal ends 3094 of the arms 3082 in the distal direction DD causes the arms 3082 to move in the outward direction OD (FIG. 61) such that the paddle frame 3024 is in an expanded position. That is, referring to FIG. 61, movement of the connection point or inner end 3083 in the distal direction DD causes the curved portions 3084 of the arms 3082 to flex outward, which causes the arms 3082 to move in the outward direction OD.

Referring to FIG. 61, movement of the connection point or inner end 3083 that connects the distal ends 3094 of the arms 3082 in the proximal direction PD causes the arms 3082 to move in the inward direction ID such that the paddle frame 3024 moves to a narrowed position. That is, referring to FIG. 61, movement of the connection point or inner end 3083 in the proximal direction PD causes the curved portions 3084 of the arms 3082 to flex inward, which causes the arms 3082 to move in the inward direction ID.

The connection point or inner end 3083 can be moved in the distal direction DD or the proximal direction PD by a user with a width adjustment element (e.g., width adjustment element 8211 shown in FIGS. 26-30, or another width adjustment elements shown or described herein). For example, the connection point or inner end 3083 can be coupled to the width adjustment element, such that the actuation element can move the connection wire in the proximal direction PD and the distal direction DD. A wide variety of mechanisms can be used to move the connection point or inner end 3083 in the proximal and distal directions to adjust the width of the paddle frames. Several examples of mechanisms that can be used to move the connection point or inner end 3083 in the proximal and distal directions to adjust the width of the paddle frames are disclosed below.

In some implementations, the paddle frames 3024 illustrated by FIGS. 60-63 can have a normal, expanded width between 5 mm and 15 mm, such as between 7 mm and 12 mm, such as between 9 mm and 11 mm, such as about 10 mm. The narrowed width of the paddle frame 3024 can be between 3 mm and 12 mm, such as between 5 mm and 10 mm, such as between 7 mm and 9 mm, such as about 8 mm A ratio of the normal, expanded width to the narrowed width can be between 10/9 and 3/1, such as between 5/4 and 2/1, such as between 4/3 and 3/2.

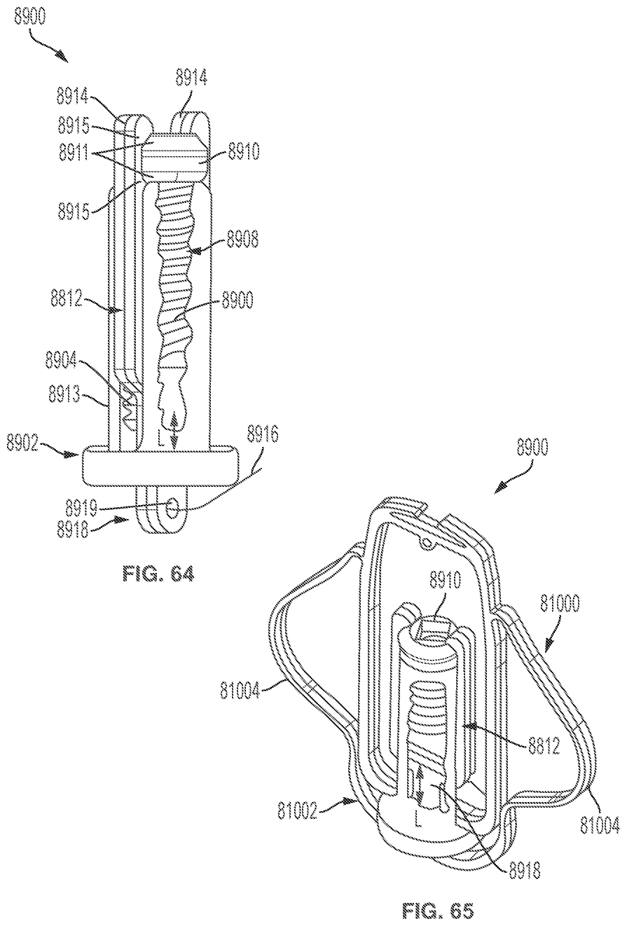

FIG. 64 illustrates an example of a width adjustment device 8900 that is configured to expand or contract the paddle frames of an implantable device or implant. The width adjustment device 8900 can take any suitable form, such as, for example, any form described in the present application. Moreover, any of the implantable device or implants and width adjustment devices described herein can incorporate features of the width adjustment device 8900. In the illustrated example, the width adjustment device 8900 includes a coupler, such as the illustrated shaft 8908 and a receiver, such as the illustrated housing 8902. In some implementations, the housing 8902 can be an integral part of an implantable device or implant. For example, the housing 8902 can be integrally formed with the distal cap or any other suitable member described herein. The shaft 8908 includes an external thread pattern that is configured to threadedly engage a female thread pattern 8904 formed in the housing 8902. A driver head 8910 is integrally formed at a proximal end of the shaft 8908 and is configured to enable rotation of the shaft 8908 by a variety of tools or drive types (e.g., Torx, slotted, Philips, etc.).

Still referring to FIG. 64, a fork-shaped carriage 8812 is disposed around the shaft 8908 and the driver head 8910. In the illustrated example, the carriage 8812 features proximal tines 8914 and a distal end 8918 which are formed as a single, unitary component. However, it is understood that the carriage 8812 can take any suitable form, such as, for example, any form described in the present application.

Still referring to FIG. 64, the driver head 8910 features mating surfaces 8911 that are configured to be complementary with surfaces 8915 of the proximal tines 8914, respectfully, for harnessing the carriage 8812 to the driver head 8910. Torque prevention cutaways 8913 formed in the housing 8902 are configured to receive and constrain the carriage 8812 to longitudinal movement in the direction L and prevent or inhibit the carriage 8812 from rotating when torque is applied to the driver head 8910. Therefore, when the driver head 8910 is rotated, the driver head 8910 will pull the carriage 8812 such that the carriage 8812 is confined to move in an upward or downward direction along a longitudinal axis of the shaft 8908 as indicated by arrows L.

Still referring to FIG. 64, the distal end 8918 of the carriage 8812 is formed with an aperture 8919 that is configured to permit a width adjustment line 1890 to pass therethrough. Opposite ends of the width adjustment line 1890 can be secured to various attachment points on the paddle frames, the paddles, the distal cap, or to any other suitable attachment point described herein. When the driver head 8910 is rotatably driven to move the carriage 8812, the distal end 8918 of the carriage 8812 will pull on the width adjustment line 1890, thereby causing the paddle frames (not shown) to contract. In the illustrated example, rotating the driver head 8910 clockwise (right-handed thread configuration) will cause the carriage 8812 to move downwards along the longitudinal axis of the shaft 8908 in the direction illustrated by arrows L. In this way, the distal end 8918 of the carriage 8812 will pull on the width adjustment line 1890 causing the paddle frames to contract. However, it is understood that other configurations are also contemplated. For example, rotating the driver head 8910 in a counterclockwise direction could apply tension to the width adjustment line 1890 for causing the paddle frames to contract. Moreover, it is appreciated that in other configurations, applying tension to the width adjustment line 1890 could cause the paddle frames to expand, rather than contract.

Referring to FIG. 65, an example of a width adjustment device 8900 and a retractable/expandable paddle frame is shown. In the illustrated example, the width adjustment device 8900 of FIG. 65 is substantially the same as that of the example shown in FIG. 64, except that the distal end 8918 of the carriage 8812 is integrally formed with a distal portion 81002 of paddle frames 81000 of an implantable device or implant. However, it should be understood that in some implementations, the carriage 8812 can be integrally formed with the distal cap, or with any other suitable member described herein.

Still referring to FIG. 65, when the driver head 8910 is rotatably driven to convey the carriage 8812, the carriage 8812 will cause a distal portion 81002 to move upward or downward. Upward movement of the distal portion 81002 causes the paddle frames 81000 to flex outward or expand and downward movement of the distal portion 81002 causes the paddle frames 81000 to flex inward or retract. For example, when rotating the driver head 8910 clockwise (right-handed thread configuration), the carriage 8812 will move in a downward direction along the longitudinal axis causing the distal portion 81002 of the paddle frames 81000 to move downward, and lateral portions 81004 of the paddle frames 81000 to contract inward, thereby reducing the overall width of the paddle frames 81000. When rotating the driver head 8910 counterclockwise (right-handed thread configuration), the carriage 8812 will move in an upward direction along the longitudinal axis causing the distal portion 81002 of the paddle frames 81000 to move upward, and lateral portions 81004 of the paddle frames 81000 to expand outward, thereby increasing the overall width of the paddle frames 81000.

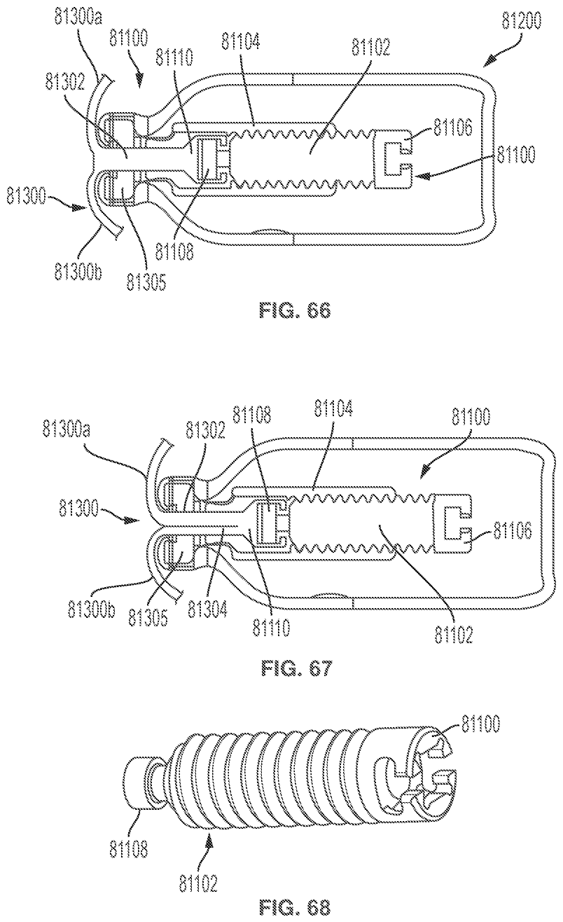

FIG. 66 illustrates an example of a width adjustment device 81100 that is configured to expand or contract the paddle frames of an implantable device or implant 81200. The width adjustment device 81100 can take any suitable form, such as, for example, any form described in the present application. Moreover, any of the implantable device or implants and width adjustment devices described herein can incorporate features of the width adjustment device 81100. In the illustrated example, the width adjustment device 81100 includes a coupler, such as the illustrated externally threaded shaft 81102 (FIG. 68) that is rotatably engaged with a receiver, such as the illustrated internally threaded element 81104 (illustrated and often referred to as a “column” or herein, but can be or comprise other types of threaded elements or threaded lumens and have a variety of different sizes and shapes as well) that is integrally formed with or connected to a distal portion of an implantable device or implant. For example, the threaded element or column 81104 can be integrally formed with the distal cap, a distal portion of the paddle assembly, or with any other suitable member described in the present application.

A driver head 81106 is disposed at a proximal end of the shaft 81102 and is configured to rotatably drive the shaft 81102 into or out of the threaded element or column 81104. The driver head 81106 can take any form, such as for example, any form described in the present application. Referring to FIG. 68, a coupler 81108 is attached to a distal end of the shaft 81102 and is configured to be retained by a receiver 81110 (FIG. 66) that is formed on an inner end or post 81302. The inner end or post member 81302 is configured to mechanically couple the expandable/retractable paddle frames 81300 to the coupler 81108. In this way, when the driver head 81106 is driven to rotate the shaft 81102 counterclockwise (e.g., right-handed thread configuration), the shaft 81102 will rotate and move toward a proximal end of the width adjustment device 81100 causing the coupler 81108 to pull on the receiver 81110 of the paddle frames 81300. As the receiver 81110 is pulled by the coupler 81108, the paddle frames 81300 will begin to contract inward and reduce the overall width of the paddle frames 81300. Conversely, rotating the shaft 81102 clockwise (e.g., into the threaded element or column 81104) will cause the paddle frames 81300 to expand outwards. However, it should be understood that other configurations are also contemplated. For instance, in some implementations, rotating the shaft 81102 clockwise will cause the paddle frames 81300 to contract inwards. Therefore, it is appreciated that a wide variety of configurations are contemplated for expanding or contracting the paddle frames.

FIG. 67 illustrates an example of a width adjustment device 81100 that is configured to expand or contract the paddle frames of an implantable device or implant is shown. The width adjustment device 81100 can take any suitable form, such as, for example, any form described in the present application. Moreover, any of the implantable device or implants and width adjustment devices described herein can incorporate features of the width adjustment device 81100. In the illustrated example, the width adjustment device 81100 of FIG. 67 is substantially the same as the example shown in FIG. 66, except that the inner end or post 81302 is partially split along partition line 81304. The split inner end or post 81302 connects the coupler 81108 to the paddle frames 81300. The paddle frames 81300 are partially retractable into a distal portion 81305 (e.g., distal cap) of an implantable device or implant. In particular, sheathable portions 81300a and 81300b of the paddle frames 81300 can be drawn in and through the distal portion 81305 and into the cavity that is formed by the internally threaded column 81104. In this way, when the driver head 81106 causes the coupler to pull on the receiver 81110 for contracting the paddle frames 81300 and drawing the sheathable portions 81300a 81300b in through the distal portion 81305. The contracting paddle frames are particularly advantageous when having to navigate an implantable device or implant through tight spaces, such as through the chordae tendineae (e.g., such as, when deploying the device).

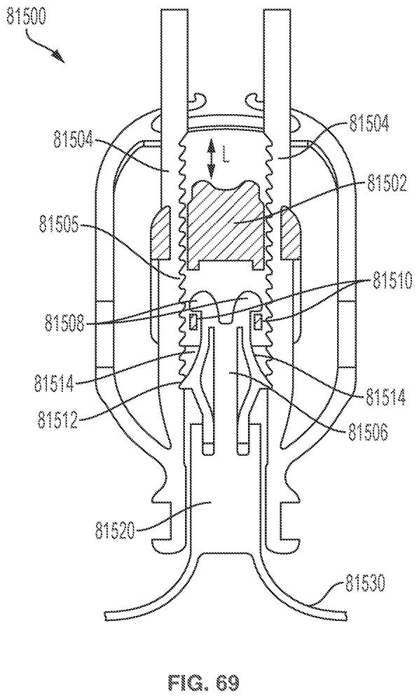

Referring to FIG. 69, an example implementation of a width adjustment device is shown. Any of the implantable devices or implants and width adjustment devices described herein can incorporate features of width adjustment device 81500. In the illustrated example, the width adjustment device 81500 includes an engagement member or actuator 81502 that is couplable to a width adjustment element (e.g., width adjustment control, width adjustment wire, width adjustment shaft, width adjustment tube, width adjustment line, width adjustment cord, width adjustment suture, width adjustment tether, etc.), a receiver, such as the illustrated parallel racks 81504, and a coupler 81506. Each rack 81504 includes teeth 81505 that are configured to limit the motion of the coupler 81506 to a single direction (e.g., a ratchet mechanism) when the coupler is in an engaged state. In the illustrated example, the coupler 81506 is coupled to paddle frames 81530 by a connection portion 81520. In some implementations, the coupler 81506, the connection portion 81520, and the paddle frames 81530 can be formed as a single, unitary component.

Still referring to FIG. 69, arms 81508 are formed on the coupler 81506 and are configured to engage projections 81510 of the actuator 81502 (e.g., see sectional view of FIG. 69). Resilient fingers 81512 are also formed on the coupler 81506 and are configured to engage the teeth 81505 of the rack 81504 for preventing the coupler 81506 from moving along the path L in a downward or distal direction of the racks 81504.

Still referring to FIG. 69, the actuator 81502 can be driven in the directions indicated by arrows L. When the actuator 81502 is driven upwards, the projections 81510 of the actuator 81502 will pull the coupler 81506 via the arms 81508 of the coupler 81506. As a result, the resilient fingers 81512 will ratchet along the teeth 81505 of the rack 81504, thereby permitting the coupler 81506 to move upwards when the actuator 81502 moves upwards. Simultaneously, the coupler 81506 will cause the connection portion 81520 to pull on the paddle frames 81530 and cause the paddle frames 81530 to contract. In such implementations, the position of the resilient fingers 81512 relative to each of the plurality of discrete positions (i.e., the teeth) on the rack 81504 can correspond to a particular width of the paddle frames, respectively.

Conversely, when the actuator 81502 is driven downwards, the projections 81510 of the actuator 81502 push against resilient, sloped surfaces 81514 of the coupler 81506. As such, the projections 81510 cause the resilient fingers 81512 to disengage from the rack 81504. As such, the coupler 81506 is disengaged from the rack 81504 when the actuator is moved in a downward or distal direction to expand the paddle frames 81530.

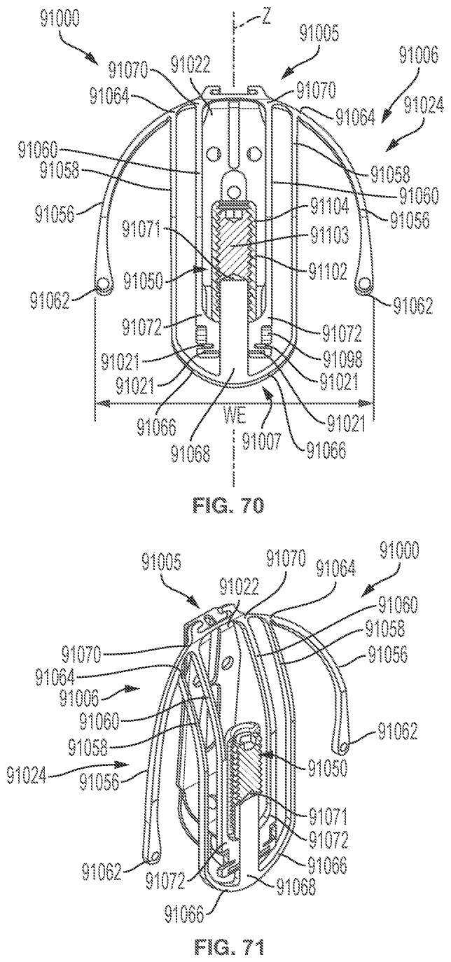

Referring to FIGS. 70-84, an example implementation of an implantable device or implant 91000 is shown. The implantable device or implant 91000 includes a proximal or attachment portion 91005, anchor portions 91006 that include paddle frames 91024, an actuation portion 91050, and a distal portion 91007. The paddle frames 91024 have a height H (FIG. 73) between the proximal portion 91005 and the distal portion 91007. The anchor portion 91006 includes inner paddles 91022 and outer paddles 91020. The attachment portion 91005, the distal portion 91007, the anchor portion 91006, and the actuation portion 91050 can be configured in a variety of ways.

The paddle frames 91024 are configured to allow the device 91000 to maneuver more easily into position for implantation in the heart by reducing the contact and/or friction between the native structures of the heart—e.g., chordae—and the device. That is, the paddle frames 91024 are configured to move between an expanded condition and a narrowed condition. When the paddle frames 91024 are in the narrowed condition, the contact between the native structures of the heart and the device 91000 is reduced. The device 91000 can include any other features for an implantable device or implant discussed in the present application or in the applications and patents incorporated by reference herein, and the device 91000 can be positioned to engage valve tissue 20, 22 as part of any suitable valve repair system (e.g., any valve repair system disclosed in the present application). In addition, any of the devices described herein can incorporate the features of the device 91000.

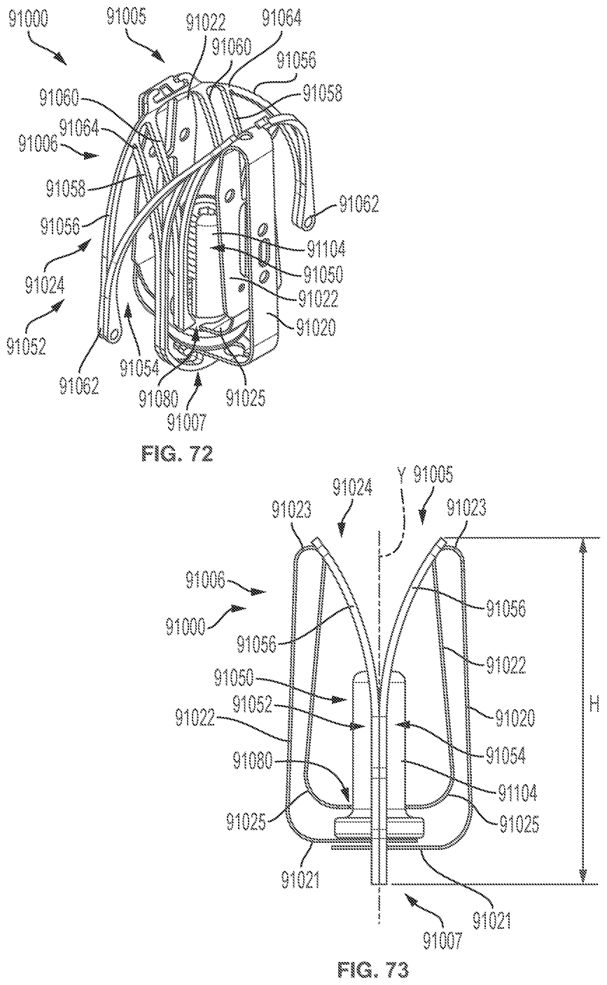

In the illustrated example of FIGS. 70-86, the paddle frames 91024 are symmetric along longitudinal plane Y (FIG. 73) and are symmetric along longitudinal plane Z (FIG. 70). In some implementations of the device or implant 91000, however, the paddle frames 91024 are not symmetric about one or both of the planes Y and Z. The paddle frames 91024 include a first frame side 91052 and a second frame side 91054 that is a minor image of the first frame side 91052 (FIGS. 72-86).

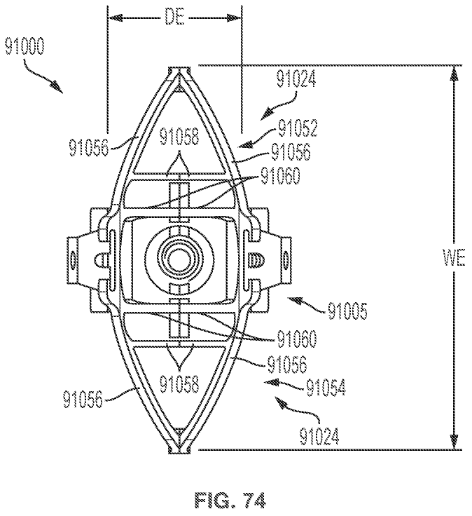

In the illustrated example in FIGS. 70-74, the paddle frames 91024 includes outer frame portions 91056, intermediate frame members 91058, and inner frame portions 91060. In FIG. 70, the outer frame portions 91056 are shown in an expanded state such that the outer frame portions 91056 define a paddle frame width WE in the expanded state and a paddle frame depth DE in the expanded state (FIG. 74). The outer frame portions 91056 are attached to the intermediate frame members 91058 at the proximal portion 91005 and include terminal distal ends 91062. The outer frame portions 91056 are curved and can form a semicircle or U-shape. In some implementations, however, the outer frame portions 91056 can be otherwise shaped.

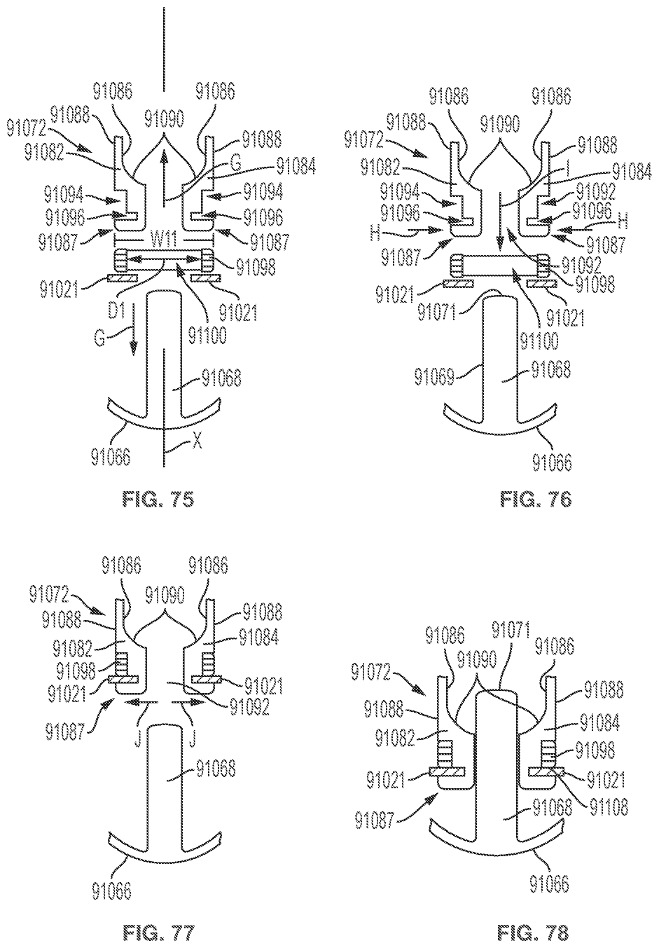

The intermediate frame members 91058 extend from a connection portion 91064 with the outer frame portions 91056 near or at the proximal portion 91005 and are attached at the distal portion 91007 via connection portions 91066. The intermediate frame members 91058 also include an inner end, which in the illustrated example is configured as a projection or post 91068 (FIGS. 70 and 71) extending axially along axis Z from the distal portion 91007 toward the proximal portion 91005. The post 91068 can be configured in a variety of ways. In the illustrated example, the post 91068 has a cylindrical outer side surface 91069 and an end surface 91071 perpendicular to the outer side surface (see FIG. 75).

The inner frame portions 91060 extend from a connection portion 91070 with the outer frame portions 91056 near or at the proximal portion 91005 and include retaining portions 91072 near or adjacent the distal portion 91007 for engaging the post 91068. The retaining portions 91072 are described below in more detail with regard to FIGS. 75-80.

The first frame side 91052 and a second frame side 91054 can optionally be in contact with each other along axis Y toward the distal portion 91007 and are separated toward the proximal end 91005 to form a V-shape, as shown, for example, in FIG. 72.

Referring to FIGS. 71-73, the outer paddles 91020 are connected to the retaining portions 91072 at the distal portion 91007 via connection portions 91021 and to the inner paddles 91022 by connection portions 91023. The inner paddles 91022 are connected to a coaptation portion or inner member (not shown) by connection portions 91025. Referring to FIGS. 71-73, the inner paddles 91022 are not connected to the retaining portions 91072. Instead, the inner paddles 91022 form an aperture or gap 91080 through which the retaining portions 91072 extend.

Referring to FIGS. 75-80, the method of assembling the retaining portions 91072 to the post 91068 is illustrated. The retaining portions 91072 include a first retaining portion 91082 and a second retaining portion 91084 spaced apart from, and a minor image of, the first retaining portion 91082. Each of the inner frame portions 91060 and retaining portions 91072 includes an inner side surface 91086, an outer side surface 91088 opposite the inner side surface 91086, and a distal end 91087.

The inner side surfaces 91086 of the inner frame portions 91060 include inward transition portions 91090 that form a seat. In the illustrated example, the inward transition portions 91090 are formed as inward curved surfaces. In some implementations, however, the inward transition portions 91090 can be formed in any suitable manner, such as for example, as angled or tapered surfaces, stepped surfaces, or any other suitable inward transition. The inner side surfaces 91086 extend axially from the inward transition portion 91090 toward the distal portion 91007 to form a gap 91092 configured to receive the post 91068.

Each of the outer side surfaces 91088 of the inner frame portions 91060 includes a first recessed portion 91094. In the illustrated example, the first recessed portion 91094 is formed axially nearer to the distal portion 91007 than the inward transition portion 91090 is located. Each of the first recessed portions 91094 include a second recessed portion 91096 that is recessed relative to the first recessed portion 91094. In the illustrated example, the second recessed portion 91096 is located at a portion of the first recessed portion 91094 that is closest to the distal portion 91007. The second recessed portions 91096 are configured to receive the connecting portions 91021 of the outer paddles 91020.

The first recessed portions 91094 are configured to receive an annular retainer 91098. The annular retainer 91098 can be a ring, a washer, a nut, or the like. The annular retainer 91098 includes an inner passage 91100 configured to receive the post 91068 therethrough. The inner passage 91100 has a diameter D1 which is less than the combined width W11 of the distal ends 91087 and gap 91092 in an uncompressed state as shown in FIG. 75.

FIG. 70 illustrates an assembled state for the device 91000 in which the post 91068 is received through the gap 91092 in the retaining portion 91072. To assemble the device 91000, as shown in FIG. 75, the post 91068 and connecting portion 91066 of the intermediate frame members 91058 and the retaining portion 91072 of the inner frame portions 91060 are pulled away from each other along the plane X, as shown by arrow G.

The paddle frames 91024 can be made from or comprise a material that allows the post 91068 and connecting portion 91066 of the intermediate frame members 91058 and the retaining portion 91072 of the inner frame portions 91060 to be pulled away from each other. For example, the paddle frames 91024, or a portion thereof, can be made of a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, some portions of the paddle frames 91024 can be stiffer or more rigid than other portions. For example, in the illustrated example of the paddle frames 91024, the inner frame portions 91060 can be configured to be stiffer than the outer frame portions 91056. The inner frame portions 91060 can be configured in a variety of ways to be stiffer or more rigid. For example, the thickness of the inner frame portions 91060 and/or the material used for the inner frame portions 91060 can provide more rigidity. In some implementations, the thickness of the inner frame portions 91060 can be greater than the outer frame portions 91056 to provide more rigidity. Further, in some implementations, the material used in the inner frame portions 91060 can be a more rigid material to provide more rigidity.

Once the post 91068 and the connecting portion 91066 are separated from the retaining portion 91072, the annular retainer 91098 and the connecting portions 91021 of the outer paddles 91020 can be placed therebetween. As shown by arrows H in FIG. 76, the distal ends 91087 of the first retaining portion 91082 and a second retaining portion 91084 can be compressed toward each other such that the gap 91092 is reduced or closed. The distal ends 91087 can be compressed such that the combined width of the distal ends 91087 and the gap 91092 is less than the diameter D1 of the passage 91100 of the annular retainer 91098. As such, the distal ends 91087 can be received through the passage 91100 and between the connection portions 91021 of the outer paddles 91020, as shown by arrow I in FIG. 76.

As shown in FIGS. 77, once the distal ends 91087 are received through the passage 91100 and between the connection portions 91021 of the outer paddles 91020, the annular retainer 91098 can be aligned with the first recessed portions 91094 and the connecting portion 91021 of the outer paddles 91020 can be aligned with the second recessed portions 91096. The distal ends 91087 can then be released to return toward the uncompressed state, as shown by arrows J, while the annular retainer 91098 is received in the first recessed portions 91094 and the connecting portions 91021 of the outer paddles 91020 are received with the second recessed portions 91096.

The distal ends 91087 can be configured to provide an outward bias on the annular retainer and/or the connecting portions 91021 of the outer paddles 91020 to provide a secure attachment between the annular retainer and/or the connecting portions 91021 of the outer paddles 91020. As shown in FIG. 78, once the annular retainer 91098 is received in the first recessed portions 91094 and the connecting portions 91021 of the outer paddles 91020 are received with the second recessed portions 91096, the post 91068 and the connecting portions 91066 of the intermediate frame members 91058 can be released, which allows the post 91068 to be received through the gap 91092 and the end surface 91071 extends past the inward transition portions 91090.

FIGS. 75-80 illustrate two connecting portions 91021 of the outer paddles 91020. For example, the outer paddles 91020 can be jointably attached at a distal portion 91007. FIGS. 70-71, however, show an implementation with the connecting portions 91021 of the outer paddles 91020 as not be jointly attached and being offset. Thus, the retaining portions 91072 can include an additional recessed portion (not shown) to receive one of the offset retaining portions 91072. The additional recessed portion (not shown) can be provided on the inner side surface 91086 or on the outer side surface 91088 of the retaining portion 91072.

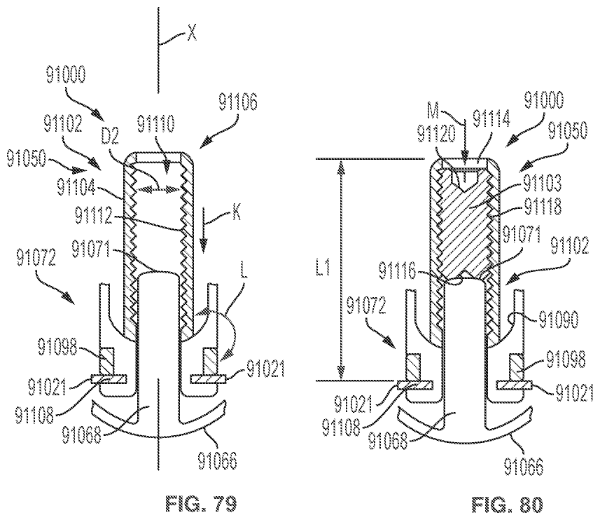

Referring to FIG. 79, the actuation portion 91050 of device or implant 91000 is configured to both facilitate moving the paddle frames 91024 between an expanded position and a narrowed position and move the paddles of the device 91000 between a closed position and an open position. The actuation portion 91050 can be configured in a variety of ways. Any structure capable of selectively moving the paddle frames 91024 between an expanded position and a narrowed position and moving the paddles of the device between a closed position and a closed position can be used. In some implementations, the actuation portion is configured such that advancing and retracting the actuation portion itself opens and closes the device and advancing and retracting the inner end or post inside the receiver narrows and widens the paddles. For example, the stiffer inner paddle frame portions are moved by the actuation portion to open and close the paddles in the same or a similar manner to that shown in FIGS. 8-15.

In the illustrated example, the actuation portion 91050 includes a receiver, such as the illustrated sleeve 91102, configured to receive a portion of the post 91068 and a coupler, such as the illustrated plug 91103 configured to move the post axially within the sleeve 91102. During assembly, the sleeve 91102 can be received onto the post 91068 as shown by arrow K. The receiver and coupler can be the same as or similar to other implementations of these described herein.

The sleeve 91102 can be configured in a variety of ways. In the illustrated example, the sleeve 91102 includes a cylindrical sidewall 91104 extending between a proximal end 91106 to a distal end 91108 of the annular retainer 91098. The sleeve 91102 can optionally be integrally formed with the annular retainer 91098. The sleeve 91102 defines an internal passage 91110.

The sleeve 91102 has a length L1 and the internal passage 91110 extends through the entire length L1 of the sleeve 91102 from the proximal end 91106 and a distal end 91108. The passage 91110 has a diameter D2 that is sufficient to allow the post 91068 to be received into the passage 91110 and includes an internal threaded portion 91112.

As shown by arrow Lin FIG. 79, the distal end 91108 of the sleeve 91102 is fixedly attached to the retaining portion 91072. The sleeve 91102 can be attached to the retaining portion 91072 in any suitable manner. In the illustrated example, annular retainer 91098 at the distal end 91108 is attached to the second recessed portion 91096 of the retaining portion 91072. The passage 91110 is aligned with the gap 91092 such that the post 91068 extends through the gap 91092 and into the passage 91110.

As shown FIG. 80, the plug 91103 is received within the passage 91110. The plug 91103 is configured to move axially within the sleeve 91102, as shown by arrow M. In the illustrated example, the plug 91103 is cylindrical and includes a proximal end 91114, a distal end 91116 opposite the proximal end, and an external threaded portion 91118. The external threaded portion 91118 is configured to threadedly engage with the internal threaded portion 91112 of the sleeve 91102.

The proximal end 91114 includes a drive interface 91120 configured to engage a drive member capable of rotating the plug 91103 to axially move the plug 91103 relative to the sleeve 91102. The drive interface 91120 can be any suitable interface. For example, the drive interface 91120 can be a drive recess, such as a slotted, hexagonal, Torx, Frearson, Phillips, square, or other suitable interface. The distal end 91116 forms an engagement surface configured to engage the proximal end 91071 of the post 91068.

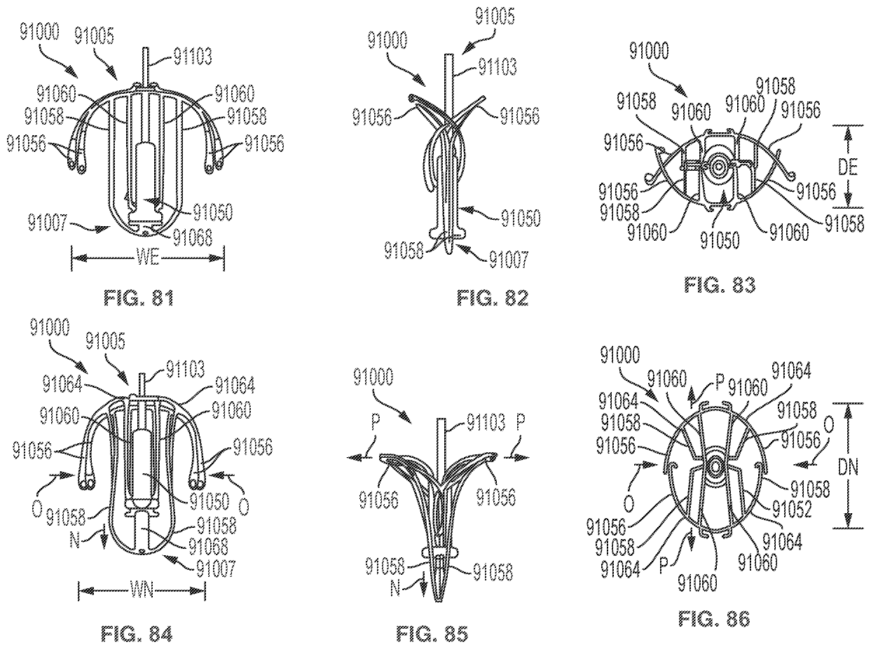

As shown in FIGS. 81-83, in the expanded state, the majority of, or most of, the post 91068 is received within the receiver 91102 and the device 91000 has the width WE defined by the positions of the outer frame portions 91056 and the depth DE. The plug 91103 is illustrated as extending out of the receiver 91102 toward the proximal portion 91005 of the device 91000. However, in some implementations, the plug does not extend past the proximal end of the receiver 91102 and an actuation rod is coupled to the plug 91103 in the receiver 91102 or at the end of the receiver 91102.

As shown in FIG. 83, for the illustrated example, in the expanded state, the top view of the device 91000 has the shape of a lens (i.e., a convex region bounded by two circular arcs that intersect at, or near, their endpoints).

Referring to FIGS. 79-80, in operation, to move the device 91000 from the expanded position to the narrowed position, a coupler, such as the illustrated plug 91103 can be moved axially relative to a receiver, such as the illustrated sleeve 91102. For example, the plug 91103 can be rotated via the drive interface 91120 to move the plug 91103 axially relative to the sleeve 91102. Movement of the plug toward the distal end 91108 of the sleeve 91102 causes the distal end 91116 of the plug 91103 to engage the proximal end 91071 of the post 91068 and move the post 91068 in the same direction (i.e., away from the proximal portion 91005 of the device).

As shown in FIGS. 84-86, movement of the post 91068 away from the proximal portion 91005 pulls the intermediate frame members 91058 in the same direction, as shown by arrow N in FIGS. 84 and 85. Due to the connection of the intermediate frame members 91058 to the outer frame portions 91056 at the connection portions 91064, movement of the intermediate frame members 91058 away from the proximal portion 91005 pulls the outer frame portions 91056 inward (i.e., to the narrowed position), as shown by arrows 0 in FIGS. 84 and 86, such that the device 91000 has a width WN in the narrowed position that is narrower than the width WE in the expanded position.

When the device 91000 narrows in width to the narrowed position, the device 91000 can also widen in the depth dimension, as shown by arrows P in FIGS. 85 and 86. As shown in FIG. 86, in the narrowed position, the device 91000 has a depth DN which is greater than the depth DE in the expanded position. In addition, the top view of the device 91000 changes from a lens shape, in the expanded position, to a circular or oval shape in the narrowed position, as shown in FIG. 86. Thus, the paddle frames 91024 can be moved between an expanded position and a narrowed position by rotating the plug 91103 within the sleeve 91102.

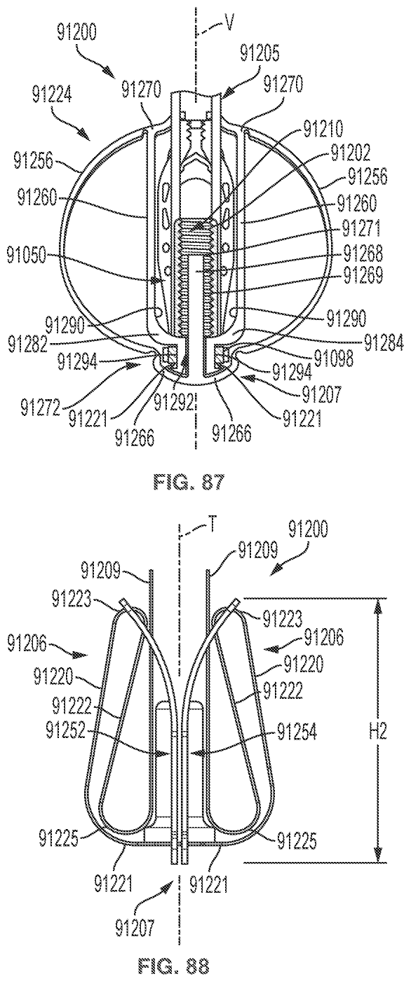

Referring to FIGS. 87-92, an example implementation of an implantable device or implant 91200 is shown. The implantable device or implant 91200 includes a proximal or attachment portion 91205, an anchor portion 91206 (FIG. 88), paddle frames 91224, an actuation portion 91050, and a distal portion 91207. The paddle frames 91224 have a height H2 (FIG. 88) between the proximal portion 91205 and the distal portion 91207. Referring to FIG. 88, the anchor portion 91206 includes inner members 91209, inner paddles 91222, and outer paddles 91220. The attachment portion 91205, the distal portion 91207, the anchor portion 91206, the actuation portion 91050, and the paddle frames 91224 can be configured in a variety of ways.

In the illustrated example of FIGS. 87-88, the paddle frames 91224 are symmetric along longitudinal axis T (FIG. 88) and are symmetric along longitudinal axis V (FIG. 87). In some implementations of the device or implant 91200, however, the paddle frames 91224 are not symmetric about one or both of the axes T and V. The paddle frames 91224 include a first frame side 91252 and a second frame side 91254 that is a minor image of the first frame side 91252 (FIG. 88).

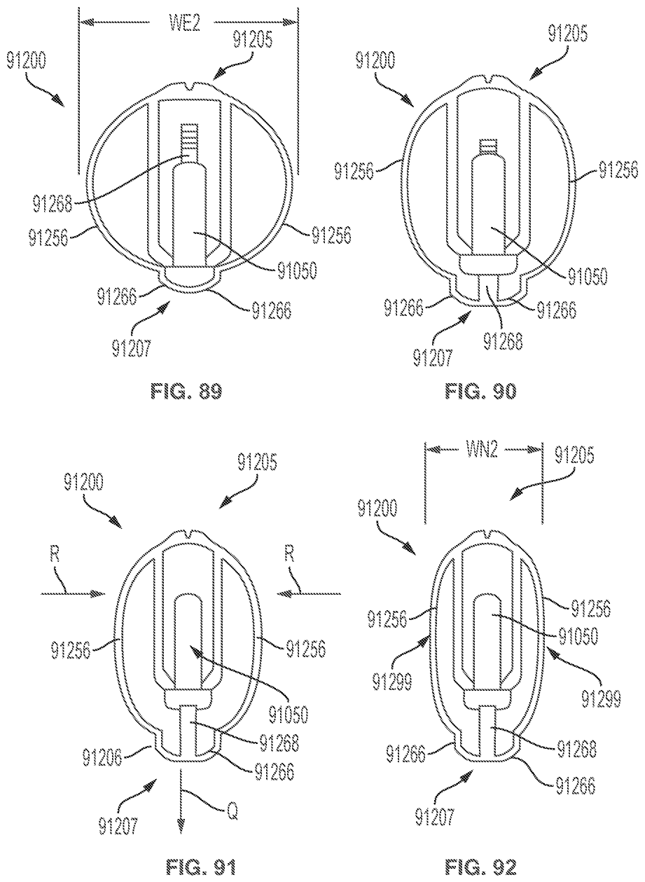

In the illustrated example, the paddle frames 91224 include outer frame portions 91256 and inner frame portions 91260. In FIG. 87, the outer frame portions 91256 are shown in an expanded state such that the outer frame portions 91256 define a paddle frame expanded width WE2 (FIG. 89).

The outer frame portions 91256 are flexibly attached at the proximal portion 91205 and are flexibly attached at the distal portion 91207. The outer frame portions 91256 are attached at the distal portion 91207 by connecting portions 91266. The outer frame portions 91256 are curved and form a generally circular or oval shape. In some implementations, however, the outer frame portions 91256 can be otherwise shaped.

The outer frame portions 91256 also include an inner end, which can be configured as a projection or post 91268 extending axially along axis V from the distal portion 91207 toward the proximal portion 91205. The inner end or post 91268 can be configured in a variety of ways. In the illustrated example, the inner end is configured as post 91268, which has an outer surface 91269 that can be formed by a plurality of side walls forming a polygonal cross section or the outer surface 91269 can have one flat side and a-half cylindrical surface. The post 91268 can have an end surface 91271 that is perpendicular to the side walls.

The inner frame portions 91260 extend from first connection portions 91270 with the outer frame portions 91256 near or at the proximal portion 91205 and include second connection portions 91272 near or adjacent the distal portion 91207 that connect to the post 91268. The first frame side 91252 and a second frame side 91254 can be in contact with each other along axis T toward the distal end 91207 and are separated toward the proximal end 91205 to form a V-shape, as shown, for example, in FIG. 88.

The inner members 91209 can be a portion of a coaptation element, such as coaptation element 210 of FIG. 22, or be attached to a coaptation element by any suitable means. As shown in FIG. 88, the outer paddles 91220 are jointably attached at the distal portion 91207 by connection portions 91221 and to the inner paddles 91222 by connection portions 91223. The inner paddles 91222 are flexibly attached to the inner members 91209 by connection portions 91225. The inner paddles 91222 and the inner members 91209 are not connected to the connection portions 91272, as shown in FIG. 88.

In this manner, the anchors are configured similar to legs in that the inner paddles 91222 are like upper portions of the legs, the outer paddles 91220 are like lower portions of the legs, and the connection portions 91223 are like knee portions of the legs

Referring to FIG. 87, the connection portions 91272 include a first retaining portion 91282 and a second retaining portion 91284, spaced apart from, and a mirror image of, the first retaining portion 91282. The inner frame portions 91260 include inward transition portions 91290. In the illustrated example, the inward transition portions 91290 are formed as inward curved surfaces. In some implementations, however, the inward transition portions 91290 can be formed in any suitable manner, such as for example, as angled or tapered surfaces, stepped surfaces, or any other suitable inward transition.

The retaining portions 91282, 91284 extend axially from the inward transition portions 91290 toward the distal end 91207 to form a gap 91292 configured to receive the post 91268. Each of the retaining portions 91282, 91284 includes an outer recessed portion 91294. In the illustrated example, the recessed portions 91294 are formed axially nearer to the distal end 91207 than the inward transition portion 91290 is located.

The recessed portions 91294 are configured to receive an annular retainer 91098 of the post 91268 and the connecting portions 91221 of the outer paddles 91220. The annular retainer 91098 can be configured similar to the annular retainer 91098; thus, the description of the annular retainer 91098 applies equally to the annular retainer 91098. The annular retainer 91098 can be a ring, a washer, a nut, or that like that is connected to the post 91268. In the illustrated example, the annular retainer 91098 is integrally formed with the post 91268.

FIG. 87 illustrates an assembled state for the device 91200 in which the post 91268 is received through the gap 91292 in the connection portion 91272 and the retainer 91098 and the connecting portions 91221 of the outer paddles 91220 are received in the recessed portions 91294. The device 91200 is assembled in the same manner as the device 91000. For example, the post 91268 and connecting portion 91266 of the outer frame portions 91256 and the connection portion 91272 of the inner frame portions 91260 are pulled away from each other along the axis V.

The paddle frames 91224 can be made from or comprise a material that allows the post 91268 and connecting portion 91266 of the outer frame portions 91256 and the connection portion 91272 of the inner frame portions 91260 to be pulled away from each other. For example, the paddle frames 91224, or a portion thereof, can be made of a laser cut or otherwise formed flexible material, such as metal, plastic, etc.

In the illustrated example of FIGS. 87-92, the connecting portions 91266 are more rigid, such that the outer frame portions 91256 and will retain their general shape more when the post 91268 is extended to narrow the outer frame portions. The connecting portions 91266 can be configured in a variety of ways to be more rigid. For example, the thickness of the connecting portions 91266 and/or the material used in the connecting portions can provide more rigidity. In some implementations, the thickness of the connecting portion 91266 can be greater than the outer frame portions 91256 to provide more rigidity. Further, in some implementations, the material used in the connecting portions 91266 can be a more rigid material to provide more rigidity.

Once the post 91268 and the connecting portion 91266 are separated from the connection portion 91272, the annular retainer 91098 and the connecting portions 91221 of the outer paddles 91220 can be placed therebetween and the distal ends of the first retaining portion 91282 and a second retaining portion 91284 can be compressed toward each other. In the compressed state, the annular retainer 91098 can be received over the first retaining portion 91282 and a second retaining portion 91284.

The annular retainer 91098 and the connecting portion 91221 of the outer paddles 91220 can be aligned with the recessed portions 91294 and the retaining portions 91282, 91284 can then be released to return toward the uncompressed state to capture the annular retainer 91098 and the connecting portion 91221 of the outer paddles 91220 in the recessed portions 91294.

Once the annular retainer 91098 and the connecting portions 91221 of the outer paddles 91220 are received in the recessed portions 91294, the post 91268 and the connecting portions 91266 of the outer frame portions 91256 can be released, which allows the post 91268 to be received through the gap 91292 and the end surface 91271 extends past the inward transition portions 91290.

The actuation portion 91050 of the device or implant 91200 is configured to both move the paddle frames 91224 between an expanded position and a narrowed position and to move the paddles between the closed position and the open position. The actuation portion 91050 can be configured in a variety of ways. Any structure capable of selectively moving the paddle frames 91224 between an expanded position and a narrowed position and opening and closing the device can be used, such as for example, the actuation portion 91050 of FIGS. 79-80. In some implementations, the actuation portion is configured such that advancing and retracting the actuation portion itself opens and closes the device and advancing and retracting a post inside the actuation portion narrows and widens the paddles. For example, the inner paddle frame portions are moved by the actuation portion to open and close the paddles in the same or a similar manner to that shown in FIGS. 23, 27, and 30-37.

Referring to FIG. 87, in the illustrated example the actuation portion 91050 includes a receiver, such as the illustrated sleeve 91202 configured to receive a portion of the post 91268 and a plug (not shown) configured to move the post axially within the sleeve 91202 to narrow and widen the paddles. The sleeve 91202 and the plug (not shown) can be configured the same or similar to the sleeve 91102 and the plug 91103 of the device 91000 of FIGS. 79-80, thus the description of the sleeve 91102 and the plug 91103 applies equally to the sleeve 91202 and the plug (not shown) of the example of FIGS. 87-92.

As shown in FIG. 87, the sleeve 91202 is fixedly attached to the connection portion 91272 such that the post 91268 can be received within a passage 91210 that extends through the sleeve 91202.

Referring to FIGS. 89-92, in operation, to move the device 91200 from the expanded position to the narrowed position, the plug (not shown) can be moved axially relative to the sleeve 91202. Movement of the plug toward the distal end 91207 causes the plug to engage the distal end 91271 of the post 91268 and move the post 91268 in the same direction (i.e., away from the proximal portion 91205 of the device).

Movement of the post 91268 away from the proximal portion 91205, as shown by arrow Q in FIG. 91, pulls the distal end portions of the outer frame portions downward while the more rigid inner frame portions maintain the positions of the proximal end portions of the outer frame portions. As a result, the outer frame portions 91256 are drawn inward (i.e., to the narrowed position), as shown by arrows R in FIGS. 91. The device 91200 has a width WN2 (FIG. 92) in the narrowed position that is narrower than the width WE2 in the expanded position.

As shown in FIGS. 89-92, when the device 91200 moves between the expanded position and the narrowed position, the more rigid connecting portions 91266 tend to retain the shape, or deform only slightly, while the outer frame portions 91256 move inward. As shown in FIG. 92, in the narrowed position, each of the outer frame portions 91256 can optionally be configured to form a recessed or concave portion 91299 proximate the mid-point between the proximal portion 91205 and the distal portion 91207 when the outer frame portions are contracted.

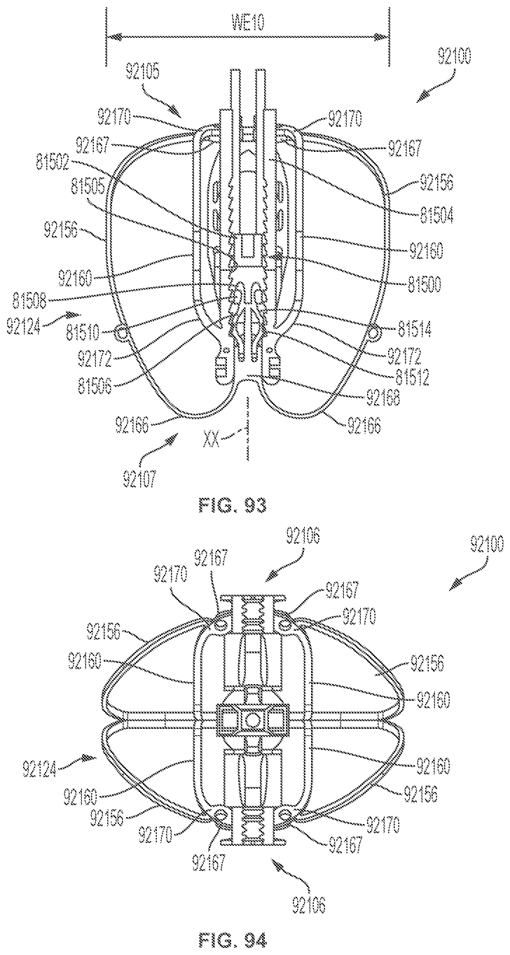

Referring to FIGS. 93-94, an example implementation of an implantable device or implant 92100 is shown. The implantable device or implant 92100 includes a proximal or attachment portion 92105, paddle frames 92124, an anchor portion 92106 attached to the paddle frames 92124, a width adjustment device 81500, and a distal portion 92107. The proximal portion 92105, the distal portion 92107, the width adjustment device 81500, and the paddle frames 92124 can be configured in a variety of ways.

In the illustrated example of FIGS. 93-94, the paddle frames 92124 are symmetric along longitudinal axis XX (FIG. 94). In some implementations of the device or implant 92100, however, the paddle frames 92124 are not symmetric about the axis WW.

In the illustrated example, the paddle frames 92124 include outer frame portions 92156 and inner frame portions 92160. In FIGS. 93-94, the outer frame portions 92156 are shown in an expanded state such that the outer frame portions 92156 define a paddle frame expanded width WE10 (FIG. 93).

The outer frame portions 92156 are flexibly attached to an attachment portion 92168 at the distal portion 92107 via connection portions 92166 and are coupled to the inner frame portions 92160 at the proximal portion 92105 via connection portions 92167. Between the connection portions 92166 and the connection portions 92167, the outer frame portions 92156 form a curved, convex shape. For example, in the illustrated example, the shape of the outer frame portions 92156 resembles an apple shape in which the outer frame portions are wider toward the proximal portion 92105 and narrower toward the distal portion 92107. In some implementations, however, the outer frame portions 92156 can be otherwise shaped.

The attachment portion 92168 is configured to attach to the width adjustment device 81500 to the outer frame portions 92156. The attachment portion 92168 can be configured in a variety of ways. Any configuration that can suitably attach the outer frame portions 92156 to the width adjustment device 81500 to allow the width adjustment device 81500 to move the outer frame portions 92156 between a narrowed position and an expanded position can be used.

The inner frame portions 92160 are jointly attached to the outer frame portions 92156 at the proximal portion 92105 via connection portions 92170 and extend from the connection portions 92170 to the distal portion 92107. The inner frame portions 92160 include retaining portions 92172 near or adjacent the distal portion 92107 for attaching to the width adjustment device 81500. The retaining portions 92172 and the width adjustment device 81500 can be configured to attach in any suitable manner.

The width adjustment device 81500 is configured to move the outer frame portions 92156 from the expanded position to the narrowed position by pulling the attachment portion 92168 and portions of the connecting portions 92166 into the width adjustment device 81500. The width adjustment device 81500 is configured to move the inner paddle frame portions 92160 to open and close the paddles in the same or a similar manner to that shown in FIGS. 23, 27, and 30-37.

The width adjustment device 81500 includes a width adjustment connection or actuator 81502, a receiver (which can be configured the as parallel racks 81504), and a coupler 81506. Each rack 81504 includes teeth 81505 that are configured to limit the motion of the coupler 81506 to a single direction (e.g., a ratchet mechanism) when the coupler is in an engaged state. In the illustrated example, the coupler 81506 is coupled to outer paddle frames 92156 by a connection portion 92168.

Still referring to FIG. 93, arms 81508 are formed on the coupler 81506 and are configured to engage projections 81510 of the actuator 81502. Resilient fingers 81512 are also formed on the coupler 81506 and are configured to engage the teeth 81505 of the rack 81504 for preventing the coupler 81506 from moving along the path L in a downward or distal direction of the racks 81504.

Still referring to FIG. 93, the actuator 81502 can be driven in either direction along the axis XX. When the actuator 81502 is driven upwards, the projections 81510 of the actuator 81502 will pull the coupler 81506 via the arms 81508 of the coupler 81506. As a result, the resilient fingers 81512 will ratchet along the teeth 81505 of the rack 81504, thereby permitting the coupler 81506 to move upwards when the actuator 81502 moves upwards. Simultaneously, the coupler 81506 will cause the connection portion 92168 to pull on the outer paddle frame portions 92156 and cause the outer frame portions 92156 to contract. In such examples, the position of the resilient fingers 81512 relative to each of the plurality of discrete positions (i.e., the teeth) on the rack 81504 can correspond to a particular width of the outer paddle frame portions.

Conversely, when the actuator 81502 is driven downwards, the projections 81510 of the actuator 81502 push against resilient, sloped surfaces 81514 of the coupler 81506. As such, the projections 81510 cause the resilient fingers 81512 to disengage from the rack 81504. As such, the coupler 81506 is disengaged from the rack 81504 when the actuator is moved in a downward or distal direction to expand the outer paddle frame portions 92156.

The paddle frames 92124 can be made from or comprise a material that allows the attachment portion 92168 and portions of the connecting portions 92166 to be pulled into the width adjustment device 81500. For example, the paddle frames 92124, or a portion thereof, can be made of a flexible metal, plastic, fabric, suture, etc. The paddle frames can be formed using a variety of different manufacturing processes, such as cutting, such as laser cutting, molding, forging, stamping, casting, bending, heat treating, shape setting, etc.

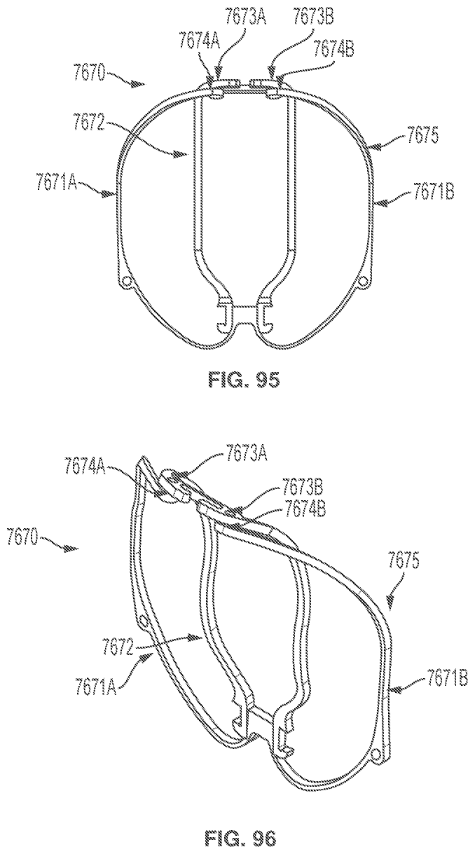

Referring to FIGS. 95 through 99, an example implementation of a connection mechanism between a rigid inner frame portion 7672 and a flexible outer frame portion 7675 of a paddle frame 7670 is shown. As shown in FIGS. 95 and 96, the proximal end of the flexible outer portion 7675 connects to the proximal end of the rigid inner portion 7672 via a pivot connection. The pivot connection can be achieved via a pin, a stitch, adhesives, or any other similar means for allowing the flexible outer portion 7675 to move relative to the rigid inner portion 7672. The proximal ends of the rigid inner portion 7672 and flexible outer portion 7675 can connect via at least one pin connection. In the example implementation, the proximal end of the rigid inner portion 7672 has first and second orifices 7673A, 7674B for receiving pins, stitches, or pin portions of the flexible outer frame 7675.

The flexible outer frame 7675 comprises a first portion 7671A and a second portion 7671B. The proximal end 7674A of the first portion 7671A is configured to connect to the rigid inner portion 7672 via the first orifice 7673A. The proximal end 7674B of the second portion 7671B is configured to connect to the rigid inner portion 7672 via the second orifice 7673B. These connections can comprise pin connections such that a pin (not shown) extends through the first orifice 7673A and the proximal end 7674A of the first portion 7671A of the flexible outer portion 7675, and a pin extends through the second orifice 7673B and the proximal end 7674B of the second portion 7671B of the flexible outer portion 7675. The pins can also be stitches, connector elements, or integral extensions of the proximal ends 7674A, 7674B of the first and second portions 7671A, 7671B of the flexible outer frame 7675.

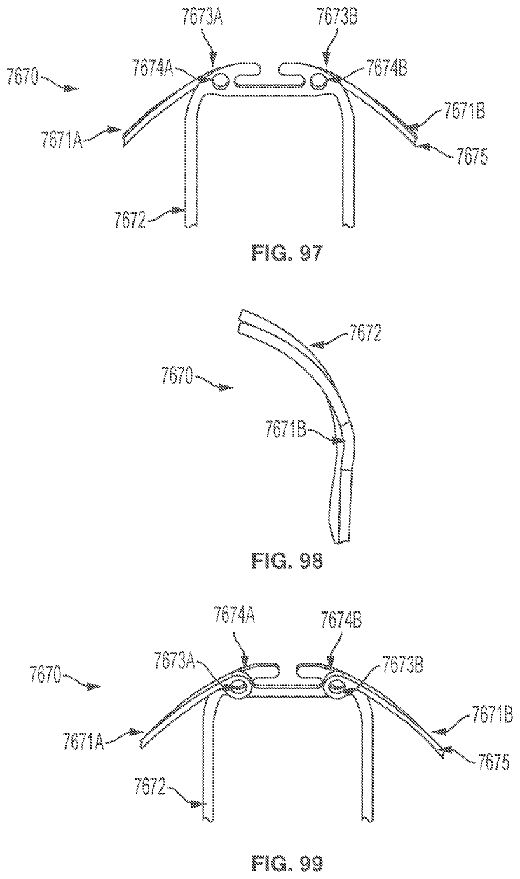

FIG. 97 shows the rigid inner portion 7672 having a first orifice 7673A and a second orifice 7673B. The first portion 7671A of the flexible outer portion 7675 connects to the rigid inner portion 7672 by affixing the proximal end 7674A of the flexible outer portion 7675 to the first orifice 7673A of the rigid inner portion 7672. The second portion 7671B of the flexible outer portion 7675 connects to the rigid inner portion 7672 by affixing the proximal end 7674B of the flexible outer portion 7675 to the second orifice 7673B of the rigid inner portion 7672. The proximal ends 7674A, 7674B can be affixed to the first and second portions 7671A, 7671B of the flexible outer portion 7675 via a pin connection, pivot connection, lamination, or any other means. The flexible outer portion 7675 of the paddle frame 7670 can be on top of the rigid inner portion 7672, as shown in FIG. 97. Alternatively, as shown in FIG. 99, the flexible outer portion 7675 can be below the rigid inner portion 7672. FIG. 98 shows a partial side view of this example of the paddle frame 7670.

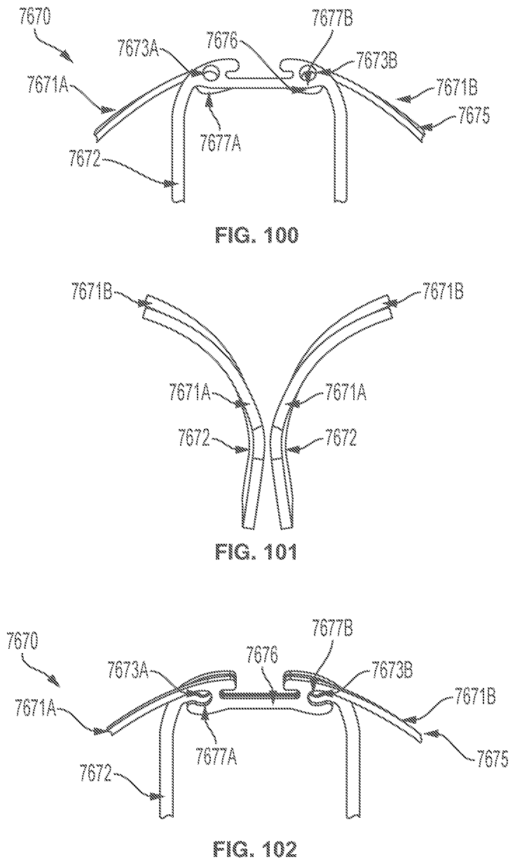

Referring to FIGS. 100 through 102, an example of a connection mechanism between a rigid inner portion 7672 and a flexible outer portion 7675 of a paddle frame 7670 is shown. As shown in FIGS. 100 and 102, the proximal end 7676 of the flexible outer portion 7675 can be laminated, or otherwise affixed, to the proximal end of the rigid inner portion 7672. The inner and outer portions 7672, 7675 can also or instead be pivotably connected via a first and second pivot point 7677A, 7677B. The pivot connection allows the flexible outer portion 7675 to bend relative to the rigid inner portion 7672. The pivot connection can be achieved via a pin, a stitch, connection element, adhesives, or any other similar means for allowing the flexible outer portion 7675 to move relative to the rigid inner portion 7672. In this example, the pivot connection is such that the flexible outer portion 7675 is on the outside of the rigid inner portion 7672. However, as shown in FIG. 102, the flexible outer portion 7675 could also be on the inside of the rigid inner portion 7672.

The proximal ends of the rigid inner portion 7672 and flexible outer portion 7675 can connect via at least one pivot. In some implementations, the proximal end of the rigid inner portion 7672 has a first and second orifice 7673A, 7673B for receiving a pin, a stitch, connection element, or pin portions of the flexible outer frame 7675. The flexible outer frame 7675 comprises a first portion 7671A and a second portion 7671B. A first pivot point 7677A of the first portion 7671A is configured to connect to the rigid inner portion 7672 via the first orifice 7673A. The second pivot point 7677B of the second portion 7671B is configured to connect to the rigid inner portion 7672 via the second orifice 7673B. These connections can comprise pin connections such that a pin (not shown) extends through the first orifice 7673A and the proximal end 7674A of the first portion 7671A of the flexible outer portion 7675, and a pin extends through the second orifice 7673B and the proximal end 7674B of the second portion 7671B of the flexible outer portion 7675. The pins can also be integral extensions of the proximal ends 7674A, 7674B of the first and second portions 7671A, 7671B of the flexible outer frame 7675. FIG. 101 shows a partial side view of this example of the paddle frame 7670.

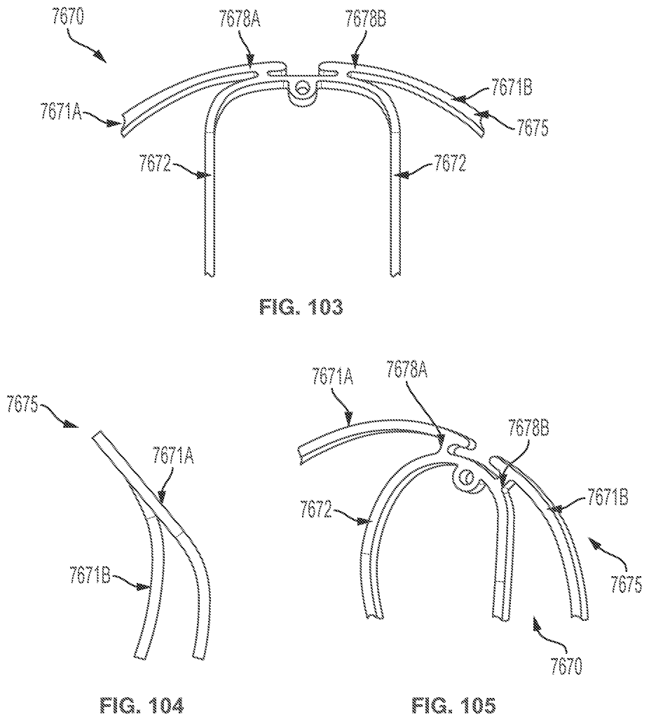

Referring to FIGS. 103-105, an example of a connection mechanism between a rigid inner portion 7672 and a flexible outer portion 7675 of a paddle frame 7670 is shown. The proximal ends of the inner and outer portions 7672, 7675 of the paddle frame 7670 are integrally joined together. The connection can be formed from one single piece having a first pivot point 7678A and a second pivot point 7678B. The first pivot point 7678A is located at the integration point between a first portion 7671A of the flexible outer portion 7675 with the rigid inner portion 7672. The second pivot point 7678B is formed from the integration point between the second portion 7671B of the flexible outer portion 7675 and the rigid inner portion 7672. Although the flexible outer portion 7675 and rigid inner portion 7672 are formed with one piece, the flexible outer portion 7675 can flex relative to the rigid inner portion 7672 via the first and second pivot points 7678A, 7678B.

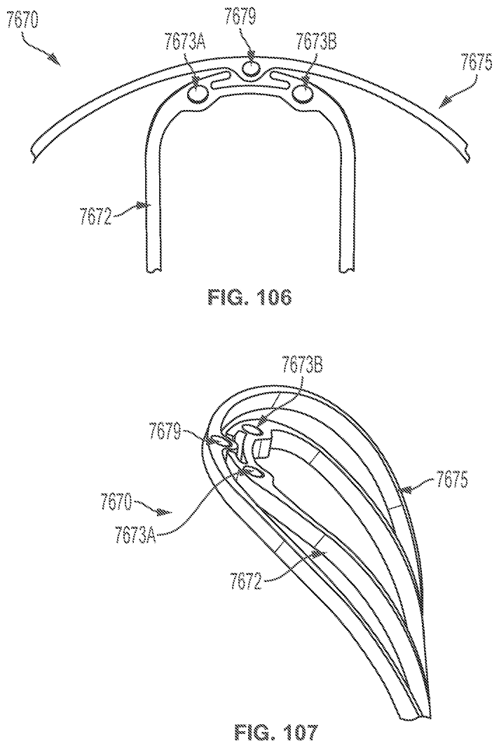

Referring to FIGS. 106 and 107, an example of a connection mechanism between a rigid inner portion 7672 and a flexible outer portion 7675 of a paddle frame 7670 is shown. In this example, the proximal ends of the outer and inner portions 7672, 7675 of the paddle frame 7670 are nested together. The flexible outer portion 7675 can have an orifice 7679 proximate to a first and second orifice 7673A, 7673B within the proximal end of the rigid inner portion 7672. The inner and outer portions 7672, 7675 of the paddle frame 7670 can be connected by any means, including but not limited to: nesting together within a cover, stitched together via the orifices 7679, 7673A, 7673B, or connected via connectors (not shown) that extend between the first and second orifices 7673A, 7673B and the orifice 7679 on the flexible outer portion 7675.

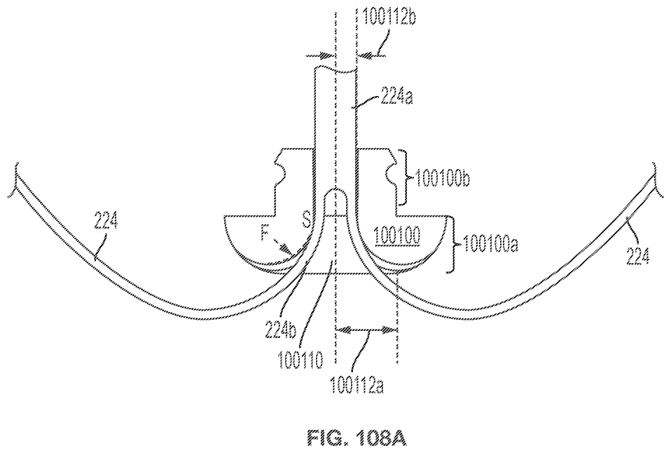

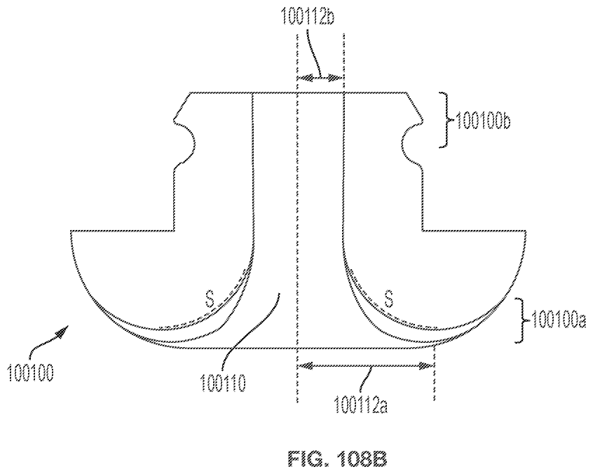

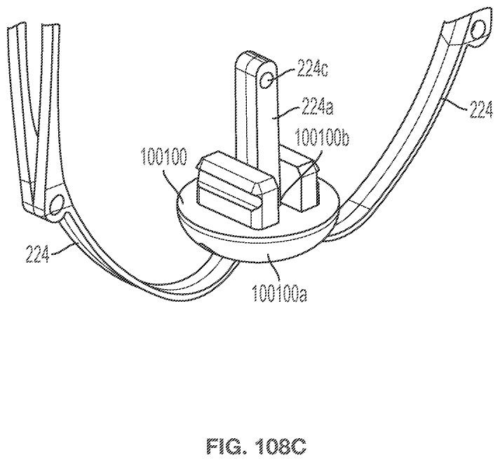

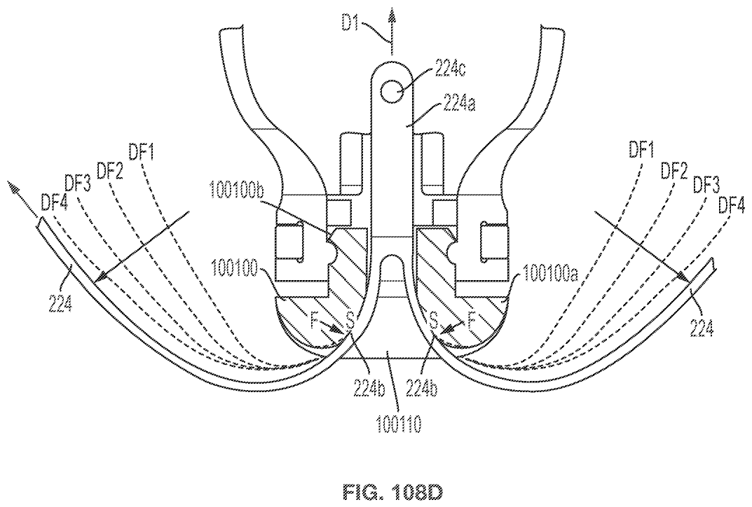

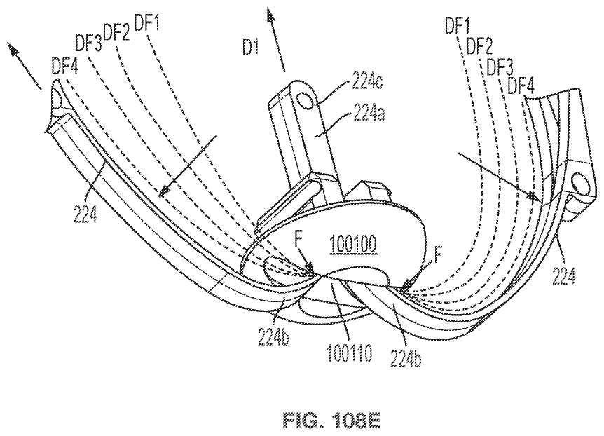

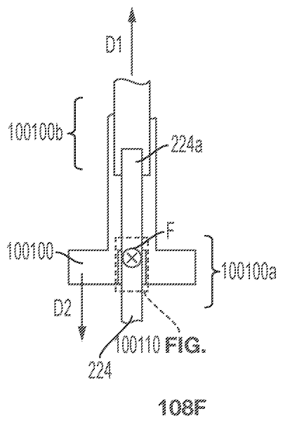

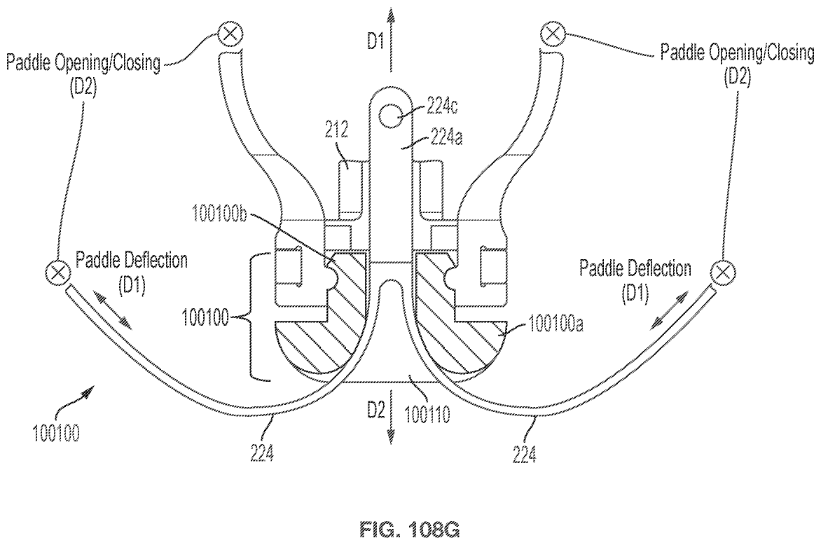

FIGS. 108A-108G illustrate an example the cap 100100 that reduces stress applied to portions of a paddle frame 224 that are pulled through the cap by providing a radiused entry point or hole into the cap 100100. This radiused entry point increases the radius of curvature of the portion of the paddle frame that is pulled into the cap and therefore reduces the stress that is introduced into the paddle frame. The radiused hole or entry point 100110 in the cap 100100 can guide the paddle frames 224 through a series of deflections.

FIG. 108A shows the cap 100100 engaged with the paddle frame 224. FIG. 108B shows a close-up of the cap 100100 without the paddle frame 224 for clarity. Both FIGS. 108A and 108B show the cap 100100 in cross-section. FIG. 108C shows an external side view of the cap 100100 and the paddle frame 224 arrangement shown in FIG. 108A. As shown in FIGS. 108A and 108B, the cap 100100 includes a radiused hole or entry point 100110 that accommodates at least an inner end 224a of the paddle frame 224.

The radiused hole or entry point 100110 provides a mechanism via which the cap 100100 can control the paddle frame 224 deflection in a manner that introduces less stress to the paddle frame 224. In particular, the radiused hole 100110 has a radius 100112a at the distal end 100100a of the cap 100100 that is larger than radius 100112b at the proximal end 100100b (FIGS. 108A and 108B). (Note—“Proximal” and “distal” are herein used to refer to relative distances with respect to the user.) The difference in radius between the radius 100112b and the radius 100112a is bridged by a slope S of the portion of the hole 100110 in contact with the paddle frame portion 224b. Because of slope S, relative motion of the cap 100100 with respect to the paddle frame 224 can impart force F (see, e.g., FIG. 108A) on portion 224b. This force F is significantly less than would be the case if the hole 100110 were cylindrical and an inside surface of the hole and a distal end of the cap form a right angle. Since the paddle frame 224 is generally made of material that can substantially hold its shape without plastically deforming, force F tends to deflect the paddle frame 224 upward/downward throughout its length.

The paddle frame 224 can include an attachment portion 224c that allows the paddle frame 224 to attach directly to another portion of the device for mechanical communication. For example, attachment portion 224c can be attached to a mechanism for pulling of the paddle frames 224 into the cap to reduce the width of the paddle frames and pushing the paddle frames 224 out of the cap to increase the width of the paddle frames.

Deflection of the paddle frame 224 via cap 100100 is shown in more detail in FIGS. 108D and 108E. FIG. 108D shows the motion in cross-section, while FIG. 108E shows the same motion from a perspective exterior, side view. FIGS. 108D and 108E show a range of deflection DF1-DF4 facilitated by moving the paddle frame 224 along direction D1 into the cap 100100 (i.e., as the paddle frames 224 are pulled into the cap). Direction D1 extends from the distal end 100100a to the proximal end 100100b of the cap 100100. The hole 100110 extends from the proximal end 100100b of the cap 100100 to the distal end 100100a in order to accommodate the inner end 224a of the paddle frame 224. In this way, moving the paddle frame 224 into the cap 100100 in direction D1 causes the paddle frame 224 to deflect toward the cap 100100 (e.g., deflecting the paddle frame 224 from DF4 to DF1). Pushing the paddle frame 224 out of the cap 100100 opposite the direction D1 causes the paddle frame 224 to deflect in the other outward (e.g., deflecting the paddle frame 224 from DF1 to DF4).

FIG. 108F shows how the cap 100100 can be used to both (simultaneously or separately) deflect (expand and contract) the paddle frames 224 (as shown in FIGS. 108D and 108E) and open and close the paddle frames 224 via an actuation element (e.g., the same as or similar to actuation element 112 shown in FIGS. 8-20 or actuation element 8102 shown in FIGS. 26-30). FIG. 108F is a cross section of the cap 100100 that is perpendicular to the cross-sectional views shown in FIGS. 108A and 108D. The difference in view is revealed by comparing the relative orientation of force F imparted by the cap 100100 to deflect the paddle frames 224 in FIG. 108F with the orientation of force F in FIGS. 108A and 108D. In FIG. 108F, force F points into the page. In contrast, force F is parallel to the page in FIGS. 108A and 108D.

In FIG. 108F, motion of a width adjustment element 100003 (e.g., width adjustment control, width adjustment wire, width adjustment shaft, width adjustment tube, width adjustment line, width adjustment cord, width adjustment suture, width adjustment tether, etc.) along direction D1 results in the same motion of the paddle frames 224 into the cap 100100 discussed in the context of FIGS. 108D and 108E above. In FIG. On the other hand, motion along D2 represents an actuation or relative motion of an actuation element (e.g., the same as or similar to actuation element 112 shown in FIGS. 8-20 or actuation element 8102 shown in FIGS. 26-30) and accompanying movement of the cap 100100 to open and close the paddle frames 224. FIG. 108F shows how motion of the paddle frames into the cap in the direction D1 can occur independently of moving the cap 100100 in direction D1, and vice versa. Motion along of the paddle frames 224 into the cap in direction D1 and movement of the cap in direction D2 can also occur concurrently. That is, motion of actuation element and cap 100100 in the direction D2 can open the paddle frames 220 (as shown in FIGS. 30-36) at the same time that motion of the paddle frames into the cap 100100 in direction D1 to deflect the paddles inward (as shown in FIGS. 108D and 108E). FIG. 108G shows the difference in the paddle motion between 1) the deflection caused by pulling the paddle into the cap 100100 in direction D1 and 2) the opening/closing of both the rigid and flexible portions of the paddle frames (into and out of the page) caused by moving the cap with the actuation element in the direction D2.

As discussed above, in some implementations, the paddle frames 224 can be narrowed/widened, stopped, locked, and held in fully expanded, fully narrowed, and in intermediate positions. Locking can also be particularly helpful prior to leaflet the capture in keeping the paddle frames 224 narrow to traverse potential obstructions, such as the chordae tendineae (CT, FIGS. 3 and 5).

In some implementations, the paddle narrowing and widening adjustment mechanism automatically holds the paddle frames 224 in the adjusted position when the mechanism is released. A wide variety of different mechanisms can be used to narrow and widen the paddle frames and automatically hold the paddle frames 224 in any adjusted position. For example, a screw mechanism, a ratchet mechanism, a cam mechanism, etc. can be used. Such mechanisms allow the user to set and maintain the paddle frames 224 at any width so that a particular the paddle width is maintained without active tensioning action by the user. Additionally, such mechanisms can facilitate more precise control of narrowing/widening.

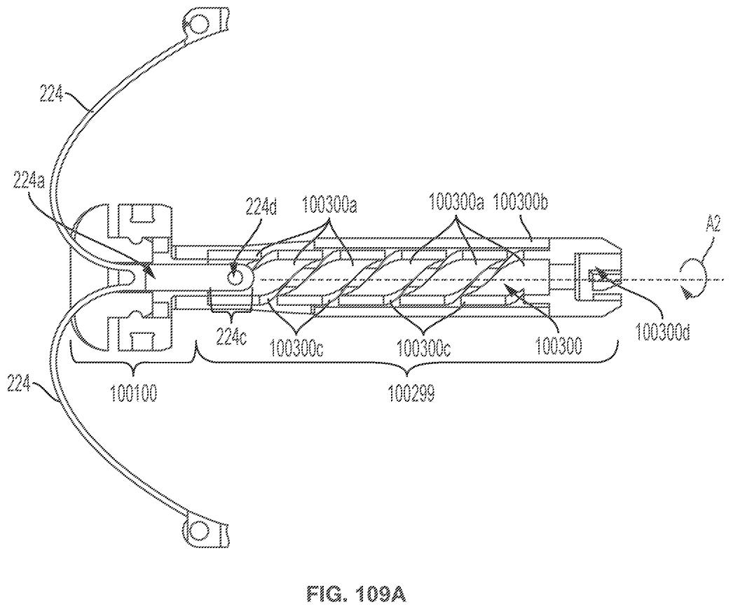

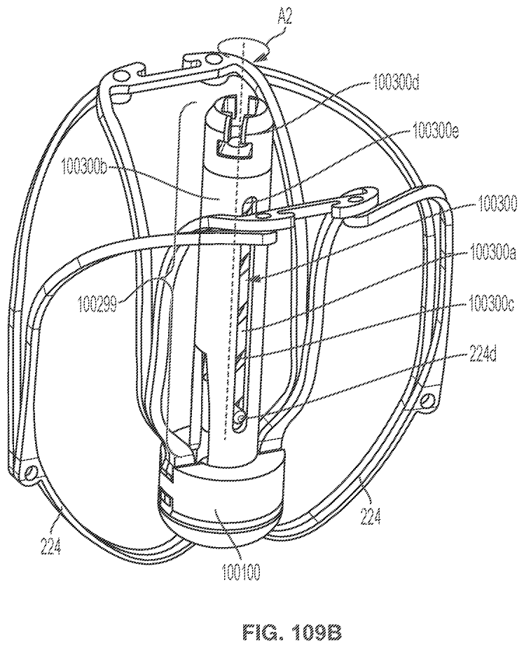

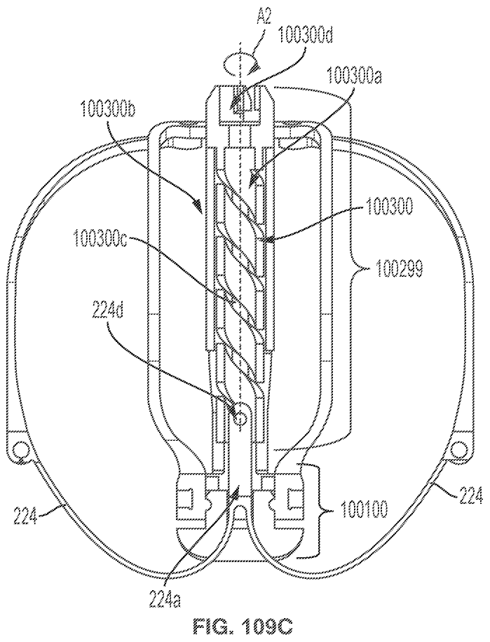

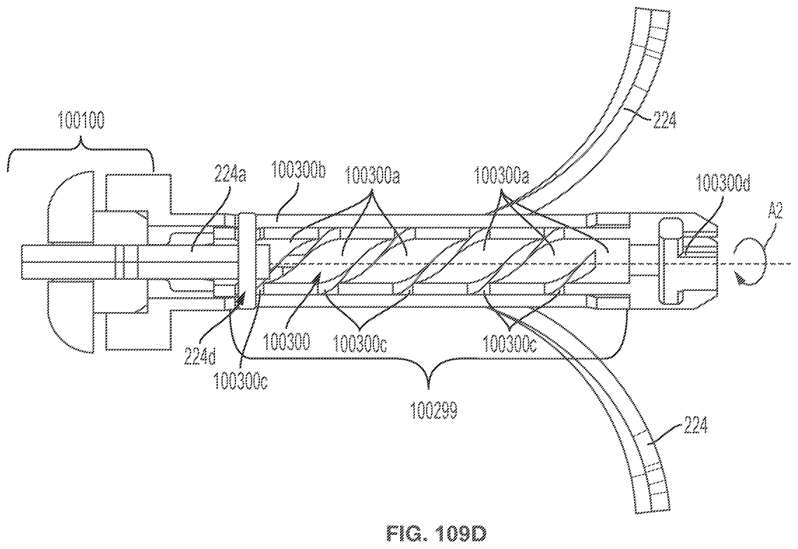

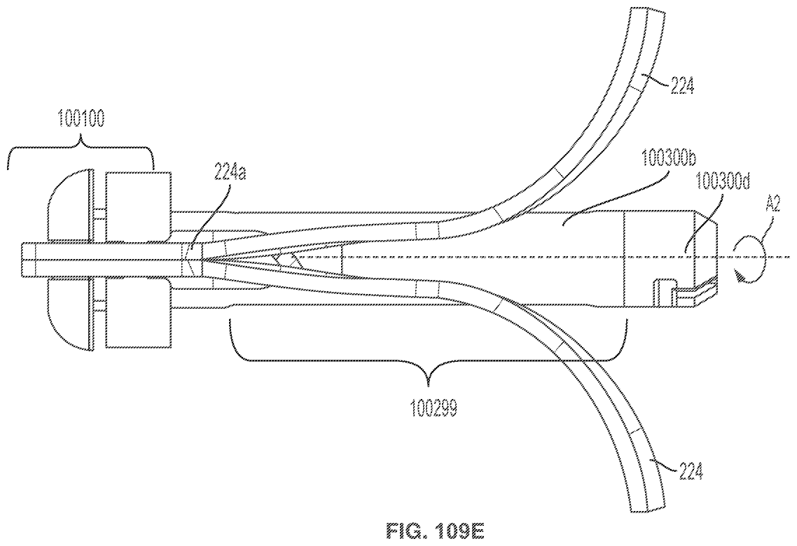

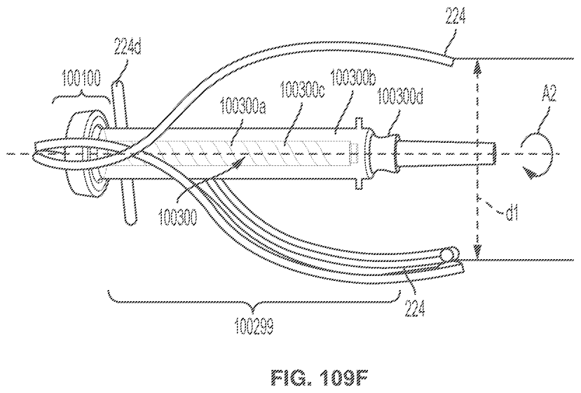

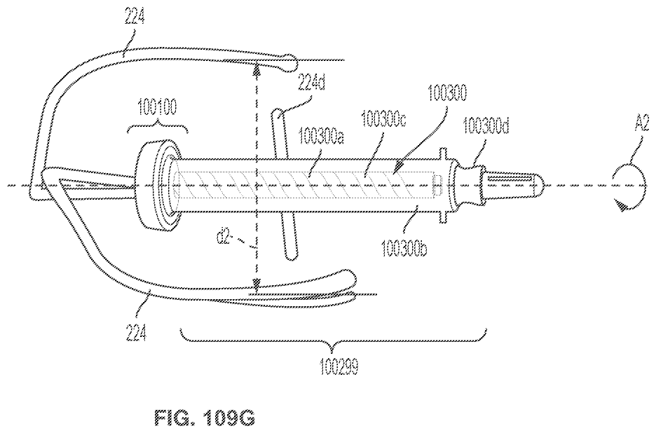

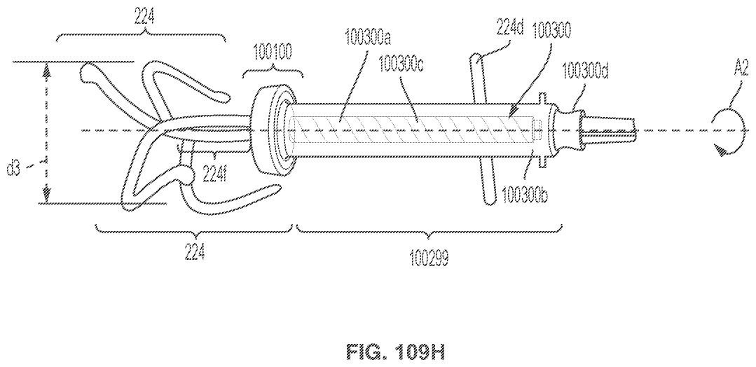

FIGS. 109A-109E illustrate an example implementation of a paddle narrowing and widening adjustment mechanism 100299 that automatically holds the paddle frames 224 in the adjusted position when the mechanism is released. The mechanism includes a coupler, such as the illustrated rotational member 100300. In addition to adjusting the width of the paddles, linear movement of the entire mechanism 100299 can be used to open and close the paddle frames with an actuation element (e.g., the same as or similar to actuation element 112 shown in FIGS. 8-20 or actuation element 8102 shown in FIGS. 26-30). One advantage of the rotational member 100300, is that the member can hold a paddle width without the use of a separate locking mechanism. Another advantage is that the rotational member 100300 can allow precise control of the paddle frame 224 width. Another advantage is that a single element can be used to both open and close the paddle frames and adjust the width of the paddle frames.

In some implementations, the paddle narrowing and widening adjustment mechanism 100299 is a helical screw system that extends the paddle frames 224 by moving the inner end 224a along the axis A2. The details of this mechanism are discussed below. The motion of rotational member 100300 can be driven by components located in the proximal portion 100300d of the device. It is to be understood that the configuration shown in FIGS. 109A-109E are merely examples. Variations in locations of components and specific construction are possible and within the scope of this disclosure. In addition, the cap 100100 is shown in FIGS. 109A-109E for illustrative purposes. It is also to be understood that other variations can use different the caps and/or other components. For example, the cap 100100 shown in FIG. 109A can be the cap 100100 shown in FIGS. 108A and 108B with one or more of the optional stress reducing features. In some implementations, the cap 100100 shown in FIG. 109A does not include the optional stress reducing features. For example, in some implementations, the cap 100100 shown in FIG. 109A can have a cylindrical hole with an inside surface that meets a distal end of the cap at a right angle (i.e., the opening of the cap is not rounded).

FIGS. 109A and 109C are cutaway, cross-sectional views of the paddle narrowing and widening adjustment mechanism 100299 to illustrate how it moves the paddle frames 224. As shown in FIGS. 109A and 109C, member 100300 includes a helical portion 100300a. The cutaway views of the helical portion 100300a in FIGS. 109A and 109C show six sections (the helical portion 100300a can have any number of sections). This is an artifact of the cutaway view. In fact, each of the six sections of helical portion 100300a are joined as a solid, continuous ribbon of material that makes a helical shape. The formation of the helix creates a slot 100300c in between each of the six sections. The helical portion 100300a is surrounded by a receiver, such as the illustrated case 100300b. The helical portion 100300a is rotatable about axis A2 as shown in FIGS. 109A-109C, while the case 100300b remains fixed. The helical portion 100300a, a slot 100300c, and the case 100300b are shown in in an exterior (as opposed to in cutaway) view in FIG. 109B.

FIG. 109A shows that an inner end 224a of the paddle frame 224 interacts with the helical portion 100300a via a protrusion 224d, such as a post. In FIGS. 109A and 109C, the protrusion 224d points from portion 224c into/out of the page. FIG. 109B shows the protrusion 224d extending away from the inner end 224a of the paddle frame 224 to which it is attached. As shown in FIG. 109B, the protrusion 224d fits into the helical slot 100300c in the helical portion 100300a and a linear slot 100300e in the case 100300b. The case 100300b is fixed with respect to rotation of the helical portion 100300a. Therefore, rotation of the helical portion 100300a about the axis A2 causes the protrusion 224d to move in a way that is guided by both slot 100300c and slot 100300e. In particular, when the helical portion 100300a is rotated around the axis A2, the slot 100300c pushes the protrusion 224d (and therefore the inner end 224a) along the 100300e and therefore along axis A2. The direction in which the protrusion 224d moves along the axis A2 (i.e., either towards the cap 100100 or in an opposite direction towards the proximal portion 100300d) depends on the direction of rotation of helical portion 100300a. As discussed above, motion of protrusion 224d causes the paddle frames 224 to either be drawn into or pushed out of the cap 100100 depending on the direction of rotation. In some implementations, whenever rotation of the helical portion 100300a stops, the paddle narrowing and widening adjustment mechanism 100299 holds the paddle frames 224 at the corresponding width.

Mechanisms for actuating the rotation of helical portion 100300a can include, for example, a user rotated rod, handle, or other fixture. The rotation can be manually activated, electronically activated, and/or controlled via software/computer interface. Other implements can include using a stepper motor, remotely or locally controlled, and/or any other suitable actuator. Such mechanisms can be coupled to the proximal portion 100300d in a wide variety of different ways, such as by any of the coupling arrangements disclosed in the present application. In some implementations, the coupling to the proximal portion 100300d facilitated both rotation of the helical portion 100300a to adjust the width of the paddle frames and linear extension of the entire paddle narrowing and widening adjustment mechanism 100299 relative to a delivery catheter and/or a coaptation element to open and close the paddles of the device.

FIGS. 109D and 109E show the rotation of helical portion 100300a from a side of the device. In both FIGS. 109D and 109E, the paddle frames 224 are in a widest position. FIG. 109D shows a cross-sectional view of helical portion 100300a. FIG. 109E shows this the paddle frame configuration, but with an exterior view of housing 100300b. As is shown in FIG. 109D, the protrusion 224d extends through the inner end 224a, through the helical slots 100300c of the helical portion 100300c, and through the elongated slot 100300e of the housing 100300b.

FIGS. 109F, 109G, and 109H show portions of the paddle frames 224 in three different width positions (i.e., three different amounts of the paddle frames drawn into the paddle narrowing and widening adjustment mechanism 100299). The different positions are created in succession by rotating the helical portion 100300a about axis A2 in order to move protrusion 224d from distal (FIG. 109F) to more proximal (FIG. 109H) positions.

FIG. 109F shows the beginning of the movement with the protrusion 224d close to the cap 100100 at the distal end of member 100300. As shown in FIG. 109F, in this position both sets of the paddle frames 224 are in the widest position. The two paddle frames 224 are a distance d1 from one another. As the helical portion 100300a is rotated around axis A2, the slot 100300c pushes the protrusion 224d toward the proximal end of rotating member 100300 (e.g., toward end 100300d).

Continuing this rotation results in the reduced amount of the paddle frames 224 extending from the cap 100100 shown in FIG. 109G. In FIG. 109G, the protrusion 224d has now been moved to a middle position between proximal and distal portions of the rotating member 100300. Correspondingly, the paddle frames 224 now have partially narrowed. In this partially extended position, the two paddle frames 224 have a distance d2 between each other. The paddle frames 224 can be configured such that the distance d2 is greater than the distance d1 (i.e., the paddle frame portions move apart as they are narrowed by retraction into the mechanism) or such that the distance d2 is less than the distance d1 (i.e., the paddle frame portions move toward one another as they are narrowed by retraction into the mechanism).

Continuing the rotation of helical portion 100300a around axis A2 further pushes protrusion 224d towards the proximal end of member 100300 (i.e., toward 100300d). The result is shown in FIG. 109H. The paddle frames 224 are further narrowed by retraction into the paddle narrowing and widening adjustment mechanism 100299. In this position, the two paddle frames 224 have a distance d3 between each other. The paddle frames 224 can be configured such that the distance d3 is greater than the distance d2 (i.e., the paddle frame portions move apart as they are narrowed by further retraction into the mechanism) or such that the distance d3 is less than the distance d2 (i.e., the paddle frame portions move toward one another as they are narrowed by further retraction into the mechanism).

In some implementations, the paddle frames 224 are actively narrowed and passively expanded. As such, the expanded condition can be the natural or substantially unstressed shape of the paddle frames. To move the paddle frames to the narrowed condition the paddle frames are stressed to flex the paddle frames from the expanded state to the narrowed state. This stress can be concentrated in certain areas of the paddle frames 224, such as at the area where the paddles enter the cap. As described above, one way of reducing this stress is to provide a radiused or tapered entry for the paddles into the cap.

In some implementations, the paddle frames are structurally modified to reduce the stress in the area where the paddle frames enter the cap. The paddle frames can be structurally modified to reduce the stress in the area where the paddle frames enter the cap in a variety of different ways. For example, the area where the paddle frames enter the cap can be moveably connected to the remainder of the paddle frame, the area where the paddle frames enter the cap can be decoupled from the remainder of the paddle frame, the area where the paddle frames enter the cap connected to the remainder of the paddle frame by a flexible component, etc.

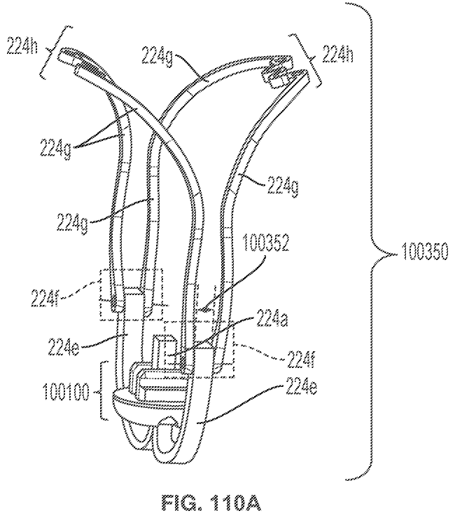

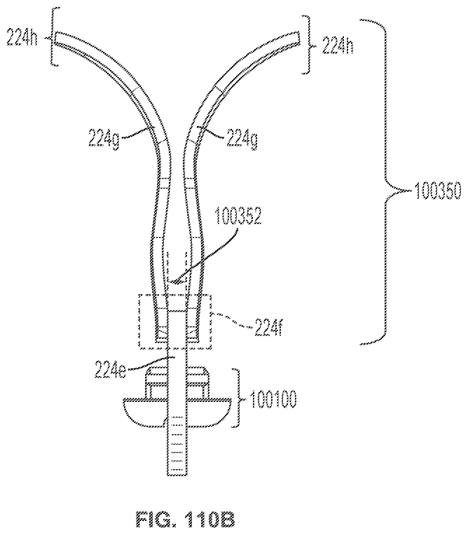

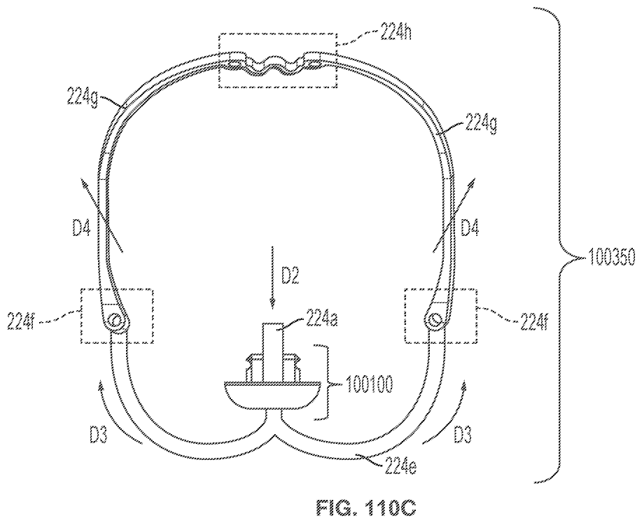

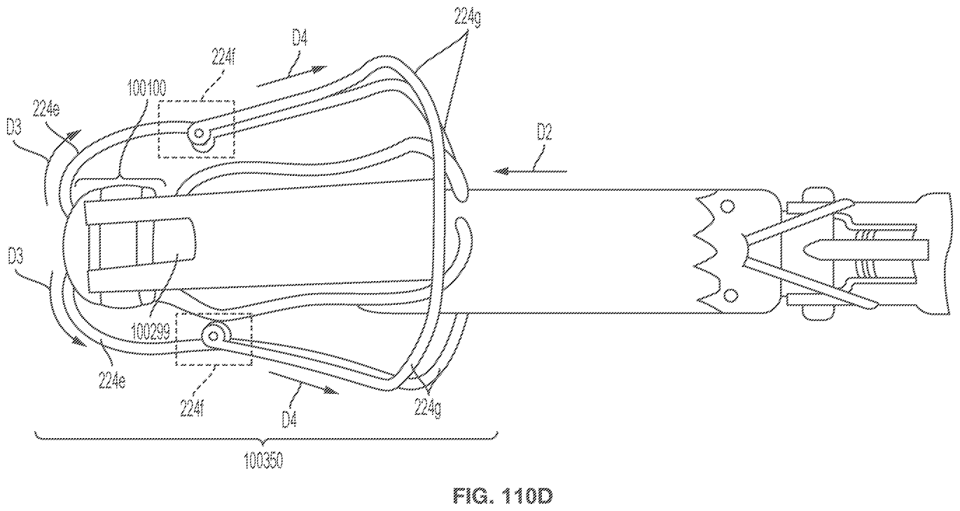

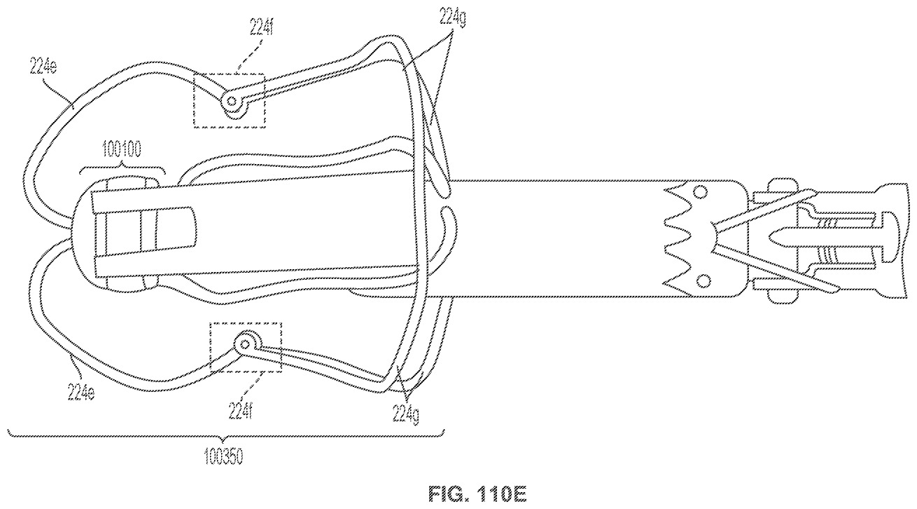

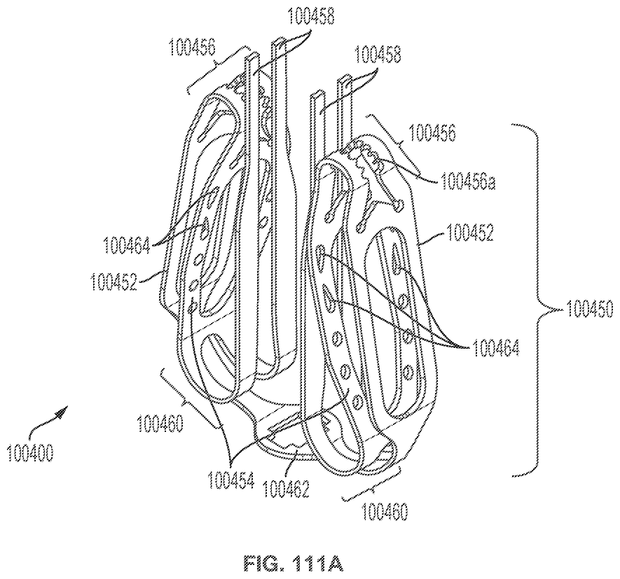

FIGS. 110A-110E show a paddle system 100350 that addresses the problem of stress concentration at the portions of the paddle frames 224 that are pulled into the cap, in part, by segmenting the paddle construction. The paddle system 100350 comprises a connector or lower portion 224e (e.g., shaped metal component, shaped plastic component, tether, wire, strut, line, cord, suture, etc.) and an upper portion 224g joined by a pivot point 224f. Although FIGS. 110A-110E show the system 100350 being used in conjunction with the cap 100100, it is to be understood that this is merely by way of example. System 100350 can be used in conjunction with other components disclosed and/or implied herein.

FIGS. 110A-110C show different views of the system 100350. FIGS. 110A-110C show the pivot point 224f in the form of a hinge. A hinge would allow the user to actively increase the paddle frame 224 width by pushing inner end 224a in direction D2 (FIG. 110C) and to decrease the paddle width by pulling the inner end 224a in the opposite direction. More specifically, pushing the inner end 224a in the direction (e.g., moving protrusion 224d pushes lower portion 224e along direction D3, while the lower portion 224e and the upper portion 224g are free to pivot relative to one another about the pivot point 224f. In turn, this causes the upper portion 224g to bow along direction D4, thus expanding the paddle frame 224. Pulling the inner end 224a in the opposite direction pulls the lower portion 224e into the cap 100100, while the lower portion 224e and the upper portion 224g are free to pivot relative to one another about the pivot point 224f. In turn, this causes the upper portion 224g to bow inward (opposite direction D4), thus narrowing the paddle frame 224.

In some implementations, the bowing of the upper portion 224g is resisted and/or provided with a countervailing restoring force by resilient element 224h. Resilient element 224h can allow the active narrowing or widening of the paddle frame 224 width to be automatically or passively reversed. The resilient element 224h can include a spring, as shown in FIGS. 110A-110C. However, it is to be understood that other variations can include other types of biasing mechanisms (e.g., leaf springs, coil springs, etc.), or any other restoring mechanism described herein can be used.

As shown in FIGS. 110A and 110B, the positioning of the lower portions 224e between the upper portions 224g at the pivot points 224f of the system 100350 creates a spacing 100352 between adjacent upper portions 224g of the paddle frame 224. The spacing 100352 can prevent or inhibits the upper portions 224g from pinching or restricting the valve leaflets of the valve being repaired. That is, the spacing 100352 can reduce pinching of the free edge of the leaflet between upper portions 224g. The spacing 100352 can be adjusted based on the selection and fabrication of lower portions 224e (e.g., their thickness) at the pivot point 224f.

Using the pivot point 224f to connect a segmented upper 224g and lower 224e portions of the paddle frames 224 can facilitate certain manufacturing advantages. Segmenting the upper 224g and lower 224e portions allows these portions to be fabricated from different materials and/or via different methods. For example, the lower frame portion 224e can be fabricated by stamping or laser cutting a ribbon of more flexible material and/or a stronger material that can withstand the application of higher strains, while upper portion 224g can be fabricated using a less flexible and/or weaker materials. The upper portion 224g can be made of a less expensive material, such as a bent wire.

FIGS. 110D and 110E show an implementation of an implantable device or valve repair device or implant that includes the hinged paddle system 100350. The valve repair device or implant can include any of the features of any of the other devices or implants disclosed in the present application. By comparing FIGS. 110D and 110E, the user moves ends of the paddle portions in the direction D2, for example out of a cap 100100 with a paddle narrowing and widening adjustment mechanism 100299. This pushes lower portion 224e along direction D3. That motion of lower portion 224e, then actuates upper portion 224g, via pivot point 224f, to move along D4.

FIG. 110E shows a result of this motion. As shown in FIG. 110E, both lower portion 224e and upper portion 224g have been extended to widen the paddle frames. As discussed above, this motion extends the paddle frames 224 with diminished stress concentration on the system 100350. Note that, although optional restoring member 224h is not shown in FIGS. 110D and 110E, it is to be understood that restoring member 224h can be included. If so, restoring member 224h can create a mechanical bias toward returning the paddle frame 224 extent to its original fully widened or fully narrowed position.

As discussed above, there are advantages to fabricating portions of the disclosed the devices (e.g., implantable device/implant 200) out of bulk materials, such a sheet material, such as a sheet of metal or plastic, rather than from a braided or woven networks of wire. Braided or woven networks can facilitate flexibility of design by which the device can elongate and compress into a tracking condition to enable delivery through a delivery system and intraprocedural maneuvering and expand into the shape of the implantable device or implant. However, devices made from or comprise a braided or woven network of wires can be expensive to manufacture. In some implementations, portions of the valve repair device or implant can be made from a flat sheet of material. For example, coaptation element supports, inner paddle portions, outer paddle portions, and/or a paddle frame connection portion can be made from a flat sheet of material.

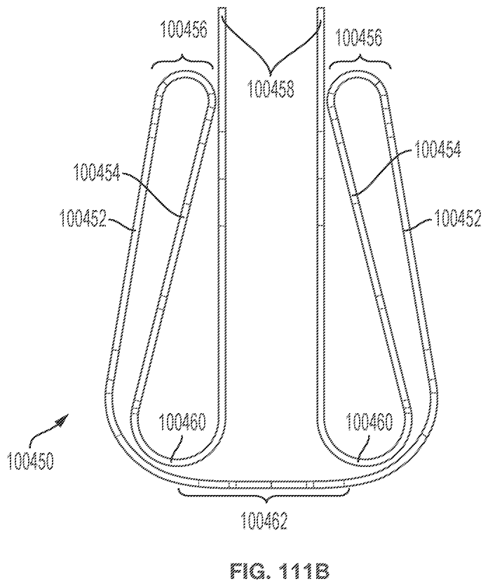

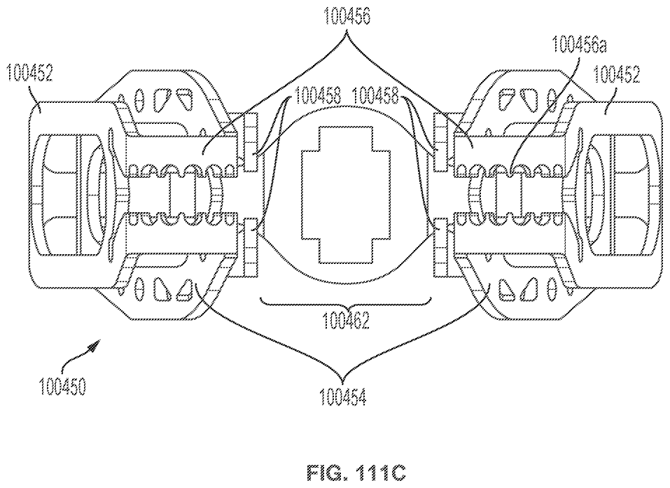

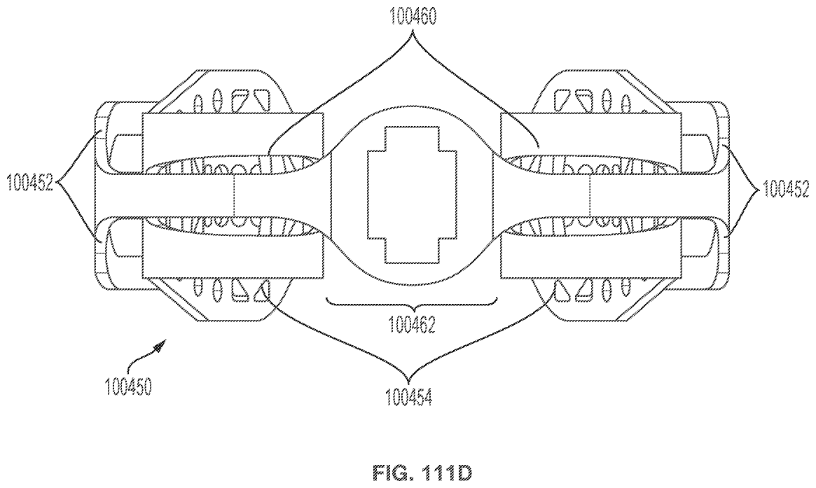

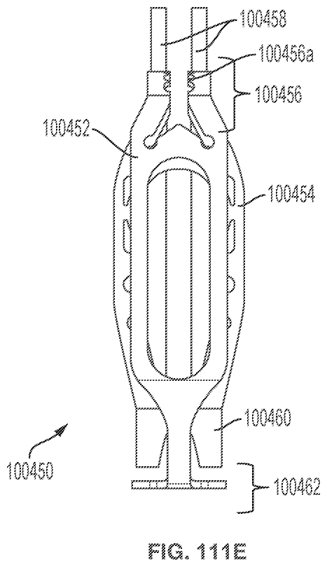

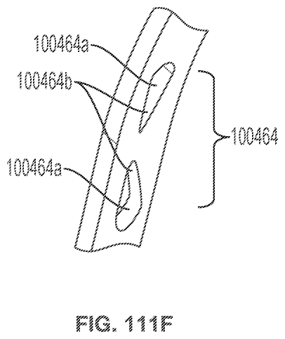

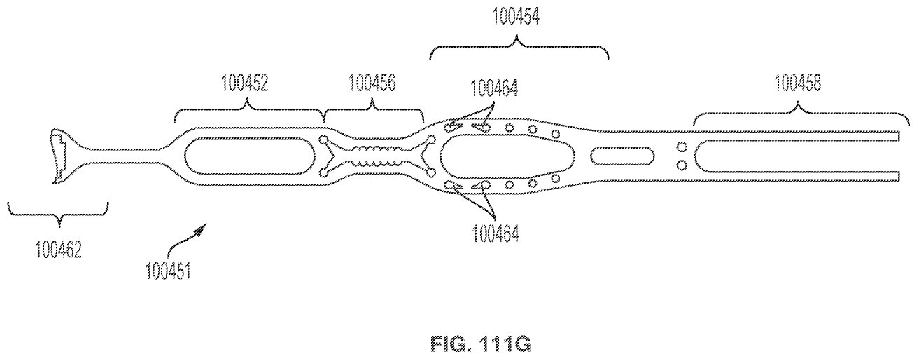

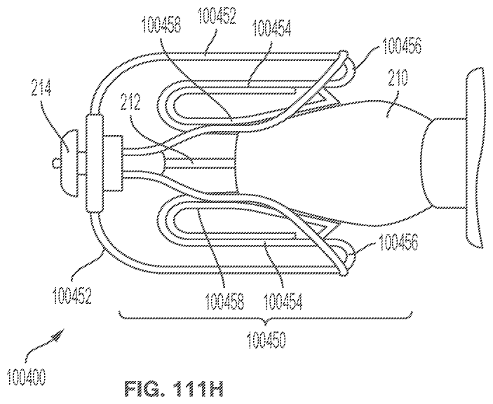

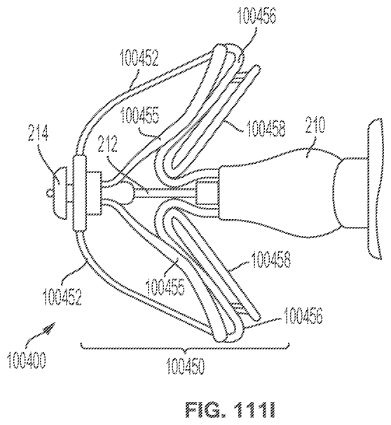

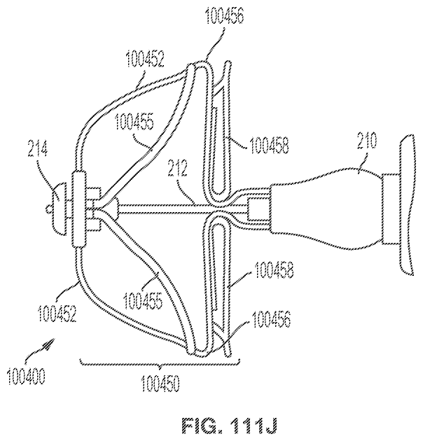

FIGS. 111H-111J show a device 100400 having a paddle structure 100450 illustrated by FIGS. 111A-111G. FIGS. 111A-111G depict a paddle structure 100450 in which a braided or wire paddle structure is replaced by a structure 100450 fabricated from a sheet of material. In the illustrated example, the paddle structure 100450 is made from a single, contiguous piece of material (e.g., a nitinol flat sheet or strip of material that can be laser cut, photo etched, or stamped as a flat part and subsequently shaped). The precise material and manufacturing method can vary.

Comparison of FIG. 111A with FIG. 22 shows that the paddle structure 100450 takes a similar form as the paddle structure 220. Table 1 below compares components in the paddle structure 100450 with functionally similar components in braided or woven variation shown in FIG. 22. The components in the paddle structure 100450 are shown in a perspective view in FIG. 111A, side view in FIG. 111B, top view in FIG. 111C, bottom view in FIG. 111D, and another side view in FIG. 111E.

TABLE 1
Correspondence between components in FIGS. 22 and 111A-111E.

| | The paddle | Braid or woven |
|---|---|---|
| | structure 100450 | the paddle |
| Component | in FIGS. 111A-111E | structure in FIG. 23 |
| Outer paddle | 100452 | 220 |
| Inner paddle | 100454 | 222 |
| Paddle frame | 100455 | 224 |
| Inner/outer the paddle | 100456 | 223 |
| connection portion | | |
| Cap/paddle connection | 100462 | 221 |
| portion | | |

The components of the paddle structure 100450 operate substantially similarly to their functional equivalents identified in Table 1. That is, the descriptions of the functional equivalents in the context of FIG. 22 apply equally well to the corresponding components in the paddle structure 100450.

As shown in FIGS. 111A-111E, inner/outer the paddle connection portion 100456 can be implemented by a cutout and series of perforations 100456a. Perforations 100456a allow connection portion 100456 to flex through a range of movement for opening and closing of the paddle structure 100450 shown in more detail below with respect to FIGS. 111H-111J. Joint portion 100460 can have a similar structure, though it is shown in FIGS. 111A-111E without perforations. More generally, either connection portion 100456 or joint portion 100460 can be fabricated in any suitable manner that creates flexibility to allow opening and closing of the paddle structure 100450. A base connection portion 100458 extends from the joint portion 100460. The base connection portion 100458 is configured to connect the paddle structure to a base, such as a central post or a coaptation element.

A cap/paddle frame connection portion 100462 in FIG. 111A connect the paddle structure 100450 to a distal the cap, such as the cap 214 and the paddle frames 224. The cap/paddle connection portion 100462 can take on a number of suitable forms. The connection portion 100462 is illustrated from above in FIG. 111C and from below in FIG. 111D. The connection portion 100462 can have any suitable configuration that fixes the paddle structure 100450 to the cap and/or the paddle frames. In the illustrated example, the connection portion includes a cutout that facilitates a snap fit connection of the paddle frames and/or the cap.

Turning back to FIG. 111A, each the paddle structure 100450 can contain eyelets 100464 that can be used to attach a cover and/or other components to the paddle structure 100450. Eyelet structure 100464 is shown in more detail in FIG. 111F. One of the purposes of the eyelets 100464 is to anchor sutures that connect the cover and/or other component sufficiently so that the suture does not pull out of the eyelet as stitching of the cover or other component to the paddle structure is started. In particular, the suture that is used to stitch the cover or other component to the paddle structure can be inserted into a wider portion 100464a of the eyelet 100464 that is wide enough to accommodate the entire diameter of suture. Then the suture can be anchored to the eyelet 100464 by moving suture from the wider portion 100464a to the narrower portion 100464b. The narrower portion 100464b has a width that is considerably less than the width of the wider portion 100464a. As a result, the narrower portion 100464b squeezes and fixes the suture into place. That is, the suture is wedged in the narrower portion 100464b.

FIG. 111G shows is a plan view of one-half of the flat, cut sheet material 100451 that is used to make the paddle structure 100450. FIG. 111G illustrates the location of the eyelets 100464 with respect to the inner/outer the paddle connection portion 100456 and other portions of the paddle structure 100450.

FIGS. 111H-111J show an example opening and closing motion of the paddle structure 100450 when used in an implementation of a valve repair device or implant. The paddle structure 100450 can have the range of motion of any of the paddle structured disclosed herein. For example, the paddle structure can also be moved to an extended position and can have the same or similar range of motion as the paddle structure of the device illustrated by FIG. 22. The valve repair device or implant that includes the paddle structure 100450 can take a variety of different forms and can include any of the features of any of the devices or implants disclosed herein.

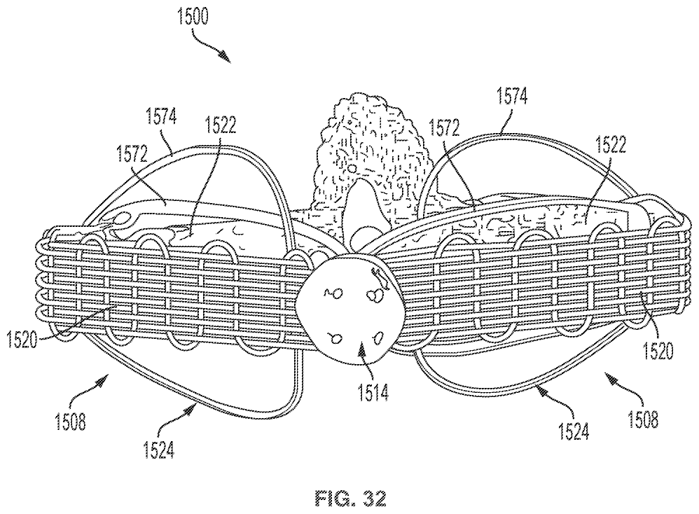

The position of the valve repair device or implant shown in FIG. 111H is a fully retracted position that corresponds to the fully retracted position shown for the example illustrated in FIG. 22. Referring to FIG. 111I, the actuation element 212 extends the cap 214 away from the coaptation element 210 to partially open the paddle assembly. The position shown in FIG. 111I corresponds to the partially open position shown in either FIG. 30 or FIG. 31. Referring to FIG. 111J, the actuation element 212 further extends the cap 214 further away from the coaptation element 210 to further open the paddle assembly. The position shown in FIG. 111J corresponds to the laterally extended or open position shown in FIG. 32.

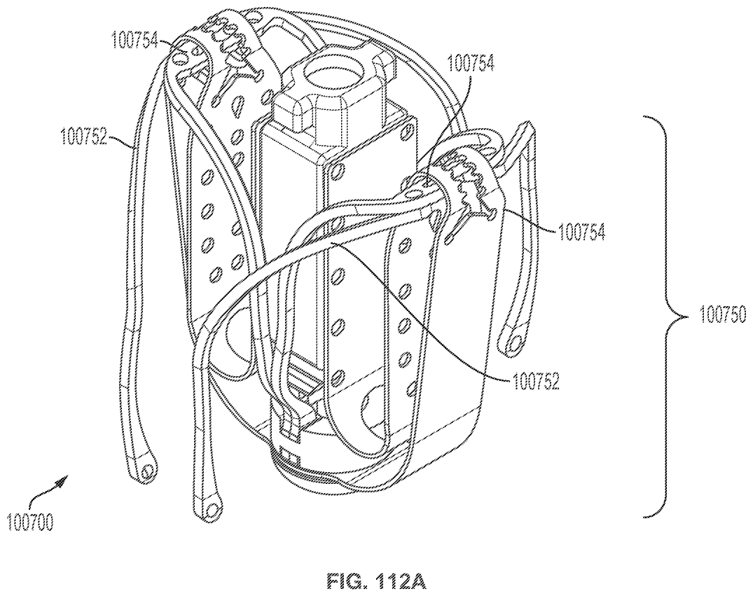

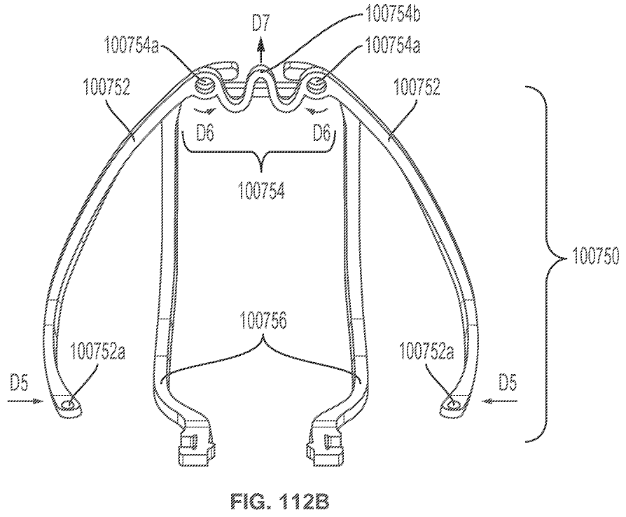

FIGS. 112A-112B show another device 100700 with outer paddle frame portions 100752 that are passively narrowed by being pressed on and passively expand back to their original state when the paddle frames are no longer being pressed on. Referring to FIG. 112B, the outer paddle frame portions 100752 include a restoring component 100754, such as a spring portion, that can passively assist restoring of the outer paddle frame portion 100752 (shown in FIGS. 112A and 112B) to its full width after the outer paddle frame portion 100752 has been flexed inward along direction D5 (FIG. 112B). In addition, the outer paddle frame portion 100752 can be fashioned out of a flexible material (e.g., shape memory alloy, nitinol, CuAlNi, NiTi, and various alloys of Zn, Cu, Au, and Fe, etc.) that does not substantially plastically deform the outer the paddle frame portion 100752 during narrowing.

More particularly, referring to FIG. 112B, the outer paddle frame portion 100752 can be passively narrowed by engaging an obstacle, such as the chordae tendineae, that applies a force on an outer portion 100752a the paddle frame portion 100752 in the direction D5 can press outer the paddle frame portions 100752 along direction D5.

In any case, force can be communicated to restoring component 100754 along direction D6 via length of outer the paddle frame portion 100752 and joint mechanism 100754a. The communicated force then pivots the paddle frame portion 100752 about the joint mechanism 100754a and compresses the center portion 100754b and/or introduces a displacement of the center portion 100754b of the restoring component 100754 along direction D7. Compression and/or displacement of center portion 100754b stores energy as a restoring force that can be used to ultimate move outer the paddle frame portion 100752 back to its original shape and configuration. Once the force causing displacement along D5 ceases (e.g., the device 100700 moves clear of an interaction with biological material), outer the paddle frame portion 100752 can tend to return to its original shape. Center portion 100754b of the restoring component 100754 assists this process by applying a restoring force in the direction opposite to D7. The restoring force is then transmitted to the outer the paddle frame portion 100752, causing it to flex in the opposite direction as D5. Subsequently, outer the paddle frame portion 100752 returns to its original shape, as shown in FIGS. 112A and 112B.

Another advantageous aspect of the paddle configuration 100750 is that the outer the paddle frame portions 100752 can still be opened and closed even during a deflection of the end(s) along the direction D5. That is to say, outer the paddle frame portions 100752 can be made of substantially stiff material such that a deflection along D5 does not prevent the user from opening or closing the paddles. For example, the paddles can still be opened and closed even when there is a substantial obstruction or interaction with biological material.

Although the restoring component 100754 shown in FIGS. 112A and 112B as an integral spring, it is to be understood that any suitable restoring force mechanism can be used. Examples include coiled wire, such as a compression spring or other similar the device, shape memory alloys, pneumatic devices, and other elastic the devices can all be used as the restoring component 100754. The restoring component 100754 can further include material cut in a patterned geometry that reduces strain during stretching or can include a polymer (e.g., rubber or elastomeric polymer). Rings or bands of polymer or other elastic material can be used. Still other examples of materials that could be used in restoring component 100754 include super-elastic nitinol, other nitinol, and/or stainless steel. Preferably, the material is biologically inert. In the illustrated example, the restoring component 100754 can be attached in a laminated configuration over the narrow inner paddle frames 100756.

The outer paddle frame portions 100752, the inner paddle frames 100756, the restoring component 100754, can be constructed using laser cutting, die casting, 3D printing, or other advanced manufacturing techniques. These components can be fabricated separately and assembled when finished. Such a manufacturing technique can be amenable to simple, scalable, mass production.

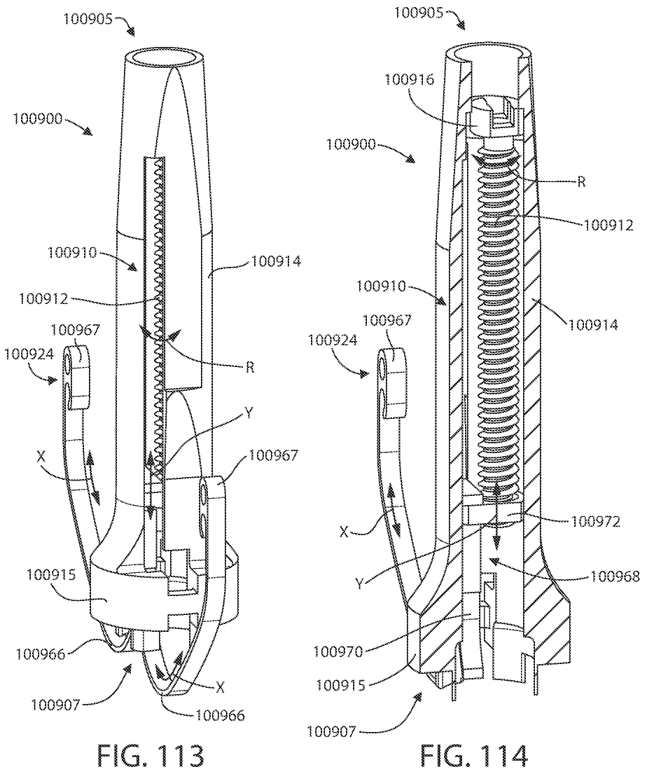

Referring to FIGS. 113-116, components of another example implementation of an implantable device or implant width adjustment assembly 100900 having paddle frame connectors is shown. The implantable device width adjustment assembly 100900 can include a proximal or attachment portion 100905, an anchor portion (e.g., any anchor portion described in the present application), paddle frame connector 100924 (e.g., shaped metal component, shaped plastic component, tether, wire, strut, line, cord, suture, etc.), an actuation portion 100910, an optional coaptation element (e.g., any spacer or coaptation element described in the present application), and a distal portion 100907. The connector 100924 forms a lower or distal portion of the paddle frames (See FIGS. 110A-110E). The attachment portion 100905, the anchor portion, the distal portion 100907, the actuation portion 100910, and the connector 100924 can be configured in a variety of ways.

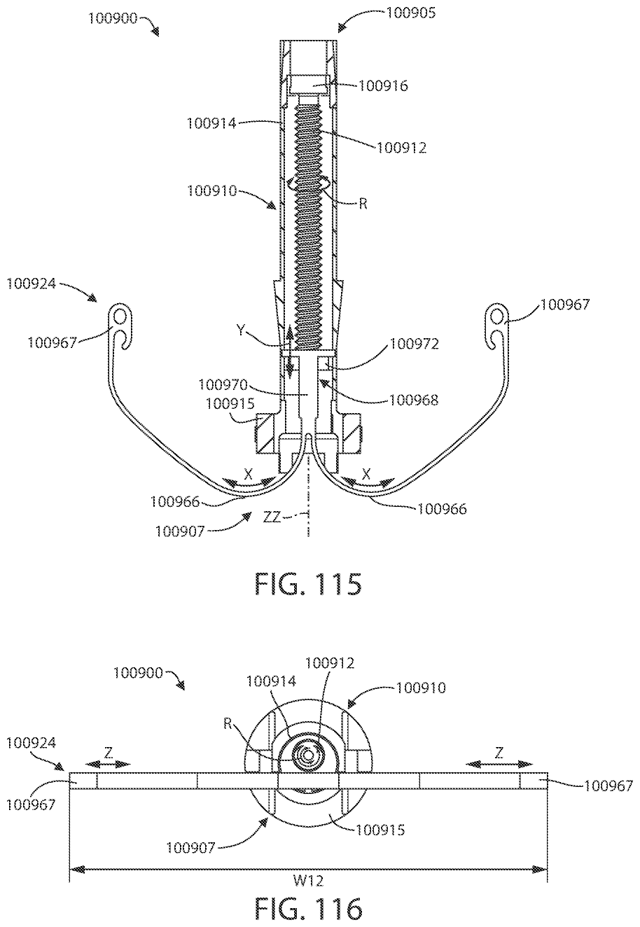

In the illustrated example, the connector 100924 is symmetric along longitudinal axis ZZ (FIG. 115). In some implementations of the implantable device width adjustment assembly 100900, however, the connector 100924 are not symmetric about the axis ZZ.

Referring to FIG. 115, in the illustrated example, the paddle frame connector 100924 comprises a W-shaped frame that has proximal ends 100967 and distal ends 100966. The connector 100924 has a width W12. The connector 100924 can be made of any suitable material that allows the connector 100924 to be moved between an expanded position and a narrowed position, such as, for example, any flexible material for paddle frames disclosed in the present application. While the connector 100924 is shown as having a W-shape, it should be understood that the connector 100924 can take any suitable form, such as, for example, any form described in the present application.

The connector 100924 has an inner end 100968 that engages with the actuation portion 100910 such that a user can move the inner end 100968 relative to the actuation portion 100910 to move the connector 100924 between a narrowed position and an expanded position, as described in more detail below. In the illustrated example, the inner end 100968 includes a post 100970 that attaches to the connector 100924 and a threaded receiving portion 100972 that extends from the post 100970. The inner end 100968 can, however, be configured in a variety of ways. Any configuration that can suitably attach the connector 100924 to the actuation portion 100910 to allow the actuation portion 100910 to move the connector 100924 between the narrowed position and the expanded position can be used.

The actuation portion 100910 allows a user to expand or contract the connector 100924 of the implantable device width adjustment assembly 100900. In the illustrated example, the actuation portion 100910 includes a coupler, such as the illustrated externally threaded shaft 100912 that is disposed within a receiver 100914 (e.g., an internally threaded element, a notched receiving portion, column, lumen, tube, shaft, sleeve, post, housing, tracks, cylinder etc.) and rotatably engaged with the threaded receiving portion 100972 of the inner end 100968 of the connector 100924. In some implementations, the receiver 100914 can be integrally formed with a distal cap 100915 of the distal portion 100907.

A driver head 100916 is disposed at a proximal end of the shaft 100912. The driver head 100916 is configured to receive a width adjustment element (e.g., width adjustment control, width adjustment wire, width adjustment shaft, width adjustment tube, width adjustment line, width adjustment cord, width adjustment suture, width adjustment tether, etc.) such that a user can rotate the width adjustment element to rotatably drive the shaft 100912 within the receiver 100914 in a direction R. The shaft 100912 extends through an opening of the receiving portion 100972 such that the external threads of the shaft 100912 engage internal threads of the opening of the receiving portion 100972. When the driver head 100916 is driven to rotate the shaft 100912, the engagement between the internal threads of the receiving portion 100972 and the external threads of the shaft 100912 causes the receiving portion 100972 (and, consequently, the post 100970) to move in a direction Y within the receiver 100914 and relative to the shaft 100912. The offset positioning between the shaft 100912 and the post 100970 of the inner end 100968 allows the post 100970 to move alongside the shaft 100912. In some implementations, rotation of the shaft 100912 in a counterclockwise direction causes the inner end 100968 to move toward the proximal end of the actuation portion 100910, and rotation of the shaft 100912 in a clockwise direction causes the inner end 100968 to move toward the distal end of the actuation portion 100910. However, it should be understood that other configurations are also contemplated.

In the illustrated example, the connection between the connector 100924 and the post 100970 of the inner end 100968 causes distal ends 100966 of the connector 100924 to move in the direction X (FIGS. 113-115) when the post 100970 moves in the direction Y, which causes the proximal ends 100967 of the connector to move in a direction Z (FIG. 116) to adjust the width W12 of the connector 100924. In the illustrated example, movement of the post 100970 toward a proximal end of the actuation portion 100910 causes the proximal ends 100967 of the connector 100924 to move in the direction Z toward the actuation portion 100910 such that the connector 100924 move to a narrowed position. Conversely, movement of the post 100970 toward a distal end of the actuation portion 100910 causes the proximal ends 100967 of the connector 100924 to move in the direction Z away from the actuation portion 100910 such that the connector 100924 moves to an expanded position. In some implementations, the distal ends 100966 of the connector 100924 can move into the receiver 100914 when the connector 100924 is moved to the narrowed position, and the distal ends 100966 can move out of the receiver 100914 when the connector 100924 is moved to the expanded position.

The movement of the connector 100924 to the narrowed position allows the device or implant to maneuver more easily into position for implantation in the heart by reducing the contact and/or friction between the native structures of the heart—e.g., chordae— and the device. The movement of the connector 100924 to the expanded position provides the anchor portion of the device or implant with a larger surface area to engage and capture leaflet(s) of a native heart valve.

In some implementations, the connector 100924 can be made from or comprise a material that allows the inner end 100968 and portions of the connector 100924 (e.g., the distal ends 100966) to be pulled into the actuation portion 100910. For example, the connector 100924, or a portion thereof, can be made of any flexible material, including but not limited to, metal, plastic, fabric, suture, etc. The connector 100924 can be made using a variety of processes, including, but not limited to, cutting, such as laser cutting, stamping, casting, molding, heat treating, shape setting, etc. The connector 100924 can be made from or comprise a shape memory material, —such as Nitinol—to provide shape-setting capability.

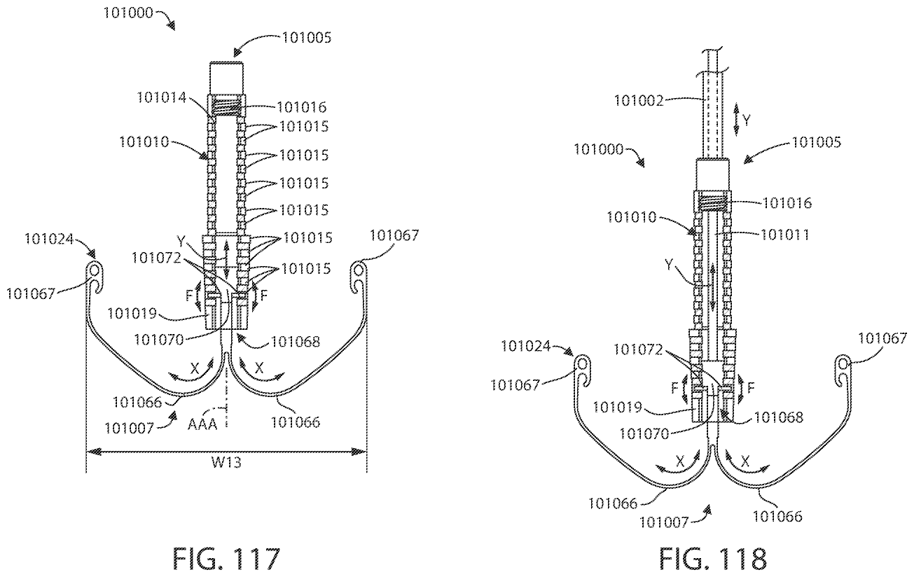

Referring to FIGS. 117-122, another example implementation of a portion of an implantable device 101000 having a connector 101024 (e.g., shaped metal component, shaped plastic component, tether, wire, strut, line, cord, suture, etc.) is shown. The implantable device 101000 includes a proximal or attachment portion 101005, an anchor portion (e.g., any anchor portion described in the present application), connector 101024, a width adjustment device 101010, an optional coaptation element (e.g., any spacer or coaptation element described in the present application), and a distal portion 101007. The connector 101024 forms a lower or distal portion of the paddle frames (See FIGS. 110A-110E). The attachment portion 101005, the anchor portion, the distal portion 101007, the width adjustment device 101010, and the connector 101024 can be configured in a variety of ways.

In the illustrated example, the connector 101024 are symmetric along longitudinal axis AAA (FIG. 117). In some implementations of the implantable device 101000, however, the connector 101024 are not symmetric about the axis AAA.

In the illustrated example, the connector 101024 are W-shaped frames that have proximal ends 101067 and distal ends 101066. The connector 101024 can have a width W13 (FIG. 117). The connector 101024 can be made of any suitable material that allows the connector 101024 to be moved between an expanded position and a narrowed position, such as, for example, any flexible material for paddle frame portions disclosed in the present application. While the connector 101024 is shown as having a W-shape, it should be understood that the connector 101024 can take any suitable form, such as, for example, any form described in the present application.

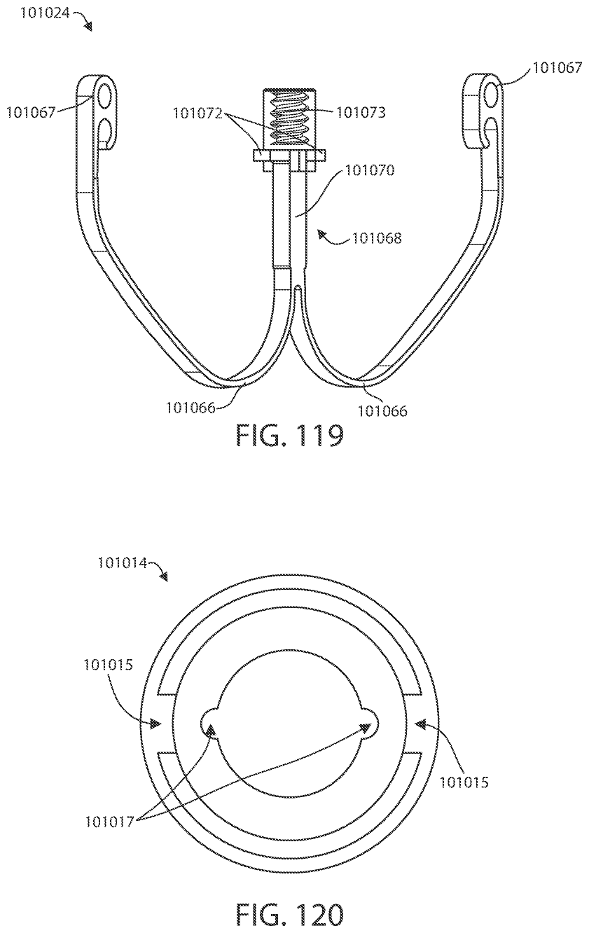

The connector 101024 has a coupler 101068 that engages with a width adjustment element 101011 (e.g., width adjustment control, width adjustment wire, width adjustment shaft, width adjustment tube, width adjustment line, width adjustment cord, width adjustment suture, width adjustment tether, etc.). The coupler 101068 can be configured in a variety of different ways. For example, the coupler 8972 can include one or more of a threaded connection, features that mate with threads, detent connections, such as outwardly biased arms or portions, flexible projections etc. The coupling between the width adjustment element 101011 and the coupler 101068 allows a user to move the coupler 101068 of the width adjustment device 101010 to move the connector 101024 between a narrowed position and an expanded position, as described in more detail below. In the illustrated example, the coupler 101068 includes a post 101070 that attaches to the connector 101024 and flexible projections 101072 that extend from the post 101070. The coupler 101068 can be configured to receive a width adjustment element 101011 (e.g., an actuation wire, actuation shaft, etc.) that allows a user to move the coupler 101068 in the direction Y. For example, the post 101070 can have a coupler, such as the illustrated threaded receiving portion 101073 (FIG. 119) that is configured to engage with threads of the width adjustment element 101011. The coupler 101068 can comprise any configuration that can suitably attach the connector 101024 to the width adjustment device 101010 to allow the width adjustment device 101010 to move the connector 101024 between the narrowed position and the expanded position can be used. The coupler can be a separate component or be integrally formed as a portion of another component of the device (e.g., as part of the connector, etc.).

The width adjustment device 101010 allows a user to expand or contract the connector 101024 of the implantable device 101000. In the illustrated example, the width adjustment device 101010 includes a receiver 101014 (e.g., an internally threaded element, a notched receiving portion, column, lumen, tube, shaft, post, housing, tracks, cylinder etc.) that has a plurality of slots 101015 (FIGS. 117 and 120) for receiving the flexible projections 101072 of the coupler 101068. That is, the post 101070 of the coupler 101068 is sized to fit and move within the receiver 101014 in the direction Y, and the flexible projections 101072 are sized to fit within the slots 101015 of the receiver 101014 to secure the coupler 101068 at a desired position within the receiver 101014. In some implementations, the receiver 101014 includes channels 101017 (FIG. 120) that connect to each of the slots 101015 and are positioned to align with the flexible projections 101072 of the coupler 101068 such that the flexible projections 101072 can move through the channels 101017 when a user moves the coupler 101068 to various positions within the receiver 101014. The channels 101017 guide the flexible projections 101072 of the coupler 101068 along the receiver 101014. In some implementations, the receiver 101014 can be integrally formed with a distal cap 101019 of the distal portion 101007.

A connection feature 101016 is disposed at a proximal end of the implantable device 101000 for receiving an actuation element 101002 (e.g., actuation shaft, actuation tube, actuation lumen, conduit, etc.) of a delivery device. In the illustrated example, the connection feature 100916, such as the illustrated driver head, includes internal threads for connecting to external threads of the actuation element 101002. The connection feature 101016 can, however, have any configuration that can receive and attach to the actuation element 101002.

A width adjustment element 101011 extends through the actuation element 101002 of the delivery device and into the receiver 101014 of the width adjustment device 101010 of the implantable device 101000. The width adjustment element 101011 removably connects to the post 101070 of the coupler 101068 such that a user can move the width adjustment element 101011 in the direction Y to cause the coupler 101068 to move in the direction Y. In the illustrated example, the width adjustment element 101011 includes external threads for connecting to internal threads of the post 101070. The width adjustment element 101011 can, however, have any configuration that can attach to the coupler 101068 and allow a user to move the coupler 101068.

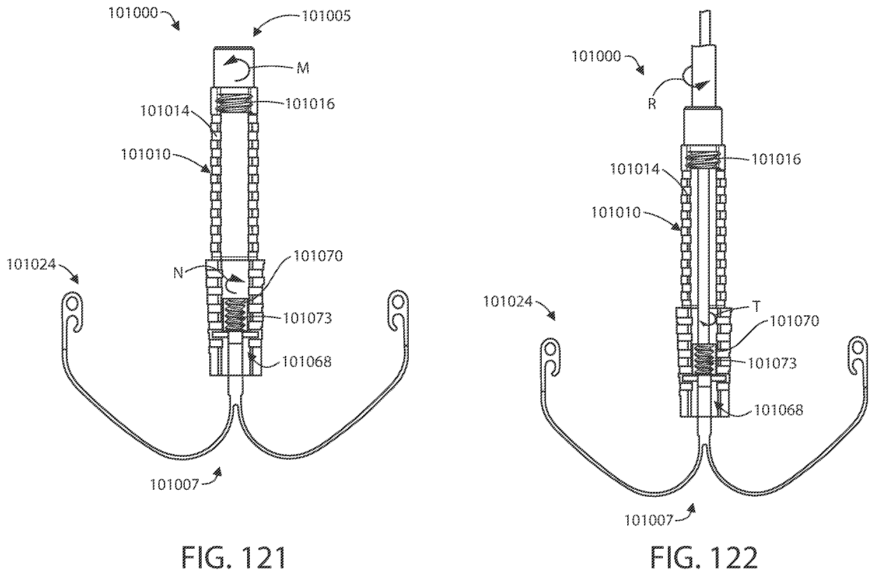

Referring to FIGS. 121 and 122, in some implementations, the connection feature 101016 at the proximal end of the device 101000 and threaded receiving portion 101073 of the coupler 101068 of the connector 101024 are threaded in opposite directions. That is, referring to FIG. 121, the threads of the connection feature 101016 are disposed in a direction M, and the threads of the receiving portion 101073 are disposed in a direction N. Consequently, referring to FIG. 122, rotation of the actuation element 101002 in the direction R causes the external threads of the actuation element 101002 to engage the connection feature 101016 and attach to the device 101000, and rotation of the width adjustment element 101011 in the direction T causes the external threads of the width adjustment element 101011 to engage the threaded receiving portion 101073 of the coupler 101068 to attach to the coupler 101068. In example, the actuation element 101002 can be disengaged from the device 101000 by rotating the actuation element 101002 in the direction T, and the width adjustment element 101011 can be disengaged from the coupler 101068 by rotating the width adjustment element 101011 in the direction R. The opposite thread directions of the connection feature 101016 and the threaded receiving portion 101073 prevents or inhibits accidental disengagement of one of the actuation element 101002 and the width adjustment element 101011 when a user attempts to disengage the other of the actuation element 101002 and the width adjustment element 101011. That is, when a user attempts to disengage one of the actuation element 101002 and the width adjustment element 101011, the other of the actuation element and actuation element will tighten (or not move at all) due to the direction of rotation caused by the user. However, it should be understood that other configurations are also contemplated.

Referring to FIGS. 117-118, after the width adjustment element 101011 is attached to the coupler 101068, a user moves the width adjustment element 101011 in the direction Y to move the connector 101024 between narrowed and expanded positions. That is, movement of the width adjustment element 101011 in the Y direction causes the post 101070 of the coupler 101068 to move in the direction Y, and the connection between the connector 101024 and the post 101070 causes distal ends 101066 of the connector 101024 to move in the direction X when the post 100970 moves in the direction Y. This movement of the distal ends 101066 in the direction X causes the proximal ends 101067 of the connector 101024 to move to adjust the width W13 of the connector 101024.

In the illustrated example, movement of the post 101070 toward a proximal end of the width adjustment device 101010 causes the proximal ends 101067 of the connector 101024 to move toward the width adjustment device 101010 such that the connector 101024 moves to a narrowed position. Conversely, movement of the post 101070 toward a distal end of the width adjustment device 101010 causes the proximal ends 100967 of the connector 100924 to move in the direction away from the width adjustment device 101010 such that the connector 101024 moves to an expanded position. However, it should be understood that other configurations are also contemplated. In some implementations, the distal ends 101066 of the connector 101024 can move into the receiver 101014 when the connector 101024 is moved to the narrowed position, and the distal ends 101066 can move out of the receiver 101014 when the connector 101024 is moved to the expanded position. However, it should be understood that other configurations are also contemplated.

Movement of the coupler 101068 in the direction Y within the receiver 101014 causes the flexible projections 101072 to flex in the direction F, which allows the flexible projections 101072 to move between a flexed position in which the flexible projections engage an interior surface of the receiver 101014 and an extended position in which the flexible projections 101072 are disposed within a slot 101015 of the receiver. When the flexible projections 101072 are in the extended position and disposed within a slot 101015, the width W13 of the connector 101024 is maintained in the position associated with the location of the coupler 101068 relative to the receiver 101014. The user can adjust the width W13 of the connector 101024 by moving the width adjustment element 101011 in the direction Y, which causes the flexible projections 101072 to flex and allow the coupler 101068 to move within the receiver 101014. In some implementations, the interior surface of the receiver includes channels 101017 (FIG. 120) that allow for movement of the flexible projections 101072 through the receiver 101014. The flexible projections 101072 of the coupler 101068 can be made of a flexible material, including but not limited to, metal, plastic, fabric, suture, etc. While described here as a receiver, other structures or openings in structures of a variety of shapes and sizes that can accomplish the same purpose can be used as well.

The movement of the connector 101024 to the narrowed position allows the device or implant 101000 to maneuver more easily into position for implantation in the heart by reducing the contact and/or friction between the native structures of the heart—e.g., chordae— and the device 101000. The movement of the connector 101024 to the expanded position provides the anchor portion of the device or implant 101000 with a larger surface area to engage and capture leaflet(s) of a native heart valve.

In some implementations, the connector 101024 can be made from or comprise a material that allows the coupler 101068 and portions of the connector 101024 (e.g., the distal ends 101066) to be pulled into the actuation portion 100910. For example, the connector 101024, or a portion thereof, can be made of any flexible material, including but not limited to, metal, plastic, fabric, suture, etc. The connector 101024 can be made using a variety of processes, including, but not limited to, cutting, such as laser cutting, stamping, casting, molding, heat treating, shape setting, etc. The connector 101024 can be made from or comprise a shape memory material, —such as Nitinol—to provide shape-setting capability.

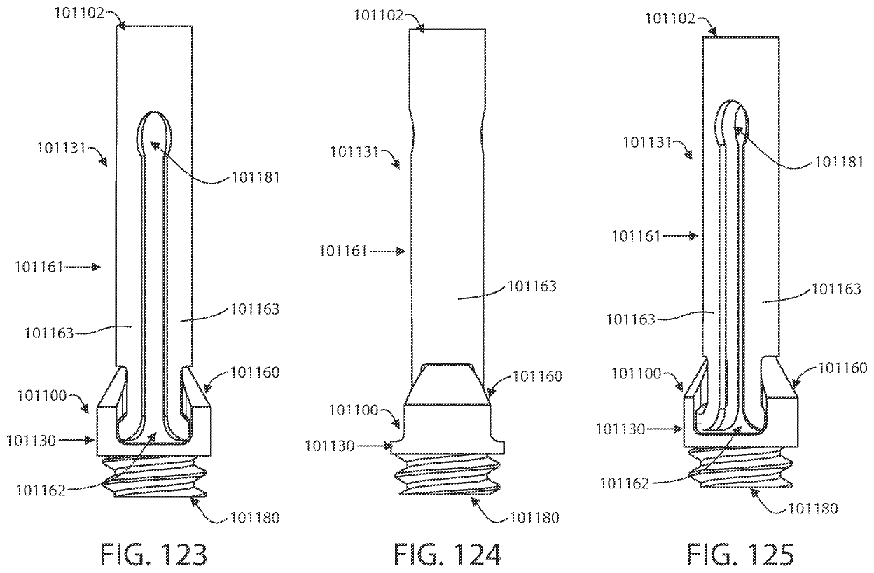

FIGS. 123-131 show an example coupling between an actuation element 101102 (e.g., actuation shaft, actuation tube, actuation lumen, conduit, etc.) of a delivery device and a component of an implantable device or implant 101100. For example, the coupling can be between the actuation element and a proximal end of the implantable device 101100. In some implementations, the coupling is between the actuation element 101102 and a receiver of the implantable device 101100 (e.g., any receiver of an implantable device described in the present application) such that a width adjustment element (e.g., width adjustment control, width adjustment wire, width adjustment shaft, width adjustment tube, width adjustment line, width adjustment cord, width adjustment suture, width adjustment tether, etc.) can extend through the actuation element 101102 to engage a connector or a paddle frame of the implantable device (e.g., any connector or paddle frames of an implantable device described in the present application).

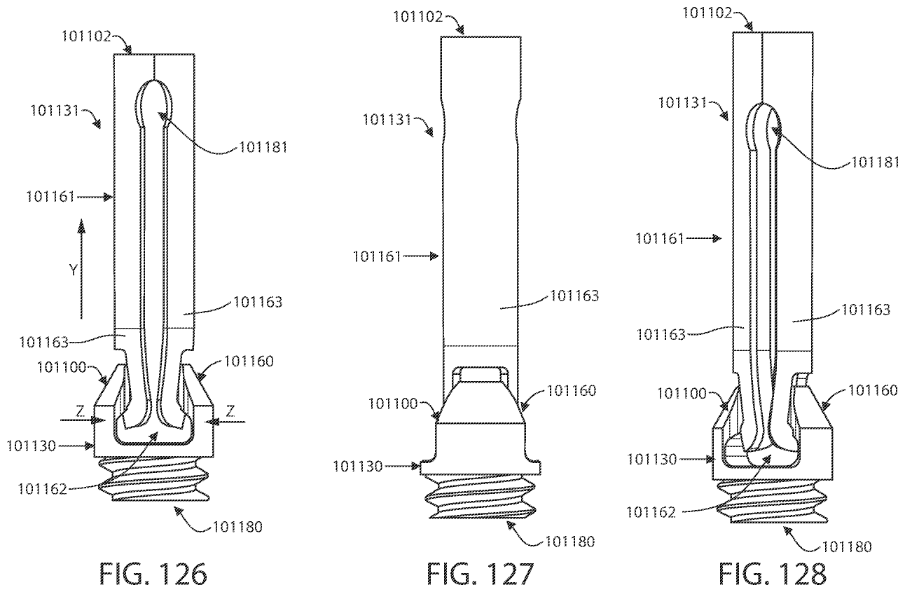

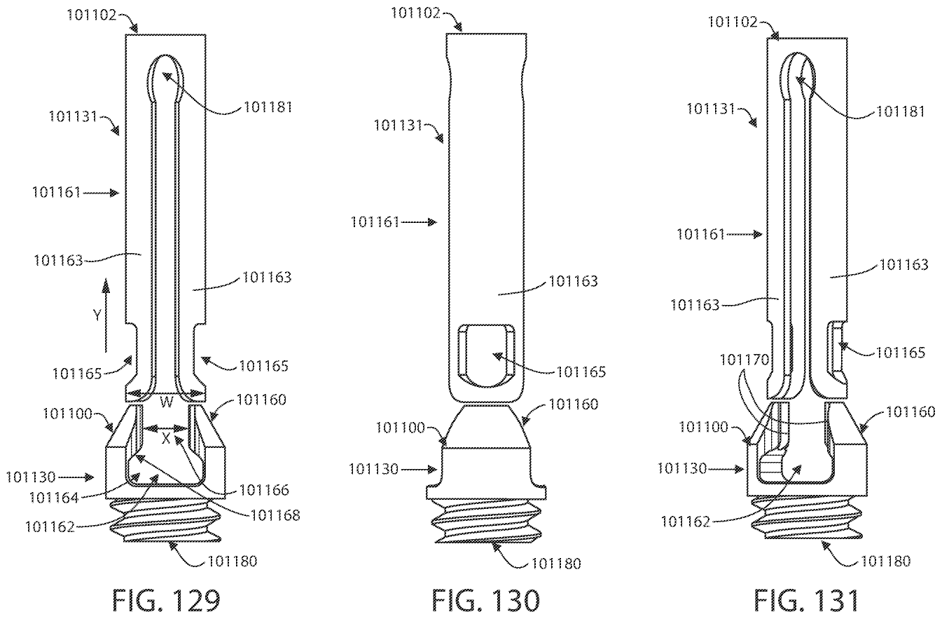

A distal end 101131 of the actuation element 101102 has a connection feature 101161 that includes a pair of arms 101163 that are movable between a normal, expanded position (e.g., as shown in FIGS. 123 and 129) and a compressed position (e.g., as shown in FIG. 126). While the illustrated example shows the connection feature 101161 having a pair of arms 101163, it should be understood that the connection feature 101161 can have any suitable number of arms. In some implementations, the arms 101163 include an opening 101165 for maintaining a secure connection between the actuation element 101102 and the implantable device 101100. That is, the opening 101165 can be sized and configured to receive an inward extension portion 101170 (See FIG. 131) of a connection feature 101160 of the implantable device 101100 to prevent or inhibit the arms 101163 from being prematurely disengaged from the implantable device 101100. The distal end 101131 can have an arched or curved opening 101181 positioned at the connection between the arms 101163 and the remainder of the actuation element 101102 that facilitates movement of the arms 101163 between the normal position and the compressed position. That is, the curved opening 101181 allows for the arms 101163 to flex more easily relative to the remainder of the actuation element 101102.

A proximal end 101130 of the implantable device 101100 has a connection feature 101160 that includes an opening 101162 for receiving the arms 101163 of the actuation element 101102. Referring to FIG. 129, the opening 101162 can include a distal portion 101164 and a proximal portion 101166, where the distal portion 101164 is wider than the proximal portion 101166. The interior of the opening 101162 can include a tapered wall 101168 that extends between the distal portion 101164 and the proximal portion 101166. In the illustrated example, the connection feature 101160 includes an inward extension portion 101170 (FIG. 131) that defines the proximal portion 101166 of the opening 101162. The connection feature 101160 can also have another connection element 101180 for attaching to the implantable device 101100. For example, the connection element 101180 can include a threaded portion that threadably attaches to the implantable device 101100. In some implementations, the connection element 101180 attaches to an actuation portion of the implantable device 101100. In some implementations the connection feature 101160 can be integral to a component of the implantable device 101100, or the connection feature 101160 can attach to the implantable device 101100 by any other suitable means.

Referring to FIGS. 123-125, when the implantable device 101100 is being delivered to a native valve of a patient by the delivery device, the actuation element 101102 is attached to the implantable device 101100. That is, the arms 101163 of the actuation element 101102 extend into the distal portion 101164 of the opening 101162 of the implantable device 101100. When the arms 101163 are in the normal position, a width W (FIG. 129) of the arms 101163 are wider than the width X (FIG. 129) of the proximal portion 101166 of the opening 101162, which prevents or inhibits the arms 101163 from moving through the proximal portion 101166 of the opening 101162 and disengaging from the implantable device 101100. In addition, the inward extension portion 101170 (FIG. 131) of the connection feature 101160 can extend into the openings 101165 of the arms 101163 of the actuation element 101102 to further secure the actuation element 101102 to the implantable device 101100. When the actuation element 101102 is attached to the implantable device 101100, an open path can extend from the actuation element and through the implantable device 101100 such that a width adjustment element (e.g., an width adjustment wire, width adjustment shaft, width adjustment tube etc.) can extend through the actuation element 101102 and implantable device 101100 to engage one or more portions of the implantable device 101100 to move a paddle frame of the implantable device between expanded and narrowed positions, or to engage the implantable device in any other desired way as the device is being implanted on the native valve of a patient and/or released from the delivery system.

Referring to FIGS. 126-128, when a force is applied to the actuation element 101102 in the direction Y (FIG. 126), the arms 101163 engage the tapered wall 101168 (FIG. 129) of the opening 101162, which facilitates movement of the arms 101163 to a compressed position. That is, the engagement between the tapered wall 101168 and the arms 101163 causes an inward force to the arms 101163 in the direction Z, which causes the arms 101163 to move to the compressed position. As the arms 101163 are moving toward the compressed position, the inward extension portion 101170 (FIG. 131) of the connection feature 101160 can remain extending into the openings 101165 of the arms 101163 of the actuation element 101102 to maintain a secure connection between the actuation element 101102 and the implantable device 101100. This prevents or inhibits an accidental disengagement between the actuation element 101102 and the implantable device 101100. That is, the connection between the actuation element 101102 and the implantable device 101100 is maintained unless a sufficient force is supplied in the direction Y to disengage the actuation element 101102 from the implantable device 101100.

Referring to FIGS. 126-131, when a sufficient force is supplied to the actuation element 101102 in the direction Y, the actuation element 101102 disengages from the implantable device 101100. That is, the force provided to the arms 101163 in the direction Z (FIG. 126) causes the arms to move to a compressed position such that a width W (FIG. 129) of the arms 101163 is less than or equal to the width X (FIG. 129) of the proximal portion 101266 of the opening 101262, which allows the arms 101163 to exit the opening 101162 of the implantable device 101100. The arms 101163 of the actuation element 101102 can be made of any suitable material that allows the arms 101163 to move to the compressed position and be removed from the implantable device 101100. For example, the arms 101163 can be made of metal, plastic, composite material, shape memory material, etc.

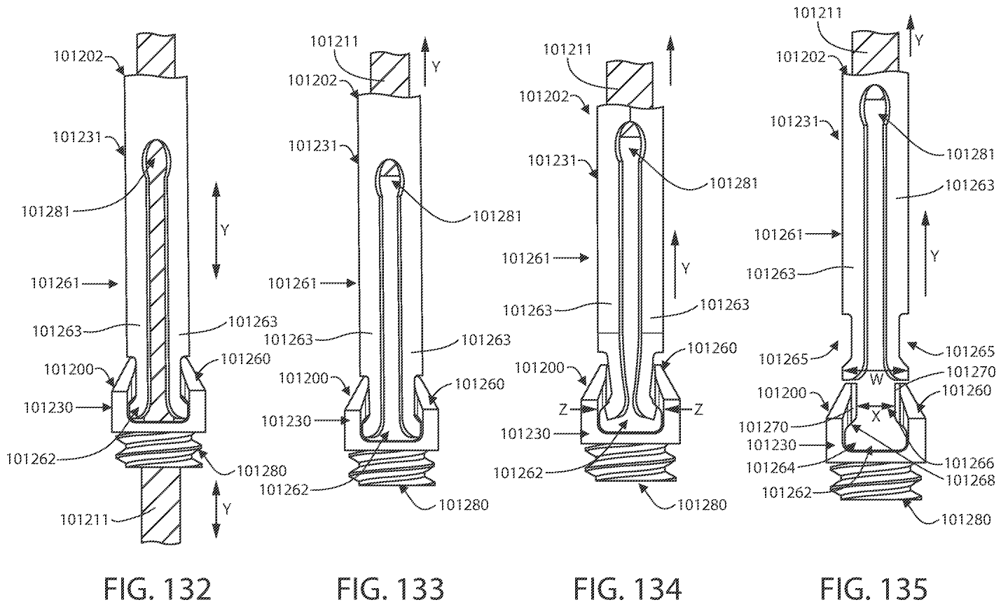

FIGS. 132-135 show an example coupling between an actuation element 101202 of a delivery device and a component of an implantable device or implant 101200. For example, the coupling can be between the actuation element 101202 and receiver at a proximal end of the implantable device 101200. In some implementations, the coupling is between the actuation element 101202 and an actuation portion of the implantable device 101200 (e.g., any actuation portion of an implantable device described in the present application) such that a width adjustment element 101211 (e.g., any width adjustment element shown or described in the present application) can extend through the actuation element 101202 to engage paddle frames of the implantable device (e.g., any paddle frames of an implantable device described in the present application).

A distal end 101231 of the actuation element 101202 has a connection feature 101261 that includes a pair of arms 101263 that are movable between a normal, expanded position (e.g., as shown in FIGS. 132 and 133) and a compressed position (e.g., as shown in FIG. 134). While the illustrated example shows the connection feature 101261 having a pair of arms 101263, it should be understood that the connection feature 101261 can have any suitable number of arms. In some implementations, the arms 101263 include an opening 101265 for maintaining a secure connection between the actuation element 101202 and the implantable device 101200. That is, the opening 101265 can be sized and configured to receive an inward extension portion 101270 of a connection feature 101260 of the implantable device 101200 to prevent or inhibits the arms 101263 from being prematurely disengaged from the implantable device 101200. The distal end 101231 can have an arched or curved opening 101281 positioned at the connection between the arms 101263 and the remainder of the actuation element 101202 that facilitates movement of the arms 101163 between the normal position and the compressed position. That is, the curved opening 101281 allows for the arms 101263 to flex more easily relative to the remainder of the actuation element 101202.

A proximal end 101230 of the implantable device 101200 has a connection feature 101260 that includes an opening 101262 for receiving the arms 101263 of the actuation element 101202. Referring to FIG. 135, the opening 101262 can include a distal portion 101264 and a proximal portion 101266, where the distal portion 101264 is wider than the proximal portion 101266. The interior of the opening 101262 can include a tapered wall 101268 that extends between the distal portion 101264 and the proximal portion 101266. In the illustrated example, the connection feature 101260 includes an inward extension portion 101270 (FIG. 135) that defines the proximal portion 101266 of the opening 101262. The connection feature 101260 can also have another connection element 101280 for attaching to the implantable device 101200. For example, the connection element 101280 can include a threaded portion that threadably attaches to the implantable device 101200. In some implementations, the connection element 101280 attaches to an actuation portion of the implantable device 101200. In some implementations the connection feature 101260 can be integral to a component of the implantable device 101200, or the connection feature 101260 can attach to the implantable device 101200 by any other suitable means.

When the implantable device 101200 is being delivered to a native valve of a patient by the delivery device, the actuation element 101202 is attached to the implantable device 101200. That is, the arms 101263 of the actuation element 101202 extend into the distal portion 101264 of the opening 101262 of the implantable device 101200. When the arms 101263 are in the normal position, a width W (FIG. 135) of the arms 101263 are wider than the width X (FIG. 135) of the proximal portion 101266 of the opening 101262, which prevents or inhibits the arms 101263 from moving through the proximal portion 101266 of the opening 101262 and disengaging from the implantable device 101200. In addition, the inward extension portion 101270 (FIG. 135) of the connection feature 101260 can extend into the openings 101265 of the arms 101263 of the actuation element 101202 to further secure the actuation element 101202 to the implantable device 101200.

A width adjustment element 101211 (which can be the same as or similar to other width adjustment elements shown or described herein) can extend through the actuation element 101202 and the connection feature 101260 of the implantable device 101200 such that the width adjustment element 101211 can be engaged by a user to control various movements of the implantable device 101200, such as the width of the paddles and decoupling of the actuation element from the device. In some implementations, the width adjustment element 101211 is sized and positioned within the actuation element 101202 to exert an outward force on the arms 101263 to maintain the arms 101263 in the distal portion 101264 of the opening 101262 and prevent or inhibit disengagement between the actuation element 101202 and the implantable device 101200. In these implementations, when the width adjustment element 101211 is extending through the actuation element 101202 and the implantable device 101200, the width adjustment element 101211 can prevent or inhibit removal of the actuation element 101202 from the implantable device 101200 even if a user provides a force to the actuation element 101202 in the direction Y. That is, the force exerted by the width adjustment element 101211 on the arms 101263 prevents or inhibits the arms 101263 from moving to a compressed position. In some implementations, the normal position of the arms 101263 of the actuation element 101202 can be biased inward (rather than biased outward as described above) for easy removal from the actuation element 101202 from the implantable device 101200, and the force by the width adjustment element 101211 on the arms 101263 is the main force used to maintain the arms in an expanded position such that the actuation element 101202 maintains a connection with the implantable device 101200.

Referring to FIG. 132, in some implementations, when the implantable device 101200 is being delivered to a native valve of a patient by the delivery device, the actuation element 101202 is attached to the implantable device 101200 and the width adjustment element 101211 is extending through the actuation element 101202 and the implantable device 101200. Referring to FIG. 133, after the user manipulates the implantable device 101200 with the width adjustment element 101211, the user can pull the width adjustment element 101211 in the direction Y to remove the width adjustment element 101211 from the implantable device 101200.

Referring to FIG. 134, after the width adjustment element 101211 is moved in the direction Y beyond the arms 101263 of the actuation element 101202 and when a force is applied to the actuation element 101202 in the direction Y, the arms 101263 engage the tapered wall 101268 (FIG. 135) of the opening 101262, which facilitates movement of the arms 101263 to a compressed position. That is, the engagement between the tapered wall 101268 and the arms 101263 causes an inward force to the arms 101263 in the direction Z, which causes the arms 101263 to move to the compressed position. As the arms 101263 are moving toward the compressed position, the inward extension portion 101270 (FIG. 135) of the connection feature 101260 can remain extending into the openings 101265 of the arms 101263 of the actuation element 101202 to maintain a secure connection between the actuation element 101202 and the implantable device 101100. This prevents or inhibits an accidental disengagement between the actuation element 101202 and the implantable device 101200. That is, the connection between the actuation element 101202 and the implantable device 101200 is maintained unless a sufficient force is supplied in the direction Y to disengage the actuation element 101202 from the implantable device 101200.

Referring to FIG. 135, when a sufficient force is supplied to the actuation element 101202 in the direction Y, the actuation element 101202 disengages from the implantable device 101200. That is, the force provided to the arms 101263 in the direction Z (FIG. 134) causes the arms to move to a compressed position such that a width W of the arms 101263 is less than or equal to the width X of the proximal portion 101266 of the opening 101262, which allows the arms 101263 to exit the opening 101262 of the implantable device 101100. The arms 101263 of the actuation element 101202 can be made of any suitable material that allows the arms 101263 to move to the compressed position and be removed from the implantable device 101200. For example, the arms 101263 can be made of metal, plastic, composite material, shape memory material, etc.

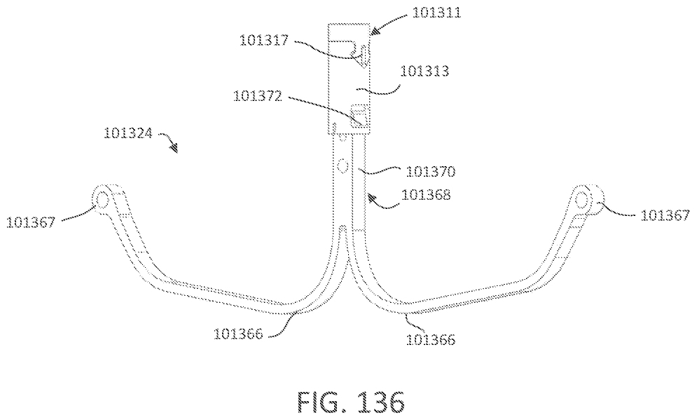

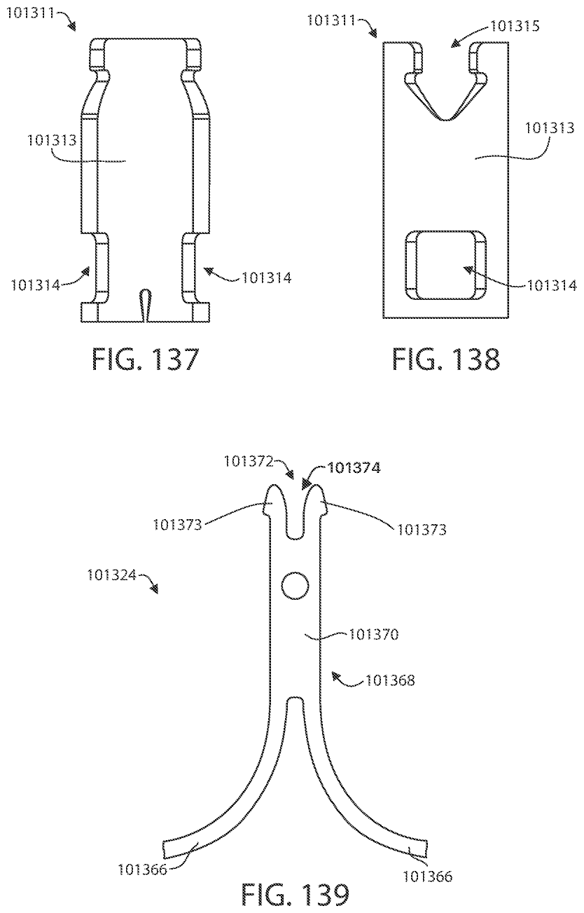

FIGS. 136-139 show an example coupling between a connector 101324 (e.g., shaped metal component, shaped plastic component, tether, wire, strut, line, cord, suture, etc.) of an implantable device or implant (not shown) and a width adjustment element 101311 (which can be the same as or similar to other width adjustment elements shown or described herein) such that a user can engage the width adjustment element 101311 to move the connector 101324 between an expanded position and a narrowed position. The connector 101324 and width adjustment element 101311 can be used with any suitable implantable device or implant, such as, for example, any implantable device or implant described in the present application.

In the illustrated example, the connector 101324 is a W-shaped frame that has proximal ends 101367 and distal ends 101366. The connector 101324 can be made of any suitable material that allows the connector 101324 to be moved between an expanded position and a narrowed position, such as, for example, any flexible material for paddle frames disclosed in the present application. While the connector 101324 are shown as having a W-shape, it should be understood that the connector 101324 can take any suitable form, such as, for example, any form described in the present application.

The connector 101324 has an inner end 101368 that is configured to be connected through an opening in a coupler 101313. The coupler 101313 releasably connects with the width adjustment element 101311. The coupler 101313 allows a user to move the width adjustment element 101311 and connected inner end 101368, which causes the paddle frame portions to be moved between the narrowed and expanded positions. For example, in some implementations, the implant can include an actuation portion (e.g., any actuation portion of an implant described in the present application), and the user can move the inner end 101368 relative to the actuation portion to move the connector 101324 between a narrowed position and an expanded position.

In the illustrated example, the inner end 101368 includes a post 101370 attached to the distal ends 101366 of the connector 101324 and a retention feature 101372 for attaching to the width adjustment element 101311. The retention feature 101372 can include flexible arms 101373 (FIG. 139) that extend from the post 101370 with an opening or slit 101374 positioned between the arms 101373. In some implementations, the retention feature 101372 does not include the slit 101374 between the arms 101373, but rather the retention feature 101372 is solid between the arms 101373. The arms 101373 can be movable between a normal, expanded position (e.g., as shown in FIG. 139) and a compressed position (not shown). While the illustrated example shows the retention feature 101372 having a pair of arms 101373, it should be understood that the retention feature 101372 can have any suitable number of arms.

The width adjustment element 101311 can include or be coupled to, for example, an actuation wire, actuation shaft, or any other suitable element that a user can engage to move the connector 101324 between the narrowed and expanded positions. A distal end of the width adjustment element 101311 can be releasably or permanently connected to the coupler 101313. The connector or connection feature can receive and connect to the retention feature 101372 of the connector 101324. The coupler 101313 include openings 101314 for receiving the arms 101373 of connector 101324. In the illustrated example, the coupler 101313 is a separate component from the remainder of the width adjustment element 101311. For example, a proximal end of the coupler 101313 can be configured to attach to the remainder of the width adjustment element 101311. In the illustrated example, the coupler 101313 includes a connection opening 101315 for receiving a connection element 101317 (FIG. 136) of the width adjustment element 101311. The connection between the connection element 101317 and the connection opening 101315 can take any suitable form, such as, for example, any form described in the present application. In some implementations, the coupler 101313 is integral to another component of the width adjustment element 101311. In yet some implementations, the coupling between the width adjustment element 101311 and the coupler 101313 is releasable like the couplings illustrated by FIGS. 123-135. In any of the implementations described above, the width adjustment element 101311 can be configured to be detached and reattached from the connector 101324.

In some implementations, the coupler 101313 is connected to the retention feature 101372 of the connector 101324 by a snap-fit connection. For example, the coupler 101313 is placed over the retention feature 101372 of the connector 101324 such that the arms 101373 move to the compressed position. As the coupler 101313 is being placed over the retention feature 101372, extended portions of the arms 101373 will move into the openings 101314 of the coupler 101313 such that the arms 101373 will move back to the normal, expanded position, which secures the connector 101324 to the coupler 101313. In some implementations, this connection between the connector 101324 and the coupler 101313 is permanent.

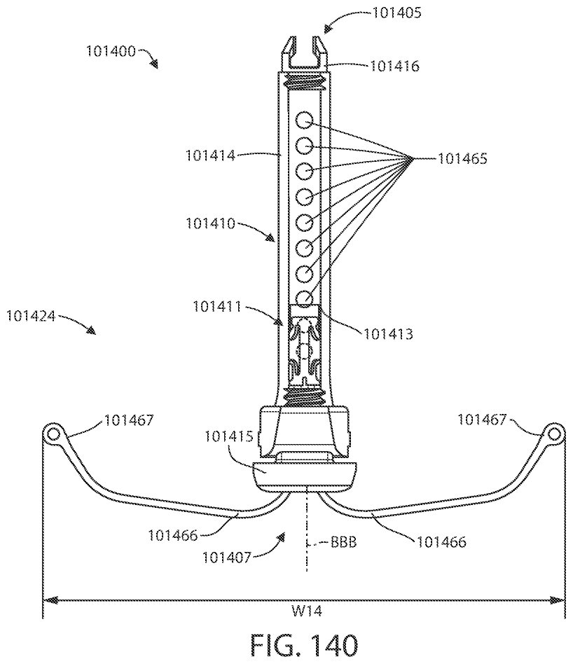

Referring to FIGS. 140-142, another example implementation of an implantable device or implant 101400 having paddle frames is shown. The implantable device 101400 includes a proximal or attachment portion 101405, an anchor portion (e.g., any anchor portion described in the present application), connector 101424 (e.g., shaped metal component, shaped plastic component, tether, wire, strut, line, cord, suture, etc.), an actuation portion 101410, an optional coaptation element (e.g., any coaptation element described in the present application), and a distal portion 101407. The attachment portion 101405, the anchor portion, the distal portion 101407, the actuation portion 101410, and the connector 101424 can be configured in a variety of ways.

In the illustrated example, the connector 101424 is symmetric along longitudinal axis BBB (FIG. 140). In some implementations of the implantable device 101400, however, the connector 101424 are not symmetric about the axis BBB.

In the illustrated example, the connector 101424 are W-shaped frames that have proximal ends 101467 and distal ends 101466. The connector 101424 can have a width W14 (FIG. 140). The connector 101424 can be made of any suitable material that allows the connector 101424 to be moved between an expanded position and a narrowed position, such as, for example, any flexible material for paddle frames disclosed in the present application. While the connector 101424 is shown as having a W-shape, it should be understood that the connector 101424 can take any suitable form, such as, for example, any form described in the present application.

The connector 101424 have an inner end (not shown) that engages with the actuation portion 101410 such that a user can move the inner end relative to the actuation portion 101410 to move the connector 101424 between a narrowed position and an expanded position, as described in more detail below. The inner end can take any suitable form, such as, for example, the form of the inner end 101368 shown in FIGS. 136 and 139, or any other form described in the present application. The inner end can, however, be configured in a variety of ways. Any configuration that can suitably couple the connector 101424 to the actuation portion 101410 to allow the actuation portion 101410 to open and close the paddle frames and to move the connector 101424 between the narrowed position and the expanded position can be used.

The actuation portion 101410 allows a user to open and close the paddle frames of the device by moving the actuation portion 101410 relative to the proximal portion of the device. The actuation portion 101410 also allows a user to expand or contract the connector 101424 of the implantable device 101400 by moving the connector 101424 into or out of the actuation portion 101410. In the illustrated example, the actuation portion 101410 includes a receiver 101414 (e.g., an internally threaded element, a notched receiving portion, column, lumen, tube, shaft, sleeve, post, housing, tracks, cylinder etc.) and a coupler 101411 that is configured to extend through the receiver 101414 and be moved by a user relative to the receiver 101414. For example, the coupler 101411 can include one or more of a threaded connection, features that mate with threads, detent connections, such as outwardly biased arms or portions, flexible projections etc. The coupler 101411 attaches to the connector 101424 such that movement of the width adjustment element relative to the receiver 101414 causes the connector 101424 between the narrowed and expanded positions. In some implementations, the receiver 101414 can be integrally formed with a distal cap 101415 of the distal portion 101407 of the implantable device 101400. While described here as a receiver, other structures or openings in structures of a variety of shapes and sizes that can accomplish the same purpose can be used as well.

The coupler 101411 can be connected to or can include, for example, an actuation wire, actuation shaft, or any other suitable element that a user can engage to move the connector 101424 between the narrowed and expanded positions. For example, a distal end, a proximal end, or another portion of the coupler 101411 can include a connection feature 101413 for receiving and connecting to a retention feature of the connector 101424 (e.g., the retention feature 101372 of the connector 101324 shown in FIG. 139). The connector or connection feature 101413 can include openings 101444 for receiving the retention feature of the connector 101324. For example, similar to the connection shown in FIGS. 136-139, the openings 101444 can be configured to receive arms of the connector 101424. In the illustrated example, the connector or connection feature 101413 is a separate component from the remainder of the coupler 101411. For example, a proximal end of the connector or connection feature 101413 can be configured to attach to the remainder of the coupler 101411. In the illustrated example, the connector or connection feature 101413 includes a connection opening 101455 for receiving another portion of the coupler 101411. The connection between the remainder of the coupler 101411 and the connection opening 101455 can take any suitable form, such as, for example, any form described in the present application. In some implementations, the connector or connection feature 101413 is integral to another component of the coupler 101411.

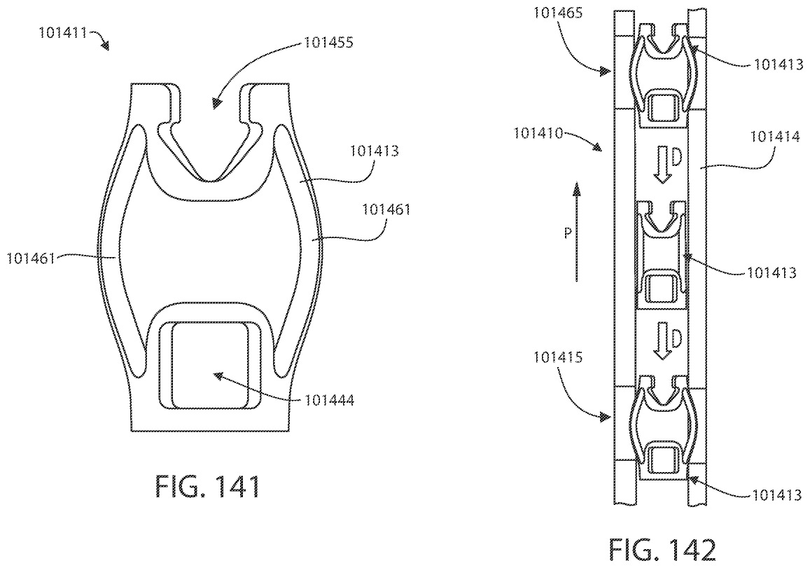

The connection feature 101413 can have one or more protruding side wall portions 101461 (FIGS. 141-142) that are movable between a normal, expanded position (as shown in FIGS. 141 and 142) and a compressed position (as shown in FIG. 142), and the receiver 101414 can have a plurality of holes or openings 101465 (shown for example in FIGS. 140 and 142; however, other similar elements like indentations, protrusions, notches, etc. could be used as well) for receiving the protruding side wall portions 101461 when the side wall portions 101461 are in the normal, expanded position. The connection between the protruding side wall portions 101461 of the connector or connection feature 101413 and the openings 101465 of the receiver 101414 secures the connection feature 101413 (and, consequently, the inner end of the connector 101424) at a desired location within the receiver 101414, which allows a user to maintain the connector 101424 at a desired width. The protruding side wall portions 101461 can be made of a flexible and/or shape set material, such as, for example, the arms 101263 can be made of metal, plastic, composite material, shape memory material, etc.

Referring to FIG. 142, a user can move the connector or connection feature 101413 in a proximal direction P or a distal direction D relative to the receiver 101414. When the connection feature 101413 is aligned with an opening 101465 of the receiver 101414, the protruding side wall portions 101461 can move to their normal, expanded position such that the protruding side wall portions 101461 extend into the aligned opening 101465, which allows a user to maintain the connection feature 101413 and, consequently, the connector 101424 (FIG. 140) in a desired position. When a user provides a force to the connection feature 101413 in either the proximal direction P or the distal direction D, the protruding side wall portions 101461 are engaged by the interior surface of the receiver 101414 such that the protruding side wall portions 101461 move to the compressed position, which allows the user to move the connection feature 101413 and, consequently, the connector 101424 relative to the receiver 101414.

While the protruding side wall portions 101461 are described as being a component of the coupler 101411, in some implementations, it should be understood that the protruding side wall portions 101461 that engage the openings 101465 of the receiver 101414 can be a component of the connector 101424. For example, the protruding side wall portions can be attached to or integral to the inner end of the connector 101424.

In some implementations, movement of the connection feature 101413 in the proximal direction P causes the connector 101424 to move such that the width W14 (FIG. 140) moves to the narrowed position, and movement of the connection feature 101413 in the distal direction D causes the connector 101424 to move such that the width W14 of the connector 101424 move to the expanded position. However, it should be understood that other configurations are also contemplated. In some implementations, the distal ends 101466 of the connector 101424 can move into the receiver 101414 when the connector 101424 is moved to the narrowed position, and the distal ends 101466 can move out of the receiver 101414 when the connector 101424 are moved to the expanded position. However, it should be understood that other configurations are also contemplated.

The movement of the connector 101424 to the narrowed position allows the device or implant 101400 to maneuver more easily into position for implantation in the heart by reducing the contact and/or friction between the native structures of the heart—e.g., chordae— and the device 101400. The movement of the connector 101424 to the expanded position provides the anchor portion of the device or implant 101400 with a larger surface area to engage and capture leaflet(s) of a native heart valve.

In some implementations, the connector 101424 can be made from or comprise a material that allows the inner end and portions of the connector 101424 (e.g., the distal ends 101066) to be pulled into the actuation portion 101410. For example, the connector 101424, or a portion thereof, can be made of any flexible material, including but not limited to, metal, plastic, fabric, suture, etc. The connector 101424 can be made using a variety of processes, including, but not limited to, cutting, such as laser cutting, stamping, casting, molding, heat treating, shape setting, etc. The connector 101424 can be made from or comprise a shape memory material, —such as Nitinol—to provide shape-setting capability.

Referring to FIG. 140, a connection feature 101416 is disposed at a proximal end of the implantable device 101400 for receiving an actuation element (not shown) of a delivery device (not shown). The connection between the connection feature 101416 and the actuation element of the delivery device can take any suitable form, such as, for example, the form of the connection between the implantable device 101100 and the actuation element 101102 shown in FIGS. 123-131, the form of the connection between the implantable device 101200 and the actuation element 101202 shown in FIGS. 132-135, or any other suitable connection described in the present application. In the illustrated example, the connection feature 101416 is threadably attached to the receiver 101414. The connection feature 101416 can, however, be attached to the receiver 101414 or any other portion of the implantable device 101400 by any suitable means. In some implementations, the connection feature 101416 can be integral to the receiver 101414.

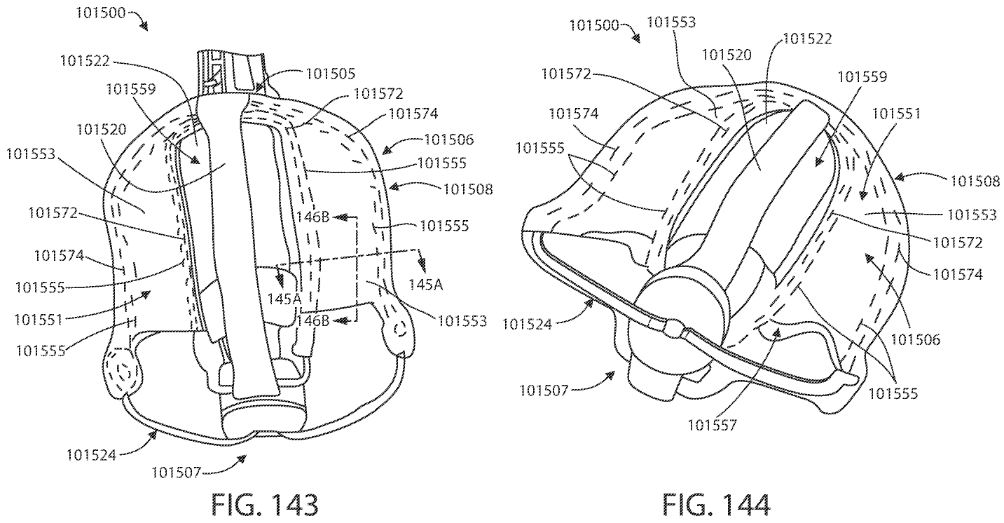

Referring to FIGS. 143-146, an example of an implantable device or implant 101500 includes an anchor portion 101506 having one or more paddle frames 101524 and a cover 101551. Referring to FIGS. 143-144, the implantable device 101500 includes a proximal or attachment portion 101505, an anchor portion 101506 having paddle frames 101524, an actuation portion (e.g., any actuation portion described in the present application), an optional spacer or coaptation element (e.g., any coaptation element described in the present application), and a distal portion 101507. The attachment portion 101505, the anchor portion 101506, and the distal portion 101507 can be configured in a variety of ways, such as, for example any way described in the present application.

In some implementations, the anchor portion 101506 can include at least one anchor 101508, where the anchor 101508 has an outer paddle 101520, an inner paddle 101522, a paddle extension member or paddle frame 101524, clasps (not shown), and a cover 101551. The outer and inner paddles 101520, 101522 can take any suitable form, such as, for example, any form described in the present application. The paddle frame 101524 can have an inner frame portion 101572 and an outer frame portion 101574, such as, for example, similar to any other paddle frame disclosed in the present application that includes an inner frame portion and an outer frame portion.

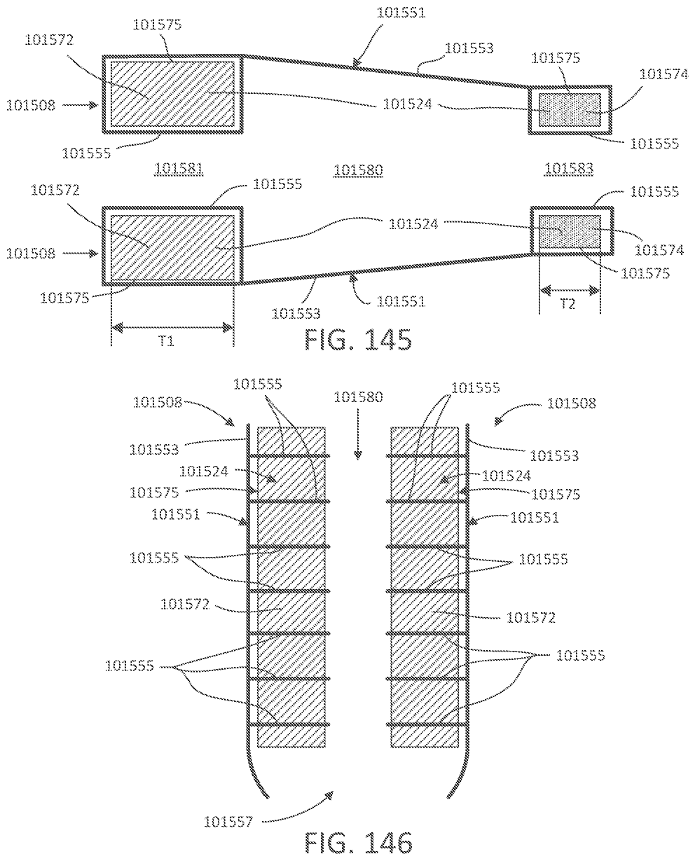

The inner frame portion 101572 can be rigid our substantially rigid such that it is configured to support the paddles and provide a sufficient force to leaflets of the native valve during capture. The inner frame portion 101572 can be made of, for example, metals, plastics, etc. The outer frame portions 101574 can be connected to the inner frame portions 101572 such that the outer frame portions 101574 define the total width of the paddle frame 101524. The outer frame portions 101574 can be flexible such that the outer frame portions can move between an expanded position and a narrowed position. For example, the outer frame portions 101574 can be operatively connected to an actuation portion of the implantable device such that a user can engage the paddle frame 101524 to move the outer frame portions 101574 between the expanded and narrowed positions. The outer frame portions 101574 can be made of, for example, metals, and plastics. FIG. 145 is a cross-sectional view taken along the plane indicated by lines 145A-145A in FIG. 143. Referring to the section of FIG. 145, in the illustrated example, the inner frame portions 101572 have a thickness T1 that is greater than a thickness T2 of the outer frame portions 101574, which causes the inner frame portions 101572 to be rigid and the outer frame portions 101574 to be flexible.

Referring to FIGS. 145 and 146, in some implementations, the implantable device 101500 (FIGS. 143-144) includes a pair of anchors 101508 having a cover 101551, where the anchors 101508 are configured to be moved to a closed position such that leaflets of a native valve can be compressed and captured in the area 101580 between the two anchors 101508. While the illustrated example is shown as having a pair of anchors 101508, it should be understood that the implantable device 101500 can have any suitable number of anchors that include a cover 101551. When the anchors 101508 are compressing the leaflets, the leaflets are engaged at the pinch points between the anchors 101508 (while the term “pinch point” is often used, this includes pinch areas or regions where the leaflet(s) are compressed by portions of the device). That is, the leaflets are engaged at a first pinch point 101581 between the inner frame portions 101572 of the anchors 101508, and the leaflets are engaged at a second pinch point 101583 between outer frame portions 101574 of the anchors 101508. FIGS. 145-146 show only the section of the paddle frame 101524 taken along the plane indicated by the lines 145A-145A and 146B-146B shown in FIG. 143. It should be understood that additional pinch points or elongated pinch areas can exist that result from the engagement between the inner frame portions 101572 for each of the anchors 101508 and/or the engagement between the outer frame portions 101574 for each of the anchors 101508.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 143-146, the cover 101551 can be attached to the paddle frame 101524. The cover can be configured to provide further engagement between the implantable device 101500 and the leaflets. Any of the covers described herein can include a sheet, material, fabric, layer, and/or membrane 101553 that is attached to the inner frame portion 101572 and outer frame portion 101574 by a plurality of connectors 101555 (e.g., stitches, adhesive, mechanical fasteners, ultrasonic welds, etc.). In the illustrated example, the sheet, material, fabric, layer, and/or membrane 101553 is attached to the frame portions 101572, 101574 such that the sheet, material, fabric, layer, and/or membrane extends across the outer surfaces 101575 (FIGS. 145-146) of the frame portions 101572, 101574. The sheet, material, fabric, layer, and/or membrane 101553 can be comprise a flexible material, a porous material, a non-porous material, and/or a material that is impermeable to (or that inhibits or impedes) blood flow, etc. In some implementations, the sheet, material, fabric, layer, and/or membrane 101553 is made from or comprises a biocompatible material, such as a woven biocompatible fabric that is configured to promote tissue ingrowth.

In the illustrated example, each anchor 101508 includes a separate sheet, material, fabric, layer, and/or membrane 101553 such that a gap 101557 (FIGS. 144 and 146) exists between the anchors 101508. In some implementations, a single sheet, material, fabric, layer, and/or membrane 101553 can be secured to the paddle frames 101524 of both anchors 101508 such that the covers 101551 of the two anchors are connected. In these implementations, the sheet, material, fabric, layer, and/or membrane 101553 (or a combination of materials) can form a canopy (e.g., similar to the canopy 101667 shown in FIG. 150 and described below) between the paddle frames 101524 of the anchors 101508.

When the anchors 101508 are compressing the leaflets, the cover 101551 engages the leaflets between the pinch points 101581, 101583 to assist in coapting the leaflets and preventing or inhibiting regurgitation of blood through the native valve. In addition to providing force to the leaflets, when the cover 101551 forms a canopy, the cover can further prevent or inhibit regurgitation of blood by acting as a barrier that blocks, inhibits, or diverts the movement of blood through the native valve. In some implementations, the sheet, material, fabric, layer, and/or membrane 101553 of the cover 101551 is flexible and/or stretchable to allow for movement of the paddle frames 101524 between narrowed and expanded positions. For example, the inner frame portions 101572 can be fixed, and the width of the outer frame portions 101574 can be adjustable, and as the outer frame portions are moved to an expanded position, the sheet, material, fabric, layer, and/or membrane 101553 of the cover 101551 can be configured to stretch or otherwise expand. The flexibility and/or resilience of the sheet, material, fabric, layer, and/or membrane 101553 can be selected to allow the cover 101551 to maintain a taut state when the paddle frames 101524 are in a narrowed position. This helps the cover prevent or inhibit leakage in whatever size the adjustable frame members are adjusted to or set and avoid gaps or openings forming as components of the device are adjusted to different sizes.

The cover 101551 can be configured to cover or not cover any component or portion of the implantable device. For example, referring to FIGS. 143 and 144, in the illustrated example, the cover 101551 extends between the inner and outer frame portions 101572, 101574 of the paddle frame 101524, but the cover 101551 does not encapsulate the outer paddle 101520 or inner paddle 101522 of the anchors 101508. That is, the sheet, material, fabric, layer, and/or membrane 101553 of the cover 101551 includes an opening or cutout 101559 such that the cover 101551 does not cover the paddles 101520, 101522. However, it should be understood that other configurations are also contemplated.

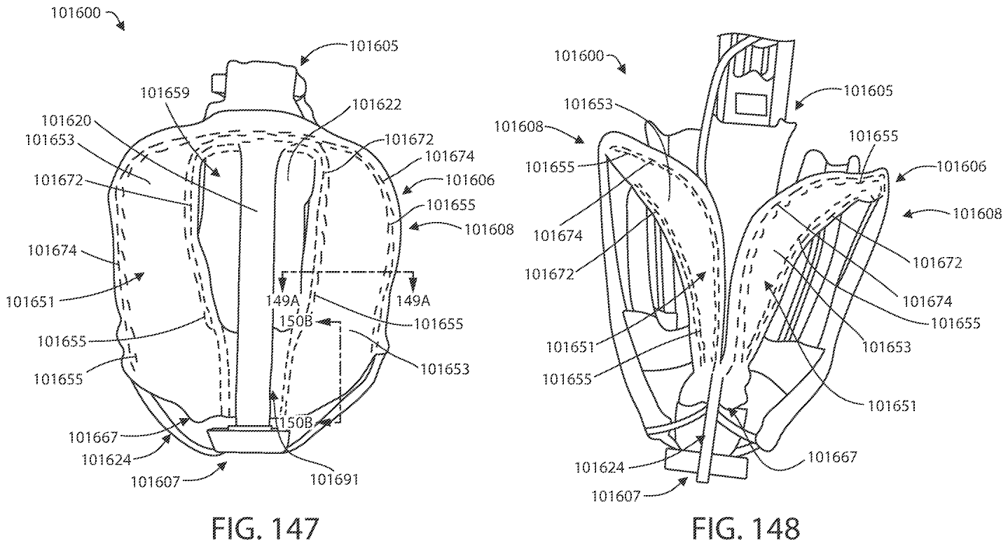

Referring to FIGS. 147-150, an example of an implantable device or implant 101600 includes an anchor portion 101606 having one or more paddle frames 101624 and a cover 101651. Referring to FIGS. 147-148, the implantable device 101600 includes a proximal or attachment portion 101605, an anchor portion 101606 having paddle frames 101624, an actuation portion (e.g., any actuation portion described in the present application), an optional spacer or coaptation element (e.g., any coaptation element described in the present application), and a distal portion 101607. The attachment portion 101605, the anchor portion 101606, and the distal portion 101607 can be configured in a variety of ways, such as, for example any way described in the present application. While the illustrated example shows the membrane 101653 connected to both the inner frame portion 101672 and the outer frame portion 101674, it should be understood that the membrane 101653 can be attached to one of the inner frame portion 101672 and the outer frame portion 101674. Any suitable number of connectors 101655 can be used to connect the membrane 101653 to the frame portions 101672, 101674.

In some implementations, the anchor portion 101606 can include at least one anchor 101608, where the anchor 101608 has an outer paddle 101620, an inner paddle 101622, a paddle extension member or paddle frame 101624, clasps (not shown), and a cover 101651. The paddles 101620, 101622 can take any suitable form, such as, for example, any form described in the present application. The paddle frame 101624 can have an inner frame portion 101672 and an outer frame portion 101674, such as, for example, similar to any other paddle frame disclosed in the present application that includes an inner frame portion and an outer frame portion.

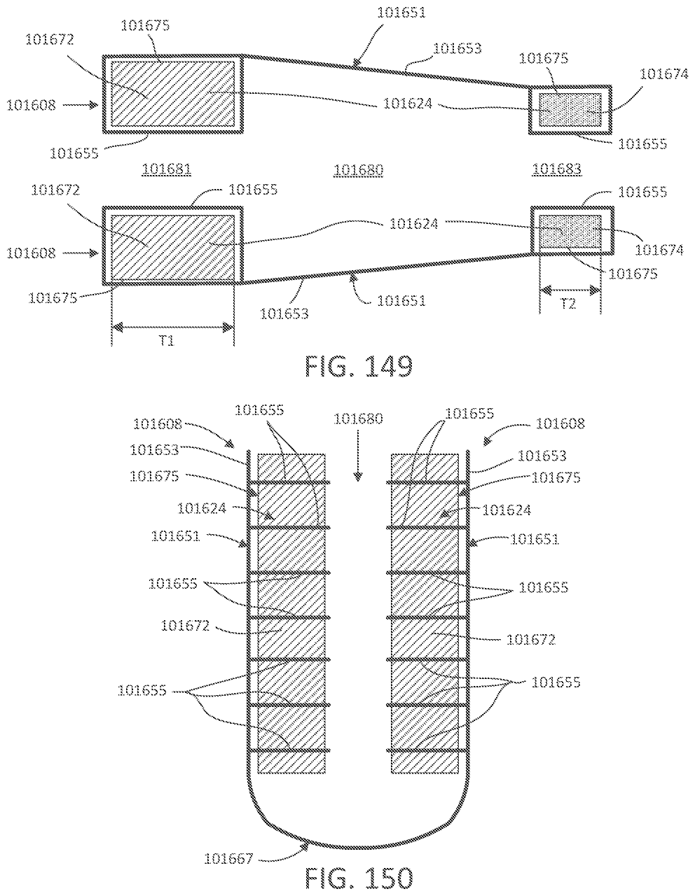

The inner frame portion 101672 can be rigid such that it is configured to support the paddles and provide a sufficient force to leaflets of the native valve during capture. The inner frame portion 101672 can be made of, for example, metals, plastics, etc. The outer frame portions 101674 can be connected to the inner frame portions 101672 such that the outer frame portions 101674 define the total width of the paddle frame 101624. The outer frame portions 101674 can be flexible such that the outer frame portions can optionally be manipulated by a user between an expanded position and a narrowed position. For example, the outer frame portions 101674 can be operatively connected to an actuation portion of the implantable device such that a user can engage the paddle frame 101624 to move the outer frame portions 101674 between the expanded and narrowed positions. The outer frame portions 101674 can be made of, for example, metals, and plastics. Referring to FIG. 149, in the illustrated example, the inner frame portions 101672 have a thickness T1 that is greater than a thickness T2 of the outer frame portions 101674, which allows the inner frame portions 101672 to be rigid and the outer frame portions 101674 to be flexible.

Referring to FIGS. 149 and 150, in some implementations, the implantable device 101600 (FIGS. 147-148) includes a pair of anchors 101608 having a cover 101651, where the anchors 101608 are configured to be moved to a closed position such that leaflets of a native valve can be compressed and captured in the area 101680 between the two anchors 101608. While the illustrated example is shown as having a pair of anchors 101608, it should be understood that the implantable device 101600 can have any suitable number of anchors that include a cover 101651. When the anchors 101608 are compressing the leaflets, the leaflets are engaged at the pinch points between the anchors 101608 (e.g., in areas/regions whether the leaflets can be compressed by portions of the device). That is, the leaflets are engaged at a first pinch point 101681 between the inner frame portions 101672 of the anchors 101608, and the leaflets are engaged at a second pinch point 101683 between outer frame portions 101674 of the anchors 101608. FIGS. 149-150 show only the of the paddle frame 101624 taken along the plane indicated by the lines 149A-149A and 150B-150B shown in FIG. 147. It should be understood that additional pinch points or pinch areas exist resulting from the engagement between the inner frame portions 101672 for each of the anchors 101608 and the engagement between the outer frame portions 101674 for each of the anchors 101608.

Referring to FIGS. 147-150, the cover 101651 can be attached to the paddle frame 101624 and configured to provide further engagement between the implantable device 101600 and the leaflets. Each cover can include a sheet, material, fabric, layer, and/or membrane 101653 that is attached to the inner frame portion 101672 and outer frame portion 101674 by a plurality of connectors 101655 (e.g., stitches, adhesive, mechanical fasteners, ultrasonic welds, etc.). In the illustrated example, the sheet, material, fabric, layer, and/or membrane 101653 is attached to the frame portions 101672, 101674 such that the sheet, material, fabric, layer, and/or membrane extends across an outer surface 101675 (FIGS. 149-150) of the frame portions 101672, 101674. The sheet, material, fabric, layer, and/or membrane 101653 can be made of a flexible material, a porous material, non-porous material, and/or a material that is impermeable to (or inhibits or impedes) blood flow. In some implementations, the sheet, material, fabric, layer, and/or membrane 101653 is made from or comprises a biocompatible material, such as a woven biocompatible fabric that is configured to promote tissue ingrowth. In the illustrated example, a single sheet, material, fabric, layer, and/or membrane 101653 is secured to the paddle frames 101624 of both anchors 101608 such that the covers 101651 of the two anchors are connected. In these implementations, the single sheet, material, fabric, layer, and/or membrane 101653 can form a canopy 101667 (FIGS. 148 and 150) between the paddle frames 101624 of the anchors 101608. In some implementations, each anchor 101608 includes a separate sheet, material, fabric, layer, and/or membrane 101653 such that the covers 101651 of the two anchors 101608 are not connected and a gap (e.g., the gap 101557 for the device 101500 shown in FIGS. 143-146) exists between the anchors 101608.

When the anchors 101608 are compressing the leaflets, the cover 101651 engages the leaflets between the pinch points 101681, 101683 to assist in coapting the leaflets and preventing or inhibiting regurgitation of blood through the native valve. In addition to providing force to the leaflets, the cover 101651 can further prevent or inhibit regurgitation of blood by acting as a barrier that prevents or inhibits the movement of blood through the native valve. The canopy 101667 can act as a further barrier for preventing or inhibiting blood from moving through the native valve. In some implementations, the sheet, material, fabric, layer, and/or membrane 101653 of the cover 101651 is flexible to allow for movement of the paddle frames 101624 between narrowed and expanded positions. For example, the inner frame portions 101672 can be fixed, and the width of the outer frame portions 101674 can be adjustable, and as the outer frame portions are moved to an expanded position, the sheet, material, fabric, layer, and/or membrane 101653 of the cover 101651 can be configured to stretch or otherwise expand. The flexibility and/or resiliency of the sheet, material, fabric, layer, and/or membrane 101653 allows the cover 101651 to maintain a taut state when the paddle frames 101624 are in a narrowed position. This helps the cover prevent or inhibit leakage in various sizes of the adjustable frame members, without forming leakage gaps or openings between components of the device.

The cover 101651 can be configured to cover or not cover any component or portion of the implantable device. For example, referring to FIGS. 147 and 148, in the illustrated example, the cover 101651 extends between the inner and outer frame portions 101672, 101674 of the paddle frame 101624, as well as in a portion of an area 101691 (FIG. 147) defined by the interior of the inner frame portions 101672, but not the entirety of the area defined by the interior of the inner frame portions 101672. The cover 101651, however, does not encapsulate the entirety of the outer paddle 101620 or a portion of the inner paddle 101622 of the anchors 101608. That is, the sheet, material, fabric, layer, and/or membrane 101653 of the cover 101651 includes an opening or cutout 101659 such that the cover 101651 does not cover at least a portion of the paddles 101620, 101622. However, it should be understood that other configurations are also contemplated.

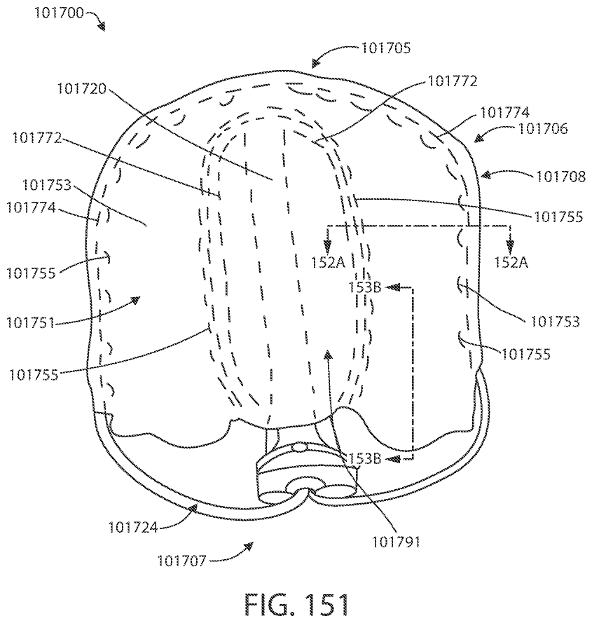

Referring to FIGS. 151-153, an example of an implantable device or implant 101700 includes an anchor portion 101706 having one or more paddle frames 101724 and a cover 101751. Referring to FIG. 151, the implantable device 101700 includes a proximal or attachment portion 101705, an anchor portion 101706 having paddle frames 101724, an actuation portion (e.g., any actuation portion described in the present application), an optional spacer or coaptation element (e.g., any coaptation element described in the present application), and a distal portion 101707. The attachment portion 101705, the anchor portion 101706, and the distal portion 101707 can be configured in a variety of ways, such as, for example any way described in the present application.

In some implementations, the anchor portion 101706 can include at least one anchor 101708, where the anchor 101708 has an outer paddle 101720, an inner paddle (not shown), a paddle extension member or paddle frame 101724, clasps (not shown), and a cover 101751. The inner and outer paddles can take any suitable form, such as, for example, any form described in the present application. The paddle frame 101724 can have an inner frame portion 101772 and an outer frame portion 101774, such as, for example, similar to any other paddle frame disclosed in the present application that includes an inner frame portion and an outer frame portion.

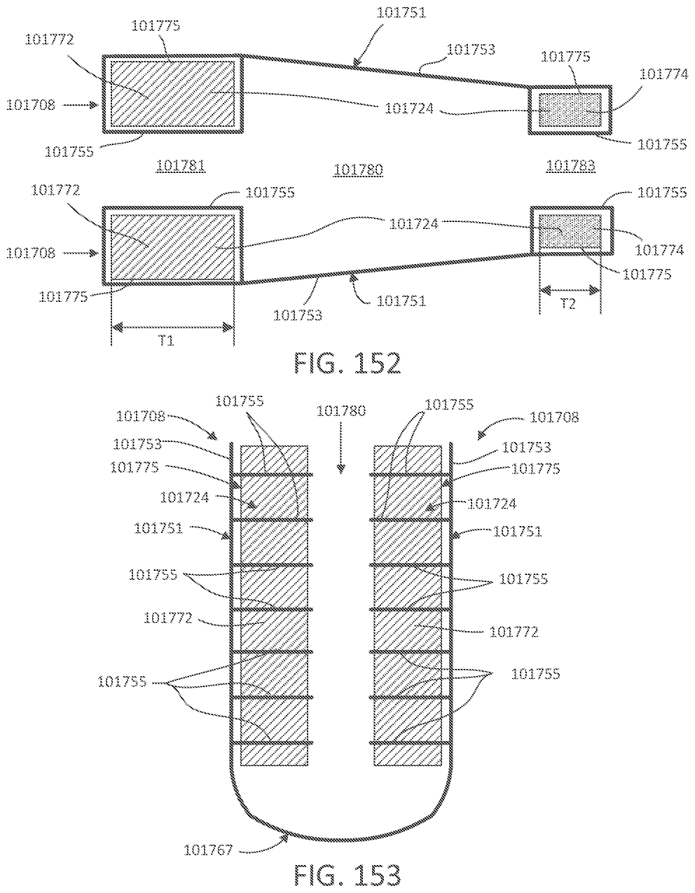

The inner frame portion 101772 can be rigid such that it is configured to support the paddles and provide a sufficient force to leaflets of the native valve during capture. The inner frame portion 101772 can be made of, for example, metals, plastics, etc. The outer frame portions 101774 can be connected to the inner frame portions 101772 such that the outer frame portions 101774 define the total width of the paddle frame 101724. The outer frame portions 101774 can be flexible such that the outer frame portions can optionally be manipulated by a user between an expanded position and a narrowed position. For example, the outer frame portions 101774 can be operatively connected to an actuation portion of the implantable device such that a user can engage the paddle frame 101724 to move the outer frame portions 101774 between the expanded and narrowed positions. The outer frame portions 101774 can be made of, for example, metals, and plastics. Referring to FIG. 152, in the illustrated example, the inner frame portions 101772 have a thickness T1 that is greater than a thickness T2 of the outer frame portions 101774, which allows the inner frame portions 101772 to be rigid and the outer frame portions 101774 to be flexible.

Referring to FIGS. 152 and 153, in some implementations, the implantable device 101700 (FIG. 151) includes a pair of anchors 101708 having a cover 101751, where the anchors 101708 are configured to be moved to a closed position such that leaflets of a native valve can be compressed and captured in the area 101780 between the two anchors 101708.

While the illustrated example is shown as having a pair of anchors 101708, it should be understood that the implantable device 101700 can have any suitable number of anchors that include a cover 101751. When the anchors 101708 are compressing the leaflets, the leaflets are engaged at the pinch points (e.g., pinch regions) between the anchors 101708. That is, the leaflets are engaged at a first pinch point 101781 between the inner frame portions 101772 of the anchors 101708, and the leaflets are engaged at a second pinch point 101783 between outer frame portions 101774 of the anchors 101708. FIGS. 152-153 show only a portion of the paddle frame 101724 taken from the lines 152A-152A and 153B-153B shown in FIG. 151. It should be understood that additional pinch points exist resulting from the engagement between the inner frame portions 101772 for each of the anchors 101708 and the engagement between the outer frame portions 101774 for each of the anchors 101708.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 151-153, the cover 101751 can be attached to the paddle frame 101724 and configured to provide further engagement between the implantable device 101700 and the leaflets. Each cover can include a sheet, material, fabric, layer, and/or membrane 101753 that is attached to the inner frame portion 101772 and outer frame portion 101774 by a plurality of connectors 101755 (e.g., stitches, adhesive, mechanical fasteners, ultrasonic welds, etc.). While the illustrated example shows the membrane 101753 connected to both the inner frame portion 101772 and the outer frame portion 101774, it should be understood that the membrane 101753 can be attached to one of the inner frame portion 101772 and the outer frame portion 101774. Any suitable number of connectors 101755 can be used to connect the membrane 101753 to the frame portions 101772, 101774.

In the illustrated example, the sheet, material, fabric, layer, and/or membrane 101753 is attached to the frame portions 101772, 101774 such that the sheet, material, fabric, layer, and/or membrane extends across an outer surface 101775 (FIGS. 152-153) of the frame portions 101772, 101774. The sheet, material, fabric, layer, and/or membrane 101753 can be made of a flexible material, a porous material, a non-porous material, and/or a material that is impermeable to (or inhibits or impedes) blood flow. In some implementations, the sheet, material, fabric, layer, and/or membrane 101753 is made from or comprises a biocompatible material, such as a woven biocompatible fabric that is configured to promote tissue ingrowth. In the illustrated example, a single sheet, material, fabric, layer, and/or membrane 101753 is secured to the paddle frames 101724 of both anchors 101708 such that the covers 101751 of the two anchors are connected. In these implementations, the single sheet, material, fabric, layer, and/or membrane 101753 can form a canopy 101767 (FIGS. 151 and 153) between the paddle frames 101724 of the anchors 101708. In some implementations, each anchor 101708 includes a separate sheet, material, fabric, layer, and/or membrane 101753 such that the covers 101751 of the two anchors 101708 are not connected and a gap (e.g., the gap 101557 for the device 101500 shown in FIGS. 143-146) exists between the anchors 101708.

When the anchors 101708 are compressing the leaflets, the cover 101751 engages the leaflets between the pinch points 101781, 101783 to assist in coapting the leaflets and preventing or inhibiting regurgitation of blood through the native valve. In addition to providing force to the leaflets, the cover 101751 can further prevent or inhibit regurgitation of blood by acting as a barrier that prevents or inhibits the movement of blood through the native valve. The canopy 101767 can act as a further barrier for preventing or inhibiting blood from moving through the native valve. In some implementations, the sheet, material, fabric, layer, and/or membrane 101753 of the cover 101751 is flexible to allow for movement of the paddle frames 101724 between narrowed and expanded positions. For example, the inner frame portions 101772 can be fixed, and the width of the outer frame portions 101774 can be adjustable, and as the outer frame portions are moved to an expanded position, the sheet, material, fabric, layer, and/or membrane 101753 of the cover 101751 can be configured to stretch or otherwise expand. The flexibility of the sheet, material, fabric, layer, and/or membrane 101753 allows the cover 101751 to maintain a taut state when the paddle frames 101724 are in a narrowed position. This helps the cover prevent or inhibit leakage in various sizes of the adjustable frame members, without forming leakage gaps or openings between components of the device.

The cover 101751 can be configured to cover or not cover any component or portion of the implantable device. For example, referring to FIG. 151, in the illustrated example, the cover 101751 extends between the inner and outer frame portions 101772, 101774 of the paddle frame 101724, as well extending an entirety of an area 101791 (FIG. 151) defined by the interior of the inner frame portions 101772. The cover 101751 encapsulates the inner and outer paddles of the anchors 101708. That is, in the illustrated example, the sheet, material, fabric, layer, and/or membrane 101653 does not include any opening or cutout. However, it should be understood that other configurations are also contemplated.

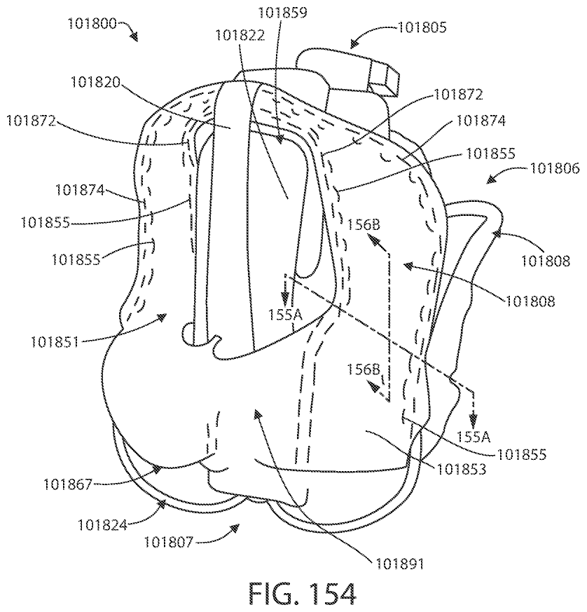

Referring to FIGS. 154-156, an example of an implantable device or implant 101800 includes an anchor portion 101806 having one or more paddle frames 101824 and a cover 101851. Referring to FIG. 154, the implantable device 101800 includes a proximal or attachment portion 101805, an anchor portion 101806 having paddle frames 101824, an actuation portion (e.g., any actuation portion described in the present application), an optional spacer or coaptation element (e.g., any coaptation element described in the present application), and a distal portion 101807. The attachment portion 101805, the anchor portion 101806, and the distal portion 101807 can be configured in a variety of ways, such as, for example any way described in the present application.

In some implementations, the anchor portion 101806 can include at least one anchor 101808, where the anchor 101808 has an outer paddle 101820, an inner paddle 101822, a paddle extension member or paddle frame 101824, clasps (not shown), and a cover 101851. The paddles 101820, 101822 can take any suitable form, such as, for example, any form described in the present application. The paddle frame 101824 can have an inner frame portion 101872 and an outer frame portion 101874, such as, for example, similar to any other paddle frame disclosed in the present application that includes an inner frame portion and an outer frame portion.

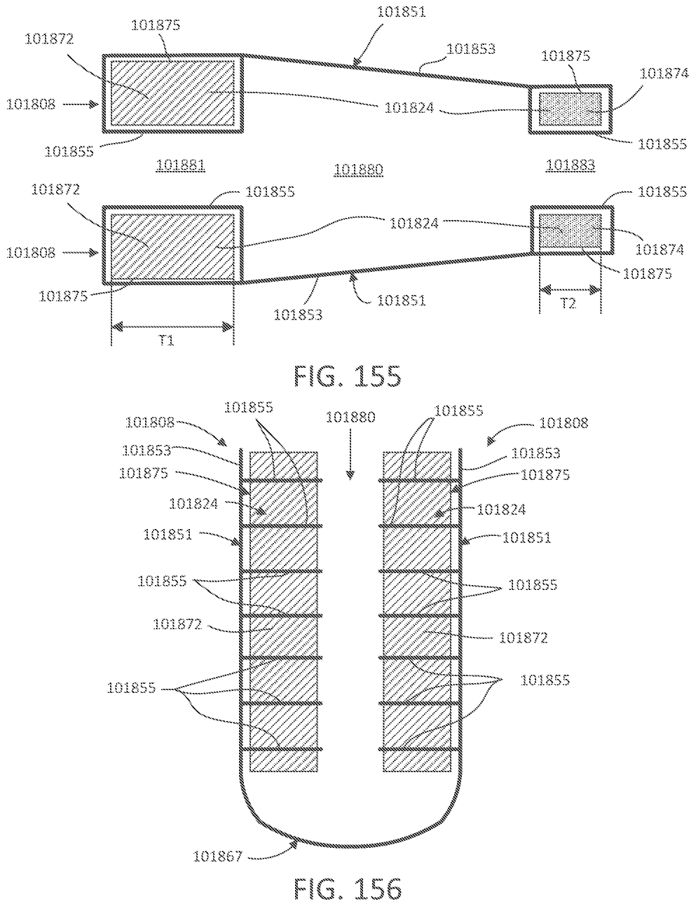

The inner frame portion 101872 can be rigid such that it is configured to support the paddles and provide a sufficient force to leaflets of the native valve during capture. The inner frame portion 101872 can be made of, for example, metals, plastics, etc. The outer frame portions 101874 can be connected to the inner frame portions 101872 such that the outer frame portions 101874 define the total width of the paddle frame 101824. The outer frame portions 101874 can be flexible such that the outer frame portions can optionally be manipulated by a user between an expanded position and a narrowed position. For example, the outer frame portions 101874 can be operatively connected to an actuation portion of the implantable device such that a user can engage the paddle frame 101824 to move the outer frame portions 101874 between the expanded and narrowed positions. The outer frame portions 101874 can be made of, for example, metals, and plastics. Referring to FIG. 155, in the illustrated example, the inner frame portions 101872 have a thickness T1 that is greater than a thickness T2 of the outer frame portions 101874, which allows the inner frame portions 101872 to be rigid and the outer frame portions 101874 to be flexible.

Referring to FIGS. 155 and 156, in some implementations, the implantable device 101800 (FIG. 154) includes a pair of anchors 101808 having a cover 101851, where the anchors 101808 are configured to be moved to a closed position such that leaflets of a native valve can be compressed and captured in the area 101880 between the two anchors 101808. While the illustrated example is shown as having a pair of anchors 101808, it should be understood that the implantable device 101800 can have any suitable number of anchors that include a cover 101851. When the anchors 101808 are compressing the leaflets, the leaflets are engaged at the pinch points/regions between the anchors 101808. That is, the leaflets are engaged at a first pinch point 101881 between the inner frame portions 101872 of the anchors 101808, and the leaflets are engaged at a second pinch point 101883 between outer frame portions 101874 of the anchors 101808. FIGS. 155-156 show only a portion of the paddle frame 101824 taken from the lines 155A-155A and 156B-156B shown in FIG. 154. It should be understood that additional pinch points exist resulting from the engagement between the inner frame portions 101872 for each of the anchors 101808 and the engagement between the outer frame portions 101874 for each of the anchors 101808.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 154-156, the cover 101851 can be attached to the paddle frame 101824 and configured to provide further engagement between the implantable device 101800 and the leaflets. Each cover can include a sheet, material, fabric, layer, and/or membrane 101853 that is attached to the inner frame portion 101872 and outer frame portion 101874 by a plurality of connectors 101855 (e.g., stitches, adhesive, mechanical fasteners, ultrasonic welds, etc.). While the illustrated example shows the membrane 101853 connected to both the inner frame portion 101872 and the outer frame portion 101874, it should be understood that the membrane 101853 can be attached to one of the inner frame portion 101872 and the outer frame portion 101874. Any suitable number of connectors 101855 can be used to connect the membrane 101853 to the frame portions 101872, 101874.

In the illustrated example, the sheet, material, fabric, layer, and/or membrane 101853 is attached to the frame portions 101872, 101874 such that the sheet, material, fabric, layer, and/or membrane extends across an outer surface 101875 (FIGS. 155-156) of the frame portions 101872, 101874. The sheet, material, fabric, layer, and/or membrane 101853 can be made of a flexible material, a porous material, a non-porous material, and/or a material that is impermeable to (or inhibits or impedes) blood flow. In some implementations, the sheet, material, fabric, layer, and/or membrane 101853 is made from or comprises a biocompatible material, such as a woven biocompatible fabric that is configured to promote tissue ingrowth. In the illustrated example, a single sheet, material, fabric, layer, and/or membrane 101853 is secured to the paddle frames 101824 of both anchors 101808 such that the covers 101851 of the two anchors are connected. In these implementations, the single sheet, material, fabric, layer, and/or membrane 101853 can form a canopy 101867 (FIGS. 154 and 156) between the paddle frames 101824 of the anchors 101808. In some implementations, each anchor 101808 includes a separate sheet, material, fabric, layer, and/or membrane 101853 such that the covers 101851 of the two anchors 101808 are not connected and a gap (e.g., the gap 101557 for the device 101500 shown in FIGS. 143-146) exists between the anchors 101808.

When the anchors 101808 are compressing the leaflets, the cover 101851 engages the leaflets between the pinch points 101881, 101883 to assist in coapting the leaflets and preventing or inhibiting regurgitation of blood through the native valve. In addition to providing force to the leaflets, the cover 101851 can further prevent or inhibit regurgitation of blood by acting as a barrier that prevents or inhibits the movement of blood through the native valve. The canopy 101867 can act as a further barrier for preventing or inhibiting blood from moving through the native valve. In some implementations, the sheet, material, fabric, layer, and/or membrane 101853 of the cover 101851 is flexible to allow for movement of the paddle frames 101824 between narrowed and expanded positions. For example, the inner frame portions 101872 can be fixed, and the width of the outer frame portions 101874 can be adjustable, and as the outer frame portions are moved to an expanded position, the sheet, material, fabric, layer, and/or membrane 101853 of the cover 101851 can be configured to stretch. The flexibility of the sheet, material, fabric, layer, and/or membrane 101853 allows the cover 101851 to maintain a taut state when the paddle frames 101824 are in a narrowed position.

The cover 101851 can be configured to cover or not cover any component or portion of the implantable device. For example, referring to FIG. 154, in the illustrated example, the cover 101851 extends between the inner and outer frame portions 101872, 101874 of the paddle frame 101824, as well as in a portion of an area 101891 defined by the interior of the inner frame portions 101872, but not the entirety of the area defined by the interior of the inner frame portions 101872. The cover 101851, however, does not encapsulate the at least a portion of the outer paddle 101820 or the inner paddle 101822 of the anchors 101808. That is, the sheet, material, fabric, layer, and/or membrane 101853 of the cover 101851 includes an opening or cutout 101859 such that the cover 101851 does not cover at least a portion of the paddles 101820, 101822. However, it should be understood that other configurations are also contemplated.

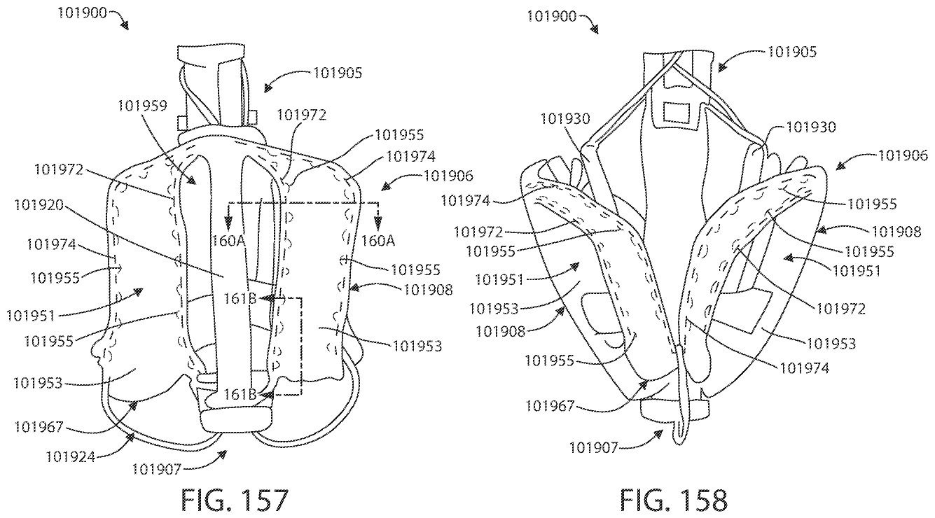

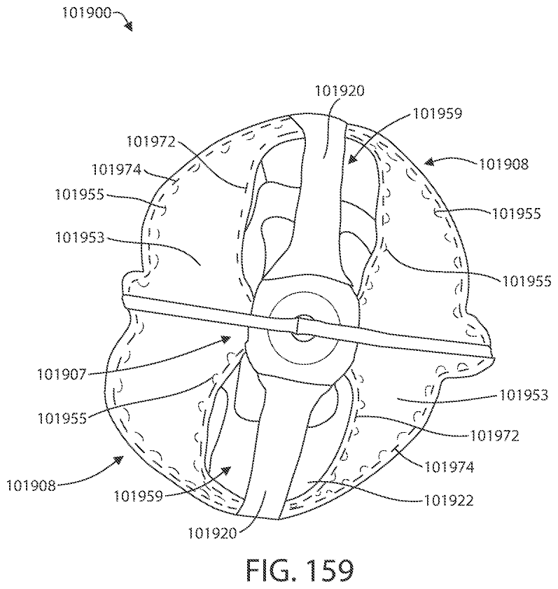

Referring to FIGS. 157-161, an example of an implantable device or implant 101900 includes an anchor portion 101906 having one or more paddle frames 101924 and a cover 101951. Referring to FIGS. 157-159, the implantable device 101900 includes a proximal or attachment portion 101905, an anchor portion 101906 having paddle frames 101924, an actuation portion (e.g., any actuation portion described in the present application), an optional spacer or coaptation element (e.g., any coaptation element described in the present application), and a distal portion 101907. The attachment portion 101905, the anchor portion 101906, and the distal portion 101907 can be configured in a variety of ways, such as, for example any way described in the present application.

In some implementations, the anchor portion 101906 can include at least one anchor 101908, where the anchor 101908 has an outer paddle 101920, an inner paddle (not shown), a paddle extension member or paddle frame 101924, clasps 101930, and a cover 101951. The paddles 101920, 101922 can take any suitable form, such as, for example, any form described in the present application. The paddle frame 101924 can have an inner frame portion 101972 and an outer frame portion 101974, such as, for example, similar to any other paddle frame disclosed in the present application that includes an inner frame portion and an outer frame portion.

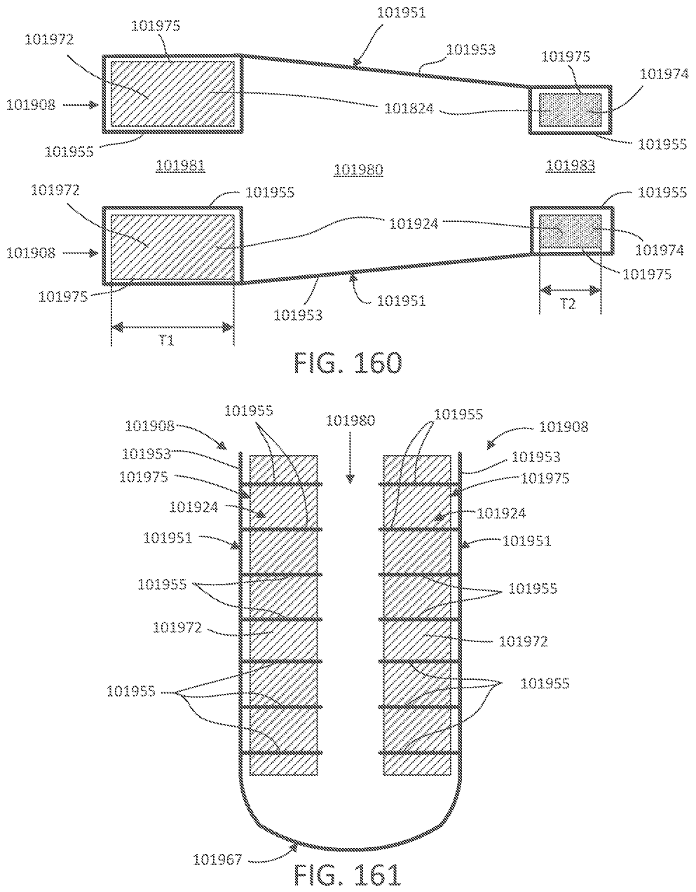

The inner frame portion 101972 can be rigid such that it is configured to support the paddles and provide a sufficient force to leaflets of the native valve during capture. The inner frame portion 101972 can be made of, for example, metals, plastics, etc. The outer frame portions 101974 can be connected to the inner frame portions 101972 such that the outer frame portions 101974 define the total width of the paddle frame 101924. The outer frame portions 101974 can be flexible such that the outer frame portions can optionally be manipulated by a user between an expanded position and a narrowed position. For example, the outer frame portions 101974 can be operatively connected to an actuation portion of the implantable device such that a user can engage the paddle frame 101924 to move the outer frame portions 101974 between the expanded and narrowed positions. The outer frame portions 101974 can be made of, for example, metals, and plastics. Referring to FIG. 160, in the illustrated example, the inner frame portions 101972 have a thickness T1 that is greater than a thickness T2 of the outer frame portions 101974, which allows the inner frame portions 101972 to be rigid and the outer frame portions 101974 to be flexible.

Referring to FIGS. 160 and 161, in some implementations, the implantable device 101900 (FIGS. 157-159) includes a pair of anchors 101908 having a cover 101951, where the anchors 101908 are configured to be moved to a closed position such that leaflets of a native valve can be compressed and captured in the area 101980 between the two anchors 101908. While the illustrated example is shown as having a pair of anchors 101908, it should be understood that the implantable device 101900 can have any suitable number of anchors that include a cover 101951. When the anchors 101908 are compressing the leaflets, the leaflets are engaged at the pinch points/regions between the anchors 101908. That is, the leaflets are engaged at a first pinch point 101981 between the inner frame portions 101972 of the anchors 101908, and the leaflets are engaged at a second pinch point 101983 between outer frame portions 101974 of the anchors 101908. FIGS. 160-161 show only a portion of the paddle frame 101924 taken from the lines 160A-160A and 161B-161B shown in FIG. 157. It should be understood that additional pinch points exist resulting from the engagement between the inner frame portions 101972 for each of the anchors 101908 and the engagement between the outer frame portions 101974 for each of the anchors 101908.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 157-161, the cover 101951 can be attached to the paddle frame 101924 and configured to provide further engagement between the implantable device 101900 and the leaflets. Each cover can include a sheet, material, fabric, layer, and/or membrane 101953 that is attached to the inner frame portion 101972 and outer frame portion 101974 by a plurality of connectors 101955 (e.g., stitches, adhesive, mechanical fasteners, ultrasonic welds, etc.). While the illustrated example shows the membrane 101953 connected to both the inner frame portion 101972 and the outer frame portion 101974, it should be understood that the membrane 101953 can be attached to one of the inner frame portion 101972 and the outer frame portion 101974. Any suitable number of connectors 101955 can be used to connect the membrane 101953 to the frame portions 101972, 101974.

In the illustrated example, the sheet, material, fabric, layer, and/or membrane 101953 is attached to the frame portions 101972, 101974 such that the sheet, material, fabric, layer, and/or membrane extends across an outer surface 101975 (FIGS. 160-161) of the frame portions 101972, 101974. The sheet, material, fabric, layer, and/or membrane 101953 can be made of a flexible material, a porous material, a non-porous material, and/or a material that is impermeable to (or inhibits or impedes) blood flow. In some implementations, the sheet, material, fabric, layer, and/or membrane 101953 is made from or comprises a biocompatible material, such as a woven biocompatible fabric that is configured to promote tissue ingrowth. In the illustrated example, a single sheet, material, fabric, layer, and/or membrane 101953 is secured to the paddle frames 101924 of both anchors 101908 such that the covers 101951 of the two anchors are connected. In these implementations, the single sheet, material, fabric, layer, and/or membrane 101953 can form a canopy 101967 (FIGS. 158 and 161) between the paddle frames 101924 of the anchors 101908. In some implementations, each anchor 101908 includes a separate sheet, material, fabric, layer, and/or membrane 101953 such that the covers 101951 of the two anchors 101908 are not connected and a gap (e.g., the gap 101557 for the device 101500 shown in FIGS. 143-146) exists between the anchors 101808.

When the anchors 101908 are compressing the leaflets, the cover 101951 engages the leaflets between the pinch points 101981, 101983 to assist in coapting the leaflets and preventing or inhibiting regurgitation of blood through the native valve. In addition to providing force to the leaflets, the cover 101951 can further prevent or inhibit regurgitation of blood by acting as a barrier that prevents or inhibits the movement of blood through the native valve. The canopy 101967 can act as a further barrier for preventing or inhibiting blood from moving through the native valve. In some implementations, the sheet, material, fabric, layer, and/or membrane 101953 of the cover 101951 is flexible to allow for movement of the paddle frames 101924 between narrowed and expanded positions. For example, the inner frame portions 101972 can be fixed, and the width of the outer frame portions 101974 can be adjustable, and as the outer frame portions are moved to an expanded position, the sheet, material, fabric, layer, and/or membrane 101953 of the cover 101951 can be configured to stretch or otherwise expand. The flexibility of the sheet, material, fabric, layer, and/or membrane 101953 allows the cover 101951 to maintain a taut state when the paddle frames 101924 are in a narrowed position. This helps the cover prevent or inhibit leakage in various sizes of the adjustable frame members, without forming leakage gaps or openings between components of the device.

The cover 101951 can be configured to cover or not cover any component or portion of the implantable device. For example, referring to FIGS. 157-159, in the illustrated example, the cover 101951 extends between the inner and outer frame portions 101972, 101974 of the paddle frame 101924, but the cover 101951 does not encapsulate the outer paddle 101920 or inner paddle (not shown) of the anchors 101908. That is, the sheet, material, fabric, layer, and/or membrane 101953 of the cover 101951 includes an opening or cutout 101959 such that the cover 101951 does not cover the paddles 101920, 101922. However, it should be understood that other configurations are also contemplated.

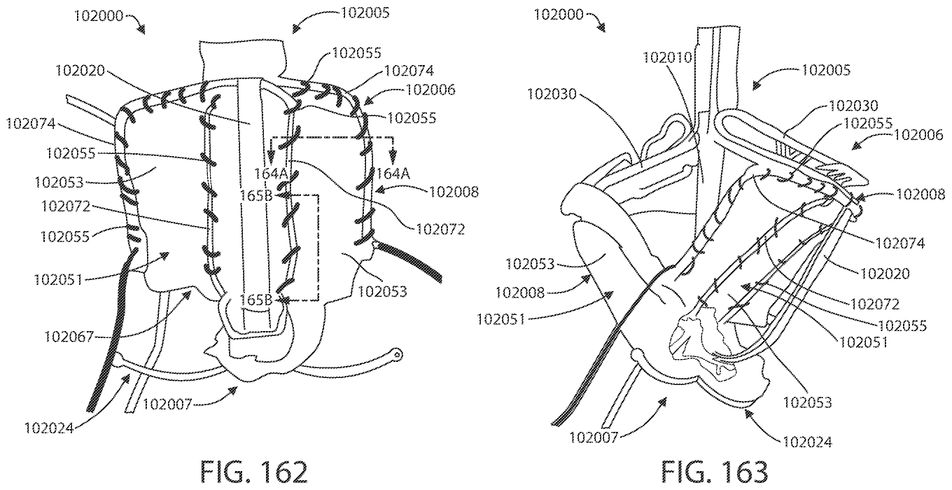

Referring to FIGS. 162-165, an example of an implantable device or implant 102000 includes an anchor portion 102006 having one or more paddle frames 102024 and a cover 102051. Referring to FIGS. 162-163, the implantable device 102000 includes a proximal or attachment portion 102005, an anchor portion 102006 having paddle frames 102024, an actuation portion (e.g., any actuation portion described in the present application), an optional spacer or coaptation element 102010 (e.g., any coaptation element described in the present application), and a distal portion 102007. The attachment portion 102005, the anchor portion 102006, and the distal portion 102007 can be configured in a variety of ways, such as, for example any way described in the present application.

In some implementations, the anchor portion 102006 can include at least one anchor 102008, where the anchor 102008 has an outer paddle 102020, an inner paddle (not shown), a paddle extension member or paddle frame 102024, clasps 102030, and a cover 102051. The paddles can take any suitable form, such as, for example, any form described in the present application. The paddle frame 102024 can have an inner frame portion 102072 and an outer frame portion 102074, such as, for example, similar to any other paddle frame disclosed in the present application that includes an inner frame portion and an outer frame portion.

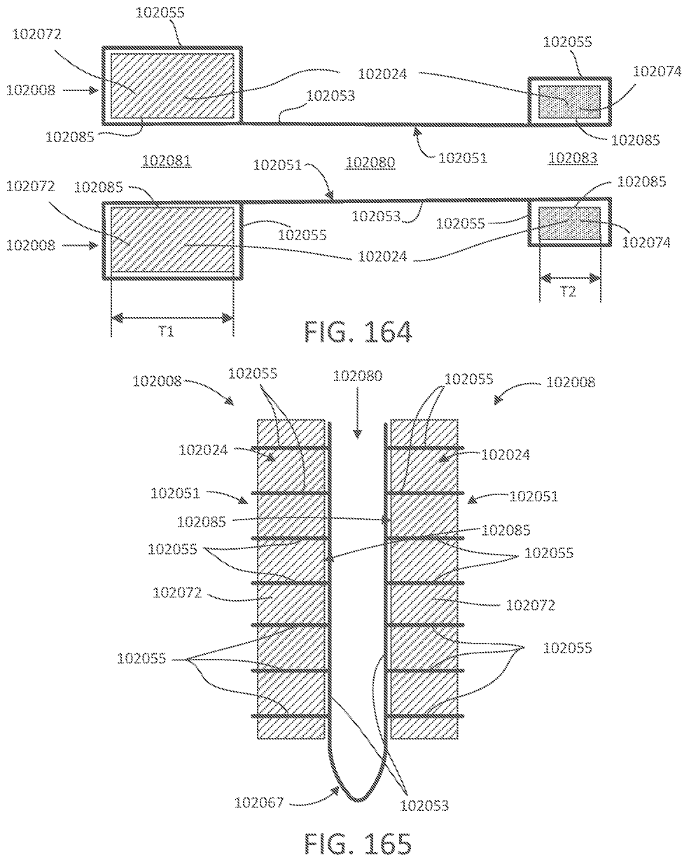

The inner frame portion 102072 can be rigid such that it is configured to support the paddles and provide a sufficient force to leaflets of the native valve during capture. The inner frame portion 102072 can be made of, for example, metals, plastics, etc. The outer frame portions 102074 can be connected to the inner frame portions 102072 such that the outer frame portions 102074 define the total width of the paddle frame 102024. The outer frame portions 102074 can be flexible such that the outer frame portions can optionally be manipulated by a user between an expanded position and a narrowed position. For example, the outer frame portions 102074 can be operatively connected to an actuation portion of the implantable device such that a user can engage the paddle frame 102024 to move the outer frame portions 102074 between the expanded and narrowed positions. The outer frame portions 102074 can be made of, for example, metals, and plastics. Referring to FIG. 164, in the illustrated example, the inner frame portions 102072 have a thickness T1 that is greater than a thickness T2 of the outer frame portions 102074, which allows the inner frame portions 102072 to be rigid and the outer frame portions 102074 to be flexible.

Referring to FIGS. 164 and 165, in some implementations, the implantable device 102000 (FIGS. 162-163) includes a pair of anchors 102008 having a cover 102051, where the anchors 102008 are configured to be moved to a closed position such that leaflets of a native valve can be compressed and captured in the area 102080 between the two anchors 102008. While the illustrated example is shown as having a pair of anchors 102008, it should be understood that the implantable device 102000 can have any suitable number of anchors that include a cover 102051. When the anchors 102008 are compressing the leaflets, the leaflets are engaged at the pinch points/regions between the anchors 102008. That is, the leaflets are engaged at a first pinch point 102081 between the inner frame portions 102072 of the anchors 102008, and the leaflets are engaged at a second pinch point 102083 between outer frame portions 102074 of the anchors 102008. FIGS. 164-165 show only a portion of the paddle frame 102024 taken from the lines 164A-164A and 165B-165B shown in FIG. 162. It should be understood that additional pinch points exist resulting from the engagement between the inner frame portions 102072 for each of the anchors 102008 and the engagement between the outer frame portions 102074 for each of the anchors 102008.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 162-165, the cover 102051 can be attached to the paddle frame 102024 and configured to provide further engagement between the implantable device 102000 and the leaflets. Each cover can include a sheet, material, fabric, layer, and/or membrane 102053 that is attached to the inner frame portion 102072 and outer frame portion 102074 by a plurality of connectors 102055 (e.g., stitches, adhesive, mechanical fasteners, ultrasonic welds, etc.). While the illustrated example shows the membrane 102053 connected to both the inner frame portion 102072 and the outer frame portion 102074, it should be understood that the membrane 102053 can be attached to one of the inner frame portion 102072 and the outer frame portion 102074. Any suitable number of connectors 102055 can be used to connect the membrane 102053 to the frame portions 102072, 102074.

In the illustrated example, the sheet, material, fabric, layer, and/or membrane 102053 is attached to the frame portions 102072, 102074 such that the sheet, material, fabric, layer, and/or membrane extends across an inner surface 102085 (FIGS. 162-165) of the frame portions 102072, 102074. The sheet, material, fabric, layer, and/or membrane 102053 can be made of a flexible material, a porous material, a non-porous material, and/or a material that is impermeable to (or inhibits or impedes) blood flow. In some implementations, the sheet, material, fabric, layer, and/or membrane 102053 is made from or comprises a biocompatible material, such as a woven biocompatible fabric that is configured to promote tissue ingrowth. In the illustrated example, a single sheet, material, fabric, layer, and/or membrane 102053 is secured to the paddle frames 102024 of both anchors 102008 such that the covers 102051 of the two anchors are connected. In these implementations, the single sheet, material, fabric, layer, and/or membrane 102053 can form a canopy 102067 (FIG. 165) between the paddle frames 102024 of the anchors 102008. In some implementations, each anchor 102008 includes a separate sheet, material, fabric, layer, and/or membrane 102053 such that the covers 102051 of the two anchors 102008 are not connected and a gap (e.g., the gap 101557 for the device 101500 shown in FIGS. 143-146) exists between the anchors 102008.

When the anchors 102008 are compressing the leaflets, the cover 102051 engages the leaflets between the pinch points 102081, 102083 to assist in coapting the leaflets and preventing or inhibiting regurgitation of blood through the native valve. In addition to providing force to the leaflets, the cover 102051 can further prevent or inhibit regurgitation of blood by acting as a barrier that prevents or inhibits the movement of blood through the native valve. The canopy 102067 can act as a further barrier for preventing or inhibiting blood from moving through the native valve. In some implementations, the sheet, material, fabric, layer, and/or membrane 102053 of the cover 102051 is flexible to allow for movement of the paddle frames 102024 between narrowed and expanded positions. For example, the inner frame portions 102072 can be fixed, and the width of the outer frame portions 102074 can be adjustable, and as the outer frame portions are moved to an expanded position, the sheet, material, fabric, layer, and/or membrane 102053 of the cover 102051 can be configured to stretch or otherwise expand. The flexibility of the sheet, material, fabric, layer, and/or membrane 102053 allows the cover 102051 to maintain a taut state when the paddle frames 102024 are in a narrowed position. This helps the cover prevent or inhibit leakage in various sizes of the adjustable frame members, without forming leakage gaps or openings between components of the device.

The cover 102051 can be configured to cover or not cover any component or portion of the implantable device. For example, referring to FIGS. 162-163, in the illustrated example, the cover 102051 extends between the inner and outer frame portions 102072, 102074 of the paddle frame 102024, as well as in an entirety an area 101891 defined by the interior of the inner frame portions 101872. In some implementations, the cover 102051 and encapsulates the inner paddles of the anchors 102008, but the cover 102051 does not encapsulate the outer paddles 101920 of the anchors 101908. However, it should be understood that other configurations are also contemplated.

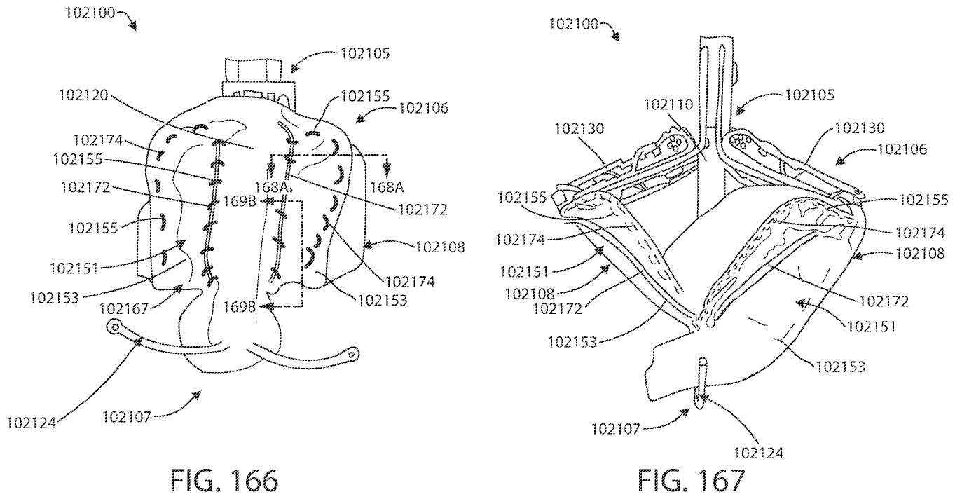

Referring to FIGS. 166-169, an example of an implantable device or implant 102100 includes an anchor portion 102106 having one or more paddle frames 102124 and a cover 102151. Referring to FIGS. 166-167, the implantable device 102100 includes a proximal or attachment portion 102105, an anchor portion 102106 having paddle frames 102124, an actuation portion (e.g., any actuation portion described in the present application), an optional coaptation element 102110 (e.g., any coaptation element described in the present application), and a distal portion 102107. The attachment portion 102105, the anchor portion 102106, and the distal portion 102107 can be configured in a variety of ways, such as, for example any way described in the present application.

In some implementations, the anchor portion 102106 can include at least one anchor 102108, where the anchor 102108 has an outer paddle 102120, an inner paddle (not shown), a paddle extension member or paddle frame 102124, clasps 102130, and a cover 102151. The paddles can take any suitable form, such as, for example, any form described in the present application. The paddle frame 102124 can have an inner frame portion 102172 and an outer frame portion 102174, such as, for example, similar to any other paddle frame members disclosed in the present application that includes an inner frame portion and an outer frame portion.

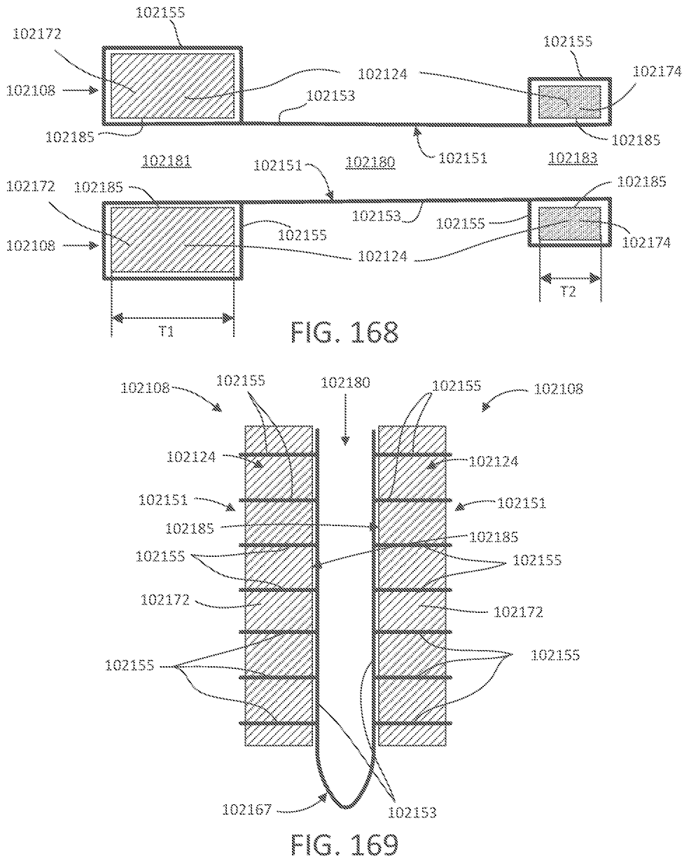

The inner frame portion 102172 can be rigid such that it is configured to support the paddles and provide a sufficient force to leaflets of the native valve during capture. The inner frame portion 102172 can be made of, for example, metals, plastics, etc. The outer frame portions 102174 can be connected to the inner frame portions 102172 such that the outer frame portions 102174 define the total width of the paddle frame 102124. The outer frame portions 102174 can be flexible such that the outer frame portions can optionally be manipulated by a user between an expanded position and a narrowed position. For example, the outer frame portions 102174 can be operatively connected to an actuation portion of the implantable device such that a user can engage the paddle frame 102124 to move the outer frame portions 102174 between the expanded and narrowed positions. The outer frame portions 102174 can be made of, for example, metals, and plastics. Referring to FIG. 168, in the illustrated example, the inner frame portions 102172 have a thickness T1 that is greater than a thickness T2 of the outer frame portions 102174, which allows the inner frame portions 102172 to be rigid and the outer frame portions 102174 to be flexible.

Referring to FIGS. 168 and 169, in some implementations, the implantable device 102100 (FIGS. 166-167) includes a pair of anchors 102108 having a cover 102151, where the anchors 102108 are configured to be moved to a closed position such that leaflets of a native valve can be compressed and captured in the area 102180 between the two anchors 102108. While the illustrated example is shown as having a pair of anchors 102108, it should be understood that the implantable device 102100 can have any suitable number of anchors that include a cover 102151. When the anchors 102108 are compressing the leaflets, the leaflets are engaged at the pinch points/regions between the anchors 102108. That is, the leaflets are engaged at a first pinch point 102181 between the inner frame portions 102172 of the anchors 102108, and the leaflets are engaged at a second pinch point 102183 between outer frame portions 102174 of the anchors 102108. FIGS. 168-169 show only a portion of the paddle frame 102124 taken from the lines 168A-168A and 169B-169B shown in FIG. 166. It should be understood that additional pinch points exist resulting from the engagement between the inner frame portions 102172 for each of the anchors 102108 and the engagement between the outer frame portions 102174 for each of the anchors 102108.

In some implementations, the pinch points/regions can be formed between other components or portions (e.g., protrusions, extensions, wings, bars, rods, clips, etc.) of anchors of the device in various different configurations. For example, other anchor designs can be used that do not include the same inner and outer frame portions but include other components or portions compressing the leaflets. The cover can be configured to extend between those other components or portions.

Referring to FIGS. 166-169, the cover 102151 can be attached to the paddle frame 102124 and configured to provide further engagement between the implantable device 102100 and the leaflets. Each cover can include a sheet, material, fabric, layer, and/or membrane 102153 that is attached to the inner frame portion 102172 and outer frame portion 102174 by a plurality of connectors 102155 (e.g., stitches, adhesive, mechanical fasteners, ultrasonic welds, etc.). While the illustrated example shows the membrane 102153 connected to both the inner frame portion 102172 and the outer frame portion 102174, it should be understood that the membrane 102053 can be attached to one of the inner frame portion 102172 and the outer frame portion 102074. Any suitable number of connectors 102155 can be used to connect the membrane 102053 to the frame portions 102172, 102174.

In the illustrated example, the sheet, material, fabric, layer, and/or membrane 102153 is attached to the frame portions 102172, 102174 such that the sheet, material, fabric, layer, and/or membrane extends across an inner surface 102185 (FIGS. 168-169) of the frame portions 102172, 102174. The sheet, material, fabric, layer, and/or membrane 102153 can be made of a flexible material, a porous material, a non-porous material, and/or a material that is impermeable to (or inhibits or impedes) blood flow. In some implementations, the sheet, material, fabric, layer, and/or membrane 102153 is made from or comprises a biocompatible material, such as a woven biocompatible fabric that is configured to promote tissue ingrowth. In the illustrated example, a single sheet, material, fabric, layer, and/or membrane 102153 is secured to the paddle frames 102124 of both anchors 102108 such that the covers 102151 of the two anchors are connected. In these implementations, the single sheet, material, fabric, layer, and/or membrane 102153 can form a canopy 102167 (FIG. 166) between the paddle frames 102124 of the anchors 102108. In some implementations, each anchor 102108 includes a separate sheet, material, fabric, layer, and/or membrane 102153 such that the covers 102151 of the two anchors 102108 are not connected and a gap (e.g., the gap 101557 for the device 101500 shown in FIGS. 143-146) exists between the anchors 102108.

When the anchors 102108 are compressing the leaflets, the cover 102151 engages the leaflets between the pinch points 102181, 102183 to assist in coapting the leaflets and preventing or inhibiting regurgitation of blood through the native valve. In addition to providing force to the leaflets, the cover 102151 can further prevent or inhibit regurgitation of blood by acting as a barrier that prevents or inhibits the movement of blood through the native valve. The canopy 102167 can act as a further barrier for preventing or inhibiting blood from moving through the native valve. In some implementations, the sheet, material, fabric, layer, and/or membrane 102153 of the cover 102151 is flexible to allow for movement of the paddle frames 102124 between narrowed and expanded positions. For example, the inner frame portions 102172 can be fixed, and the width of the outer frame portions 102174 can be adjustable, and as the outer frame portions are moved to an expanded position, the sheet, material, fabric, layer, and/or membrane 102153 of the cover 102151 can be configured to stretch. The flexibility of the sheet, material, fabric, layer, and/or membrane 102153 allows the cover 102151 to maintain a taut state when the paddle frames 102124 are in a narrowed position. This helps the cover prevent or inhibit leakage in various sizes of the adjustable frame members without forming leakage gaps.

The cover 102151 can be configured to cover or not cover any component or portion of the implantable device. For example, referring to FIGS. 166-167, in the illustrated example, the cover 102151 extends between the inner and outer frame portions 102172, 102174 of the paddle frame 102124 and encapsulates the inner and outer paddles of the anchors 102008. However, it should be understood that other configurations are also contemplated.

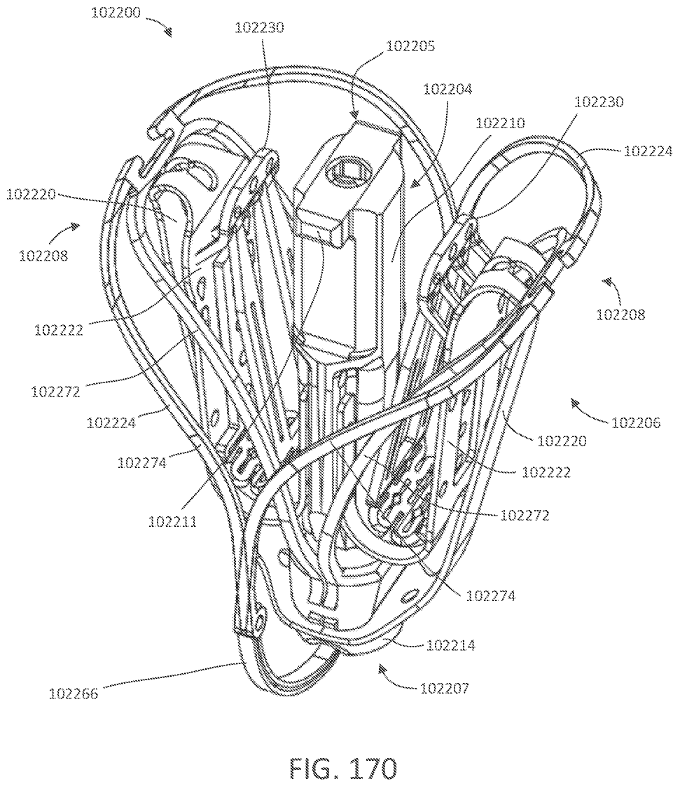

Referring now to FIGS. 170-178, an example of an implantable device or implant 102200 is shown that includes a smaller coaptation element. The implantable device or implant 102200 is one of the many different configurations that the device 100 that is schematically illustrated in FIGS. 8-14 can take. For example, in some implementations, the device/implant 102200 includes a coaptation portion 102204, a proximal or attachment portion 102205, an anchor portion 102206, and a distal portion 102207. The device 102200 can include any other features for an implantable device or implant discussed in the present application and/or in U.S. Provisional Patent Application No. 63/217,622 filed on Jul. 1, 2021, which is incorporated herein by reference in its entirety, and the device 102200 can be positioned to engage valve tissue 20, 22 as part of any suitable valve repair system (e.g., any valve repair system disclosed in the present application and/or U.S. Provisional Patent Application No. 63/217,622). The device/implant 102200 can be a prosthetic spacer device, valve repair device, or another type of implant that attaches to leaflets of a native valve.

In some implementations, the anchor portion 102206 includes a plurality of anchors 102208. The anchors 102208 can be configured in a variety of ways, such as, for example, any of the ways described in the present application. In the illustrated example, the anchor portion 102206 includes two anchors 102208 with each anchor 102208 having outer paddles 102220, inner paddles 102222, paddle extension members or paddle frames 102224, and clasps 102230. The outer paddles 102220, inner paddles 102222, paddle frames 102224, and clasps 102230 can take any suitable form, such as, for example, any form described in the present application. In some implementations, the paddle frames 102224 can have an inner frame portion 102272 and an outer frame portion 102274, such as, for example, similar to any other paddle frame disclosed in the present application that includes an inner frame portion and an outer frame portion. The anchors 102208 can include an optional cover, such as, for example, any of the covers disclosed in FIGS. 143-169 of the present application, where the cover can be configured to assist in coapting the leaflets and preventing or inhibiting regurgitation of blood through the native valve. The cover can be attached to the anchors 102208 in any suitable manner and by any suitable means, such as, for example, any manner and/or means described in the present application.

The attachment portion 102205 and distal portion 102207 can take any suitable form, such as, for example, any form described in the present application. In the illustrated example, the attachment portion 102205 includes a first or proximal collar 102211 (or other attachment element) for engaging with a coupler of a delivery system. In the illustrated example, the distal portion 102207 includes a cap 102214 that is operatively connected to the anchor portion 102206 of the device 102200 such that movement of the cap 102214 relative to a spacer or coaptation element 102210 of the coaptation portion 102204 causes the anchors 102208 to move between open and closed positions.

The coaptation portion 102204 of the device can include a coaptation element 102210 (e.g., a spacer, coaption element, bushing, etc.) for implantation between leaflets of a native valve. The coaptation element 102210 extends from the proximal collar 102211 (or other attachment element) to a distal end 102251 and includes a lumen 102253 for receiving the actuation element (e.g., actuation shafts, actuation rods, actuation tubes, actuation wires, actuation lines, etc.) and the width adjustment element (e.g., width adjustment control, width adjustment wire, width adjustment shaft, width adjustment tube, width adjustment line, width adjustment cord, width adjustment suture, width adjustment tether, etc.) of the implantable device 102200. In the illustrated example, the proximal collar 102211 of the attachment portion 102205 is integral with the coaptation element 102210.

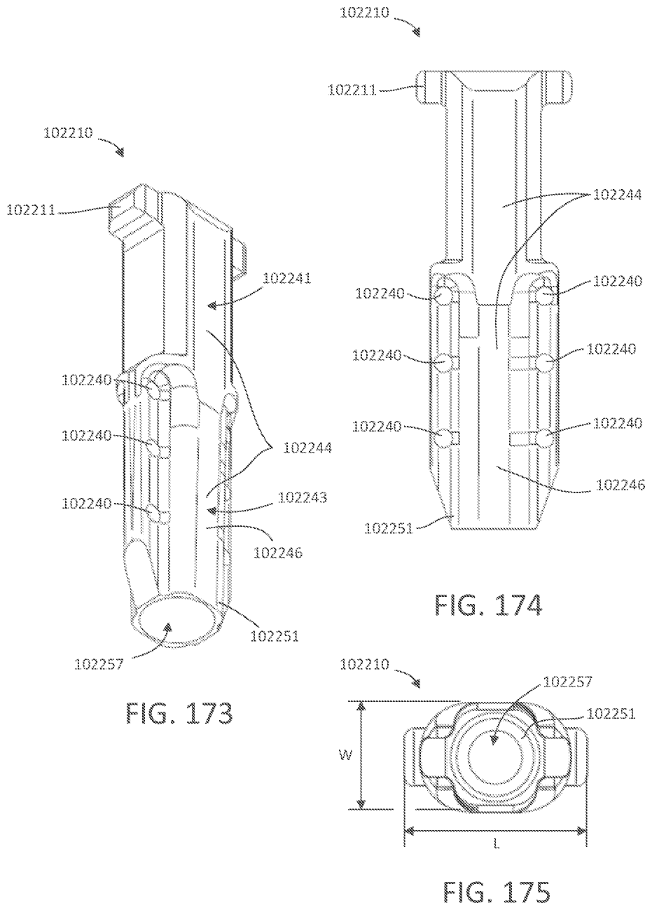

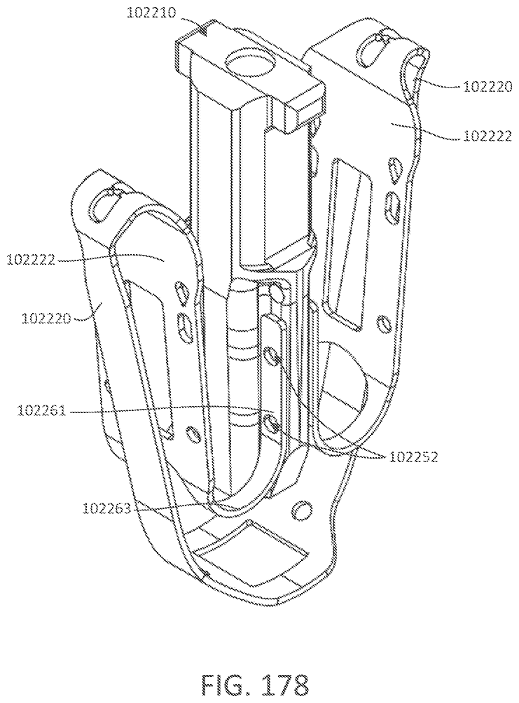

The coaptation element 102210 can have attachment openings 102240 (FIGS. 173-174 and 177) such that the anchors 102208 can be attached to the coaptation element 102210. In the illustrated example, the inner paddles 102222 of the anchors 102208 are coupled to the coaptation element 102210 at the attachment openings 102240. Referring to FIG. 178, mounting posts 102261 are connected to the inner paddles 102222 by transition portions or curved segments 102263. The mounting posts 102261 can have attachment openings 102252 (FIG. 178) that are configured to align with the attachment openings 102240 of the coaptation element 102210 such that a connection component (e.g., a suture, fastener, etc.) can be inserted through the attachment openings 102240, 102252 to secure the mounting posts 102261 to the coaptation element 102210. The inner paddles 102222 can, however, be coupled to the coaptation element 102210 by any other suitable means.

The proximal end of the coaptation element 102210 has a proximal opening 102255 that allows an actuation element and/or a width adjustment element to move relative to the lumen 102253. The proximal opening 102255 allows the actuation element and/or the width adjustment element to engage one or more controllable components of the device or implant 102200. The distal end 102251 of the coaptation element 102210 can include a distal opening 102257. An actuation element and/or one or more components that couple the cap to the actuation element can extend through the distal opening 102257 and attach to the cap. In some implementations, an actuation element extends from the delivery system to engage and enable actuation of the device 102200 between the open and closed positions and/or a width adjustment element extends from the delivery system to adjust widths of the paddles. Movement of the cap 102214 away from the coaptation element 102210 can cause the anchors 102208 to move to the open position, and movement of the cap 102214 toward the coaptation element 102210 can cause the anchors 102208 to move to the closed position. The actuation element, the cap, and any components that couple the cap to the actuation element can be configured to removably engage the cap 102214 in any suitable manner that allows the actuation element to be disengaged and removed from the device 102200 after implantation, such as, for example, any manner described in the present application.

In some implementations, one or more width adjustment elements extend through the lumen 102253 of the coaptation element 102210 and engage the paddle frames 102224 to move the paddle frames 102224 between a narrowed position and an expanded position. For example, a connector 102266 or other component that is coupled to the paddle frames 102224 can extend through the cap 102214, through the distal opening 102257 and into the lumen 102253 of the coaptation element 102210. The width adjustment(s) can be configured to engage and move the connector 102266 or other component of the paddle frames 102224 relative to the cap 102214. In some implementations, movement of the connector 102266 or other component of the paddle frames 102224 in a distal direction relative to the cap 102214 can cause the paddle frames 102224 to move to an expanded position, and movement of the connector 102266 or other component of the paddle frames 102224 in a proximal direction relative to the cap 102214 can cause the paddle frames 102224 to move to a narrowed position. The width adjustment element can be configured to removably engage the paddle frames 102224 in any suitable manner that allows the width adjustment element to be disengaged and removed from the device 102200 after implantation, such as, for example, any manner described in the present application.

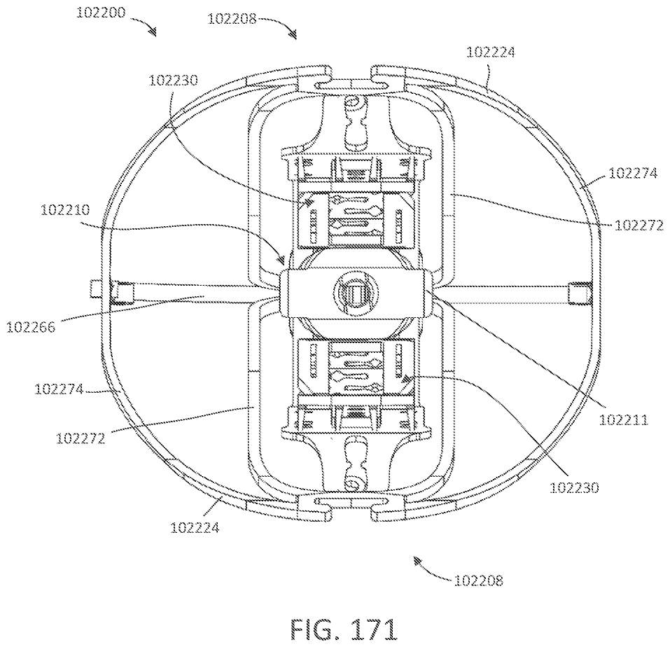

In some implementations, when viewed from above (as shown in FIG. 171), the coaptation element 102210 has a smaller size relative to the outer periphery of the device 102200, such as, for example, relative to the outer periphery of the paddle frames 102224 of the device 102200. For example, a ratio of an area defined by the outer periphery of the paddle frames 102224 when view from above relative to the outer periphery of the coaptation element 102210 when view from above can be greater than or equal to 2 to 1, such as greater than or equal to 3 to 1, such as greater than or equal to 4 to 1, such as greater than equal to 5 to 1, such as greater than or equal to 6 to 1, such as greater than or equal to 10 to 1. In implementations with a smaller coaptation element, the optional cover described above can be attached to the paddle frames 102224 to cover any open portions between the paddle frames 102224 and the coaptation element 102210 to block regurgitative blood flow between the paddle frames 102224 and the coaptation element 102210.

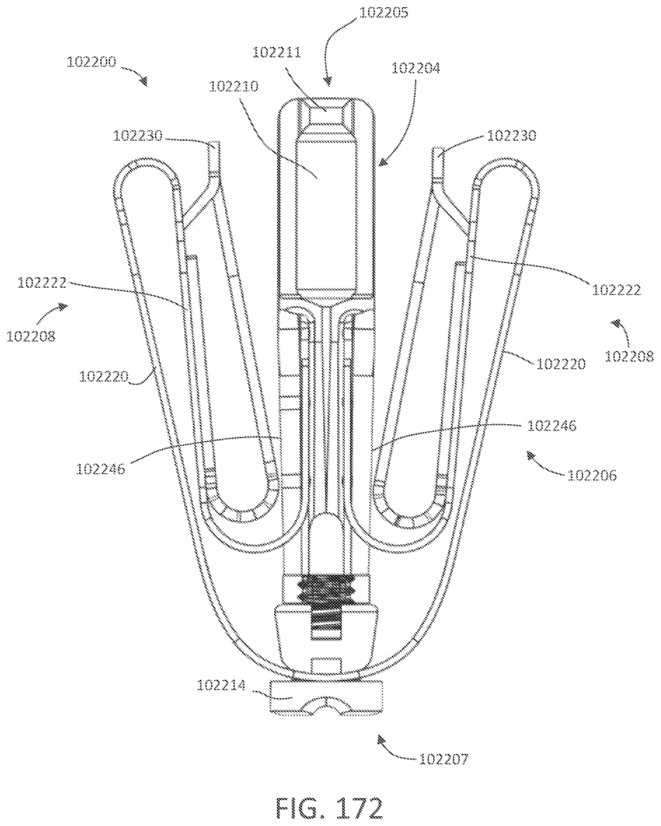

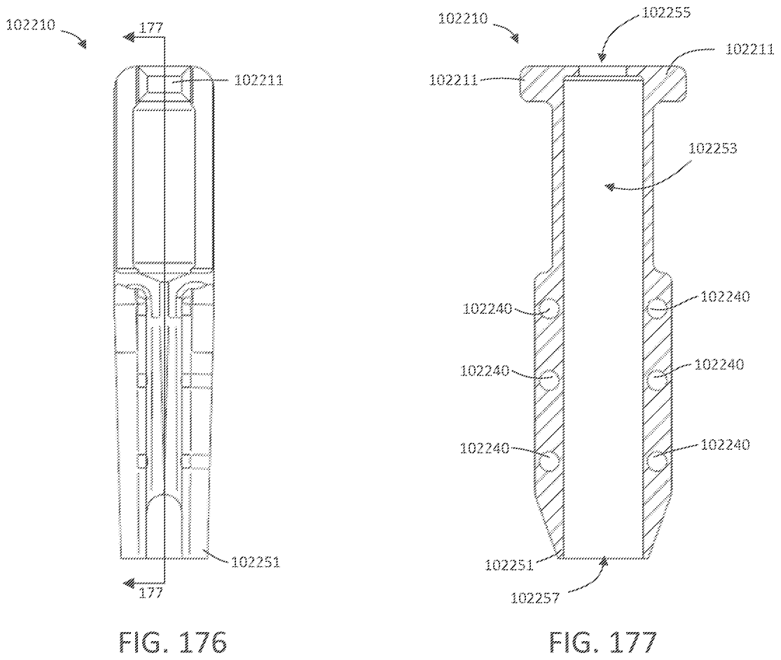

The smaller size of the coaptation element 102210 can allow for easier movement of the various components of the anchors 102208 as the anchors 102208 are moved between the open and closed positions. For example, the coaptation element 102210 can include planar surfaces 102244 (FIGS. 173-174) for allowing the clasps 102230 to rest on when the device 102200 is in the closed position. In some implementations, the coaptation element 102210 can have tapered walls 102246 (FIGS. 172 and 176) that extend inward from the proximal end to the distal end of the coaptation element 102210 to create additional space for the anchors 102208 to move when the device is moved to the opened position. That is, as the device 102200 is moved to the opened position, one or more components of the anchors 102208 (e.g., the paddle frames 102224) pivot downward, and the tapered walls provide for additional space for the components of the anchors to move.

Referring to FIGS. 173-175, in the illustrated example, the coaptation element 102210 has a proximal portion 102241 (FIG. 173) having a generally rectangular shape and a distal portion 102243 (FIG. 173) having a generally rounded shape. However, it should be understood that other configurations are also contemplated. In some implementations, the coaptation element 102210 can be made by injection molding. However, it should be understood that other configurations are also contemplated. In some implementations, the coaptation element can be made from or comprise any known polymer material or other non-polymer material(s). In some implementations, the coaptation element 102210 can be made from or comprise a metal material or any other suitable material.

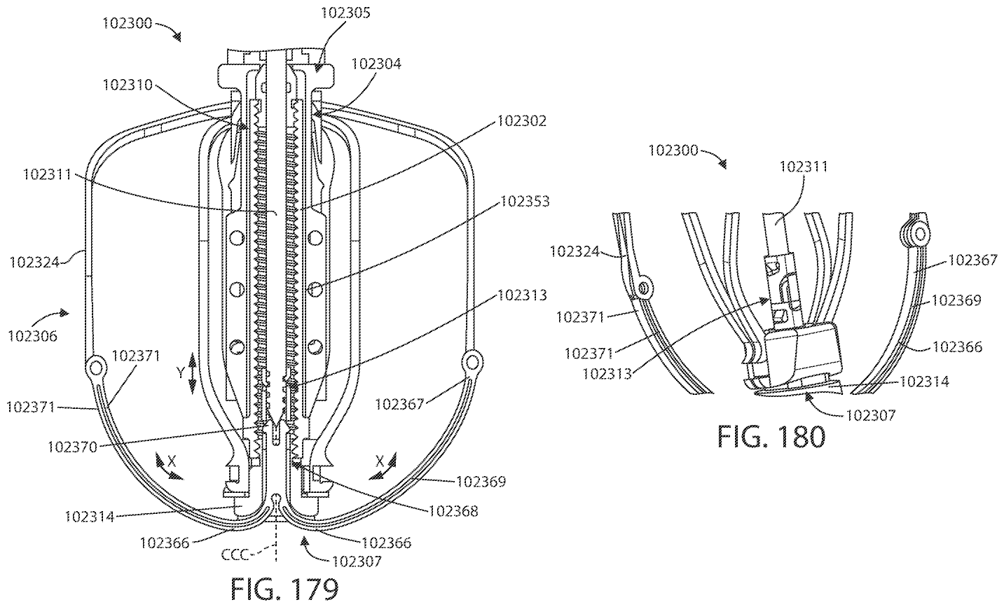

Referring to FIGS. 179-184, components of another example implementation of an implantable device or implant 102300 having paddle frames is shown, where a coupler or connection feature 102313 is used to allow a width adjustment element (e.g., width adjustment control, width adjustment wire, width adjustment shaft, width adjustment tube, width adjustment line, width adjustment cord, width adjustment suture, width adjustment tether, etc.) to engage the paddle frames and move the paddle frames between narrowed and expanded positions. The implantable device 102300 can include a proximal or attachment portion 102305, an anchor portion 102306 (e.g., any anchor portion described in the present application), a coaptation portion 102304, and a distal portion 102307. The coaptation portion 102304, proximal portion 102305, the anchor portion 102306, and the distal portion 102307 can be configured in a variety of ways, such as, for example, any of the ways described in the present application.

In some implementations, for example, in the illustrated example, the coaptation portion 102304 includes a coaptation element 102310 that takes the form of the coaptation element 102210 shown in FIGS. 170-178 or otherwise shown or described herein. The coaptation element 102310 includes a column or lumen 102353 that accepts a receiver 102302 (e.g., an internally threaded element, a notched receiving portion, column, lumen, tube, shaft, post, housing, tracks, cylinder etc.). The receiver 102302 is connected to the cap and is moveable inside the lumen 102353 to open and close the paddles. For example, the receiver 102302 can be coupled to the actuation element 8102 shown in FIGS. 26-30. Movement of the actuation element can open and close the paddles in the manner described in FIGS. 26-30. The actuation element 8102 can be coupled and decoupled from the receiver as shown in FIGS. 132-135 or any other manner described herein. The coupler 102313 is moveable inside the receiver 102302. The coupler 102313 is connected to a width adjustment element 102311 (e.g., width adjustment control, width adjustment wire, width adjustment shaft, width adjustment tube, width adjustment line, width adjustment cord, width adjustment suture, width adjustment tether, etc.) and to a connector 102366 that is connected to the paddle frames 102324. As such, movement of the width adjustment element 102311, moves the coupler 102313 and the connector 102366 inside the receiver 102302 to move the paddle frames 102324 between the narrowed and expanded positions. The paddle frames 102324 can include outer frame portions and inner frame portions that are the same as or similar to other outer frame portions and inner frame portions described elsewhere herein.

In the illustrated example, the paddle frames 102324 are symmetric along longitudinal axis CCC (FIG. 179). In some implementations of the implantable device 102300, however, the paddle frames 102324 are not symmetric about the axis CCC. In the illustrated example, the two paddle frames 102324 are connected to the single connector 102366 at the sides of the paddle frame portions. As a result, the single movement of the connector 102366 uniformly controls the width of the two paddle frames 102324. In some implementations, more than one, such as two or four, connectors 102366 can be used to non-uniformly adjust the paddle frames 102324 (differently between the two paddle frame portions and/or differently between the two sides of a single paddle frame portion.

Referring to FIG. 179, in the illustrated example, the connector 102366 is W-shaped. The connector 102366 is configured to be drawn/flexed into the cap 102314 to pull the paddle frames 102324 to the narrowed configuration. The connector 102366 is configured to be advanced out of the cap 102314 to allow the paddle frames 102324 to widen. The connector 102366 can take a wide variety of different forms. The connector 102366 can be made from or comprise a flexible material. In the illustrated example, the connector 102366 includes arms 102367 with optional slits or cutouts 102369. The cutouts 102369 allow the sides 102371 to move or flex toward one another as the arms 102367 are drawn into the cap 102314. This increases the flexibility of the arms 102367. The connector 102366 can be made from or comprise any material that can be drawn into and pushed out of the cap 102314 to move the paddle frames 102324 between an expanded position and a narrowed position. While the connector 102366 is shown as having a W-shape, it should be understood that the connector 102366 can take any suitable form that can move the paddle frames 102324 between an expanded position and a narrowed position.

In the illustrated example, a connector 102366 (e.g., shaped metal component, shaped plastic component, tether, wire, strut, line, cord, suture, etc.) includes an inner end or post 102368 that is engaged by a paddle width adjustment element 102311 (e.g., width adjustment control, width adjustment wire, width adjustment shaft, width adjustment tube, width adjustment line, width adjustment cord, width adjustment suture, width adjustment tether, or any other suitable type of adjustment element) such that a user can move the inner end or post 102368 relative to the receiver 102302 and the cap 102314. Movement of the paddle width adjustment element 102311 relative to the receiver 102302 and the cap 102314 moves the paddle frames 102324 between the narrowed and expanded positions. as described in more detail below. In the illustrated example, the inner end or post 102368 includes prongs 102370 that attach to a coupler 102313 that is releasably attachable to the paddle width adjustment element 102311. The inner end or post 102368 of the connector 102366 can take any suitable form, such as, for example, the form of the inner end 101368 of the connector 101324 shown in FIG. 139, or any other form described in the present application. The slit 101374 illustrated in FIG. 139 is optional.

The width adjustment element 102311 can be releasably connected to the coupler or connection feature 102313, and thus, the prongs 102370. For example, the width adjustment element 102311 can be releasably connected to the coupler 102313 by a threaded connection. When connected to the coupler 102313, the width adjustment element can move the coupler 102313 to move relative to the receiver 102302.

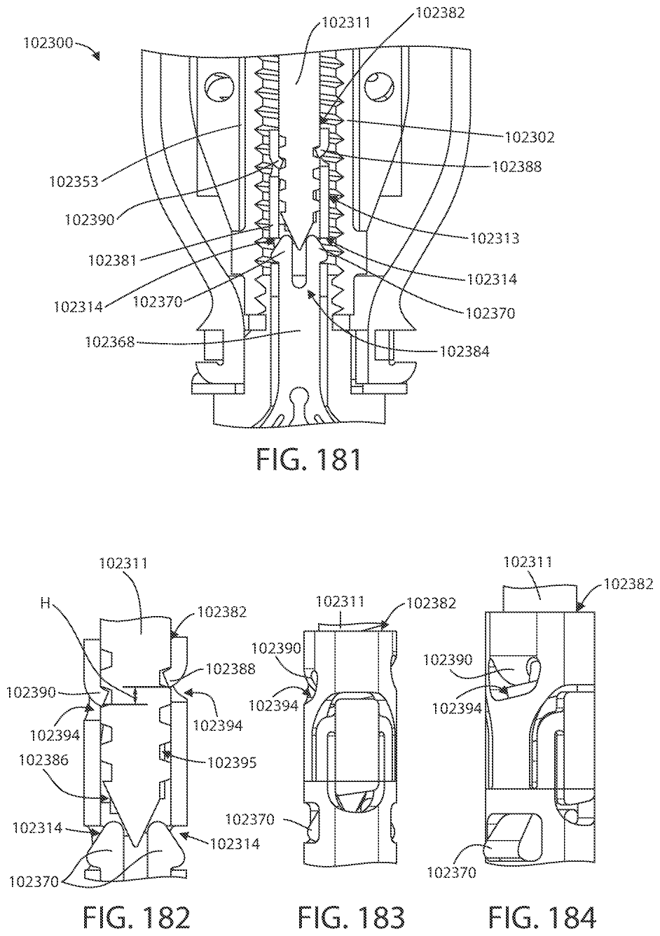

In some implementations, referring to FIGS. 181-184, the coupler 102313 has a body 102381 that includes a proximal opening 102382, a distal opening 102384, a lumen 102386 (FIG. 182) that extends from the proximal to distal opening, a first attachment projection 102388, and a second attachment projection 102390. The first and second attachment projections 102388, 102390 extend from cutouts 102394 of the body 102381 such that the attachment projections 102388, 102390 extend at an inward angle relative to the body 102381. The first attachment projection 102388 is offset from the second attachment projection 102390 by a height H (FIG. 182) such that the attachment projections 102388, 102390 are configured to connect the width adjustment element 102311 to the coupler 102313 by a threaded connection. That is, the attachment projections 102388, 102390 are configured to align with the pitch of external threads 102395 of the width adjustment element 102311. Accordingly, the width adjustment element 102311 can be connected to the coupler 102313 by rotating the width adjustment element 102311 relative to the coupler 102313 in a first direction, and the width adjustment element 102311 can be disconnected from the coupler 102313 by rotating the width adjustment element 102311 relative to the coupler 102313 in an opposite direction. In the illustrated example, the attachment projections 102388, 102390 are configured to connect to square threads of the width adjustment element 102311. However, the attachment projections 102388, 102390 can be configured to connect to any type of threads. However, it should be understood that other configurations are also contemplated. For example, the proximal end of the coupler can include traditional female threads instead of the projections 102388, 102390.

The prongs 102370 of the paddle frames 102324 can connect to the coupler 102313 by any suitable means, such as, for example, any means described in the present application. In the illustrated example, the prongs 102370 connects to the coupler 102313 similar to the implementation shown in FIGS. 136-139. That is, the coupler 102313 includes openings for receiving the prongs 102370 of paddle frames 102324. However, it should be understood that other configurations are also contemplated.

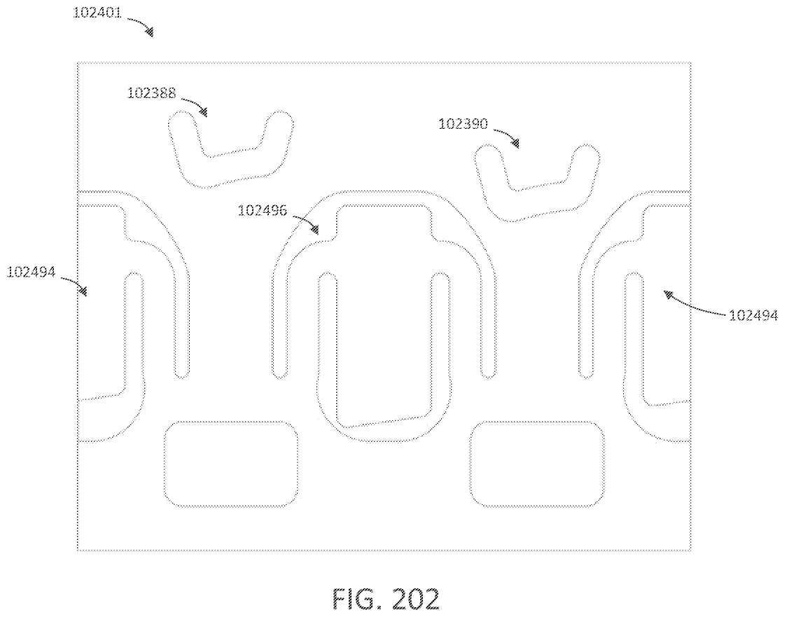

The coupler 102313 can be made of any suitable material that allows for the attachment projections 102388, 102390 to be positioned to engage the external threads of the width adjustment element 102311. For example, the coupler 102313 can be made from or comprise metal or polymer materials. In some implementations, the coupler can be made from or comprise a sheet metal material that includes the attachment projections 102388, 102390, such as, for example, the sheet metal material 102401 shown in FIG. 202.

Referring to FIG. 179, in in some implementations, the connector 102366 (e.g., shaped metal component, shaped plastic component, tether, wire, strut, line, cord, suture, etc.) attaches to the paddle frames 102324 at outer ends of the connector 102366 and to a coupler 102313 at an inner end of the connector 102366 (See FIG. 179). Prongs 102370 of the inner end 102368 are connected to the coupler 102313. Movement of the coupler 102313 and prongs 102370 in direction Y causes the arms 102367 of the connector 102366 to move in the directions X. This movement causes the paddle frames 102324 to move between the narrowed and expanded positions. In the illustrated example, movement of the prongs 102370 away from the cap 102314 causes the paddle frames 102324 to move to a narrowed position. Conversely, movement of the prongs 102370 toward the cap 102314 causes the paddle frames 102324 to move to an expanded position. In some implementations, the arms 102367 of the connector 102366 move into the receiver 102302 when the paddle frames 102324 are moved to the narrowed position, and the distal ends of the connector 102366 move out of the receiver 102302 when the paddle frames 102324 are moved to the expanded position.

The movement of the paddle frames 102324 to the narrowed position allows the device or implant 102300 to maneuver more easily into position for implantation in the heart by reducing the contact and/or friction between the native structures of the heart—e.g., chordae— and the device 102300. The movement of the paddle frames 102324 to the expanded position provides the anchor portion of the device or implant 102300 with the flexibility to select a range of surface areas to engage and capture leaflet(s) that best suits the individual native heart valve.

In some implementations, the paddle frames 102324 can be made from or comprise a material that allows the paddle frames 102324 to be pulled and flexed between narrow and wide configurations. For example, the paddle frames 102324, or a portion thereof, can be made of any flexible material, including but not limited to, metal, plastic, fabric, suture, etc. The paddle frames 102324 can be made using a variety of processes, including, but not limited to, cutting, such as laser cutting, stamping, casting, molding, heat treating, shape setting, etc. The paddle frames 102324 can be made from or comprise a shape memory material, —such as Nitinol—to provide shape-setting capability.

In some implementations, the adjustable width paddle frames can be adjusted to a selected width and be set or fixed at the selected width to remain at the selected width. The adjustable width paddle frames can be adjusted to a selected width and be set or fixed at the selected width to remain at the selected width in a variety of different ways. Any configuration that allows the width of the paddle frames to be adjusted between a narrow configuration and a wide configuration and then set or fixed at the selected configuration can be used. In some implementations, connecting a paddle width adjustment element to a coupler allows the coupler to move to adjust the width of the adjustable width paddle frame(s) and disconnecting the paddle width adjustment element from the coupler fixes the position of the coupler relative to one or more components of the valve repair device to set the width of the adjustable width paddle frame(s). For example, connecting a paddle width adjustment element to a coupler can allow the coupler to move relative to the cap and/or receiver to adjust the width of the adjustable width paddle frame(s) and disconnecting the paddle width adjustment element from the coupler fixes the position of the coupler relative to the cap and/or receiver to set the width of the adjustable width paddle frame(s).

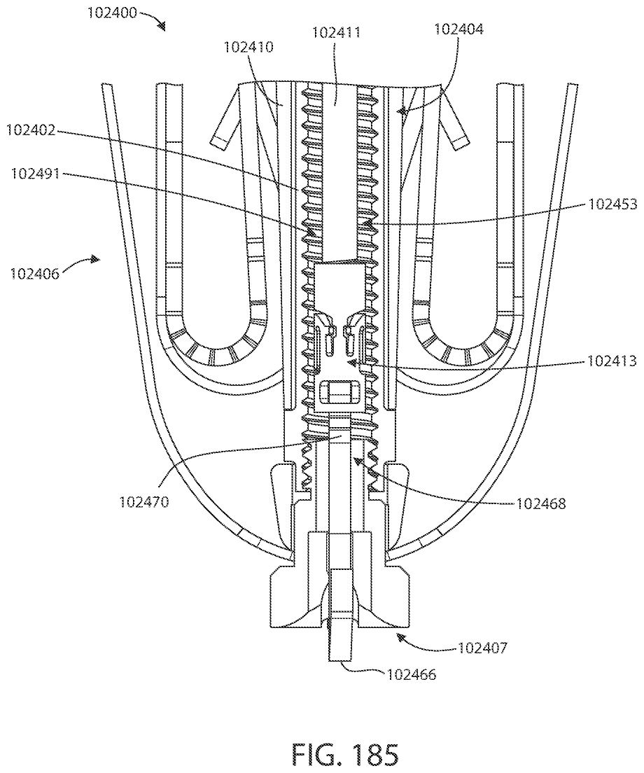

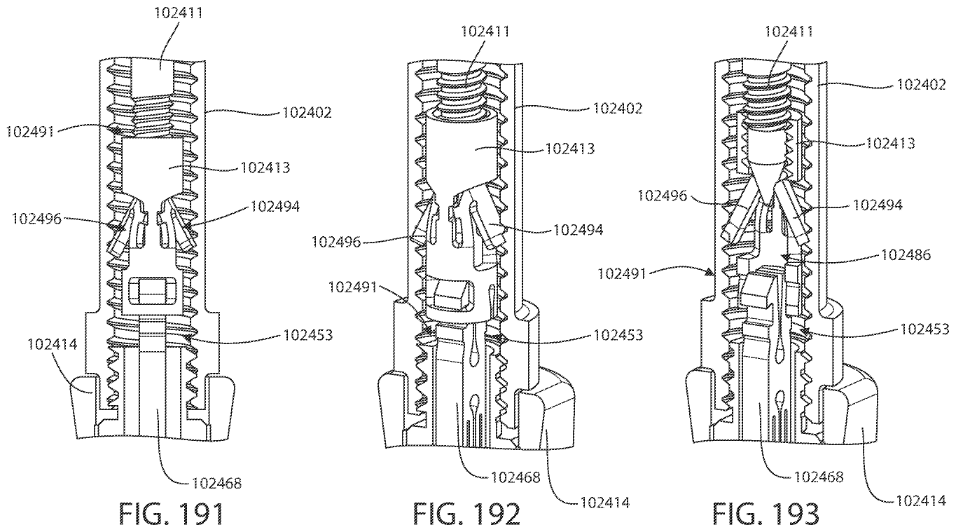

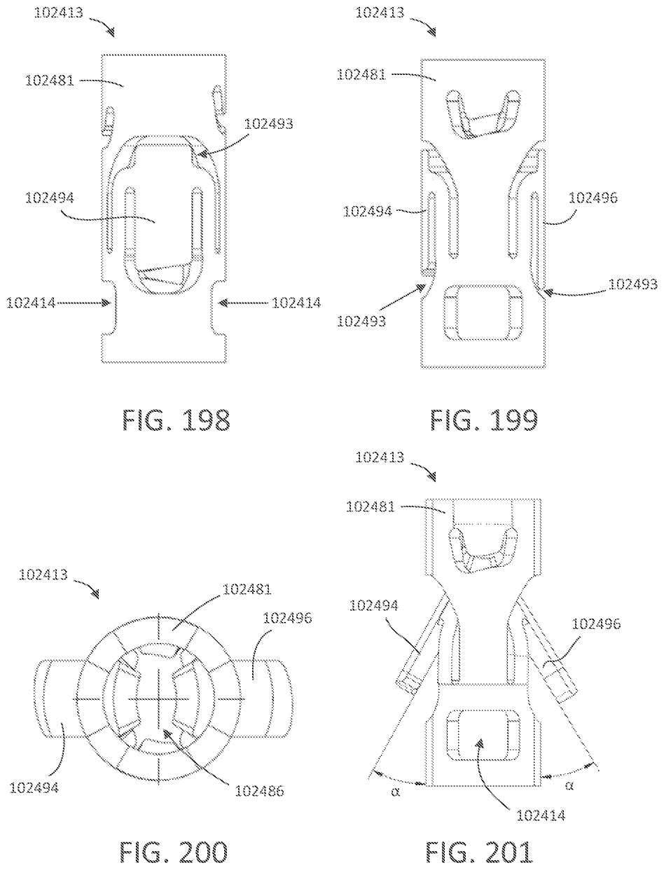

Referring to FIGS. 185-202, components of an example implementation of an implantable device or implant 102400 where a connector 102466 and connected adjustable width paddle frames (See FIGS. 170, 171, and 179) can be adjusted to a selected width and be set or fixed at the selected width to remain at the selected width are illustrated. A coupler or connection feature 102413 is used to allow a width adjustment element (e.g., width adjustment control, width adjustment wire, width adjustment shaft, width adjustment tube, width adjustment line, width adjustment cord, width adjustment suture, width adjustment tether, etc.) to engage the paddle frames and move the paddle frames between narrowed and expanded positions and to set the position of the paddle frames relative to a cap 102414 and/or receiver 102402 (e.g., an internally threaded element, a notched receiving portion, column, lumen, tube, shaft, post, housing, tracks, cylinder etc.). The implantable device 102400 can include a proximal or attachment portion (not shown), an anchor portion 102406 (e.g., any anchor portion described in the present application), paddle frames (attached to the connector—See FIGS. 170, 171, and 179), a coaptation or spacer portion 102404, and a distal portion 102407. The coaptation portion 102404, the proximal portion, the anchor portion 102406, and the distal portion 102407 can be configured in a variety of ways, such as, for example, any of the ways described in the present application. In the illustrated example, the coaptation portion 102404 includes a coaptation element 102410 that takes the form of the coaptation element 102210 shown in FIGS. 170-178.

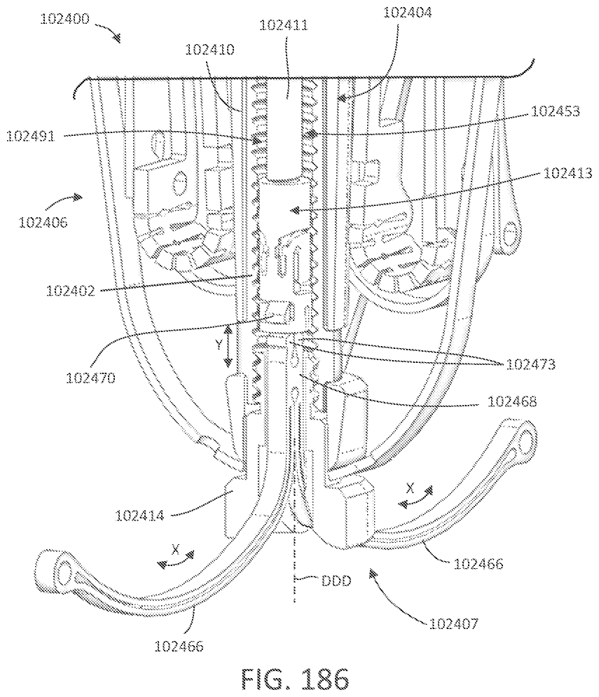

In the illustrated example, the connector 102466 is symmetric along longitudinal axis DDD (FIG. 186). In some implementations of the implantable device 102400, however, the connector 102466 is not symmetric about the axis DDD. In the illustrated example, the connector 102466 has an inner end or post 102468 that is engaged by a width adjustment element 102411 (e.g., width adjustment control, width adjustment wire, width adjustment shaft, width adjustment tube, width adjustment line, width adjustment cord, width adjustment suture, width adjustment tether, or any other suitable type of actuation element) such that a user can move the inner end 102468 relative to the receiver 102402 to move the connector 102466 and attached paddle frame portions between the narrowed and expanded positions. In the illustrated example, the inner end 102468 includes prongs 102470 or other connecting structure that attaches to the coupler 102413. The inner end or post 102468 of the connector 102466 can take any suitable form, such as, for example, the form of the inner end 101368 of the connector 101324 shown in FIG. 139 with or without the optional slit 101374, or any other form described in the present application.

The width adjustment element 102411 can be releasably connected to the coupler 102413, and the inner end 102468 is connected to the coupler 102413. When the width adjustment element 102411 is connected to the coupler 102413, the coupler disengages from the receiver 102402. When the coupler 102413 is disengaged from the receiver 102402, movement of the width adjustment element causes the coupler 102413 and, consequently, the prongs 102470 to move relative to the receiver 102402 to widen and narrow the paddle frames. When the width adjustment element 102411 is disconnected from the coupler 102413, the coupler engages the receiver 102402. When the coupler 102413 is engaged with the receiver 102402, movement of the width adjustment element 102411, the coupler 102413, and the prongs 102470 is prevented, thereby setting or fixing the width of the paddle frame.

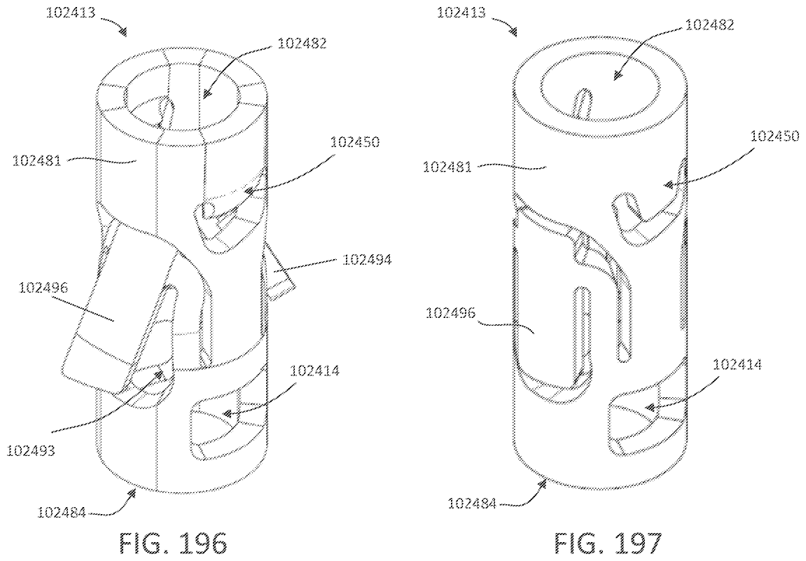

In the illustrated example, referring to FIGS. 196-201, the coupler 102413 has a body 102481 that includes a proximal opening 102482, a distal opening 102484, a lumen 102486 that extends from the proximal to distal opening. The proximal end of the coupler 102413 has a connection portion 102453 for connecting to the width adjustment element 102411 (FIGS. 185-194). The connection portion 102453 can take any suitable form that allows for a connection between the width adjustment element 102411 and the coupler 102413, such as, for example, a threaded connection, a snap-fit connection, or any other suitable type of connection described in the present application. In the illustrated example, the connection portion 102453 takes the form for connecting the width adjustment element 102311 and coupler 102313 shown in FIGS. 179-184. However, it should be understood that other configurations are also contemplated.

The post or inner end 102468 of the paddle frame connector 102466 can connect to the coupler 102413 by any suitable means, such as, for example, any means described in the present application. In the illustrated example, the prongs 102470 on the inner end 102468 connect to the coupler 102413 in the same or similar manner as the implementation shown in FIGS. 136-139. That is, the coupler 102413 can includes opening 102415 for receiving the prongs 102470 (FIG. 186). In the illustrated example, arms 102473 connect the prongs 102470 to the inner end 102468 and allow the prongs 102470 to be pressed toward one another to enter the openings 102415.

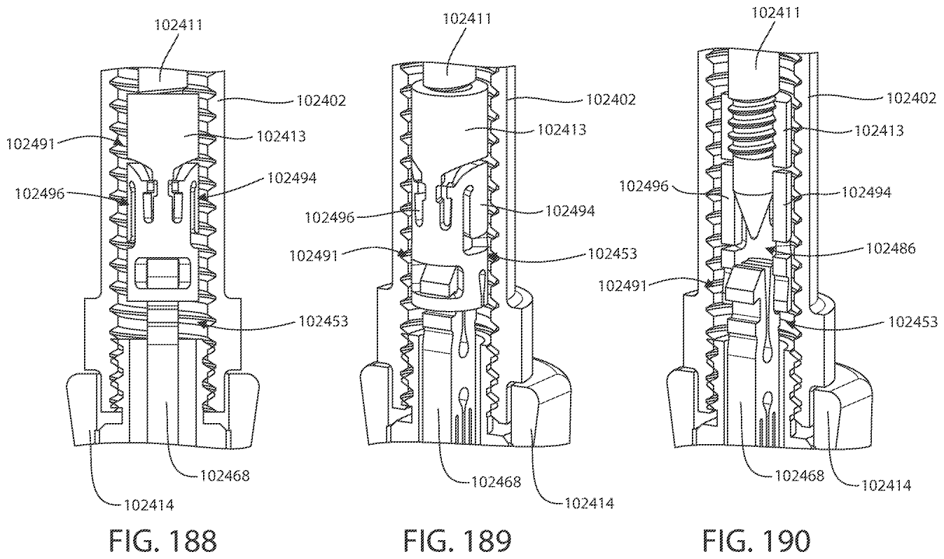

The coupler 102413 can also include a first arm 102494 and a second arm 102496 for engaging internal threads 102491 or other recesses or cutouts (FIGS. 188-195) of the receiver 102402 to maintain the coupler 102413 in a selected position. Setting the position of the coupler 102413 sets the position of the connector 102466 in a desired position, which maintains the paddle frame portions (See FIGS. 170, 171, and 179) at a desired width. Since the position of the coupler 102413 is set relative to the receiver 102402 and attached cap 102414, the width of the paddle frame portions is fixed as the paddles moved between closed, open, and extended positions by the receiver 102402 and the cap 102414.

The first and second arms 102494, 102496 extend from cutouts of the body 102481. Referring to FIGS. 196 and 200-201, when the coupler 102413 is in the normal position (i.e., disconnected from the width adjustment element 102411), the arms 102494, 102496 extend at an angle relative to the body 102481, where a portion of the arms 102494, 102496 extend within the lumen 102486 of the coupler 102413 and another portion of the arms 102494, 102496 extend away from an exterior of the body 102481. In the illustrated example, the proximal ends of the arms 102494, 102496 extend into the lumen 102486 of the coupler 102413, and the distal ends of the arms 102494, 102496 extend away from the body 102481. Alternatively, the proximal ends of the arms 102494, 102496 can extend away from the exterior of the body 102481, and the distal ends of the arms 102494, 102496 can extend into the lumen 102486.

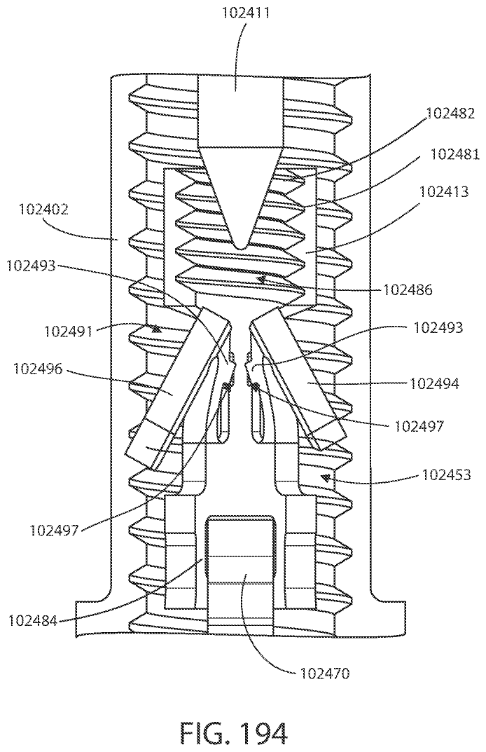

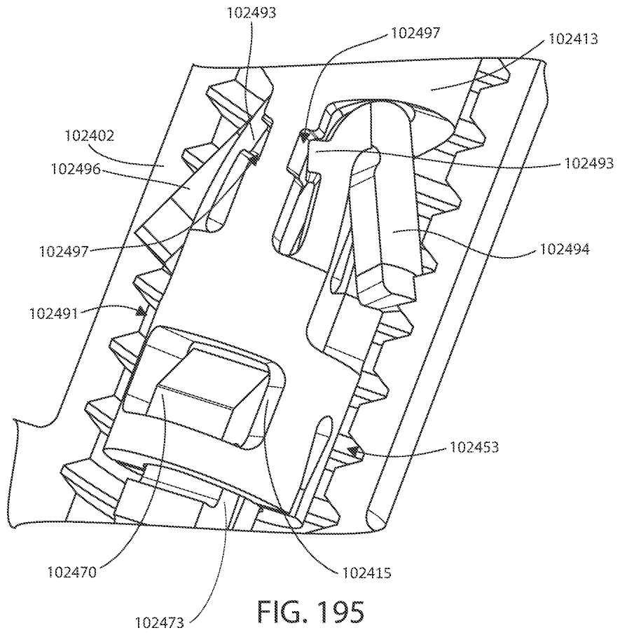

Referring to FIGS. 196-201, the first arm 102494 is offset from the second arm 102496 by a height H (FIG. 199) such that the arms 102494, 102496 are configured to connect to the internal threads 102491 (FIGS. 188-195) of the receiver 102402 when in the normal position (as shown in FIGS. 194-195). That is, the arms 102494, 102496 are configured to align with the pitch of internal threads 102491 of the receiver 102402. Accordingly, when the arms 102494, 102496 are in the normal position, the portion of the arms 102494, 102496 that extend away from the exterior of the body 102481 engage the internal threads 102491 of the receiver 102402 to prevent or inhibit movement of the coupler 102413 relative to the receiver 102402. In the illustrated example, the arms 102494, 102496 are configured to connect to square threads of the receiver 102402. However, it should be understood that other configurations are also contemplated, such as traditional internal threads, recesses, cutouts, etc. Referring to FIG. 201, in some implementations, the arms 102494, 102496 extend away from the body 102481 by an angle a when the arms 102494, 102496 are in the normal position. The angle α can be between about 10 degrees and about 45 degrees, such as between 15 degrees and about 30 degrees.

Referring to FIGS. 194 and 195, the arms 102494, 102496 can also have one or more tabs 102493 and the body 102481 can have one or more slots 102497 for receiving the tabs 102493 when the arms 102494, 102496 are in the normal position. The engagement between the tabs 102493 and slots 102497 prevents the arms 102494, 102496 from disengaging from the internal threads 102491 of the receiver 102402 due to forces provided on the arms 102494, 102496 resulting from the engagement with the internal threads 102491. In some implementations, each arm 102494, 102496 has two tabs 102493 (e.g., on opposite sides of the arm), and the body has slots 102497 that correspond to each of these tabs 102493. However, it should be understood that other configurations are also contemplated.

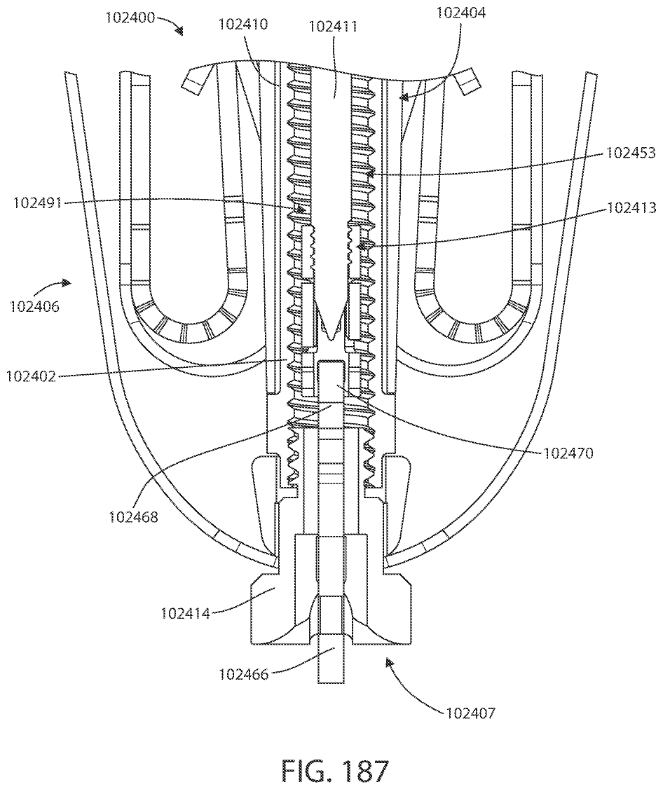

Referring to FIGS. 197-199, when the coupler 102413 is in the engaged position (i.e., connected to the width adjustment element 102411), the arms 102494, 102496 are engaged by the width adjustment element 102411 such that the arms pivot to a substantially aligned position with the body 102481 of the coupler 102413 (See FIGS. 187 and 190). That is, the connection between the width adjustment element 102411 and the coupler 102413 causes the width adjustment element to engage the portion of the arms 102494, 102496 that extends into the lumen 102486 of the coupler 102413, which causes the arms 102494, 102496 to pivot relative to the body 102481 such that the arms are substantially aligned with the body 102481. When the width adjustment element 102411 is connected to the coupler 102413, and the arms 102494, 102496 are in the engaged position, the arms 102494, 102496 do not engage the internal threads 102491 (FIGS. 188-195) of the receiver 102402, which allows a user to move the width adjustment element 102411 within the receiver 102402 to move the paddle frame members between the narrowed and expanded positions.

The coupler 102413 can be made using a variety of processes, including, but not limited to, cutting, such as laser cutting, stamping, casting, molding, heat treating, shape setting, etc. The coupler 102413 can be made of any suitable material that allows for the arms 102494, 102496 to be moved from the normal to expanded positions. For example, referring to FIG. 202, in some implementations, a sheet metal material 102401 can include the various features of the coupler 102413 such that the sheet metal material can be used to create the coupler 102413. In the illustrated example, the sheet metal material 102401 includes the arms 102494, 102496 of the implementation shown in FIGS. 185-201 and the attachment projections 102388, 102390 of the implementation shown in FIGS. 179-184. It should be understood that the sheet metal material 102401 can include the features of one or both of the arms 102494, 102496 and the attachment projections 102388, 102390.

Referring to FIG. 186, in the illustrated example, the connection between the width adjustment element or shaft 102411 and the connector 102466 causes the connector 102466 to move in the direction X when the width adjustment element 102411 moves in the direction Y. This causes the connector 102466 and attached paddle frames to move between the narrowed and expanded positions. In the illustrated example, movement of the width adjustment element or shaft 102411 proximally causes the paddle frame portions to narrow. Conversely, movement of the width adjustment element or shaft 102411 distally causes the paddle frame portions to expand or widen. Once the connector 102466 and paddle frames are in the desired position, the user can disconnect the width adjustment element 102411 from the coupler 102413, which will cause the arms 102494, 102496 of the coupler 102413 to move to the normal position and engage the internal threads 102491 of the receiver 102402 such that the paddle frame portions are set or maintained in the desired position.

The movement of the paddle frame portions to the narrowed position allows the device or implant 102400 to maneuver more easily into position for implantation in the heart by reducing the contact and/or friction between the native structures of the heart—e.g., chordae— and the device 102400. The movement of the connector 102466 and paddle frames to the expanded position provides the anchor portion of the device or implant 102400 with a larger surface area to engage and capture leaflet(s) of a native heart valve.

In some implementations, the connector 102466 can be made from or comprise a material that allows arms of the connector 102466 to be pulled into the receiver 102402. For example, the connector 102466, or a portion thereof, can be made of any flexible material, including but not limited to, metal, plastic, fabric, suture, etc. The connector 102466 can be made using a variety of processes, including, but not limited to, cutting, such as laser cutting, stamping, casting, molding, heat treating, shape setting, etc. The connector 102466 and paddle frames can be made from or comprise a shape memory material, —such as Nitinol—to provide shape-setting capability.

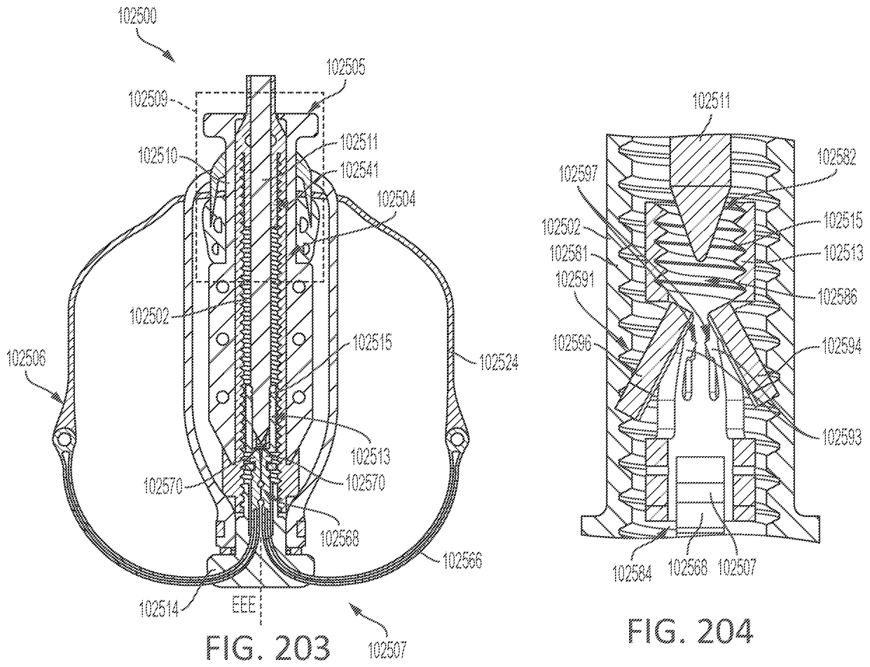

Referring to FIGS. 203-208, components of an example implementation of an implantable device or implant 102500 where a connector 102566 (e.g., shaped metal component, shaped plastic component, tether, wire, strut, line, cord, suture, etc.) and connected adjustable width paddle frames 102524 can be adjusted to a selected width and be set or fixed at the selected width to remain at the selected width are illustrated (See also FIGS. 170, 171, and 179). A coupler or connection feature 102513 is used to allow a width adjustment element to engage the connector 102566 and move the paddle frames 102524 between narrowed and expanded positions and to set the position of the paddle frames 102524 relative to a cap 102514 and/or receiver 102502 (e.g., an internally threaded element, a notched receiving portion, column, lumen, tube, shaft, post, housing, tracks, cylinder etc.). The implantable device 102500 and coupler 102513 can take the form of the implantable device 102400 and coupler 102413 shown in FIGS. 185-202. However, it should be understood that other configurations are also contemplated.

The implantable device 102500 can include a proximal or attachment portion 102505, an anchor portion 102506 (e.g., any anchor portion described in the present application) having paddle frames 102524 (attached to the connector—See FIGS. 170, 171, and 179), a coaptation or spacer portion 102504, and a distal portion 102507. The coaptation portion 102504, the proximal portion 102505, the anchor portion 102506, and the distal portion 102507 can be configured in a variety of ways, such as, for example, any of the ways described in the present application. In the illustrated example, the coaptation portion 102504 includes a coaptation element 102510 that takes the form of the coaptation element 102210 shown in FIGS. 170-178. However, it should be understood that other configurations are also contemplated.

In the illustrated example, the connector 102566 is symmetric along longitudinal axis EEE (FIG. 203). In some implementations of the implantable device 102500, however, the connector 102566 is not symmetric about the axis EEE. In the illustrated example, an inner end 102568 of the connector 102566 is engaged by a width adjustment element 102511 (e.g., width adjustment control, width adjustment wire, width adjustment shaft, width adjustment tube, width adjustment line, width adjustment cord, width adjustment suture, width adjustment tether, etc.) such that a user can move the inner end 102568 relative to the receiver 102502 to move the connector 102566 and attached paddle frames 102524 between the narrowed and expanded positions. In the illustrated example, the inner end 102568 of the connector 102566 includes prongs 102570 or other connecting structure that attaches to the coupler 102513. The inner end or post 102568 of the connector 102566 can take any suitable form, such as, for example, the form of the inner end 101368 of the connector 101324 shown in FIG. 139 with or without the optional slit 101374, or any other form described in the present application.

Referring to FIG. 203, the width adjustment element 102511 can be releasably connected to the coupler 102513, and the inner end 102568 of the connector 102566 can be connected to the coupler 102513. When the width adjustment element 102511 is connected to the coupler 102513, the coupler disengages from the receiver 102502. When the coupler 102513 is disengaged from the receiver 102502, movement of the width adjustment element 102511 causes the coupler 102513 and, consequently, the connector 102566 to move relative to the receiver 102502 to widen and narrow the paddle frames 102524.

The coupler 102513 has a body 102581 that includes a proximal opening 102582, a distal opening 102584, and a lumen 102586 that extends from the proximal to distal opening. The proximal end of the coupler 102513 has a connection portion 102515 for removably connecting to the width adjustment element 102511 (FIGS. 185-194). The connection portion 102515 can take any suitable form that allows for a connection between the width adjustment element 102511 and the coupler 102513, such as, for example, a threaded connection, a snap-fit connection, or any other suitable type of connection described in the present application. The connection portion 515 can be an internally threaded portion or can take the form for connecting the width adjustment element 102311 and coupler 102313 shown in FIGS. 179-184. However, it should be understood that other configurations are also contemplated and usable. The inner end 102568 of the connector 102566 can connect to the coupler 102513 by any suitable means, such as, for example, any means described in the present application. In the illustrated example, the prongs 102570 on the connector 102566 connect to the coupler 102513 in the same or similar manner as the implementation shown in FIGS. 136-139. However, it should be understood that other configurations are also contemplated.

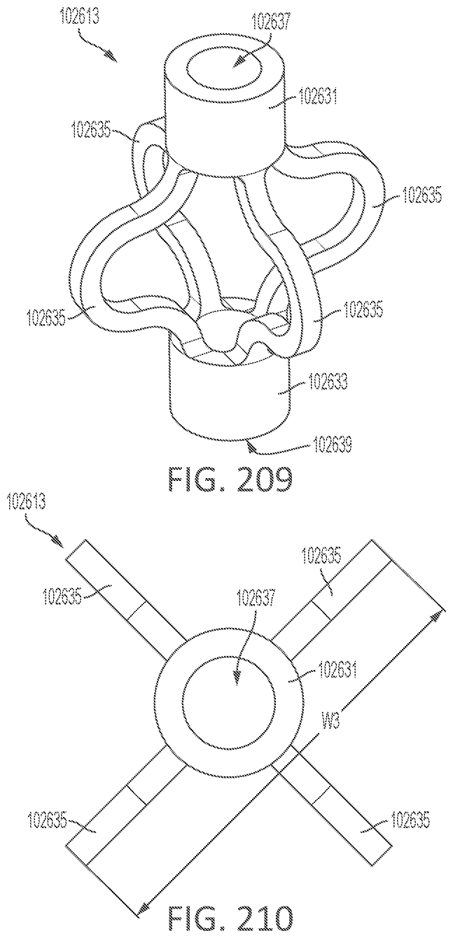

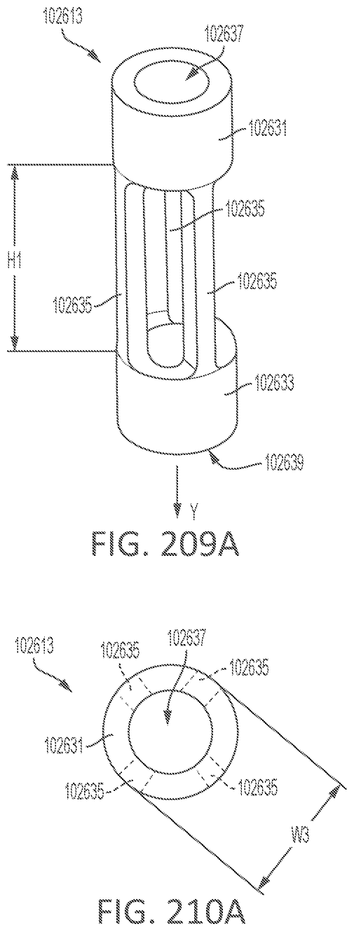

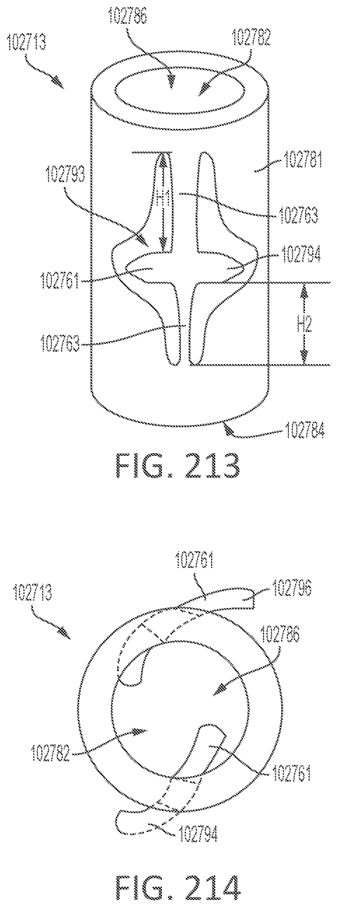

Referring to FIG. 204, in some implementations, when the width adjustment element 102511 is disconnected from the coupler 102513, the coupler engages the receiver 102502. When the coupler 102513 is engaged with the receiver 102502, movement of the coupler 102513 and the inner end 102568 of the connector 102566 is prevented, thereby setting or fixing the width of the paddle frame 102524. In the illustrated example, the coupler 102513 includes a first arm 102594 and a second arm 102596 for engaging internal threads 102591 or other recesses or cutouts (FIGS. 188-195) of the receiver 102502 to maintain the coupler 102513 in a selected position. Setting the position of the coupler 102413 sets the connector 102566 in a desired position, which maintains the paddle frame 102524 at a desired width (See FIGS. 170, 171, and 179). Since the position of the coupler 102513 is set relative to the receiver 102502 and attached cap 102514, the width of the paddle frame 102524 is fixed as the paddles move between closed, open, and extended positions by a user engaging an actuation element (e.g., actuation element 8102 shown in FIGS. 26-30) to cause the receiver 102502 and the cap 102514 to move relative to the coaptation element 102514. In some implementations, the arms 102594, 102596 can also have one or more tabs 102593 and the body 102581 can have one or more slots 102597 for receiving the tabs 102593 when the arms 102594, 102596 are in the normal position, which prevents the arms 102594, 102596 from disengaging from the internal threads 102591 of the receiver 102502 due to forces provided on the arms 102594, 102596 resulting from the engagement with the internal threads 102591. In some implementations, the coupler 102513 can take the form of the coupler 102413 shown in FIGS. 185-202. However, it should be understood that other configurations for the coupler are also contemplated that allows the coupler to be removably engaged with the receiver 102502, such as the configurations of the coupler 102613 shown in FIGS. 209-212 and the coupler 102713 shown in FIGS. 213-214.

Referring to FIGS. 203 and 204, connection of the width adjustment element 102511 to the coupler 102513 can cause the width adjustment element 102511 to engage and move the arms 102594, 102596 such that the arms pivot to a substantially aligned position with the body 102581 of the coupler 102513. This movement of the arms 102594, 102596 to the substantially aligned position with the body 102581 causes the arms to disengage with the internal threads 102591 of the receiver 102502, which allows the coupler 102513 to move relative to the receiver 102502 and cap 102514. This movement of the coupler 102513 relative to the receiver 102502 and cap 102514 allows a user to move the width adjustment element 102511 within the receiver 102502 to move the paddle frames 102524 between the narrowed and expanded positions.

In the illustrated example, movement of the width adjustment element 102511 proximally causes the paddle frame 102524 to narrow. Conversely, movement of the width adjustment element 102511 distally causes the paddle frame 102524 to expand or widen. Once the paddle frame 102524 is in the desired position, the user can disconnect the width adjustment element 102511 from the coupler 102513, which will cause the arms 102594, 102596 of the coupler 102513 to move to the normal position and engage the internal threads 102591 of the receiver 102502 such that the paddle frame 102524 is set or maintained at the desired width.

The movement of the paddle frames 102524 to the narrowed position allows the device or implant 102500 to maneuver more easily into position for implantation in the heart by reducing the contact and/or friction between the native structures of the heart—e.g., chordae— and the device 102500. The movement of the paddle frames 102524 to the expanded position provides the anchor portion of the device or implant 102500 with a larger surface area to engage and capture leaflet(s) of a native heart valve and/or to block a larger area of regurgitant flow through the native heart valve.

In some implementations, the connector 102566 can be made from a material that allows the connector 102566 to be pulled into the receiver 102502. For example, the connector 102566, or a portion thereof, can be made of any flexible material, including but not limited to, metal, plastic, fabric, suture, etc. The connector 102566 can be made using a variety of processes, including, but not limited to, cutting, such as laser cutting, stamping, casting, molding, heat treating, shape setting, etc. The paddle frame 102524 (attached to the connector) can be made from a shape memory material, —such as Nitinol—to provide shape-setting capability.

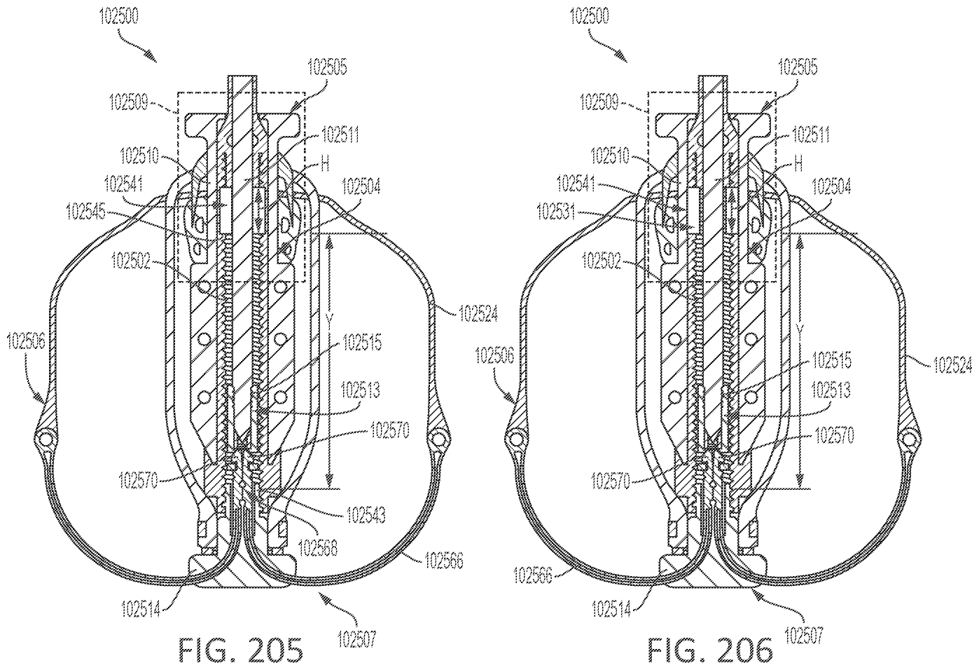

Referring to FIGS. 203-206, in some implementations, the receiver 102502 can have an unattachable portion 102541 that does not allow the coupler 102513 to engage or couple to the receiver 102502 when the width adjustment element 102511 is disengaged from the coupler 102513. The unattachable portion 102541 can take a wide variety of different forms. For example, the unattachable portion 102541 can be a filled or plugged portion of the receiver, a portion of the receiver without threads, and/or a portion of the receiver with windows or cutouts, any combination of these etc. Any configuration that prevents the coupler 102513 from engaging the receiver 102502 in the unattachable portion can be used. FIG. 203 illustrates an example where the unattachable portion comprises an extended plug. The length of the plug can be selected to limit the travel of the coupler. FIG. 205 illustrates an example where a portion of the internal threads of the receiver are removed or not included. The coupler 102513 can temporarily move in the receiver 102513 but will not latch in the unthreaded portion of the receiver. FIG. 205 illustrates an example where the receiver includes windows or cutouts. The coupler 102513 can temporarily move in the portion of the receiver 102513 with the windows or cutouts but will not latch in the portion of the receiver with the windows or cutouts.

In the illustrated examples, the unattachable portion 102541 is located at a proximal end 102509 of the receiver 102502. For example, a distance Y between a bottom 102543 of the receiver 102502 and a bottom 102545 of the unattachable portion 102541 can be between about 4 mm and about 10 mm or any subrange between 3 mm and 10 mm. In s implementations, a height H of the unattachable portion 102541 can be between about 0.5 mm and about 5 mm or any subrange between 0.5 mm and 5 mm. The unattachable portion 102541 prevents the paddle frames 102524 from being locked in a position that may cause a sustained stress or strain on the paddle frames 102524 that exceeds a maximum allowable permanent or set level of stress or strain, while also allowing the paddle frames 102524 to be temporarily moved to a fully narrowed position by moving the coupler 102513 through the unattachable portion 102541 of the receiver 102502. That is, the paddle frames 102524 can be moved to a narrower width during positioning of the device than the final width that the paddle frames can be set at.

Referring to FIG. 205, in some implementations, the unattachable portion 102541 includes a non-threaded portion such that the receiver 102502 does not have threads for the coupler 102513 to engage with when the coupler is in the unattachable portion 102541. For example, the unattachable portion 102541 of the receiver 102502 can be made by boring such that at least a portion of the unattachable portion 102541 does not include threads. In the illustrated example, an entirety of the unattachable portion 102541 does not include threads. In some implementations, only the portion of the unattachable portion 102541 that aligns with the arms 102594, 102596 (or other connection elements) of the coupler 102513 does not include threads.

Referring to FIG. 206, in some implementations, the unattachable portion 102541 includes windows or openings 102531 in the receiver 102502 such that the arms 102594, 102596 (or other connection elements) of the coupler 102513 extend through the opening 102531 if disengaged by the width adjustment element 102511 rather than engaging the receiver 102502. In the illustrated example, the openings 102531 are bisected by the cross-section of the view. However, it should be understood that other configurations and orientations are also contemplated. For example, the openings 102531 can be located along the outer surface of the receiver 102502 at a position that is 90 degrees from the illustrated position (i.e., extend into and out of the page) along the outer surface of the receiver 102502. In some implementations, unattachable portion 102541 can include more than one opening 102531 (e.g., two openings) to prevent or inhibit attachment of the coupler 102513 in the unattachable portion 102541.

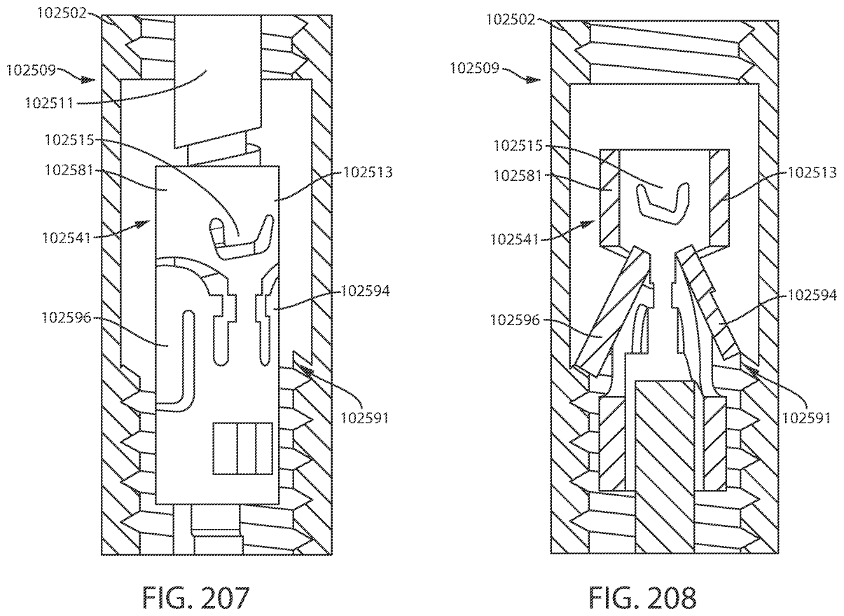

FIGS. 207-208 show the coupler 102513 moving through the unattachable portion 102541 of the receiver 102502. In the illustrated example, the coupler 102513 is shown moving through an unattachable portion 102541 that includes a non-threaded portion (as shown in FIG. 205), but it should be understood that the same principles apply to the unattachable portion 102541 having an opening 102531 shown in FIG. 206. Referring to FIG. 207, when the width adjustment element 102511 is connected to the coupler 102513, the arms 102594, 102596 are substantially aligned with the body 102581 of the coupler 102513, which allows a user to move the coupler 102513 relative to the receiver 102502 to move the paddle frames 102524 between narrowed and expanded positions. Referring to FIG. 208, when the width adjustment element 102511 is disconnected from the coupler 102513 and the coupler 102513 is positioned within the unattachable portion 102541, the arms 102594, 102596 pivot outward, but the unattachable portion 102541 does not have internal threads for the arms 102594, 102596 to engage with. Consequently, the coupler 102513 continues moving downward within the receiver 102502 until the arms 102594, 102596 engage the internal threads 102591 positioned below the unattachable portion 102541. Once the arms engage the internal threads 102591, the coupler 102513 is set relative to the receiver 102502, which sets the connector 102566 in a desired position and maintains the paddle frame 102524 at a desired width (See FIGS. 170, 171, and 179).

In some implementations, the receiver 102502 can include a window (not shown) at its distal end that allows a user to view the connection between coupler 102513 and width adjustment element 102511. In these implementations, the window can take a form similar to the window 102531 shown in FIG. 206, but rather than being positioned to prevent attachment between the width adjustment element 102511 and the coupler 102513, the window is positioned to allow a user to view a connection between the width adjustment element 102511 and the coupler 102513 when the width adjustment element 102511 is moved through the receiver 102502 to engage the coupler 102513. This window for viewing the connection between the coupler 102513 and width adjustment element 102511 can be included on either of the receivers 102502 shown in FIGS. 205-206, or the viewing window can be included on any other receiver described in the present application and can be used to view the connection between any coupler and any width adjustment element described in the present application.

Referring to FIGS. 209-212, an example implementation of a coupler 102613 used to allow a width adjustment element to engage a connector (e.g., shaped metal component, shaped plastic component, tether, wire, strut, line, cord, suture, etc. —see connector 102466 shown in FIGS. 185-187) and connected paddle frame (See FIGS. 170, 171, and 179) of an implantable device is shown. The coupler 102613 allows the width adjustment element to move the paddle frame between narrowed and expanded positions and to set the position of the paddle frame relative to a cap and/or receiver (e.g., an internally threaded element, a notched receiving portion, column, lumen, tube, shaft, post, housing, tracks, cylinder etc.) of the implantable device. The coupler 102613 can be used with any suitable implantable device, such as, for example, any implantable device described in the present application. In some implementations, the coupler 102613 is used with the implantable device 102400 shown in FIGS. 185-187, where the coupler 102613 takes the place of the coupler 102413, and such that the implantable device includes a proximal or attachment portion, an anchor portion, a connector connected to a paddle frame, a coaptation or spacer portion (that includes a coaptation element that takes the form of the coaptation element 102210 shown in FIGS. 170-178), and a distal portion. The width adjustment element can take any suitable form, such as, for example, the form of the width adjustment element 102411 shown in FIGS. 185-187 or any other width adjustment element shown or described herein. The receiver can take any suitable form, such as, for example, the form of the receiver 102402 shown in FIGS. 185-187 or the receiver 102502 shown in FIGS. 203-208. The cap can take any suitable form, such as, for example, the form of the cap 102414 shown in FIGS. 185-187. However, it should be understood that other configurations are also contemplated.

The coupler 102613 can have an upper body portion 102631, a lower body portion 102633, and a plurality of struts 102635 extending between and connected to both of the upper and lower body portions. The upper body portion 102631 can have an opening 102637 for receiving the width adjustment element of the implantable device, and the lower body portion 102633 can have an opening 102639 for receiving an inner end of the connector connected to the paddle frame. In some implementations, the opening 102637 of the upper body portion 102631 and the opening 102639 of the lower body portion 102633 can be aligned such that the width adjustment element extends through the upper body portion 102631 and is received by the opening 102639 of the lower body portion 102633. The upper body portion 102631 and/or the lower body portion 102633 can have a width adjustment element connection feature(s) that allows the width adjustment element to be removably connected to the coupler 102613. The width adjustment element connection feature(s) can be, for example, a threaded connection, a snap-fit connection, the form for connecting the width adjustment element 102311 and coupler 102313 shown in FIGS. 179-184, or any other suitable type of connection described in the present application. The lower body portion 102633 can have a paddle frame connection feature(s) that allows an inner end of the connector connected to the paddle frame to be connected to the coupler 102613. The paddle frame connection feature(s) can be, for example, a threaded connection, a snap fit connection, the form for connecting the paddle frame portions and the coupler shown in FIGS. 136-139, or any other suitable type of connection described in the present application.

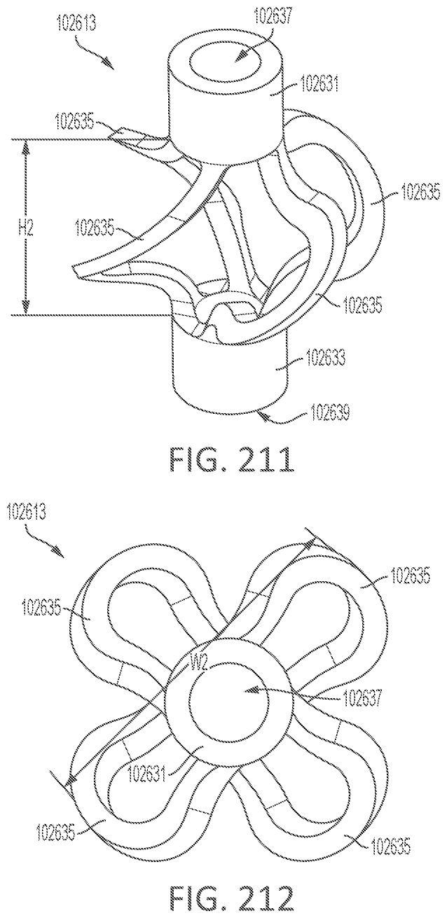

In some implementations, the coupler 102613 is movable between one or more unlocked positions and one or more locking positions. When in an unlocked position, the coupler 102613 can be in a position in which the struts 102635 can be in a substantially untwisted configuration (as shown in FIGS. 209-210) or a substantially straight configuration (as shown in FIGS. 209A-210A). When in a locking position, the coupler 102613 can be in a compressed position in which the struts 102635 are in a twisted or spiraled configuration (as shown in FIGS. 211-212). When in the unlocking position shown in FIGS. 209-210, the coupler 102613 has an overall width W1 (FIG. 210) and, when in the locking position shown in FIGS. 211-212, the coupler 102613 has an overall width W2 (FIG. 212). In some implementations, the width W2 is greater than the width W1. For example, the width W1 can be sized to be less than or equal to a diameter of the crest of the internal threads of the receiver to which the coupler 102613 is removably attached, and the width W2 can be sized to be greater than or equal to the diameter of the root of the internal threads of the receiver. When the coupler 102613 is in the unlocked position and the struts 102635 are in the entirely straight configuration, the coupler 102613 can have an overall width W3 (FIG. 210A) that is less than each of the widths W1, W2. Referring to FIG. 210A, when the struts 102635 are in the entirely straight configuration, outer surfaces of the struts 102635 can align with the upper and lower bodies 102631, 102635. Referring to FIG. 209A, in some implementations, the struts 102635 have a height H1. Referring to FIG. 211, in some implementations, the struts 102635 can have a height H2 (e.g., the distance between the upper and lower bodies 102631, 102633) when in the spiraled configuration. The height H1 is greater than the height H2 and the spiral of the coupler can be the same or substantially the same as the spiral of threads of the receiver.

In some implementations, the coupler 102613 is normally in or biased to the locking position such that the struts 102635 are in the compressed, spiraled configuration, and attachment of the width adjustment element to the coupler 102613 causes the coupler 102613 to move to the unlocked position. For example, the width adjustment element can be attached to one or both of the upper and lower body portions 102631, 102633 of the coupler 102613 and rotation of the width adjustment element provides torque to the coupler 102613 that causes the struts 102635 to move from the spiraled configuration illustrated by FIG. 211 to the configuration illustrated by FIGS. 209 and 210 and/or the configuration illustrated by FIGS. 209A and 210A. In some implementations, referring to FIGS. 209A and 210A, the width adjustment element can be attached to the upper body portion 102631 such that the width adjustment element extends beyond the upper body portion 102631 and engages the lower body portion 102633 or a component attached to the lower body portion (e.g., a connector attached to paddle frames) to cause the lower body portion 102633 to move in a direction Y relative to the upper body portion 102633. This movement of the lower body portion 102633 in the direction Y causes the struts 1026355 to move to the unlocked position such that the struts 102635 are in the substantially or entirely straight configuration.

In some implementations, the upper body portion 102631 has a width adjustment element connection feature, and the lower body portion 102633 has both a width adjustment element connection feature and a connection feature that attaches to a connector that attaches to adjustable width paddle frames or that attaches directly to adjustable width paddle frames. In these implementations, the width adjustment element can be attached to both the upper and lower body portions 102631, 102633 by a threaded connection. In some implementations, the continued rotation of the width adjustment element after being connected to the coupler 102613 provides torque to the coupler 102613 that causes the struts 102635 to move from the locking position to the unlocked position. The direction of the threaded connection between the width adjustment element and the coupler 102613 can be opposite the direction of the spirals of the struts 102635. For example, when the struts 102635 are normally spiraled in a counterclockwise direction, rotation of the width adjustment element in the clockwise direction connects the width adjustment element to the coupler 102613 and moves the coupler 102613 from the locking position to the unlocked position, and vice versa. The threaded connection between the width adjustment element and both the upper and lower body portions 102631, 102633 can promote elongation of the coupler 102613 such that the coupler moves from the configuration illustrated by FIG. 211 to the configurations illustrated by FIGS. 209 and/or 209A or any position in between. In some implementations, the upper body portion 102631 has a width adjustment element connection feature, and the lower body portion 102633 has a paddle frame connection feature (without having a width adjustment element connection feature). In this implementation, the width adjustment element can extend through the upper body portion 102631 and push against the lower body portion 102633 to move the coupler from the configuration illustrated by FIG. 211 to the configurations illustrated by FIGS. 209 and/or 209A or any position in between. In some implementations, the lower body portion has both a width adjustment element connection feature and a paddle frame connection feature, and the upper body portion 102631 does not include either of the width adjustment element or paddle frame connection features.

In some implementations, when the coupler 102613 is in the unlocked position, a user can move the width adjustment element and, consequently, the coupler 102613 and connector connected to the paddle frame within the receiver to move the paddle frame between narrowed and expanded positions. When the width adjustment element is disconnected from the coupler 102613, the coupler 102613 moves to the locking position FIGS. 211 and 212) such that the struts 102635 engage internal threads or other recesses or cutouts (FIGS. 188-195) of the receiver to set the position of the paddle frame portions relative to a cap and/or receiver. In some implementations, the connection between the width adjustment element and the upper body 102631 of the coupler 102613 allows the coupler 102613 to be moved to the locking position prior to the width adjustment element being disconnected from the coupler 102613. That is, at least a portion of the removable connection between the coupler 102613 and the width adjustment element does not cause the coupler 102613 to be moved to the unlocked position, which allows for a continued connection between the coupler 102613 and width adjustment element when the coupler 102613 is in the locking position. This is advantageous because it allows a user to confirm that the coupler 102613 is in a desired position before finally removing the width adjustment element from the coupler 102613. The struts 102635 can be configured to align with the pitch of internal threads of the receiver when in the spiraled configuration. This engagement between the struts 102635 and the receiver sets the position of the paddle frame portions of the implantable device relative to the cap and/or receiver.

The coupler 102613 can be made of any suitable material that allows for the upper and lower body portions 102631, 102633 to be moved between the compressed and expanded configurations and allows the struts 102635 to be moved between the spiraled and straight configurations. In some implementations, the coupler 102613 is made from a shape memory material, —such as Nitinol—to provide shape-setting capability. For example, the coupler 102613 can be a laser cut, Nitinol hypotube that is shape set in the compressed, spiraled configuration.

Referring to FIGS. 213-214, an example implementation of a coupler 102713 used to allow a width adjustment element to engage a connector (e.g., shaped metal component, shaped plastic component, tether, wire, strut, line, cord, suture, etc. —see connector 102466 shown in FIGS. 185-187) and connected paddle frame (See FIGS. 170, 171, and 179) of an implantable device is shown. The coupler 102713 allows the width adjustment element to move the paddle frame between narrowed and expanded positions and to set the position of the paddle frame relative to a cap and/or receiver (e.g., an internally threaded element, a notched receiving portion, column, lumen, tube, shaft, post, housing, tracks, cylinder etc.) of the implantable device. The coupler 102713 can be used with any suitable implantable device, such as, for example, any implantable device described in the present application. In some implementations, the coupler 102713 is used with the implantable device 102400 shown in FIGS. 185-187, where the coupler 102713 takes the place of the coupler 102413, and such that the implantable device includes a proximal or attachment portion, an anchor portion, a connector connected to paddle frame portions, a coaptation or spacer portion (that includes a coaptation element that takes the form of the coaptation element 102210 shown in FIGS. 170-178), and a distal portion. The width adjustment element can take any suitable form, such as, for example, the form of the width adjustment element 102411 shown in FIGS. 185-187 or any other width adjustment element shown or described herein. The receiver can take any suitable form, such as, for example, the form of the receiver 102402 shown in FIGS. 185-187 or the receiver 102502 shown in FIGS. 203-208. The cap can take any suitable form, such as, for example, the form of the cap 102414 shown in FIGS. 185-187. However, it should be understood that other configurations are also contemplated.

In the illustrated example, the coupler 102713 has a body 102781 that includes a proximal opening 102782, a distal opening 102784, a lumen 102786 that extends from the proximal to distal opening. The coupler 102713 can have a width adjustment element connection feature (not shown) for connecting to the width adjustment element. The width adjustment element connection feature can take any suitable form that allows for a connection between the width adjustment element and the coupler 102713, such as, for example, a threaded connection, a snap-fit connection, or any other suitable type of connection described in the present application. In some implementations, the width adjustment element connection feature takes the form for connecting the width adjustment element 102311 and coupler 102313 shown in FIGS. 179-184. However, it should be understood that other configurations are also contemplated. In some implementations, the coupler 102713 can have a paddle frame connection feature (not shown) for connecting to the connector and connected paddle frame portions of the implantable device. The paddle frame connection feature can be, for example, a threaded connection, a snap fit connection, the form for connecting the paddle frame portions and the coupler shown in FIGS. 136-139, or any other suitable type of connection described in the present application.

The coupler 102713 can also include a first arm 102794 and a second arm 102796 that extend from cutouts 102793 of the body 102781. When the coupler 102713 is disconnected from the width adjustment element and in the locking, normal position (as shown in FIG. 214), a portion of each of the arms 102794, 102796 extends within the lumen 102786 of the coupler 102713 and another portion of each of the arms 102794, 102796 extends away from an exterior of the body 102781. Connection of the width adjustment element to the coupler 102713 causes the width adjustment element to engage the arms 102794, 102796 and move the arms 102794, 102796 to be substantially aligned with the body 102781 of the coupler 102713 (as shown in FIG. 213).

Each of the arms 102794, 102796 can have a central portion 102761 and connection members 102763, that connect the central portion 102761 to the proximal and distal ends of the body 102781. In some implementations, the connection members 102763 are normally in torsion, such that at least a portion of the central portion 102761 of the arms 102794, 102796 is disposed within the lumen 102786 and another portion of the central portion 102761 of the arms 102794, 102796 extends away from the body 102781 when the coupler 102713 is in the locking, normal position. In some implementations, the heights H1, H2 of the upper lower connection members 102763 are identical. In some implementations, the height H1 of the upper connection member is greater than the height H2 of the lower connection member, or vice versa. In the illustrated example, the arms 102794, 102796 have a “t” shape, and the cutouts 102793 are configured to allow the central portion 102761 of the arms 102794, 102796 to move from the normal position to a substantially aligned position with the body 102781. However, it should be understood that other configurations/shapes are also contemplated.

In some implementations, the first arm 102794 is offset from the second arm 102796 such that the arms 102794, 102796 are configured to connect to the internal threads of the receiver when in the normal position (e.g., the position shown in FIG. 214). That is, the central portions 102761 of the arms 102794, 102796 are configured to align with the pitch of internal threads of the receiver. Accordingly, when the arms 102794, 102796 are in the normal position, the portion of the arms 102794, 102796 that extend away from the exterior of the body 102781 engage the internal threads of the receiver to prevent or inhibit movement of the coupler 102713 relative to the receiver. The arms 102794, 102796 can be configured to connect to any type of thread, recess, or cutout of the receiver.

Referring to FIG. 213, when the coupler 102713 is in an engaged and unlocked position (i.e., when the coupler 102713 is connected to the width adjustment element), the arms 102794, 102796 are engaged by the width adjustment element such that the arms pivot to a substantially aligned position with the body 102781 of the coupler 102713. That is, the connection between the width adjustment element and the coupler 102713 causes the width adjustment element to engage the portion of the arms 102794, 102796 that extends into the lumen 102786 of the coupler 102713, which causes the arms 102794, 102796 to pivot relative to the body 102781 such that the arms are substantially aligned with the body 102781. In the illustrated example, the arms 102794, 102796 pivot in a clockwise direction to move to the aligned position. In some implementations, the arms 102794, 102796 can pivot in a counter-clockwise direction to move to the aligned position. When the width adjustment element is connected to the coupler 102713, and the arms 102794, 102796 are in the engaged position, the arms 102794, 102796 do not engage the internal threads of the receiver, which allows a user to move the actuation element and, consequently, the connector connected to the paddle frame within the receiver such that the paddle frame moves between the narrowed and expanded positions.

Once the coupler 102713 is in the desired position relative to the receiver, the width adjustment element is disconnected from the coupler 102713 such that the coupler 102713 moves to the normal, locking position. In some implementations, the connection between the width adjustment element and the body 102781 of the coupler 102713 allows the coupler 102713 to be moved to the normal, locking position prior to the width adjustment element being disconnected from the coupler 102713. That is, at least a portion of the removable connection between the coupler 102713 and the width adjustment element does not cause the coupler 102713 to be moved to the unlocked position (e.g., because the width adjustment element is not engaging the arms 102794, 102796 when the width adjustment element is still connected to the body 102781), which allows for a continued connection between the coupler 102713 and width adjustment element when the coupler 102713 is in the locking position. This is advantageous because it allows a user to confirm that the coupler 102713 is in a desired position before finally removing the width adjustment element from the coupler 102713.

The coupler 102713 can be made of any suitable material that allows for the arms 102794, 102796 to move between the normal position and the engaged position. In some implementations, the coupler 102713 is made from a shape memory material, —such as Nitinol—to provide shape-setting capability. For example, the coupler 102713 can be a laser cut, Nitinol hypotube that is shape set in the locking position (as shown in FIG. 214).

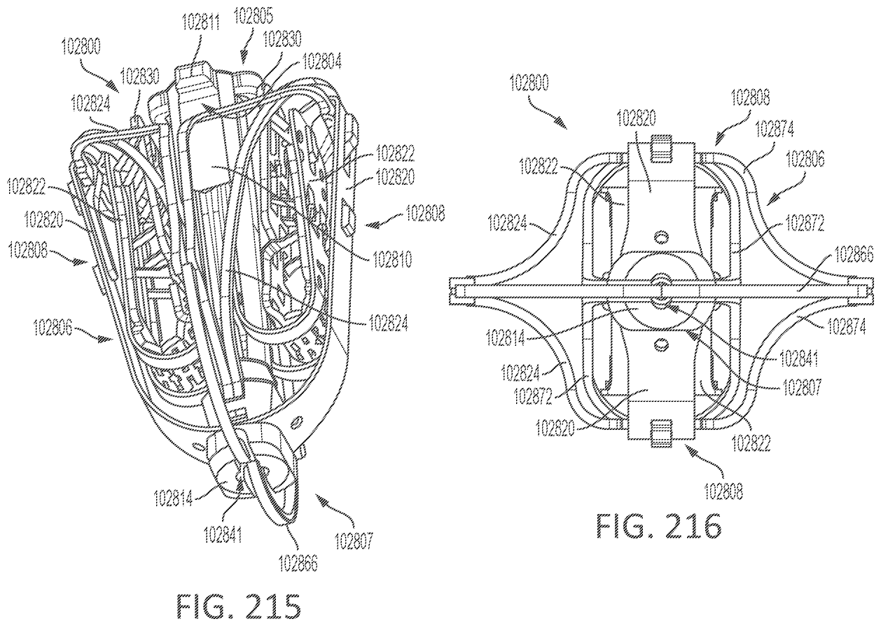

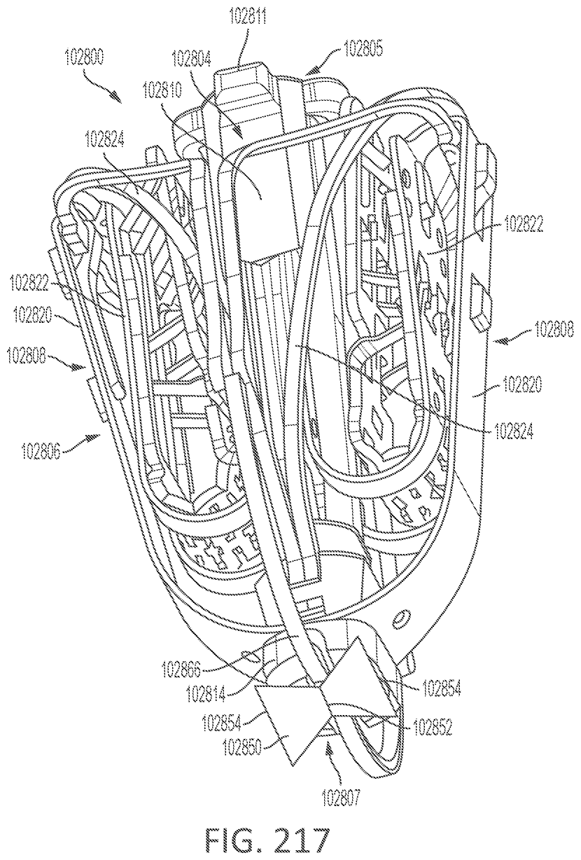

Referring now to FIGS. 215-219, an example of an implantable device or implant 102800 is shown that includes a cover element 102850 (FIGS. 217-219) for preventing or inhibiting regurgitation of blood through an interior of the implantable device 102800. The implantable device 102800 is one of the many different configurations that the device 100 that is schematically illustrated in FIGS. 8-14 can take. For example, in some implementations, the implantable device 102800 includes a coaptation portion 102804, a proximal or attachment portion 102805, an anchor portion 102806, and a distal portion 102807. The device 102800 can include any other features for an implantable device or implant discussed in the present application and/or in U.S. Provisional Patent Application No. 63/217,622 filed on Jul. 1, 2021, which is incorporated herein by reference in its entirety, and the device 102800 can be positioned to engage valve tissue 20, 22 as part of any suitable valve repair system (e.g., any valve repair system disclosed in the present application and/or U.S. Provisional Patent Application No. 63/217,622). The device 102800 can be a prosthetic spacer device, valve repair device, or another type of implant that attaches to leaflets of a native valve.

In some implementations, the anchor portion 102806 includes a plurality of anchors 102808. The anchors 102808 can be configured in a variety of ways, such as, for example, any of the ways described in the present application. In the illustrated example, the anchor portion 102806 includes two anchors 102208 with each anchor 102808 having outer paddles 102820, inner paddles 102822, paddle frames 102824, and clasps 102830. The outer paddles 102820, inner paddles 102822, paddle frames 102824, and clasps 102830 can take any suitable form, such as, for example, any form described in the present application. In some implementations, the paddle frames 102824 can have inner frame portions 102872 (FIG. 216) and outer frame portions 102874 (FIG. 216), such as, for example, similar to any other paddle frame disclosed in the present application that includes inner and outer frame portions. The anchors 102808 can include an optional cover, such as, for example, any of the covers disclosed in FIGS. 143-169 of the present application, where the cover can be configured to assist in coapting the leaflets and preventing or inhibiting regurgitation of blood through the native valve. The cover can be attached to the anchors 102808 in any suitable manner and by any suitable means, such as, for example, any manner and/or means described in the present application.

The attachment portion 102805 and distal portion 102807 can take any suitable form, such as, for example, any form described in the present application. In the illustrated example, the attachment portion 102805 includes a first or proximal collar 102811 (or other attachment element) for engaging with a coupler of a delivery system. In the illustrated example, the distal portion 102807 includes a cap 102814 that is operatively connected to the anchor portion 102806 of the device 102800 such that movement of the cap 102814 relative to a spacer or coaptation element 102810 of the coaptation portion 102804 causes the anchors 102808 to move between open and closed positions.

The coaptation portion 102804 of the device can include a coaptation element 102810 (e.g., a spacer, coaption element, bushing, etc.) for implantation between leaflets of a native valve. The coaptation element 102810 can take any suitable form, such as, for example, the form shown in FIGS. 170-178, or any other form described in the present application. In some implementations, the coaptation element 102810 extends from the proximal collar 102811 (or other attachment element) and includes a lumen (not shown) for receiving one or more actuation elements (e.g., actuation shaft(s), actuation rod(s), receiver(s), actuation wire(s), actuation line(s), etc.) of the implantable device 102800. In the illustrated example, the proximal collar 102811 of the attachment portion 102805 is integral with the coaptation element 102810.

The proximal end of the coaptation element 102810 has a proximal opening (not shown) that allows one or more actuation elements to move relative to the lumen of the coaptation element 102810. The proximal opening allows one or more actuation elements to engage one or more controllable components of the device or implant 102800. The distal end of the coaptation element 102810 can include a distal opening (not shown). An actuation element and/or one or more components that couple the cap 102814 to the actuation element can extend through the distal opening and attach to the cap 102814. In some implementations, an actuation element extends from the delivery system to engage and enable actuation of the device 102800 between the open and closed positions and/or a width adjustment member extends from the delivery system to adjust widths of the paddles. Movement of the cap 102814 away from the coaptation element 102810 can cause the anchors 102808 to move to the open position, and movement of the cap 102814 toward the coaptation element 102810 can cause the anchors 102808 to move to the closed position. The actuation element, the cap, and any components that couple the cap to the actuation element can be configured to removably engage the cap 102814 in any suitable manner that allows the actuation element to be disengaged and removed from the device 102800 after implantation, such as, for example, any manner described in the present application.

In some implementations, one or more width adjustment elements extend through the lumen of the coaptation element 102810 and engage the paddle frames 102824 to move the paddle frames 102824 between a narrowed position and an expanded position. For example, a connector 102866 (e.g., shaped metal component, shaped plastic component, tether, wire, strut, line, cord, suture, etc.) or other component that is coupled to the paddle frames 102824 can extend through an opening 102841 of the cap 102814 and into the lumen of the coaptation element 102810. The width adjustment element(s) can be configured to engage and move the connector 102866 or other component of the paddle frames 102824 relative to the cap 102814. In some implementations, movement of the connector 102866 or other component of the paddle frames 102824 in a distal direction relative to the cap 102814 can cause the paddle frames 102824 to move to an expanded position, and movement of the connector 102866 or other component of the paddle frames 102824 in a proximal direction relative to the cap 102814 can cause the paddle frames 102824 to move to a narrowed position. The paddle frames 102824 can be configured to move between the expanded and narrowed positions by any suitable means, such as, for example, any means described in the present application.

Referring to FIGS. 217-219, the implantable device 102800 includes a cover element 102850 (FIGS. 217-219) for preventing or inhibiting regurgitation of blood through an interior of the implantable device 102800. For example, the cover element 102850 is configured to prevent or inhibit blood from moving through the opening 102841 of the cap and the openings and lumen of the coaptation element 102810. The cover element 102850 can, however, be configured to prevent or inhibit blood from moving through any other interior portion of the implantable device 102800. The cover element 102850 can be connected to the cap or other component of the device and be spaced apart from the connector. This prevents or inhibits blood flow through the opening 102841 and allows the connector 102866 to freely move into and out of the cap to narrow and widen the paddles.

The cover element 102850 can be positioned at a distal end of the device 102800 and cover the opening 102841 (FIGS. 215-216) of the cap 102814. In the illustrated example, the cover element 102850 is connected to the cap 102814. The cover element 102850, however, can be attached to the paddle frames 102824, to the paddles, or any other portion of the device 102800 that allows the connector 102866 to move relative to the cap 102814 such that the paddle frames 102824 can be moved between expanded and narrowed positions. The cover element 102850 can be connected to the device 102800, such as, for example, by one or more connectors (e.g., stitches, adhesive, mechanical fasteners, ultrasonic welds, etc.), or by any other suitable means that secures the cover element 102850 in a position to prevent or inhibit blood from regurgitating through an interior of the device 102800.

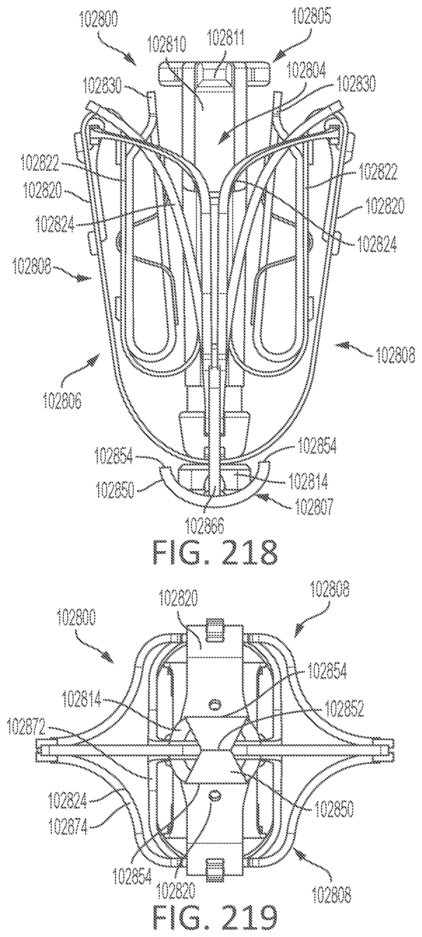

In the illustrated example, referring to FIGS. 217 and 219, the cover element 102850 has a width that expands from a center 102852 to the side edges 102854. Having a smaller width proximate the opening 102841 (FIGS. 215-216) of the cap 102814 can allow the connector 102866 to more freely move in and out of the opening 102841 as the paddle frame 102824 is moved between narrowed and expanded positions. The cover element 102850 can, however, have any suitable shape, such as, for example, a rectangular shape, a circular shape, oval shape, a triangular shape, or any other shape that is capable of covering the opening 102841 of the cap 102814. Referring to FIG. 218, the cover element 102850 can be curved (or otherwise formed) such that the side edges 102854 extend upward, which could be advantageous in preventing or inhibiting blood from entering the opening 102841 of the cap 102814.

The cover element 102850 can be made of any suitable type of material that prevents or inhibits movement of blood. For example, the cover element 102850 can be made from a cloth material, such as flexible material, a porous material, and/or a material that is impermeable to blood flow. In some implementations, the cover element 102850 is made from a biocompatible material, such as a woven biocompatible fabric that is configured to promote tissue ingrowth.

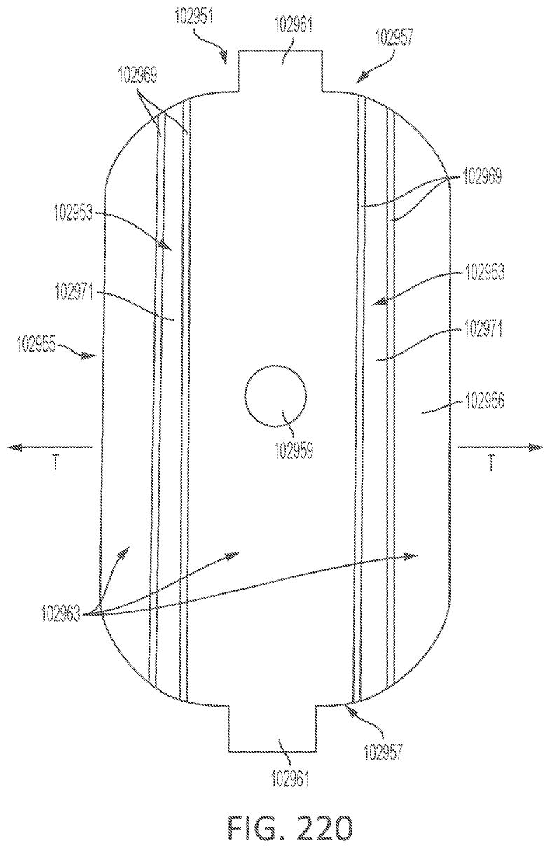

Referring to FIGS. 220-224, an example of a cover 102951 for an implantable device or implant is shown. The cover 102951 can be used with any suitable implantable device, such as, for example, any implantable device described in the present application. In some implementations, the cover 102951 is configured to be attached to paddle frames (e.g., any paddle frames described in the present application) of the implantable device. For example, the cover 102951 can be configured to connect to inner and outer paddle frame portions of the paddle frames, where the paddle frames are movable between narrowed and expanded positions, such as the paddle frames 8224 shown in FIGS. 26-30 of the present application. However, it should be understood that the cover 102951 can be configured for connecting to any component of the implantable device.

The cover 102951 can be attached to the paddle frame and configured to provide further engagement between the implantable device and the leaflets when implanted. The cover 102951 can include a sheet, material, fabric, layer, or membrane that is attached to the paddle frames of the implantable device by a plurality of connectors (e.g., stitches, adhesive, mechanical fasteners, ultrasonic welds, etc.). The sheet, material, fabric, layer, or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth. In implementations in which the implantable device has paddle frames having inner and outer paddle frame portions, it should be understood that the cover 102951 can be attached to one or both of the inner and outer paddle frame portions. The cover 102951 can be configured to cover or not cover any component or portion of the implantable device. The cover 102951 can be attached to and positioned relative to the inner and/or outer paddle frame portions in any way described in the present application.

The cover 102951 can have one or more stretchable portions 102953 allow the cover 102951 to maintain a substantially taut state when in a normal position, while also allowing the cover 102951 to stretch to an expanded state. This is advantageous for situations in which the cover 102951 is attached to paddle frames that are movable between narrowed and expanded positions. That is, the cover 102951 maintaining a taut state when in a normal position, and when paddle frames are in a narrowed position, reduces any excess material on the implantable device that can contact vasculature. The stretchability of the cover 102951 then allows the paddle frames to move to an expanded state with the cover 102951 maintaining a substantially taut state and maintaining a covering for the implantable device that prevents or inhibits regurgitation of blood by acting as a barrier that prevents or inhibits the movement of blood through the native valve. The stretchable portions 102953 can take a wide variety of different forms. Any material that can stretch and return to its original size or substantially to its original size can be used.

Referring to FIG. 220, the cover 102951 is shown cut from a flat sheet of material or formed as a flat sheet. The cover 102951 includes different shaped segments or portions to attach to different portions of an implantable device. The cover 102951 can be shaped to smooth transitions between various portions of the implantable device to reduce catch points and provide a smoother exterior to the device. In the illustrated example, the cover 102951 includes a main body portion 102955 having a center portion 102956 and rounded edge portions 102957, where the main body portion 102955 is configured to attach to opposing paddle frames of a pair of anchors. The center portion 102956 includes an opening 102959 for receiving and attaching the cover to a cap and/or other portion of the implantable device. The cover 102951 further includes end portions 102961 for further attaching the cover 102951 to another cover or any other component of the implantable device. It should be understood, however, that the cover 102951 can take any suitable shape or form and be configured to attach to any portion of an implantable device. For example, the cover 102951 can take any shape and form described in the present application.

Referring to FIG. 220, in the illustrated example, the cover 102951 includes a plain weave 102963 with a pair of stretchable portions 102953. Referring to FIG. 222, the plain weave 102963 can include weft yarns 102965 and warp yarns 102967 that are woven in a perpendicular weaving pattern. However, it should be understood that the plain weave 102963 can take any other suitable form, or the cover 102951 can include any other type of main weaving pattern with the stretchable portions 102953 positioned therein. In some implementations, the plain weave 102963 is made of a woven biocompatible fabric that is configured to promote tissue ingrowth. The plain weave 102963 can also be configured to reduce blood regurgitation.

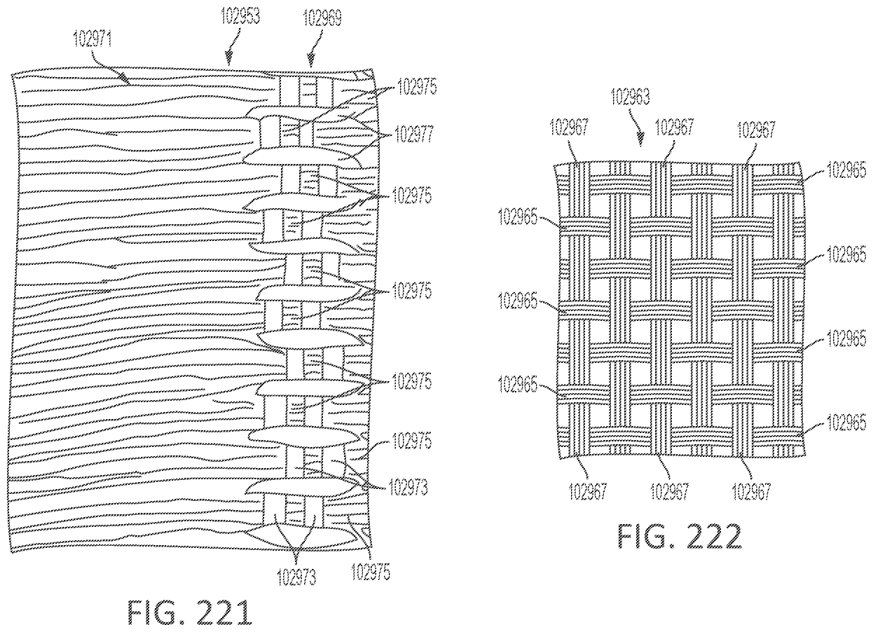

Referring to FIGS. 220-21 and 223-224, the stretchable portions 102953 can include a pair of transition portions 102969, such as Leno weaves, with a stretchable material or float 102971 connected therebetween. The transition portion, such as a Leno weave, provides a transition, connection, or interface between the plain weave 102963 and the stretchable material or float. Referring to FIG. 221, the float 102971 can include a portion of threads that are not interlaced with any other threads. In some implementations, the floats 102971 include textured yarn. However, it should be understood that other configurations are contemplated. Still referring to FIG. 221, the Leno weaves 102969 can include warp yarns 102973 and weft yarns 102975 that are woven in a perpendicular weaving pattern with at least one Leno yarn 102977 wrapping the warp yarns 102973. In the illustrated example, a single Leno yarn 102977 wraps around four warp yarns 102973, and the weft yarns 102975 are extensions of the yarns from the float 102971. In some implementations, the weft yarns 102975 from the Leno weave 102969 extends to the weft yarns 102965 (FIG. 222) of the plain weave 102963 (FIG. 222). In some implementations, the plain weave 102963 is separate from the Leno weave 102969, and the weft yarns 102975 of the Leno weave 102969 are folded back over or otherwise connected to the warp yarns 102973 and/or Leno yarn 102977 of the Leno weave 102969. However, it should be understood that other configurations for the stretchable portions 102953 are contemplated. The Leno weave 102969 produces an open fabric that prevents or inhibits slippage or misplacement of threads. The Leno weave 102969 can provide further resistance to blood regurgitation.

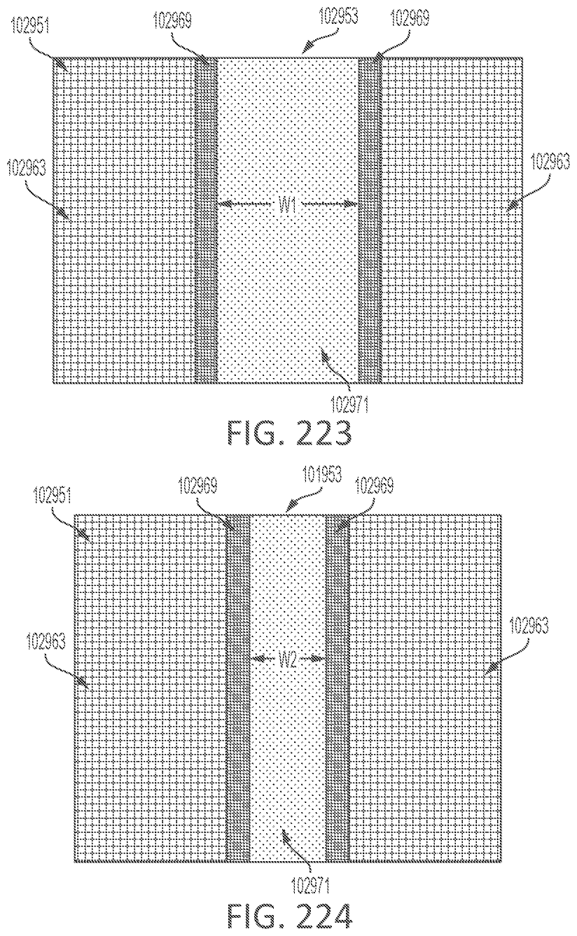

Referring to FIGS. 223-224, the stretchable portions 102953 can be created by taking a cover 102951 that includes the plain weave 1029663, two or more Leno weaves 102969, and one or more floats 102971 described above in an initial state (as shown in FIG. 223) and heating the cover 102951 such that the floats 102971 shrink to a narrowed state (as shown in FIG. 224). For example, the yarn of the floats 102971 can crimp and/or curl during heating and cause the floats 102971 to move to the narrowed state. Heat setting or heat pressing the cover 102951 can cause the floats 102971 to shrink to the narrowed state. Once the floats 102971 shrink to the narrowed state, the cover 102951 normally has a width W (FIG. 220) that is based on the floats being in the narrowed state, but the floats can be pulled to expand to a stretched state such that allows the width W of the cover 102951 to expand when tension T is applied to the cover. For example, pulling the stretchable material or float can temporarily straighten out the curled and/or kinked strands of the stretchable material or float. Removal of the tension T from the cover 102951 causes the floats to move back to the narrowed state such that the cover moves back to the normal position having the width W. That is, the strands pull back to the curled and/or kinked heat set configuration. Heating the cover 102951 can also provide advantages to the plain weave 102963. For example, heat setting or heat pressing the plain weave 102963 can reduce the pore size and/or increase the density of the plain weave 102963, which can be advantageous in preventing or inhibiting blood regurgitation.

In some implementations, the floats 102971 can have a width W1 (FIG. 223) when in the pre-heated state and a width W2 (FIG. 224) after being heated. In some implementations, the width W1 can be between about 3 mm and about 7 mm or any subrange, and the width W2 can be between about 1 mm and about 3 mm or any subrange. The ratio of the width W1 to the width W2 can be between about 1.1 to 1 and about 7 to 1 or any subrange. However, it should be understood that the widths W1, W2 can take any other suitable sizes based on the portion of the implantable device that the cover 102951 is connected to or the desired amount of stretch for the cover 102951.

In some implementations, the cover 102951 is configured to maintain a substantially taut state when in a normal position, while also allowing the cover 102951 to stretch to an expanded state, but the cover 102951 does not include the discrete stretchable portions 102953. The cover 102951 can be configured to be stretchable and/or resilient in a variety of different ways. In some implementations, the cover 102951 is made to be stretchable by rotating the material of the cover such that the horizontal and vertical yarns of the weave are no longer horizontal and vertical before the cover is cut from the material. For example, the material that forms the cover can be rotated between 30 degrees and 60 degrees, such as between 40 and 50 degrees, such as about 45 degrees or 45 degrees. The rotation of the fabric that forms the material of the cover allows the cover to stretch as the paddle frames are moved between the narrow and wide configurations. It should be understood, however, that the cover 102951 can be configured to stretch in a wide variety of different ways.

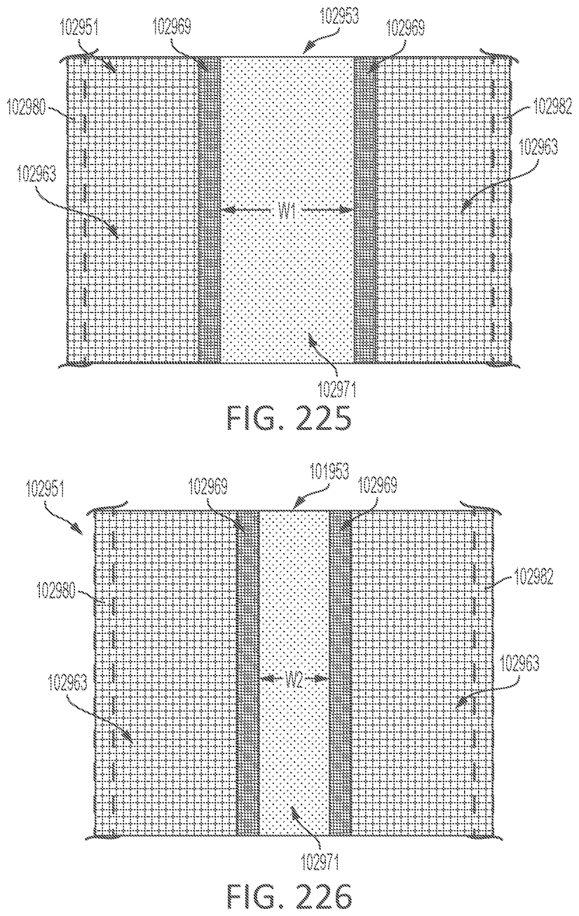

Referring to FIGS. 225-226, the cover 102951 can be attached to a first paddle frame portion 102980 and a second paddle frame portion 102982 of a paddle frame of an implantable device. The paddle frame portions 102980, 102982 can take any suitable form, such as, for example, any form for a paddle frame portion described in the present application or any application cited herein. For example, the paddle frame portions 102980, 102982 can both be inner paddle frame portions or outer paddle frame portions, or one can be an inner paddle frame portion and the other can be an outer paddle frame portion. In the illustrated example, the cover 102951 includes a single stretchable portion 102953 that takes the form of the stretchable portion shown in FIGS. 223-224. However, it should be understood that the cover 102951 can have any suitable number of stretchable portions 102953 between the first and second paddle frame portions 102980, 102982. Referring to FIG. 225, when the paddle frame portions 102980, 102982 are in an expanded state, the stretchable portion 102953 can stretch such that the float 102971 has a width W1, which is substantially identical to the width W1 of the pre-heated float 102971 shown in FIG. 223. Referring to FIG. 226, when the paddle frame portions 102980, 102982 are in a narrowed state, the stretchable portion 102953 move back to the normal position such that the float 102971 has a width W2, which is substantially identical to the width W2 of the heated float 102971 shown in FIG. 224.

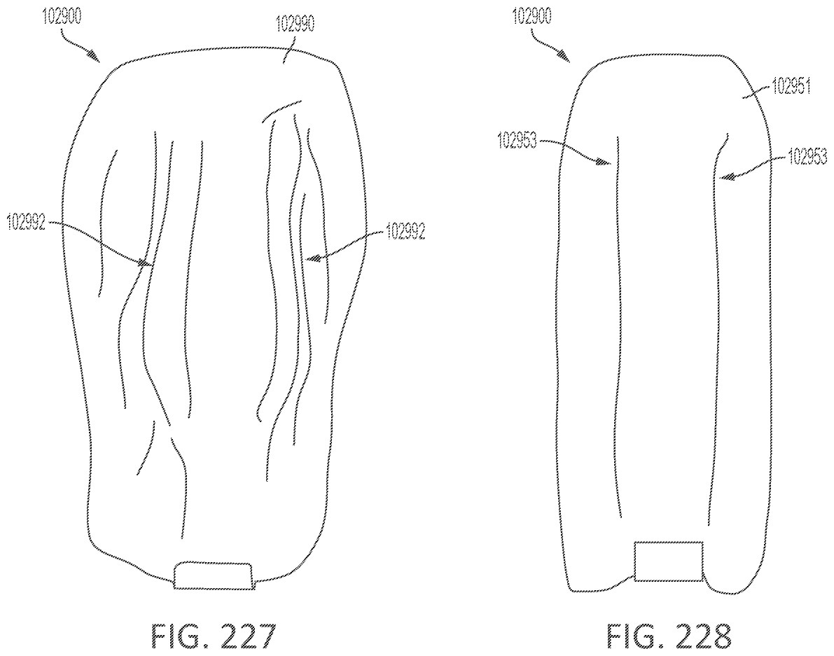

The cover 102951 is advantageous in preventing or inhibiting bunching or wrinkling as the paddle frames are moved from a wide configuration to a narrow configuration. For example, referring to FIG. 227, a cover 102990 is shown attached to an implantable device 102900 that is movable between narrowed and expanded positions, where the cover 102990 does not include the stretchable portions 102953 described above. In this example, the device is shown in the narrowed position and the cover 102990 has various folds or excess material 102992 because the cover needs to be able to move to accommodate the device 102900 moving to the expanded position. Referring to FIG. 228, the device 102990 includes the cover 102951 having stretchable portions 102953. In this implementation, the cover 102951 maintains a taut state when the device 102900 is in the narrowed position such that excess material extending from the cover 102951 is minimal or non-existent, but the stretchable portions 102953 allow the cover 10251 to expand when the device 102900 is moved to the expanded position.

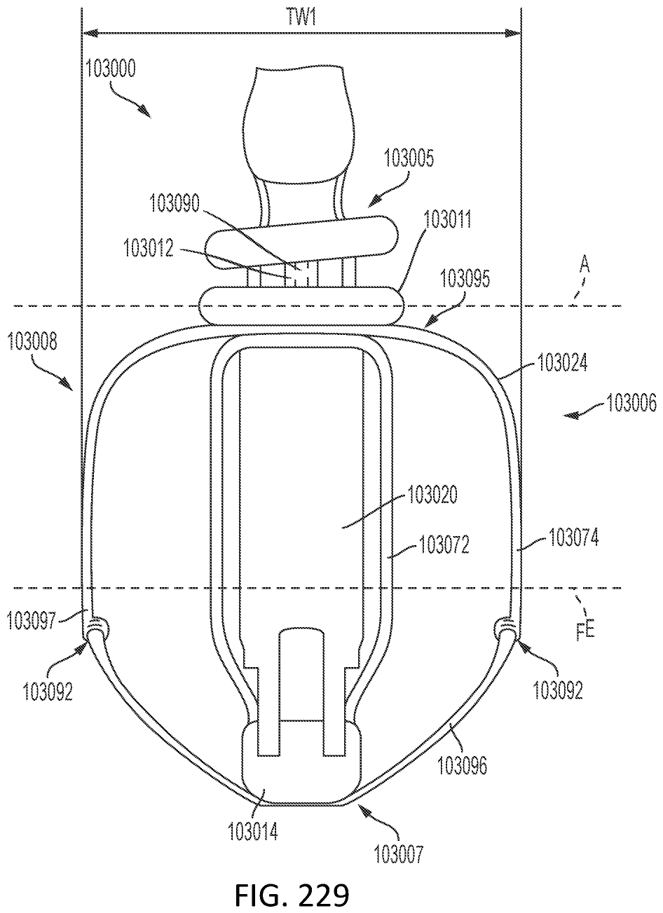

FIGS. 229-233 illustrate an example implementation of an implantable device or implant 103000 that includes an anchor portion 103006 having one or more paddle frames 103024 that are movable to a narrowed position. For example, the paddle frames can be moved to the narrowed position to allow the device 103000 to maneuver more easily into position for implantation in the heart by reducing the contact and/or friction between the native structures of the heart—e.g., chordae—and the device 103000. A width adjustment element 103090 can be controlled by a user to control a width of the paddle frames 103024. For example, the width adjustment element 103090 can pull a connector 103096 to move the paddle frames 103024 to a narrowed position. The paddle frames 103024 can be narrowed as the device 103000 is being positioned for implantation on the leaflets of a native valve. The device 103000 can include any other features for an implantable device or implant discussed in the present application or in the applications and patents incorporated by reference herein, and the device 103000 can be positioned to engage valve tissue 20, 22 as part of any suitable valve repair system (e.g., any valve repair system disclosed in the present application). In addition, any of the devices described herein can incorporate the features of the device 103000.

The implantable device or implant 103000 can include an optional coaptation portion, a proximal or attachment portion 103005, an anchor portion 103006, and a distal portion 103007. The coaptation portion, attachment portion 103005, and distal portion 103007 can take any suitable form, such as, for example, any form described in the present application. In some implementations, the coaptation portion optionally includes a coaptation element (e.g., a spacer, coaption element, gap filler, etc.) that can be used, for example, for implantation between the leaflets 20, 22 of the native mitral valve MV. The coaptation element, etc. can take any suitable form, such as, for example, any form described in the present application.

In some implementations, the attachment portion 103005 includes a first or proximal collar 103011 for engaging with a capture mechanism of a delivery system. The capture mechanism and the delivery system can take any suitable form, such as, for example, any form described in the present application. The delivery system can be the same as or similar to other delivery systems herein, e.g., 102, 402, 502, etc. and can comprise one or more of a catheter, a sheath, a guide catheter/sheath, a delivery catheter/sheath, a steerable catheter, an implant catheter, a tube, a channel, a pathway, combinations of these, etc. The proximal collar 103011 can take any suitable form, such as, for example, any form described in the present application.

In some implementations, the distal portion 103007 includes a cap 103014 that is attached to anchors 103008 of the anchor portion 103006 such that movement of the cap 103014 causes the anchors 103008 to move between open and closed positions. The cap 103014 can take any suitable form, such as, for example, any form described in the present application. In the illustrated example, an actuation element 103012 (e.g., the same as or similar to actuation element 112 shown in FIGS. 8-20 or actuation element 8102 shown in FIGS. 26-30) extends from a delivery system (e.g., any delivery system described in the present application) and engages the cap 103014 to move the cap 103014. The actuation element can engage and move the cap by any suitable means, such as, for example, any means provided in the present application.

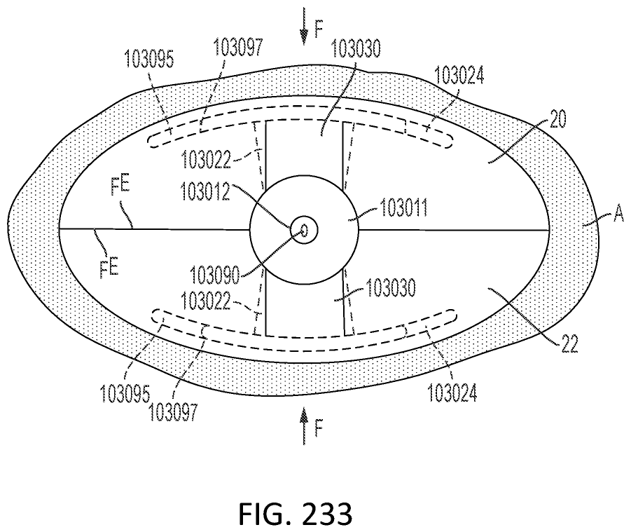

The anchor portion 103006 can take any suitable form, such as, for example, the form of the anchor portion 206 of the device 200 shown in FIG. 22 or any other form described in the present application. In some implementations, the anchor portion 103006 can include at least one anchor 103008. The anchor 103008 can have an outer paddle 103020, an inner paddle 103022 (FIG. 233), a paddle extension member or paddle frame 103024, and/or clasps 103030 (FIG. 233). The inner and outer paddles 103020, 103022 can take any suitable form, such as, for example, any form described in the present application. The clasps 103030 can take any suitable form, such as, for example, any form described in the present application. In some implementations, the implantable device 103000 includes a cover (not shown) that is attached to the anchor portion 103006. The cover can take any suitable form, such as, for example, any form described in the present application.

In the illustrated example, the paddle frame 103024 has an optional inner frame portion 103072 and an outer frame portion 103074. The inner frame portion 103072 can be rigid such that it is configured to support the paddles 103020, 103022 and provide a sufficient force to leaflets of the native valve during capture. The inner frame portion 103072 can be made of, for example, metals, plastics, etc.

In some implementations, the outer frame portions 103074 can be connected to the optional inner frame portions 103072 such that the outer frame portions 103074 define the total width of the paddle frame 103024. The outer frame portions 103074 can be flexible such that the outer frame portions can optionally be manipulated by a user between an expanded position and a narrowed position. For example, the outer frame portions 103074 can be operatively connected to width adjustment element(s) 103090 by a connector 103096 of the implantable device. A user can engage the paddle frame 103024 by moving a portion of the connector 103096 into/out of the cap 103014 to move the outer frame portions 103074 between the expanded and narrowed positions. In the illustrated example, the outer frame portion 103074 has a pair of openings 103092 for connection to the connector 103096. The outer frame portions 103074 can be made of, for example, metals, and plastics.

In some implementations, the paddle frame 103024 can be configured to connect to the cap 103014 such that movement of the cap relative to other portions of the device 103000 (e.g., the coaptation element) causes the anchors 103008 to move between the open and closed positions. The paddle frame 103024 can be connected to the cap 103014 by any suitable means, such as, for example, any means described in the present application.

Referring to FIG. 229, the paddle frame 103024 is in the fully expanded or widest position. When in the expanded or widest position, a proximal end 103095 of the paddle frame 103024 has a total width TW1, where the total width TW1 is defined by a proximal end 103095 of the outer portions 103074 of the paddle frame 103024.

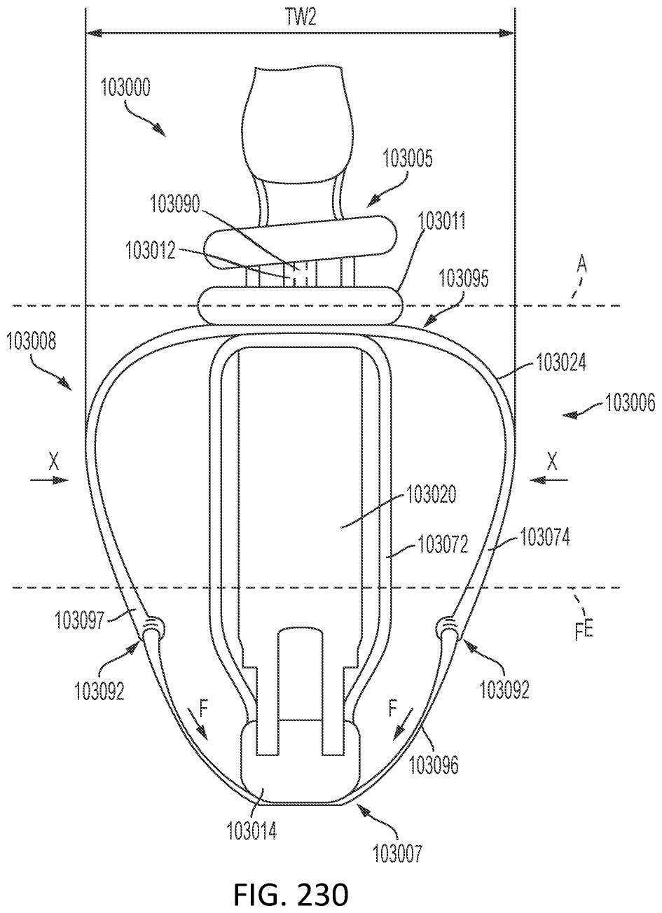

Referring to FIG. 230, the paddle frame 103024 is moved to a narrower, intermediate position by a user engaging the width adjustment element 103090. The width adjustment element 103090 pulls a portion of the connector 103096 into the cap to cause a tensioning force F on the paddle frame 103024. This tensioning force causes the outer portion 103074 to move in the inward direction X. When in this narrowed position, the paddle frame has a total width TW2, where the total width TW2 is defined by the proximal end 103095 of the paddle frame 103024. As is illustrated in FIG. 230, the distal end of the device has narrowed more than the proximal end of the device in this intermediate position.

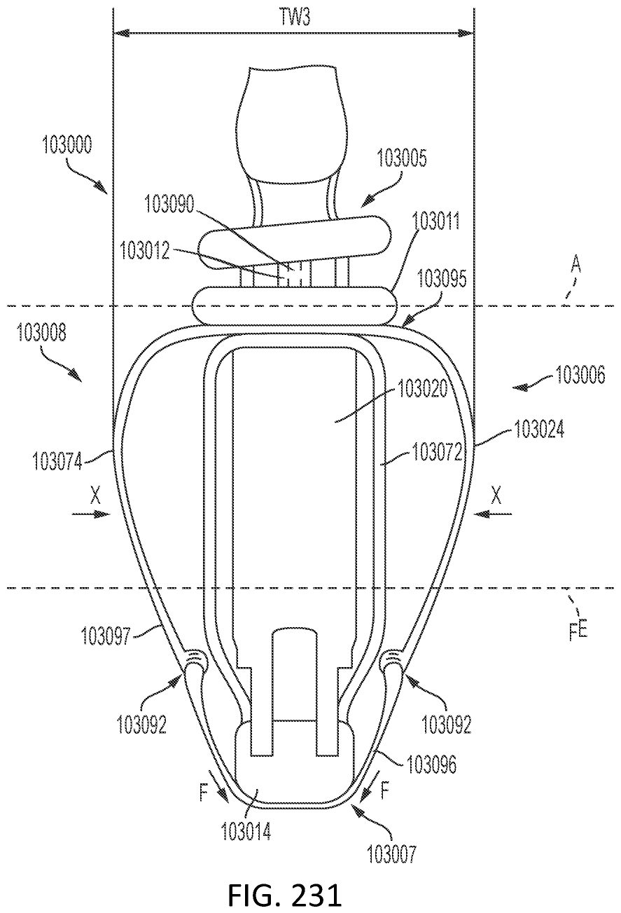

Referring to FIG. 231, the paddle frame 103024 is moved to a further narrowed, intermediate position by a user engaging the width adjustment element 103090. The width adjustment element 103090 pulls an additional portion of the connector 103096 into the cap to cause a further tensioning force F on the paddle frame 103024. This tensioning force causes the outer portion 103074 to move further in the inward direction X. When in this further narrowed position, the paddle frame has a total width TW3, where the total width TW3 is defined by the proximal end 103095 of the paddle frame 103024. As is illustrated in FIG. 231, the distal end of the device has continued to narrow more than the proximal end of the device in this intermediate position.

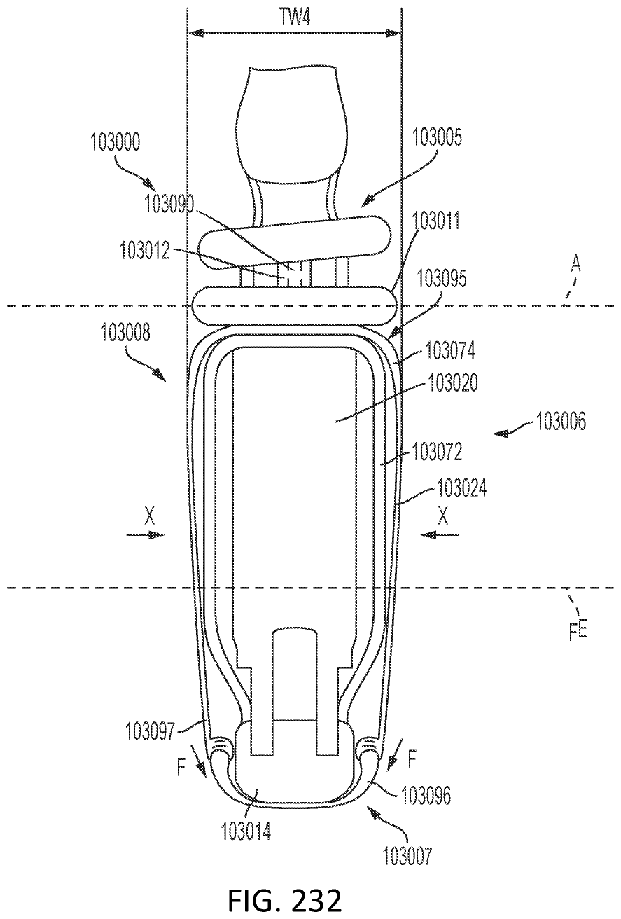

Referring to FIG. 232, the paddle frame 103024 is moved to a fully narrowed position by a user engaging the width adjustment element 103090. The width adjustment element 103090 pulls an additional portion of the connector 103096 into the cap to cause a further tensioning force F on the paddle frame 103024. This tensioning force causes the outer portion 103074 to move further in the inward direction X. When in the fully narrowed position, the paddle frame has a total width TW4.

In the illustrated example, the inner and outer portions 103072, 103074 are substantially the same size when the paddle frame 103024 is in the fully narrowed position. As such, the proximal end of the device has narrowed more than the distal end of the device when the device moved from the second intermediate position to the full narrowed position. Said another way, the paddle frame 103024 changes shape in two different phases. In the first phase, starting at the widest width, the distal end of the device narrows more quickly than the proximal end of the paddle frame 103024. In a second phase, starting at an intermediate width, the proximal end of the paddle frame 103024 narrows more quickly than the distal end of the device.

In the illustrated example, the total width TW1 (FIG. 229) is greater than the total width TW2 (FIG. 230), the total width TW2 is greater than the total width TW3 (FIG. 231), and the total width TW3 is greater than the total width TW4 (FIG. 232). In some implementations, the total widths TW1, TW2, TW3, for the fully wide configuration and the two intermediate width configurations can be close to one another. For example, the width TW3 can correspond to a position of the width adjustment element 103090 that is halfway between the position of the width adjustment element for the fully widened paddle frame (FIG. 229-width TW1) and the position of the width adjustment element for the fully narrowed paddle frame (FIG. 232-width TW4) and the width TW3 can be within 30% of the width TW4, such as within 25% of the width TW4, such as within 20% of the width TW4, such as within 10% of the width TW4. In some implementations, the width TW2 can correspond to a position that is ¼ of the way between the fully widened position and the fully narrowed position of the width adjustment element and the width TW2 can be within 20% of the width TW4, such as within 10% of the width TW4, such as within 5% of the width TW4, such as within 3% of the width TW4.

In some implementations, when the width adjustment element 103090 is halfway between the fully widened position and the fully narrowed position, the width TW2 can be substantially the same as the with TW1. In some implementations, when the width adjustment element 103090 is halfway between the fully widened position and the fully narrowed position, the width TW3 can be substantially the same as the with TW1.

Referring to FIG. 229, the total width TW1 can be between 5 mm and 20 mm, such as between 7 mm and 17 mm, such as between 9 mm and 14 mm, such as between 10 mm and 12 mm Referring to FIG. 232, the total width TW4 can be between 3 mm and 8 mm, such as between 4 mm and 7 mm, such as between 5 mm and 6 mm. The ratio of the total width TW1 to the total width TW4 can be between 10/9 and 3/1, such as between 5/4 and 2/1, such as between 4/3 and 3/2.

Referring to FIGS. 230-231, in the illustrated example, the proximal end 103095 of the paddle frame 103024 defines the total width of the paddle frame 103024 in the intermediate positions. That is, when the paddle frame 103024 is between the fully expanded, widest position and the fully narrowed position, the proximal end 103095 is the widest portion of the paddle frame 103024. This allows more force to be placed by the device 10300 to the portion of the leaflet that is proximate to the annulus A of the native valve, as compared to the free edge FE of the leaflets 20, 22, when the implantable device 103000 is attached to the native valve and the paddle frames 103024 are in an intermediate position. That is, referring to FIGS. 229-231, the proximal end 103095 is proximate the annulus A and the distal end 103097 is proximate the free edge FE of the leaflets. The larger width of the proximal end 103095 of the paddle frame 103024 can cause the paddle frames to apply force to a larger area proximate the annulus A of the native valve.

Referring to FIG. 233, the device 103000 can optionally be implanted on a native valve in the fully wide width TW1 illustrated by FIG. 229 or one of the widths TW2, TW3 illustrated by FIGS. 230, 231. With the wider widths of the paddle frames 103024 near the annulus A, the paddle frame 103024 can apply more force to larger portions of the leaflets that are closest to the annulus A to reshape the annulus A. For example, the device can cause a force F on the annulus A that causes the annulus A to contract and consequently causes the entirety of the leaflets 20, 22 to move closer together. This contraction of the annulus A can help the leaflets 20, 22 to coapt and prevent or inhibit regurgitation of blood during the systolic phase.

An implantable device 103000 capable of having a paddle frame 103024 with a wider proximal end 103095 is also advantageous for securing the implantable device 103000 to the native valve when the leaflets 20, 22 are not inserted deep within anchors 103008 of the implantable device 103000 as the anchors 103008 are moved from the opened position to the closed position. That is, the larger area of the proximal end 103095 of the paddle frame 103024 allows for the implantable device 103000 to engage a larger area of the leaflets 20, 22 even if the leaflets are not inserted deep within the implantable device 103000 during closing of the anchors 103008.

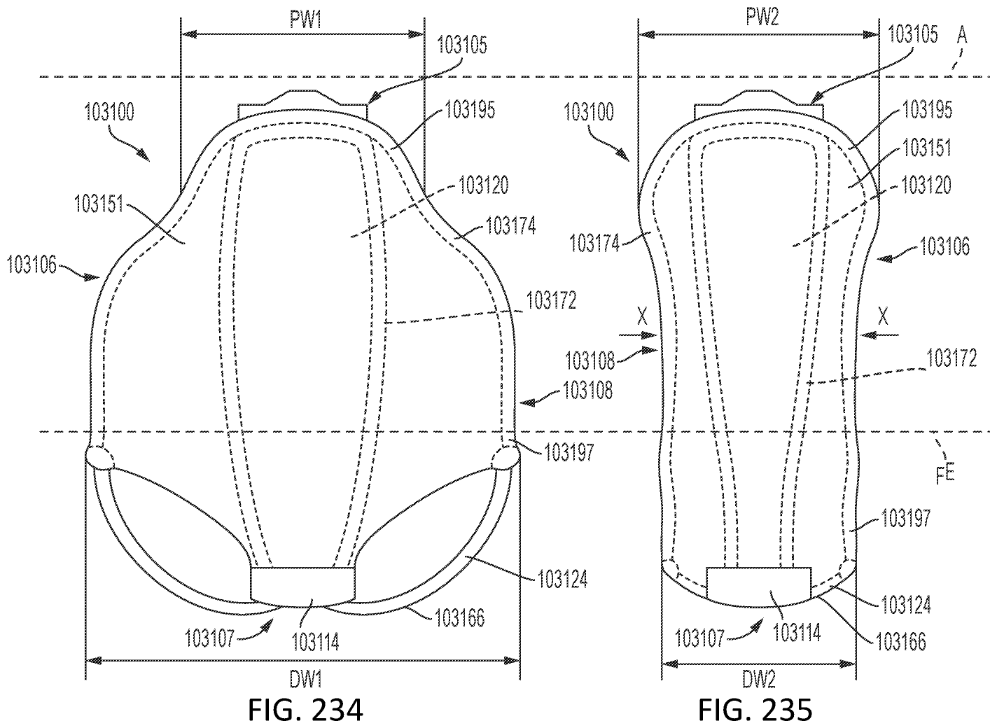

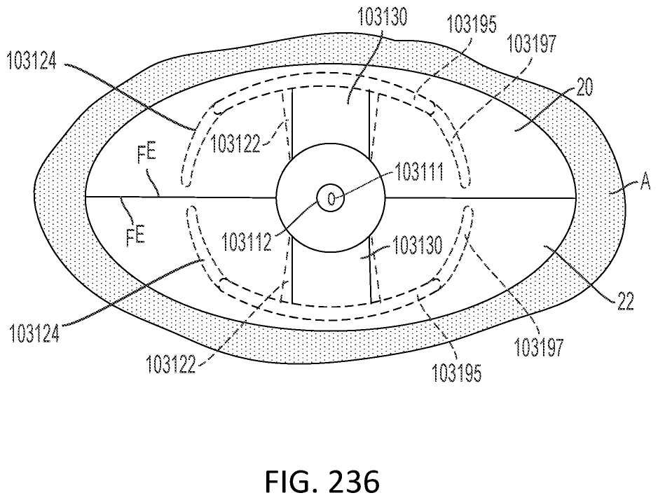

FIGS. 234-236 illustrate another example implementation of an implantable device or implant 103100 that includes an anchor portion 103106 having one or more paddle frames 103124 that are movable to a narrowed position. The narrowed position can allow the device 103100 to maneuver more easily into position for implantation in the heart by reducing the contact and/or friction between the native structures of the heart—e.g., chordae—and the device 103100. That is, a width adjustment element 103111 (FIG. 236) can be controlled by a user to cause the paddle frames 103024 to move to a narrowed position as the device 103100 is being positioned for implantation on the leaflets of a native valve. The width adjustment element 103111 can take any suitable form, such as, for example, any form described in the present application. The device 103100 can include any other features for an implantable device or implant discussed in the present application or in the applications and patents incorporated by reference herein, and the device 103100 can be positioned to engage valve tissue 20, 22 as part of any suitable valve repair system (e.g., any valve repair system disclosed in the present application). In addition, any of the devices described herein can incorporate the features of the device 103100.

The implantable device or implant 103100 can include an optional coaptation portion (not shown), a proximal or attachment portion 103105, an anchor portion 103106, and a distal portion 103107. The coaptation portion, attachment portion 103105, and distal portion 103107 can take any suitable form, such as, for example, any form described in the present application. In some implementations, the coaptation portion optionally includes a coaptation element (e.g., a spacer, coaption element, gap filler, etc.) that can be used, for example, for implantation between the leaflets 20, 22 of the native mitral valve MV. When included, the coaptation element, etc. can take any suitable form, such as, for example, any form described in the present application.

In some implementations, the attachment portion 103105 can include a first or proximal collar for engaging with a capture mechanism of a delivery system. The capture mechanism and the delivery system can take any suitable form, such as, for example, any form described in the present application. The delivery system can be the same as or similar to other delivery systems herein, e.g., 102, 402, 502, etc. and can comprise one or more of a catheter, a sheath, a guide catheter/sheath, a delivery catheter/sheath, a steerable catheter, an implant catheter, a tube, a channel, a pathway, combinations of these, etc. The proximal collar can take any suitable form, such as, for example, any form described in the present application.

In some implementations, the distal portion 103107 includes a cap 103114 that is attached to anchors 103108 of the anchor portion 103106 such that movement of the cap 103114 causes the anchors 103108 to move between open and closed positions. The cap 103114 can take any suitable form, such as, for example, any form described in the present application. Referring to FIG. 236, an actuation element 103112 (e.g., the same as or similar to actuation element 112 shown in FIGS. 8-20 or actuation element 8102 shown in FIGS. 26-30) extends from a delivery system (e.g., any delivery system described in the present application) and engages the cap 103114 to move the cap 103114 relative to the coaptation element to enable actuations of the device 103100. The actuation element can engage and move the cap by any suitable means, such as, for example, any means provided in the present application.

The anchor portion 103106 can take any suitable form, such as, for example, the form of the anchor portion 206 of the device 200 shown in FIG. 22 or any other form described in the present application. In some implementations, the anchor portion 103106 can include at least one anchor 103108, where the anchor 103108 has an outer paddle 103120, an inner paddle 103122 (FIG. 236), a paddle extension member or paddle frame 103124, and clasps 103130 (FIG. 236). The inner paddle 103122 and outer paddle can take any suitable form, such as, for example, any form described in the present application. The clasps 103130 can take any suitable form, such as, for example, any form described in the present application. In some implementations, the implantable device 103100 includes a cover 103151 that is attached to the anchor portion 103106. The cover 103151 can take any suitable form, such as, for example, any form described in the present application.

In the illustrated example, the paddle frame 103124 has an optional inner frame portion 103172 and an outer frame portion 103174. The inner frame portion 103172 can be rigid such that it is configured to support the paddles and provide a sufficient force to leaflets of the native valve during capture. The inner frame portion 103172 can be made of, for example, metals, plastics, etc. The outer frame portions 103174 can be connected to the inner frame portions 103172 such that the outer frame portions 103174 define the total width of the paddle frame 103124. The outer frame portions 103174 can be flexible such that the outer frame portions can optionally be manipulated by a user between an expanded position and a narrowed position. For example, the outer frame portions 103174 can be operatively connected to the width adjustment element 103111 (e.g., any width adjustment element shown or described in the present application) such that a user can engage the paddle frame 103124 to move the outer frame portions 103174 between the expanded and narrowed positions. In the illustrated example, paddle frame 103124 includes a connector 103166 that is engaged by the width adjustment element 103111 such that the connector 103166 moves relative to (into/out of) the cap 103114. This movement of the connector 103166 relative to the cap 103114 causes the outer portion 103174 of the paddle frame 103124 to move between the narrowed and expanded positions. The outer frame portions 103074 can be made of, for example, metals, and plastics.

In some implementations, the paddle frame 103124 can be configured to connect to the cap 103114 such that movement of the cap relative to other portions of the device 103100 (e.g., the coaptation element) causes the anchors 103108 to move between the open and closed positions. The paddle frame 103124 can be connected to the cap 103114 by any suitable means, such as, for example, any means described in the present application.

Referring to FIG. 234, the paddle frame 103124 is shown in the fully expanded or widest position. When in the expanded or widest position, a distal end 103197 of the paddle frame 103124 has a distal width DW1, where the distal width DW1 defines a total width of the paddle frame 103124. Referring to FIG. 235, in the illustrated example, the paddle frame 103124 is moved to a fully narrowed position by a user engaging the connector 103166 with a width adjustment element 103111 to pull the connector 103166 through a distal opening (not shown) of the cap 103114 such that the outer portion 103174 moves in an inward direction X. When in the fully narrowed position, the distal end 103197 of the paddle frame 103124 has a distal width DW2. In the illustrated example, a proximal end 103195 has a proximal width PW2 that defines the total width of the paddle frame 103124 when the paddle frame 103124 is in the fully narrowed position. However, the distal width of the distal end 103197 of the paddle frame 103124 is greater than the proximal width of the proximal end of the paddle frame 103124 in most, if not all, positions between the fully expanded and fully narrowed positions, such that the distal width of the distal end 103197 defines the total width of the paddle frame 103124. In some implementations, the distal width DW2 of the distal end 103197 can define the total width of the paddle frame 103124 when in the fully narrowed position.

The distal end 103197 of the paddle frame 103124 can be maintained at any distal width between the distal widths DW1 and DW2 as the device is being implanted on a native valve of a patient. In certain implementations, the proximal width of the proximal end 103195 of the paddle frame 103124 is substantially constant as the paddle frame 103124 is moved between the fully expanded and fully narrowed positions. That is, PW1 (FIG. 234) is substantially equal to PW2 (FIG. 235).

In some implementations, the proximal widths PW1, PW2 for the fully wide configuration and the narrowest width configurations can be close to one another. For example, widths PW1, PW2 can be within 20% of one another, such as within 10% of one another, such as within 5% of one another.

In the illustrated example, the distal width DW1 (FIG. 234) is greater than the distal width DW2 (FIG. 235). Referring to FIG. 234, the distal width DW1 can be between 5 mm and 15 mm, such as between 7 mm and 12 mm, such as between 9 mm and 11 mm, such as about 10 mm. The ratio of the distal width DW1 to the proximal width PW1 when the paddle frame 103124 is in the fully expanded, widest position can be between about 4/1 and about 4/3, such as between about 3/1 and about 2/1. Referring to FIG. 235, the distal width DW2 can be between 3 mm and 12 mm, such as between 4 mm and 10 mm, such as between 5 mm and 9 mm, such as about 6 mm. The ratio of the distal width DW1 to the distal width DW2 can be between 4/3 and 3/1, such as between 5/4 and 2/1, such as between 10/9 and 3/2

Referring to FIGS. 234-236, in the illustrated example, the distal end 103197 of the paddle frame 103124 defines the total width of the paddle frame 103024 in each position from and including the fully expanded position (FIG. 234) until slightly before the paddle frame 103124 reaches the fully narrowed position (FIG. 235). That is, as the paddle frame 103124 is moved between the fully expanded position and the fully narrowed position, the distal end 103197 is maintained as the widest portion of the paddle frame 103124. This allows more stress to be placed proximate the free ends FE of the leaflets 20, 22 of the native valve, as compared to the annulus A of the native valve, when the implantable device 103100 is attached to the native valve. That is, referring to FIGS. 234-235, the distal end 103197 is proximate the free edge FE of the leaflets 20, 22, and the proximal end 103195 is proximate the annulus A. The larger width of the distal end 103197 of the paddle frame 101024 applies a force on a larger area proximate the free edge FE of the leaflets than proximate the valve annulus A.

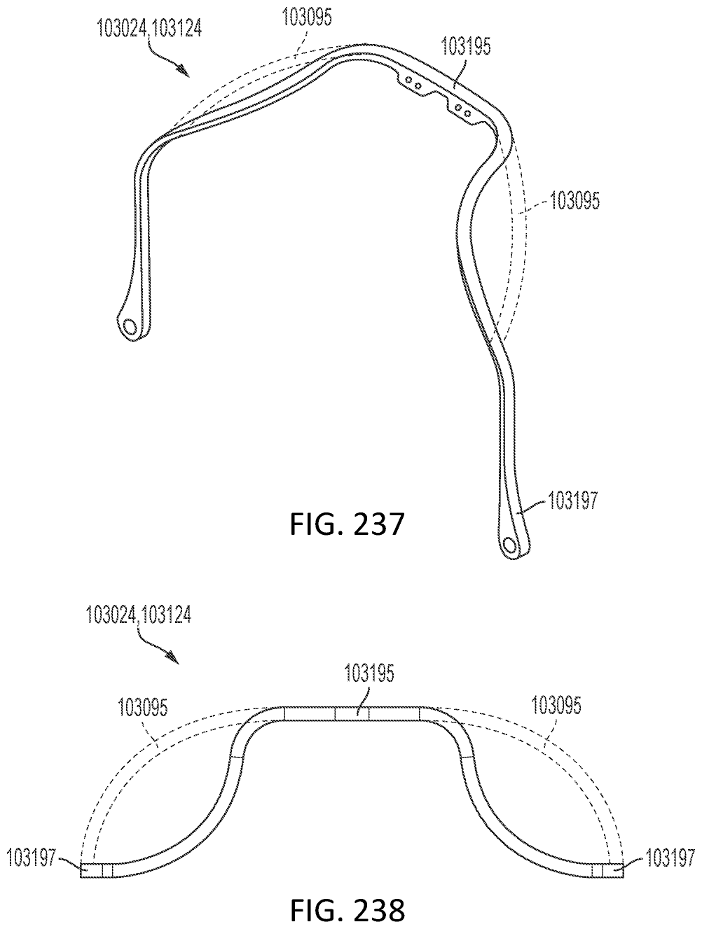

FIGS. 237-238 show an example frame 103124 in the expanded position, where the distal end 103197 is wider than the proximal end 103195 such that the distal end 103197 defines a total width of the paddle frame 103124. In the illustrated example, the solid line portion of the proximal end 103195 is for the paddle frame 103124 (FIGS. 234-235), and the dashed line portion of the proximal end 103095 is for the paddle frame 103024 (FIGS. 229-233). In the illustrated example the paddle frame 103124 has a rounded shape that corresponds to the shape of a coaptation element such that the anchors 103108 conform around the coaptation element to better secure the leaflet tissue between the anchors 103108 and the coaptation element. The paddle frames 103124 can be formed by shape setting a material such that the paddle frames have a normal position in which the distal end 103197 is wider than the proximal end 103195 such that more force is applied near the free edge FE (FIGS. 234-236) of the leaflets 20, 22 of the native valve when the implantable device 103100 (FIGS. 234-236) is attached to the native valve. For example, the shape set paddle frames 103124 can be made of metals, such as steel, nitinol, etc., plastics, etc.

Referring to FIG. 236, can be advantageous to implant a device 103100 having a paddle frame 103124 that applies more force on the free edges FE of the native valve because the paddle frame 103124 creates pinch points at the free edges FE of the native valve that causes the free edges FE to be pressed together. An implantable device 103100 having a paddle frame 103124 with a wider distal end 103197 can also be advantageous because less of the device contacts the leaflets. That is, in this implementation, a majority of the contact is at the free edges FE of the leaflets 20, 22, which can allow for better blood flow during the diastole phase.

Referring again to FIGS. 237-238, the dashed line example frame 103024 is in the expanded position. The proximal end 103095 defines a total width of the paddle frame 103024. The paddle frames 103024 can be formed by shape setting a material such that the paddle frames have a normal position in which the proximal end 103095 defines a total width of the paddle frames 103024 such that more force is applied near the annulus A (FIGS. 229-233) of the native valve when the implantable device 103000 (FIGS. 229-233) is attached to the native valve. For example, the shape set paddle frames 103024 can be made of metals, such as steel, nitinol, etc., plastics, etc.

### Examples

Example 1. An implantable device, comprising: (i) an anchor portion comprising a first anchor and a second anchor, each of the first and second anchors comprising a paddle frame that includes an inner frame portion and an outer frame portion, wherein the first and second anchors are configured to be moved to a closed position in which the inner frame portion of each of the first and second anchors compress native valve leaflets between an inner pinch point and the outer frame portion of each of the first and second anchors compress the leaflets between an outer pinch point such that the implantable device is secured to the native valve leaflets; and (ii) a cover attached to at least one of the inner frame portion and the outer frame portion of the first and second anchors such that a compressive force can be applied against the leaflets in an area between the inner and outer pinch points when the implantable device is secured to the native valve leaflets.

Example 2. The implantable device according to example 1, wherein the inner frame portion is rigid, and the outer frame portion is flexible.

Example 3. The implantable device according to any one of examples 1-2, wherein the outer frame portion is movable between a narrowed position and an expanded position.

Example 4. The implantable device according to any one of examples 1-3, wherein the cover is connected to the paddle frame by one or more stitches.

Example 5. The implantable device according to any one of examples 1-4, wherein the cover extends across an inner surface of the paddle frame.

Example 6. The implantable device according to any one of examples 1-5, wherein the cover extends across an outer surface of the paddle frame.

Example 7. The implantable device according to any one of examples 1-6, wherein the cover is attached to both the inner frame portion and the outer frame portion.

Example 8. The implantable device according to any one of examples 1-7, wherein the cover is made of a flexible material.

Example 9. The implantable device according to example 8, wherein the outer frame portion is movable between a narrowed position and an expanded position, wherein the cover is configured to be in a taut state when the paddle frame is in the narrowed position, and wherein the cover is configured to stretch when the paddle frame is in the expanded position.

Example 10. The implantable device according to example 8, wherein the inner frame portion is rigid and the outer frame portion is flexible, wherein the outer frame portion is configured to be moved between a narrowed position and an expanded position, and wherein the cover is attached to both the inner frame portion and the outer frame portion such that the cover is in a taut state when the outer frame portion is in the narrowed position and the cover stretches when the outer frame portion is moved to the expanded position.

Example 11. The implantable device according to any one of examples 1-10, wherein the cover is positioned between at least a portion of an area defined by an interior surface of the inner frame portion.

Example 12. The implantable device according to any one of examples 1-11, wherein the cover extends across an entirety of an area defined by an interior surface of the inner frame portion.

Example 13. The implantable device according to any one of examples 1-12, wherein the cover encapsulates at least a portion of an outer paddle of each of the first and second anchors.

Example 14. The implantable device according to any one of examples 1-13, wherein the cover encapsulates an entirety of an outer paddle of each of the first and second anchors.

Example 15. The implantable device according to any one of examples 1-14, wherein the cover comprises a first membrane that attaches to the paddle frame of the first anchor and a second membrane that attaches to the paddle frame of the second anchor.

Example 16. The implantable device according to example 15, wherein the first and second membranes are not connected such that a gap exists between bottom edges of the first membrane and the second membrane.

Example 17. The implantable device according to any one of examples 1-16, wherein the cover comprises a single membrane that attaches to the paddle frame of both of the first and second anchors.

Example 18. The implantable device according to example 17, wherein the single membrane creates a canopy that extends between the first and second anchors.

Example 19. The implantable device according to any one of examples 1-18, wherein the cover is made of a porous material.

Example 20. The implantable device according to any one of examples 1-19, wherein the cover is made of a material that is impermeable to blood flow.

Example 21. An implantable device, comprising: (i) an anchor portion comprising a first anchor and a second anchor, each of the first and second anchors comprising a paddle frame that includes an inner frame portion and an outer frame portion, wherein the outer frame portion is configured to be moved between a narrowed position and expanded position, wherein the first and second anchors are configured to be moved to a closed position in which the inner frame portion of each of the first and second anchors compress the leaflets between an inner pinch point and the outer frame portion of each of the first and second anchors compress the leaflets between an outer pinch point such that the implantable device is secured to the native valve; and (ii) a flexible cover attached to the inner frame portion and the outer frame portion of the first and second anchors such that the cover can apply a compressive force against the leaflets in an area between the inner and outer pinch points when the implantable device is secured to the native valve, wherein the cover is configured to be in a taut state when the outer frame portion is in the narrowed position, and wherein the cover is configured to stretch when the outer frame portion is in the expanded position.

Example 22. The implantable device according to example 21, wherein the inner frame portion is rigid, and the outer frame portion is flexible.

Example 23. The implantable device according to any one of examples 21-22, wherein the cover is connected to the paddle frame by one or more stitches.

Example 24. The implantable device according to any one of examples 21-23, wherein the cover extends across an inner surface of the paddle frame.

Example 25. The implantable device according to any one of examples 21-24, wherein the cover extends across an outer surface of the paddle frame.

Example 26. The implantable device according to any one of examples 21-25, wherein the cover is positioned between at least a portion of an area defined by an interior surface of the inner frame portion.

Example 27. The implantable device according to any one of examples 21-26, wherein the cover extends across an entirety of an area defined by an interior surface of the inner frame portion.

Example 28. The implantable device according to any one of examples 21-27, wherein the cover encapsulates at least a portion of an outer paddle of each of the first and second anchors.

Example 29. The implantable device according to any one of examples 21-28, wherein the cover encapsulates an entirety of an outer paddle of each of the first and second anchors.

Example 30. The implantable device according to any one of examples 21-29, wherein the cover comprises a first membrane that attaches to the paddle frame of the first anchor and a second membrane that attaches to the paddle frame of the second anchor.

Example 31. The implantable device according to example 30, wherein the first and second membranes are not connected such that a gap exists between bottom edges of the first membrane and the second membrane.

Example 32. The implantable device according to any one of examples 21-31, wherein the cover comprises a single membrane that attaches to the paddle frame of both of the first and second anchors.

Example 33. The implantable device according to example 32, wherein the single membrane creates a canopy that extends between the first and second anchors.

Example 34. The implantable device according to any one of examples 21-33, wherein the cover is made of a porous material.

Example 35. The implantable device according to any one of examples 21-34, wherein the cover is made of a material that is impermeable to blood flow.

Example 36. An implantable device, comprising: (i) an anchor portion comprising a first anchor and a second anchor, each of the first and second anchors comprising a paddle frame that includes an inner frame portion and an outer frame portion, wherein the first and second anchors are configured to be moved to a closed position in which the inner frame portion of each of the first and second anchors compress the leaflets between an inner pinch point and the outer frame portion of each of the first and second anchors compress the leaflets between an outer pinch point such that the implantable device is secured to the native valve; and (ii) a cover attached to the inner frame portion and the outer frame portion of the first and second anchors, wherein the cover extends between the first anchor and the second anchor.

Example 37. The implantable device according to example 36, wherein the inner frame portion is rigid, and the outer frame portion is flexible.

Example 38. The implantable device according to any one of examples 36-37, wherein the cover is connected to the paddle frame by one or more stitches.

Example 39. The implantable device according to any one of examples 36-38, wherein the cover extends across an inner surface of the paddle frame.

Example 40. The implantable device according to any one of examples 36-39, wherein the cover extends across an outer surface of the paddle frame.

Example 41. The implantable device according to any one of examples 36-40, wherein the cover is positioned between at least a portion of an area defined by an interior surface of the inner frame portion.

Example 42. The implantable device according to any one of examples 36-41, wherein the cover extends across an entirety of an area defined by an interior surface of the inner frame portion.

Example 43. The implantable device according to any one of examples 36-42, wherein the cover encapsulates at least a portion of an outer paddle of each of the first and second anchors.

Example 44. The implantable device according to any one of examples 36-43, wherein the cover encapsulates an entirety of an outer paddle of each of the first and second anchors.

Example 45. The implantable device according to any one of examples 36-44, wherein the cover comprises a first membrane that attaches to the paddle frame of the first anchor and a second membrane that attaches to the paddle frame of the second anchor.

Example 46. The implantable device according to example 30, wherein the first and second membranes are connected such together to block blood flow between the first and second anchors.

Example 47. The implantable device according to any one of examples 21-31, wherein the cover comprises a single membrane that attaches to the paddle frames of both of the first and second anchors.

Example 48. The implantable device according to example 32, wherein the single membrane creates a canopy that extends between the first and second anchors.

Example 49. The implantable device according to any one of examples 21-33, wherein the cover is made of a porous material that becomes impermeable to blood flow over time.

Example 50. The implantable device according to any one of examples 21-34, wherein the cover is made of a material that is impermeable to blood flow.

Example 51. An implantable device comprising: (i) a coaptation element that defines a first area when viewed from above; (ii) one or more anchors coupled to the coaptation element, the anchors being movable between an open position and a closed position and configured to attach to one or more leaflets of a native heart valve, each of the anchors comprising a paddle frame, wherein the paddle frame defines an outer portion of the implantable device when viewed from above and the anchors are in the closed position, wherein the outer portion of the device has a second area when viewed from above, wherein a ratio of the second area to the first area is greater than or equal to 2 to 1.

Example 52. The implantable device according to example 51, wherein the ratio of the second area to the first area is greater than or equal to 3 to 1.

Example 53. The implantable device according to example 51, wherein the ratio of the second area to the first area is greater than or equal to 4 to 1.

Example 54. The implantable device according to example 51, wherein the ratio of the second area to the first area is greater than or equal to 5 to 1.

Example 55. The implantable device according to example 51, wherein the ratio of the second area to the first area is greater than or equal to 6 to 1.

Example 56. The implantable device according to any one of examples 51-55, wherein the coaptation element comprises one or more planar side surfaces.

Example 57. The implantable device according to any one of examples 51-56, wherein the coaptation element comprises one or more tapered side surfaces.

Example 58. The implantable device according to any one of examples 51-57, wherein the coaptation element comprises a first portion that is rectangular and a second portion that is rounded.

Example 59. The implantable device according to any one of examples 51-58, wherein the coaptation element is injection molded.

Example 60. The implantable device according to any one of examples 51-59, wherein the coaptation element comprises a polymer material.

Example 61. The implantable device according to any one of examples 51-60, further comprising an attachment portion having a collar that is configured to attach to a delivery device.

Example 62. The implantable device according to example 61, wherein the collar of the attachment portion is integral to the coaptation element.

Example 63. The implantable device according to any one of examples 51-62, wherein the coaptation element comprises one or more attachment openings for aligning with one or more openings of a component of the anchors such that the component of the anchors can be attached to the coaptation element.

Example 64. The implantable device according to any one of examples 51-63, wherein the anchors further comprise an inner paddle and an outer paddle.

Example 65. The implantable device according to any one of examples 51-64, wherein the anchors further comprise a clasp.

Example 66. The implantable device according to any one of examples 51-65, wherein the paddle frame of the each of the anchors comprises an inner paddle frame and an outer paddle frame.

Example 67. The implantable device according to example 66, wherein the outer paddle frame defines the outer portion of the implantable device when viewed from above and the anchors are in the closed position.

Example 68. The implantable device according to any one of examples 51-67, wherein the coaptation element has a length of between about 10 mm and about 40 mm.

Example 69. The implantable device according to any one of examples 51-68, wherein the coaptation element has a width of between about 3 mm and about 10 mm.

Example 70. The implantable device according to any one of examples 51-69, further comprising a distal portion having a cap.

Example 71. The implantable device according to example 70, wherein the cap is operative connected to the anchors such that movement of the cap away from the coaptation element causes the anchors to move to the opened position and movement of the cap toward the coaptation element causes the anchors to move to the closed position.

Example 72. The implantable device according to any one of examples 51-71, further comprising a flexible cover attached to the paddle frame.

Example 73. The implantable device according to example 72, wherein the cover creates a canopy that extends between the first and second anchors.

Example 74. The implantable device according to any one of examples 51-73, wherein the cover comprises a single piece of material.

Example 75. The implantable device according to any one of examples 51-74, wherein the cover comprises a multiple pieces of material.

Example 76. The implantable device according to any one of examples 51-75, wherein the cover is made of a porous material that becomes impermeable to blood flow over time.

Example 77. The implantable device according to any one of examples 51-75, wherein the cover is made of a material that is impermeable to blood flow.

Example 78. A valve repair system for repairing a native valve of a patient, the valve repair system comprising: (A) a delivery device having a width adjustment element that includes an external threaded portion; (B) an implantable device configured to be implanted on the native valve of the patient, the implantable device having: (i) an anchor portion having one or more anchors, each of the anchors having a paddle frame that includes an inner end, wherein the one or more anchors are configured to attach to one or more leaflets of a native valve; (ii) a coupler for removably connecting the width adjustment element of the delivery device to the inner end of the anchors; wherein the coupler comprises one or more attachment projections that extend inward from a body of the coupler; wherein the attachment projections are configured to removably attach to the external threaded portion of the width adjustment element; and wherein the width adjustment element is configured to move the paddle frame between a narrowed position and an expanded position.

Example 79. The valve repair system according to example 78, wherein the one or more attachment projections includes a first attachment projection and a second attachment projection.

Example 80. The valve repair system according to example 79, wherein the first attachment projection is offset from the second attachment projection along a height of the body of the coupler.

Example 81. The valve repair system according to any one of examples 78-80, wherein the coupler comprises at least two arms that are movable between a normal position and an engaged position, wherein the arms are in the normal position when the coupler is disconnected from the implantable device, and wherein the arms are in the engaged position when the coupler is connected to the width adjustment element.

Example 82. The valve repair system according to example 81, wherein the arms are configured to attach to internal threads when the arms are in the normal position.

Example 83. The valve repair system according to example 81, wherein the arms comprise one or more tabs that are configured to be inserted into one or more slots of the body of the coupler when the arms are in the normal position.

Example 84. The valve repair system according to example 81, wherein the arms are configured to allow the coupler to move within a lumen of a receiver when in the engaged position.

Example 85. The valve repair system according to any one of examples 78-84 wherein a coaptation element defines a first area when viewed from above, wherein the paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 86. The valve repair system according to example 85, wherein the ratio of the second area to the first area is greater than or equal to 3 to 1.

Example 87. The valve repair system according to example 85, wherein the ratio of the second area to the first area is greater than or equal to 4 to 1.

Example 88. The valve repair system according to example 85, wherein the ratio of the second area to the first area is greater than or equal to 5 to 1.

Example 89. The valve repair system according to example 85, wherein the ratio of the second area to the first area is greater than or equal to 6 to 1.

Example 90. The valve repair system according to any one of examples 85-89, wherein the coaptation element comprises one or more planar side surfaces.

Example 91. The valve repair system according to any one of examples 85-90, wherein the coaptation element comprises one or more tapered side surfaces.

Example 92. The valve repair system according to any one of examples 85-91, wherein the coaptation element comprises a first portion that is rectangular and a second portion that is rounded.

Example 93. The valve repair system according to any one of examples 85-92, wherein the coaptation element is injection molded.

Example 94. The valve repair system according to any one of examples 85-93, wherein the coaptation element comprises a polymer material.

Example 95. The valve repair system according to any one of examples 85-94, wherein the coaptation element has a length of between about 10 mm and about 40 mm.

Example 96. The valve repair system according to any one of examples 85-95, wherein the coaptation element has a width of between about 3 and about 10 mm.

Example 97. The valve repair system according to any one of examples 78-96, further comprising a distal portion having a cap.

Example 98. The valve repair system according to example 97, wherein the cap is operative connected to the anchors such that movement of the cap away from a coaptation element causes the anchors to move to an opened position and movement of the cap toward the coaptation element causes the anchors to move to a closed position.

Example 99. The valve repair system according to any one of examples 78-98, further comprising a flexible cover attached to the paddle frame.

Example 100. The valve repair system according to example 99, wherein the cover creates a canopy that extends between first and second anchors of the one or more anchors.

Example 101. The valve repair system according to any one of examples 99-100, wherein the cover comprises a single piece of material.

Example 102. The valve repair system according to any one of examples 99-100, wherein the cover comprises multiple pieces of material.

Example 103. The valve repair system according to any one of examples 99-102, wherein the cover is made of a porous material that becomes impermeable to blood flow over time.

Example 104. The valve repair system according to any one of examples 99-102, wherein the cover is made of a material that is impermeable to blood flow.

Example 105. The valve repair system according to any one of examples 78-104, wherein the paddle frame comprises a connector that connects the width adjustment element to the paddle frames.

Example 106. A valve repair system for repairing a native valve of a patient, the valve repair system comprising: (A) a delivery device having a width adjustment element; (B) an implantable device configured to be implanted on the native valve of the patient, the implantable device having: (i) a receiver defining a lumen that includes internal threads; (ii) an anchor portion having one or more anchors, each of the anchors having a paddle frame, wherein the one or more anchors are configured to attach to one or more leaflets of a native valve; and (iii) a coupler for removably connecting the width adjustment element of the delivery device to an inner end of the anchors, wherein the coupler comprises at least two arms that are movable between a normal position and an engaged position, wherein the arms are in the normal position when the coupler is disconnected from the width adjustment element, and wherein the arms are in the engaged position when the coupler is connected to the width adjustment element, wherein the arms are configured to attach to internal threads of the lumen of the receiver when the arms are in the normal position.

Example 107. The valve repair system according to example 106, wherein the arms comprise one or more tabs that are configured to be inserted into one or more slots of the body of the coupler when the arms are in the normal position.

Example 108. The valve repair system according to example 106, wherein the arms are configured to allow the coupler to move within the lumen of the receiver when in the engaged position.

Example 109. The valve repair system according to any of examples 106-108, wherein a first arm of the at least two arms is offset from a second arm along a height of a body of the coupler.

Example 110. The valve repair system according to any of examples 106-109, wherein a first portion of each arm extends into a lumen of the coupler when the arms are in the normal position, and wherein a second portion of each arm extends away from an exterior of a body of the coupler when the arms are in the normal position.

Example 111. The valve repair system according to any of examples 106-110, wherein the arms extend away from an exterior surface of a body of the coupler by between about 20 degrees and about 45 degrees when the arms are in the normal position.

Example 112. The valve repair system according to any of examples 106-111, wherein the coupler further comprises one or more attachment projections that extend inward from a body of the coupler to removably attach to external threads of the width adjustment element by a threaded connection.

Example 113. The valve repair system according to example 112, wherein the one or more attachment projections includes a first attachment projection and a second attachment projection.

Example 114. The valve repair system according to example 113, wherein the first attachment projection is offset from the second attachment projection along a height of a body of the coupler.

Example 115. The valve repair system according to any one of examples 106-114, wherein a coaptation element defines a first area when viewed from above, wherein the paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 116. The valve repair system according to example 115, wherein the ratio of the second area to the first area is greater than or equal to 3 to 1.

Example 117. The valve repair system according to example 115, wherein the ratio of the second area to the first area is greater than or equal to 4 to 1.

Example 118. The valve repair system according to example 115, wherein the ratio of the second area to the first area is greater than or equal to 5 to 1.

Example 119. The valve repair system according to example 115, wherein the ratio of the second area to the first area is greater than or equal to 6 to 1.

Example 120. The valve repair system according to any one of examples 115-119, wherein the coaptation element comprises one or more planar side surfaces.

Example 121. The valve repair system according to any one of examples 115-120, wherein the coaptation element comprises one or more tapered side surfaces.

Example 122. The valve repair system according to any one of examples 115-121, wherein the coaptation element comprises a first portion that is rectangular and a second portion that is rounded.

Example 123. The valve repair system according to any one of examples 115-122, wherein the coaptation element is injection molded.

Example 124. The valve repair system according to any one of examples 115-123, wherein the coaptation element comprises a polymer material.

Example 125. The valve repair system according to any one of examples 115-124, wherein the coaptation element has a length of between about 10 mm and about 40 mm.

Example 126. The valve repair system according to any one of examples 115-125, wherein the coaptation element has a width of between about 3 mm and about 10 mm.

Example 127. The valve repair system according to any one of examples 106-126, further comprising a distal portion having a cap.

Example 128. The valve repair system according to example 127, wherein the cap is operative connected to the anchors such that movement of the cap away from a coaptation element causes the anchors to move to an opened position and movement of the cap toward the coaptation element causes the anchors to move to a closed position.

Example 129. The valve repair system according to any one of examples 106-128, further comprising a flexible cover attached to the paddle frame.

Example 130: The valve repair system according to example 129, wherein the flexible cover includes a plurality of discreet flexible portions.

Example 131. The valve repair system according to example 129, wherein the cover creates a canopy that extends between first and second anchors of the one or more anchors.

Example 132. The valve repair system according to example 131, wherein the cover comprises a single piece of material.

Example 133. The valve repair system according to any one of examples 131-132, wherein the cover comprises multiple pieces of material.

Example 134. The valve repair system according to any one of examples 131-133, wherein the cover is made of a porous material that becomes impermeable to blood flow over time.

Example 135. The valve repair system according to any one of examples 131-133, wherein the cover is made of a material that is impermeable to blood flow.

Example 136. The valve repair system according to any one of examples 106-135, wherein a connector is connected to the paddle frames and the connector includes the inner end.

Example 137. A valve repair system for repairing a native valve of a patient, the valve repair system comprising: (A) a delivery device having a width adjustment element; (B) an implantable device configured to be implanted on the native valve of the patient, the implantable device having: (i) a receiver defining a lumen that includes internal threads and an unattachable portion; (ii) an anchor portion having one or more anchors, each of the anchors having a paddle frame, wherein the one or more anchors are configured to attach to one or more leaflets of a native valve; and (iii) a coupler for removably connecting the width adjustment element of the delivery device to an inner end of the anchors, wherein the coupler is configured to be removably coupled to the internal threads of the receiver to secure the inner end of the anchors in a desired position relative to the receiver, wherein the unattachable portion of the receiver prevents the coupler from connecting to the receiver when the coupler is disposed within the unattachable portion.

Example 138. The valve repair system according to example 137, wherein the unattachable portion comprises a non-threaded portion.

Example 139. The valve repair system according to example 138, wherein the unattachable portion comprises a window that extends through the receiver.

Example 140. The valve repair system according to any of examples 137-139, wherein the coupler is movable within the unattachable portion.

Example 141. The valve repair system according to any of examples 137-140, wherein the coupler is movable within the unattachable portion when arms of the coupler are extended.

Example 142. The valve repair system according to any of examples 137-141, wherein the coupler comprises at least two arms that are movable between a normal position and an engaged position, wherein the arms are in the normal position when the coupler is disconnected from the width adjustment element, and wherein the arms are in the engaged position when the coupler is connected to the width adjustment element, wherein the arms are configured to attach to internal threads of the lumen of the receiver when the arms are in the normal position.

Example 143. The valve repair system according to example 142, wherein the arms comprise one or more tabs that are configured to be inserted into one or more slots of the body of the coupler when the arms are in the normal position.

Example 144. The valve repair system according to example 142, wherein the arms are configured to allow the coupler to move within the lumen of the receiver when in the engaged position.

Example 145. The valve repair system according to any of examples 142-144, wherein a first arm of the at least two arms is offset from a second arm along a height of a body of the coupler.

Example 146. The valve repair system according to any of examples 142-145, wherein a first portion of each arm extends into a lumen of the coupler when the arms are in the normal position, and wherein a second portion of each arm extends away from an exterior of a body of the coupler when the arms are in the normal position.

Example 147. The valve repair system according to any of examples 142-146, wherein the arms extend away from an exterior surface of a body of the coupler by between about 20 degrees and about 45 degrees when the arms are in the normal position.

Example 148. The valve repair system according to any of examples 142-147, wherein the coupler further comprises one or more attachment projections that extend inward from a body of the coupler to removably attach to external threads of the width adjustment element by a threaded connection.

Example 149. The valve repair system according to example 148, wherein the one or more attachment projections includes a first attachment projection and a second attachment projection.

Example 150. The valve repair system according to example 149, wherein the first attachment projection is offset from the second attachment projection along a height of a body of the coupler.

Example 151. The valve repair system according to example 142, wherein each of the arms comprise a central portion, a first connection member that connects the central portion to a proximal end of the coupler, and a second connection member that connects the central portion to a distal end of the coupler.

Example 152. The valve repair system according to example 151, wherein each of the arms has a “t” shape.

Example 153. The valve repair system according to any of examples 151-152, wherein the first and second connection members are normally in torsion such that a first portion of the central portion is disposed within a lumen of the coupler and a second portion of the central portion extends away from a body of the coupler when the coupler is in the normal position.

Example 154. The valve repair system according to any of examples 151-153, wherein the central portion of each of the arms is substantially aligned with a body of the coupler when the coupler is in the engaged position.

Example 155. The valve repair system according to any of examples 137-141, wherein the coupler comprises an upper body, a lower body, and a plurality of struts connected to the upper and lower bodies.

Example 156. The valve repair system according to example 155, wherein the coupler is movable between a first position in which the plurality of struts is in a straight configuration and a second position in which the plurality of struts are in a spiraled configuration.

Example 157. The valve repair system according to example 156, wherein a first width of the coupler when the coupler is in the first position is less than a second width of the coupler when the coupler is in the second position.

Example 158. The valve repair system according to any of examples 156-157, wherein the coupler is configured to attach to the receiver when the plurality of struts are in the spiraled configuration.

Example 159. The valve repair system according to any of examples 156-158, wherein the coupler is configured to move within the lumen of the receiver when the plurality of struts are in the straight configuration.

Example 160. The valve repair system according to any of examples 156-159, wherein the coupler is in an expanded configuration when in the first position and a compressed configuration when in the second position.

Example 161. The valve repair system according to example 160, wherein the coupler is normally in the compressed configuration.

Example 162. The valve repair system according to any of examples 155-161, wherein the coupler comprises four struts.

Example 163. The valve repair system according to any of examples 155-162, wherein the upper body comprises an upper opening for receiving the width adjustment element of the delivery device.

Example 164. The valve repair system according to any of examples 155-163, wherein the lower body comprises a lower opening for receiving the inner end of the paddle frame.

Example 165. The valve repair system according to example 164, wherein the lower opening of the lower body is further configured to receive the width adjustment element.

Example 166. The valve repair system according to any one of examples 137-165, wherein a coaptation element defines a first area when viewed from above, wherein the paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 167. The valve repair system according to example 166, wherein the ratio of the second area to the first area is greater than or equal to 3 to 1.

Example 168. The valve repair system according to example 166, wherein the ratio of the second area to the first area is greater than or equal to 4 to 1.

Example 169. The valve repair system according to example 166, wherein the ratio of the second area to the first area is greater than or equal to 5 to 1.

Example 170. The valve repair system according to example 166, wherein the ratio of the second area to the first area is greater than or equal to 6 to 1.

Example 171. The valve repair system according to any one of examples 166-170, wherein the coaptation element comprises one or more planar side surfaces.

Example 172. The valve repair system according to any one of examples 166-171, wherein the coaptation element comprises one or more tapered side surfaces.

Example 173. The valve repair system according to any one of examples 166-172, wherein the coaptation element comprises a first portion that is rectangular and a second portion that is rounded.

Example 174. The valve repair system according to any one of examples 166-173, wherein the coaptation element is injection molded.

Example 175. The valve repair system according to any one of examples 166-174, wherein the coaptation element comprises a polymer material.

Example 176. The valve repair system according to any one of examples 166-175, wherein the coaptation element has a length of between about 10 mm and about 40 mm.

Example 177. The valve repair system according to any one of examples 166-176, wherein the coaptation element has a width of between about 3 mm and about 10 mm.

Example 178. The valve repair system according to any one of examples 137-177, further comprising a distal portion having a cap.

Example 179. The valve repair system according to example 178, wherein the cap is operative connected to the anchors such that movement of the cap away from a coaptation element causes the anchors to move to an opened position and movement of the cap toward the coaptation element causes the anchors to move to a closed position.

Example 180. The valve repair system according to any off examples 178-179, wherein the cap has a distal opening that allows at least a portion of the paddle frame of each anchor to move in and out of the implantable device.

Example 181. The valve repair system according to example 180, wherein the implantable device further comprises a distal cover element that is positioned to inhibits blood from moving through the distal opening of the cap and into an interior of the implantable device.

Example 182. The valve repair system according to any one of examples 137-181, further comprising a cover attached to the paddle frame.

Example 183. The valve repair system according to example 182, wherein the cover creates a canopy that extends between first and second anchors of the one or more anchors.

Example 184. The valve repair system according to any of examples 182-183, wherein the cover comprises a single piece of material.

Example 185. The valve repair system according to any one of examples 182-183, wherein the cover comprises multiple pieces of material.

Example 186. The valve repair system according to any one of examples 182-185, wherein the cover is made of a porous material that becomes impermeable to blood flow over time.

Example 187. The valve repair system according to any one of examples 182-185, wherein the cover is made of a material that is impermeable to blood flow.

Example 188. The valve repair system according to any one of examples 182-187, wherein the cover comprises one or more stretchable portions.

Example 189. The valve repair system according to example 188, wherein the stretchable portions comprise a pair of Leno weaves with a float positioned therebetween.

Example 190. The valve repair system according to any one of examples 137-189, wherein the paddle frame comprises a connector that includes the inner end.

Example 191. A valve repair system for repairing a native valve of a patient, the valve repair system comprising: (A) a delivery device having a width adjustment element; (B) an implantable device configured to be implanted on the native valve of the patient, the implantable device having: (i) a receiver defining a lumen that includes internal threads; (ii) an anchor portion having one or more anchors, each of the anchors having a paddle frame, wherein the one or more anchors are configured to attach to one or more leaflets of a native valve; and (iii) a coupler for removably connecting the width adjustment element of the delivery device to an inner end of the anchors, wherein the coupler comprises an upper body, a lower body, and a plurality of struts connected to the upper and lower bodies, wherein the coupler is movable between an unlocked position in which the plurality of struts are positioned to be disengaged from the internal threads of the receiver and a locking position in which the plurality of struts are positioned to engage with the internal threads of the receiver.

Example 192. The valve repair system according to example 191, wherein a first width of the coupler when the coupler is in the locked position is less than a second width of the coupler when the coupler is in the locking position.

Example 193. The valve repair system according to any of examples 191-192, wherein the coupler is configured to move within the lumen of the receiver when the plurality of struts are disengaged with the internal threads of the receiver.

Example 194. The valve repair system according to any of examples 191-193, wherein the coupler is in an expanded configuration when in the unlocked position and a compressed configuration when in the locking position.

Example 195. The valve repair system according to example 194, wherein the coupler is normally in the compressed configuration.

Example 196. The valve repair system according to any of examples 191-195, wherein the coupler comprises four struts.

Example 197. The valve repair system according to any of examples 191-196, wherein the upper body comprises an upper opening for receiving the width adjustment element of the delivery device.

Example 198. The valve repair system according to any of examples 191-197, wherein the lower body comprises a lower opening for receiving the inner end of the paddle frame.

Example 199. The valve repair system according to example 198, wherein the lower opening of the lower body is further configured to receive the width adjustment element.

Example 200. The valve repair system according to any of examples 191-199, wherein the coupler is movable from the locking position to the unlocked position by removably connecting the width adjustment element of the delivery device to both of the upper and lower bodies of the coupler.

Example 201. The valve repair system according to example 200, wherein the width adjustment element is removably connected to the upper and lower bodies of the coupler by a threaded connection.

Example 202. The valve repair system according to any of examples 191-201, wherein the plurality of struts are spiraled in a counter-clockwise direction when the coupler is in the locking position.

Example 203. The valve repair system according to any of examples 191-201, wherein the plurality of struts are spiraled in a clockwise direction when the coupler is in the locking position.

Example 204. The valve repair system according to any one of examples 191-203, wherein a coaptation element defines a first area when viewed from above, wherein the paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 205. The valve repair system according to example 204, wherein the ratio of the second area to the first area is greater than or equal to 3 to 1.

Example 206. The valve repair system according to example 204, wherein the ratio of the second area to the first area is greater than or equal to 4 to 1.

Example 207. The valve repair system according to example 204, wherein the ratio of the second area to the first area is greater than or equal to 5 to 1.

Example 208. The valve repair system according to example 204, wherein the ratio of the second area to the first area is greater than or equal to 6 to 1.

Example 209. The valve repair system according to any one of examples 204-208, wherein the coaptation element comprises one or more planar side surfaces.

Example 210. The valve repair system according to any one of examples 204-209, wherein the coaptation element comprises one or more tapered side surfaces.

Example 2114. The valve repair system according to any one of examples 204-210, wherein the coaptation element comprises a first portion that is rectangular and a second portion that is rounded.

Example 212. The valve repair system according to any one of examples 204-211, wherein the coaptation element is injection molded.

Example 213. The valve repair system according to any one of examples 204-212, wherein the coaptation element comprises a polymer material.

Example 214. The valve repair system according to any one of examples 204-213, wherein the coaptation element has a length of between about 10 mm and about 40 mm.

Example 215. The valve repair system according to any one of examples 204-214, wherein the coaptation element has a width of between about 3 mm and about 10 mm.

Example 216. The valve repair system according to any one of examples 191-215, further comprising a distal portion having a cap.

Example 217. The valve repair system according to example 216, wherein the cap is operatively connected to the anchors such that movement of the cap away from a coaptation element causes the anchors to move to an opened position and movement of the cap toward the coaptation element causes the anchors to move to a closed position.

Example 218. The valve repair system according to any of examples 216-217, wherein the cap has a distal opening that allows at least a portion of the paddle frame of each anchor to move in and out of the implantable device.

Example 219. The valve repair system according to example 218, wherein the implantable device further comprises a distal cover element that is positioned to inhibit blood from moving through the distal opening of the cap and into an interior of the implantable device.

Example 220. The valve repair system according to any one of examples 191-219, further comprising a cover attached to the paddle frame.

Example 221. The valve repair system according to example 220, wherein the cover creates a canopy that extends between first and second anchors of the one or more anchors.

Example 222. The valve repair system according to any of examples 220-221, wherein the cover comprises a single piece of material.

Example 223. The valve repair system according to any one of examples 220-221, wherein the cover comprises multiple pieces of material.

Example 224. The valve repair system according to any one of examples 220-223, wherein the cover is made of a porous material that becomes impermeable to blood flow over time.

Example 225. The valve repair system according to any one of examples 220-223, wherein the cover is made of a material that is impermeable to blood flow.

Example 226. The valve repair system according to any one of examples 220-225, wherein the cover comprises one or more stretchable portions.

Example 227. The valve repair system according to example 226, wherein the stretchable portions comprise a pair of Leno weaves with a float positioned therebetween.

Example 228. The valve repair system according to any one of examples 189-227, wherein the paddle frame comprises a connector that includes the inner end.

Example 229. A valve repair system for repairing a native valve of a patient, the valve repair system comprising: (A) a delivery device having a width adjustment element; (B) an implantable device configured to be implanted on the native valve of the patient, the implantable device having: (i) a receiver defining a lumen that includes internal threads; (ii) an anchor portion having one or more anchors, each of the anchors having a paddle frame that includes an inner end, wherein the one or more anchors are configured to attach to one or more leaflets of a native valve; and (iii) a coupler for removably connecting the width adjustment element of the delivery device to the inner end of the anchors, wherein the coupler comprises at least two arms that are movable between a normal position and an engaged position, each of the arms comprise a central portion, a first connection member that connects the central portion to a proximal end of the coupler, and a second connection member that connects the central portion to a distal end of the coupler, wherein the arms are configured to attach to internal threads of the lumen of the receiver when the arms are in the normal position.

Example 230. The valve repair system according to example 229, wherein the central portion of a first arm is offset from the central portion of a second arm along a height of a body of the coupler such that the first and second arms are configured to connect to the internal threads of the receiver.

Example 231. The valve repair system according to any of examples 229-230, wherein each of the arms has a “t” shape.

Example 232. The valve repair system according to any of examples 229-231, wherein the first and second connection members are normally in torsion such that a first portion of the central portion is disposed within a lumen of the coupler and a second portion of the central portion extends away from a body of the coupler when the coupler is in the normal position.

Example 233. The valve repair system according to any of examples 229-232, wherein the central portion of each of the arms is substantially aligned with a body of the coupler when the coupler is in the engaged position.

Example 234. The valve repair system according to any of examples 229-233, wherein the width adjustment element of the delivery device removably connects to the coupler by a threaded connection.

Example 235. The valve repair system according to any one of examples 229-234, wherein a coaptation element defines a first area when viewed from above, wherein the paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 236. The valve repair system according to example 235, wherein the ratio of the second area to the first area is greater than or equal to 3 to 1.

Example 237. The valve repair system according to example 235, wherein the ratio of the second area to the first area is greater than or equal to 4 to 1.

Example 238. The valve repair system according to example 235, wherein the ratio of the second area to the first area is greater than or equal to 5 to 1.

Example 239. The valve repair system according to example 235, wherein the ratio of the second area to the first area is greater than or equal to 6 to 1.

Example 240. The valve repair system according to any one of examples 235-239, wherein the coaptation element comprises one or more planar side surfaces.

Example 241. The valve repair system according to any one of examples 235-240, wherein the coaptation element comprises one or more tapered side surfaces.

Example 242. The valve repair system according to any one of examples 235-241, wherein the coaptation element comprises a first portion that is rectangular and a second portion that is rounded.

Example 243. The valve repair system according to any one of examples 235-242, wherein the coaptation element is injection molded.

Example 244. The valve repair system according to any one of examples 235-243, wherein the coaptation element comprises a polymer material.

Example 245. The valve repair system according to any one of examples 235-244, wherein the coaptation element has a length of between about 10 mm and about 40 mm.

Example 246. The valve repair system according to any one of examples 235-245, wherein the coaptation element has a width of between about 3 mm and about 10 mm.

Example 247. The valve repair system according to any one of examples 229-246, further comprising a distal portion having a cap.

Example 248. The valve repair system according to example 247, wherein the cap is operative connected to the anchors such that movement of the cap away from a coaptation element causes the anchors to move to an opened position and movement of the cap toward the coaptation element causes the anchors to move to a closed position.

Example 249. The valve repair system according to any off examples 247-248, wherein the cap has a distal opening that allows at least a portion of the paddle frame of each anchor to move in and out of the implantable device.

Example 250. The valve repair system according to example 249, wherein the implantable device further comprises a distal cover element that is positioned to inhibit blood from moving through the distal opening of the cap and into an interior of the implantable device.

Example 251. The valve repair system according to any one of examples 229-250, further comprising a cover attached to the paddle frame.

Example 252. The valve repair system according to example 251, wherein the cover creates a canopy that extends between first and second anchors of the one or more anchors.

Example 253. The valve repair system according to any of examples 251-252, wherein the cover comprises a single piece of material.

Example 254. The valve repair system according to any one of examples 251-252, wherein the cover comprises multiple pieces of material.

Example 255. The valve repair system according to any one of examples 251-254, wherein the cover is made of a porous material that becomes impermeable to blood flow over time.

Example 256. The valve repair system according to any one of examples 251-254, wherein the cover is made of a material that is impermeable to blood flow.

Example 257. The valve repair system according to any one of examples 251-256, wherein the cover comprises one or more stretchable portions.

Example 258. The valve repair system according to example 257, wherein the stretchable portions comprise a pair of Leno weaves with a float positioned therebetween.

Example 259. The valve repair system according to any one of examples 229-258, wherein the paddle frames are connected to the coupler by a connector that includes the inner end.

Example 260. A valve repair system for repairing a native valve of a patient, the valve repair system comprising: (A) a delivery device having one or more actuation elements; (B) an implantable device configured to be implanted on the native valve of the patient, the implantable device having: (i) a coaptation element having a lumen for receiving the one or more actuation elements of the delivery device; (ii) an anchor portion having one or more anchors for attaching to one or more leaflets of a native valve, wherein the anchors are movable between an open position and a closed position; and (iii) a cap operatively connected to the one or more anchors such that movement of the cap relative to the coaptation element by one or more actuation elements of the delivery device causes the anchors to move between the open and closed positions, wherein the cap comprises a distal opening that is in communication with the lumen of the coaptation element; and (iv) a distal cover element that is positioned to inhibit blood from moving through the distal opening of the cap and into an interior of the implantable device.

Example 261. The valve repair system according to example 260, wherein the implantable device further comprises a receiver disposed within the lumen of the coaptation element, wherein the receiver is connected to the cap, and wherein a first actuation element of the one or more actuation elements of the delivery device is configured to engage the receiver to move the receiver and cap relative to the coaptation element to move the anchors between the open and closed positions.

Example 262. The valve repair system according to example 261, wherein an inner end portion of the anchor portion extends through the distal end of the cap.

Example 263. The valve repair system according to example 262, wherein the paddle frames connected to the coupler by a connector that includes the inner end.

Example 264. The valve repair system according to any of examples 262-263, wherein the inner end is connected to the receiver by a coupler, wherein the coupler is configured to be removably connected to a width adjustment element of the one or more actuation elements of the delivery device such that the width adjustment element can cause the inner end to move relative to the receiver to move the paddle frame between narrowed and expanded positions.

Example 265. The valve repair system according to any of examples 260-264, wherein the distal cover element is attached to paddle frames of the anchor portion.

Example 266. The valve repair system according to any of examples 260-264, wherein the distal cover element is attached to the cap.

Example 267. The valve repair system according to any of examples 260-266, wherein the distal cover element has a center portion having a first width and opposing side edges that extend from the center portion and have a second width, and wherein the second width is greater than the first width.

Example 268. The valve repair system according to any of examples 260-267, wherein the distal cover element is curved such that edges of the distal cover element extend upward from a center portion.

Example 269. The valve repair system according to any one of examples 260-268, wherein the coaptation element defines a first area when viewed from above, wherein paddle frames of the anchors define an outer portion of the implantable device when viewed from above and the anchors are in the closed position, wherein the outer portion of the device has a second area when viewed from above, and wherein a ratio of the second area to the first area is greater than or equal to about 2 to 1.

Example 270. The valve repair system according to example 269, wherein the ratio of the second area to the first area is greater than or equal to 3 to 1.

Example 271. The valve repair system according to example 269, wherein the ratio of the second area to the first area is greater than or equal to 4 to 1.

Example 272. The valve repair system according to example 269, wherein the ratio of the second area to the first area is greater than or equal to 5 to 1.

Example 273. The valve repair system according to example 269, wherein the ratio of the second area to the first area is greater than or equal to 6 to 1.

Example 274. The valve repair system according to any one of examples 269-273, wherein the coaptation element comprises one or more planar side surfaces.

Example 275. The valve repair system according to any one of examples 269-274, wherein the coaptation element comprises one or more tapered side surfaces.

Example 276. The valve repair system according to any one of examples 269-275, wherein the coaptation element comprises a first portion that is rectangular and a second portion that is rounded.

Example 277. The valve repair system according to any one of examples 269-276, wherein the coaptation element is injection molded.

Example 278. The valve repair system according to any one of examples 269-277, wherein the coaptation element comprises a polymer material.

Example 279. The valve repair system according to any one of examples 269-278, wherein the coaptation element has a length of between about 10 mm and about 40 mm.

Example 280. The valve repair system according to any one of examples 269-279, wherein the coaptation element has a width of between about 3 mm and about 10 mm.

Example 281. The valve repair system according to any one of examples 260-280, further comprising a cover attached to the anchor portion.

Example 282. The valve repair system according to example 281, wherein the cover is attached to a paddle frame of at least one of the anchors.

Example 283. The valve repair system according to at least one of examples 281-282, wherein the cover creates a canopy that extends between first and second anchors of the one or more anchors.

Example 284. The valve repair system according to any of examples 281-283, wherein the cover comprises a single piece of material.

Example 285. The valve repair system according to any one of examples 281-283, wherein the cover comprises multiple pieces of material.

Example 286. The valve repair system according to any one of examples 281-285, wherein the cover is made of a porous material that becomes impermeable to blood flow over time.

Example 287. The valve repair system according to any one of examples 281-285, wherein the cover is made of a material that is impermeable to blood flow.

Example 288. The valve repair system according to any one of examples 281-287, wherein the cover comprises one or more stretchable portions.

Example 289. The valve repair system according to example 288, wherein the stretchable portions comprise a pair of Leno weaves with a float positioned therebetween.

Example 290. An implantable device, comprising: (i) an anchor portion comprising a first anchor and a second anchor, each of the first and second anchors comprising a paddle frame that is configured to be moved between a narrowed position and expanded position, wherein the first and second anchors are configured to be moved from open position to a closed position to secure the implantable device to leaflets of a native valve; and (ii) a cover attached to the paddle frame of the first and second anchors, wherein the cover is sized to be in a taut state when the paddle frame is in the narrowed position, and wherein the cover comprises one or more stretchable portions that allow the cover to stretch when the paddle frame is in the expanded position.

Example 291. The implantable device according to example 2890, wherein each of the stretchable portions comprise a pair of Leno weaves with a float positioned therebetween.

Example 292. The implantable device according to any of examples 290-291, wherein the cover comprises at least two stretchable portions.

Example 293. The implantable device according to any of examples 290-292, wherein the cover comprises a single piece of material.

Example 294. The implantable device according to any one of examples 290-293, wherein the cover comprises multiple pieces of material.

Example 295. The implantable device according to example 294, wherein one or more pieces of the multiple pieces of material comprise at least one stretchable portion.

Example 296. The implantable device according to any one of examples 290-295 wherein the cover is made of a porous material that becomes impermeable to blood flow over time.

Example 297. The implantable device according to any one of examples 290-295, wherein the cover is made of a material that is impermeable to blood flow.

Example 298. The implantable device according to any one of examples 291-297, wherein the float of the stretchable portion of the cover comprises unwoven threads.

Example 299. The implantable device according to any one of examples 291-298, wherein each of the Leno weaves of the stretchable portion of the cover comprises warp threads and weft threads that are woven in a perpendicular weaving patter and a Leno thread that wraps around the warp threads.

Example 300. The implantable device according to example 299, wherein each of the Leno weaves of the stretchable portion of the cover comprises four warp threads.

Example 301. The implantable device according to any one of examples 290-300, wherein the paddle frame of each anchor includes an inner frame portion and an outer frame portion, wherein the outer frame portion is configured to be moved between the narrow and expanded position.

Example 302. The implantable device according to example 301, wherein the cover is attached to the outer frame portion of the paddle frame.

Example 303. The implantable device according to any of examples 301-302, wherein the cover is attached to the inner frame portion of the paddle frame.

Example 304. The implantable device according to any of examples 301-303, wherein the inner frame portion is rigid, and the outer frame portion is flexible.

Example 305. The implantable device according to any of examples 230-304, wherein the cover creates a canopy that extends between the first and second anchors.

Example 306. The implantable device according to any one of examples 290-305, wherein a coaptation element defines a first area when viewed from above, wherein the paddle frames of the first and second anchors define an outer portion of the implantable device when viewed from above and the first and second anchors are in the closed position, wherein the outer portion of the device has a second area when viewed from above, and wherein a ratio of the second area to the first area is greater than or equal to about 2 to 1.

Example 307. The implantable device according to example 306, wherein the ratio of the second area to the first area is greater than or equal to 3 to 1.

Example 308. The implantable device according to example 306, wherein the ratio of the second area to the first area is greater than or equal to 4 to 1.

Example 309. The implantable device according to example 306, wherein the ratio of the second area to the first area is greater than or equal to 5 to 1.

Example 310. The implantable device according to example 306, wherein the ratio of the second area to the first area is greater than or equal to 6 to 1.

Example 311. The implantable device according to any one of examples 306-310, wherein the coaptation element comprises one or more planar side surfaces.

Example 312. The implantable device according to any one of examples 306-311, wherein the coaptation element comprises one or more tapered side surfaces.

Example 313. The implantable device according to any one of examples 306-312, wherein the coaptation element comprises a first portion that is rectangular and a second portion that is rounded.

Example 314. The implantable device according to any one of examples 306-313, wherein the coaptation element is injection molded.

Example 315. The implantable device according to any one of examples 306-314, wherein the coaptation element comprises a polymer material.

Example 316. The implantable device according to any one of examples 306-315, wherein the coaptation element has a length of between about 10 mm and about 40 mm.

Example 317. The implantable device according to any one of examples 306-316, wherein the coaptation element has a width of between about 3 mm and about 10 mm.

Example 318. The implantable device according to any one of examples 290-317, further comprising a distal portion having a cap.

Example 319. The implantable device according to example 318, wherein the cap is operative connected to the anchors such that movement of the cap away from a coaptation element causes the anchors to move to an opened position and movement of the cap toward the coaptation element causes the anchors to move to a closed position.

Example 320. The implantable device according to any of examples 318-319, wherein the cap has a distal opening that allows at least a portion of the paddle frame of each anchor to move in and out of the implantable device.

Example 321. The implantable device according to example 320, wherein the implantable device further comprises a distal cover element that is positioned to inhibit blood from moving through the distal opening of the cap and into an interior of the implantable device.

Example 322. An implantable device, comprising an anchor portion comprising a first anchor and a second anchor, wherein each of the first and second anchors comprising a paddle frame that includes an outer frame portion, wherein the outer frame portion has a proximal end and a distal end, wherein the outer frame portion is configured to be moved between a narrowed position and an expanded position, wherein a proximal width of the proximal end of the outer frame portion is greater than a distal width of the distal end of the outer frame portion when the outer frame portion is in the expanded position, and wherein the first and second anchors are configured to be moved to a closed position such that the implantable device is secured to the native valve.

Example 323. The implantable device according to example 322, wherein the proximal width of the proximal end of the outer frame portion when the outer frame portion is in the expanded position is between about 8 mm and about 12 mm.

Example 324. The implantable device according to any one of claims 322-323, wherein a ratio of the proximal width of the proximal end of the outer frame portion to a distal width of a distal end of the outer frame portion is between about 1.1 and about 1.5 when the outer frame portion is in the expanded position.

Example 325. The implantable device according to any one of claims 322-3234, wherein engagement between the paddle frame and the native valve when the implantable device is secured to the native valve causes a force on an annulus of the native valve that reshapes the annulus.

Example 326. The implantable device according to any one of claims 322-325, further comprising a cover attached to the outer frame portion.

Example 327 The implantable device according to any one of examples 322-326, where the distal width is maintained as greater than the proximal width as the outer frame portion moves from the expanded position to the narrowed position.

Example 328. The implantable device according to example 326, wherein the cover is sized to be in a taut state when the paddle frame is in the narrowed position, and wherein the cover stretches to move to the expanded position.

Example 329. The implantable device according to any one of claims 326-328, wherein the cover creates a canopy that extends between the first and second anchors.

Example 330. The implantable device according to any one of claims 322-329, wherein a coaptation element defines a first area when viewed from above, wherein the paddle frames of the first and second anchors define an outer portion of the implantable device when viewed from above and the first and second anchors are in the closed position, wherein the outer portion of the device has a second area when viewed from above, and wherein a ratio of the second area to the first area is greater than or equal to about 2 to 1.

Example 331. The implantable device according to any one of claims 322-330, in a first phase, starting at the expanded position and moving to an intermediate position, the distal width narrows at a faster rate than the proximal width.

Example 332. The implantable device according to example 331, in a first second phase, starting at the intermediate position and moving to the narrowed position, the distal width narrows at a faster rate than the proximal width.

Example 333. An implantable device, comprising an anchor portion comprising a first anchor and a second anchor, wherein each of the first and second anchors comprise a paddle frame that includes an outer frame portion, wherein the outer frame portion has a proximal end and a distal end, wherein the outer frame portion is configured to be moved between a narrowed position and an expanded position, wherein a distal width of the distal end of the outer frame portion is greater than a proximal width of the proximal end of the outer frame portion when the outer frame portion is in the expanded position, and wherein the first and second anchors are configured to be moved to a closed position such that the implantable device is secured to the native valve.

Example 334. The implantable device according to example 333, wherein the distal width of the distal end of the outer frame portion when the outer frame portion is in the expanded position is between about 8 mm and about 14 mm.

Example 335. The implantable device according to any one of claims 333-334, wherein a ratio of the distal width of the distal end of the outer frame portion to a proximal width of a proximal end of the outer frame portion is between about 4/1 and about 4/3 when the outer frame portion is in the expanded position.

Example 336. The implantable device according to any one of claims 333-335, wherein the engagement between the paddle frame and the native valve when the implantable device is secured to the native valve causes free edges of leaflets of the native valve to be pinched together.

Example 337. The implantable device according to any one of claims 333-336, further comprising a cover attached to at least one of the inner frame portion and the outer frame portion.

Example 338. The implantable device according to example 337, wherein the cover is sized to be in a taut state when the paddle frame is in the narrowed position, and wherein the cover stretches when the paddle frame moves to the expanded position.

Example 339. The valve repair system according to any one of claims 337-338, wherein the cover creates a canopy that extends between the first and second anchors.

Example 340. The implantable device according to any one of claims 333-339, wherein a coaptation element defines a first area when viewed from above, wherein the paddle frames of the first and second anchors define an outer portion of the implantable device when viewed from above and the first and second anchors are in the closed position, wherein the outer portion of the device has a second area when viewed from above, and wherein a ratio of the second area to the first area is greater than or equal to about 2 to 1.

Example 341. The implantable device according to any one of claims 333-340, further comprising a distal portion having a cap.

Example 342. The implantable device according to example 341, wherein the cap is operatively connected to the anchors such that movement of the cap away from a coaptation element causes the anchors to move to an opened position and movement of the cap toward the coaptation element causes the anchors to move to a closed position.

Example 343. The implantable device according to any of claims 341-342, wherein the cap has a distal opening that allows at least a portion of the paddle frame of each anchor to move in and out of the implantable device.

Example 344. The implantable device according to example 343, wherein the implantable device further comprises a distal cover element that is positioned to inhibit blood from moving through the distal opening of the cap and into an interior of the implantable device.

Example 345. The implantable device according to any one of claims 331-342, where the distal width is maintained as the widest portion of the outer paddle frame when the outer paddle frame moves from the expanded position to the narrowed position.

Example 346. An implantable device, comprising an anchor portion comprising a first anchor and a second anchor, each of the first and second anchors comprising a paddle frame, wherein the paddle frame is configured to be moved between a narrowed configuration and expanded configuration, wherein the first and second anchors are configured to be moved to a closed position in which the first and second anchors compress one or more native leaflets such that the implantable device is secured to the native valve.

Example 347. The implantable device according to example 346, wherein the paddle frame comprises an inner frame portion and an outer frame portion, and wherein the inner frame portion is rigid, and the outer frame portion is flexible.

Example 348. The implantable device according to any one of examples 346-347, further comprising a flexible cover attached to the paddle frame, wherein the flexible cover is configured to be in a taut state when the paddle frame is in the narrowed configuration, and wherein the flexible cover is configured to stretch when the paddle frame is in the expanded configuration.

Example 349. The implantable device according to example 348, wherein the flexible cover comprises a first membrane that attaches to the paddle frame of the first anchor and a second membrane that attaches to the paddle frame of the second anchor.

Example 350. The implantable device according to any one of examples 348-349, wherein the flexible cover comprises a single membrane that attaches to the paddle frame of both of the first and second anchors.

Example 351. The implantable device according to any one of examples 346-350, wherein the paddle frame of each of the first and second anchors includes an inner frame portion and an outer frame portion, and wherein the paddle frame is configured such that the outer frame portion of the paddle frame changes shape as the paddle frame is moved between the narrowed configuration and the expanded configuration.

Example 352. The implantable device according to any one of examples 346-351, wherein the paddle frame of each of the first and second anchors includes an inner frame portion and an outer frame portion, and wherein the first and second anchors are configured such that, in the closed position, the inner frame portion of each of the first and second anchors can compress the one or more native leaflets between an inner pinch point and the outer frame portion of each of the first and second anchors can compress the one or more native leaflets between an outer pinch point such that the implantable device is secured to the native valve.

Example 353. An implantable device, comprising an anchor portion comprising an anchor, the anchor comprising a paddle frame, wherein the paddle frame is configured to be moved between a narrowed configuration and expanded configuration, and wherein the anchor is configured to be moved to a closed position in which the anchor compresses at least one native leaflet such that the implantable device is secured to a native valve.

Example 354. The implantable device according to example 353, wherein the paddle frame includes an inner frame portion and an outer frame portion, and wherein the inner frame portion is rigid, and the outer frame portion is flexible.

Example 355. The implantable device according to any one of examples 353-354, further comprising a cover attached to the paddle frame, wherein the cover is configured to be in a taut state when the paddle frame is in the narrowed configuration, and wherein the cover is configured to stretch when the paddle frame is in the expanded configuration.

Example 356. The implantable device according to example 355, wherein the cover extends across at least a portion of an inner surface of the paddle frame.

Example 357. The implantable device according to any one of examples 355-356, wherein the cover extends across an entirety of an area defined by an inner surface of the paddle frame.

Example 358. The implantable device according to any one of examples 355-357, wherein the anchor is a first anchor and the implantable device also includes a second anchor, and wherein the cover comprises a single membrane that attaches to the paddle frame of the first anchor and the second anchor.

Example 359. The implantable device according to example 358, wherein the single membrane creates a canopy that extends between the first anchor and the second anchor.

Example 360. The implantable device according to any one of examples 355-359, wherein the anchor is a first anchor and the implantable device includes a second anchor that also comprises a paddle frame, wherein the paddle frame of both the first anchor and the second anchor includes an inner frame portion and an outer frame portion, and wherein the cover is attached to the inner frame portion and the outer frame portion of the first and second anchors, and wherein the cover extends between the first anchor and the second anchor.

Example 361. The implantable device according to any one of examples 353-360, wherein the paddle frame includes an inner frame portion and an outer frame portion, and wherein the paddle frame is configured such that when the paddle frame is moved between the narrowed configuration and the expanded configuration, the outer frame portion of the paddle frame changes shape between the narrowed configuration and the expanded configuration.

Example 362. The implantable device according to any one of examples 353-361, wherein the paddle frame includes an inner frame portion and an outer frame portion, and wherein the anchor is configured such that, in the closed position, the inner frame portion of the anchor can compress the at least one native leaflet between an inner pinch point and the outer frame portion of each of the anchor can compress the at least one native leaflet between an outer pinch point such that the implantable device is secured to the native valve.

Example 363. A valve repair system for repairing a native valve of a patient, the valve repair system comprising (A) a delivery device having a width adjustment element, and (B) an implantable device configured to be implanted at the native valve of the patient, the implantable device having: (i) an anchor portion having one or more anchors, each of the one or more anchors having a paddle frame, wherein the one or more anchors are configured to attach to one or more leaflets of a native valve; and (ii) a coupler for removably connecting the width adjustment element of the delivery device to an end of the one or more anchors, wherein the coupler is configured such that it can secure the end of the one or more anchors in one of multiple potential positions within the implantable device such that the anchors are held in one of multiple potential configurations selected from the group comprising a narrowed configuration, an extended configuration, and an intermediate configuration between the narrowed configuration and the extended configuration.

Example 364. The valve repair system according to example 363, wherein the coupler comprises at least two arms that are movable between a normal position and an engaged position, wherein the arms are in the normal position when the coupler is disconnected from the width adjustment element, and wherein the arms are in the engaged position when the coupler is connected to the width adjustment element.

Example 365. The valve repair system according to any of examples 363-364, wherein the coupler comprises an upper body, a lower body, and a plurality of struts connected to the upper and lower bodies.

Example 366. The valve repair system according to any one of examples 363-365, wherein a coaptation element defines a first area when viewed from above, wherein the paddle frames of the one or more anchors define an outer region of the implantable device when viewed from above and the one or more anchors are in a closed position, wherein the outer region of the implantable device has a second area when viewed from above, and wherein a ratio of the second area to the first area is greater than or equal to about 2 to 1.

Example 367. The valve repair system according to any one of examples 363-366, wherein the paddle frame comprises a connector that includes the end.

Example 368. The valve repair system according to any one of examples 363-367, wherein the implantable device further comprises a cover attached to the paddle frame.

Example 369. The valve repair system according to example 368, wherein the cover is configured to be in a taut state when the paddle frame is in the narrowed configuration, and wherein the cover is configured to stretch as the paddle frame transitions from the narrowed configuration to the expanded configuration.

Example 370. The valve repair system according to any one of examples 368-369, wherein the cover comprises a single membrane that attaches to the paddle frame of both of the first and second anchors.

Example 371. The valve repair system according to any one of examples 368-370, wherein the cover is configured to form a canopy that extends between the first and second anchors.

Example 372. The valve repair system according to any one of examples 363-371, wherein the paddle frame includes an inner frame portion and an outer frame portion, and wherein the paddle frame is configured such that when the paddle frame is moved between the narrowed configuration and the expanded configuration, the outer frame portion of the paddle frame changes shape between the narrowed configuration and the expanded configuration.

Example 373. The valve repair system according to any one of examples 363-372, wherein the paddle frame includes an inner frame portion and an outer frame portion, and wherein the anchor is configured such that the anchor can be moved to a closed position in which the inner frame portion of the anchor can compress the one or more native leaflets between an inner pinch point and the outer frame portion of each of the anchor compresses the one or more native leaflets between an outer pinch point.

Example 374. The valve repair system according to any one of examples 363-373, wherein the width adjustment element includes an external threaded portion.

Example 375. The valve repair system according to any one of examples 363-374, wherein the coupler comprises one or more attachment projections that extend inward from a body of the coupler.

Example 376. The valve repair system according to example 375, wherein the one or more attachment projections are configured to removably attach to an external threaded portion of the width adjustment element.

Example 377. The valve repair system according to any one of examples 363-376, wherein the system includes a receiver that defines a lumen that includes internal threads.

Example 378. The valve repair system according to any one of examples 363-377, wherein the coupler comprises at least two arms that are movable between a normal position and an engaged position, wherein the arms are in the normal position when the coupler is disconnected from the width adjustment element, wherein the arms are in the engaged position when the coupler is connected to the width adjustment element.

Example 379. The valve repair system according to example 378, wherein the at least two arms are configured to attach to internal threads of a lumen of a receiver when the arms are in the normal position.

Example 380. The valve repair system according to any one of examples 363-379, further comprising a receiver including an unattachable portion.

Example 381. The valve repair system according to example 380, wherein the coupler is configured to be removably coupled to the receiver to secure the end of the one or more anchors in a desired position relative to the receiver.

Example 382. The valve repair system according to any one of examples 378-381, wherein the unattachable portion of the receiver prevents the coupler from connecting to the receiver when the coupler is disposed within the unattachable portion.

Example 383. The valve repair system according to any one of examples 363-382, wherein the implantable device further comprises a coaptation element.

Example 384. The valve repair system according to any one of examples 363-383, wherein the implantable device further comprises a cap operatively connected to the one or more anchors such that movement of the cap relative to another portion of the implantable device by one or more actuation elements of the delivery device causes the one or more anchors to move between an open position and a closed position.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. An implantable device comprising:
a coaptation element that defines a first area when viewed from above;
one or more anchors coupled to the coaptation element, the one or more anchors being movable between an open position and a closed position and configured to attach to one or more leaflets of a native heart valve, each of the one or more anchors comprising a paddle frame, wherein the paddle frame defines an outer portion of the implantable device when viewed from above and the one or more anchors are in the closed position, wherein the outer portion of the implantable device has a second area when viewed from above; and
wherein a ratio of the second area to the first area is greater than or equal to 2 to 1.

2. The implantable device according to claim 1, wherein the ratio of the second area to the first area is greater than or equal to 3 to 1.

3. The implantable device according to claim 1, wherein the coaptation element is injection molded.

4. The implantable device according to claim 1, wherein the coaptation element comprises a polymer material.

5. The implantable device according to claim 1 wherein each of the first and second paddle frames comprise:
an inner frame portion and an outer frame portion;
wherein the first and second anchors are configured to be moved to a closed position in which the inner frame portion of each of the first and second anchors compress native valve leaflets between an inner pinch point and the outer frame portion of each of the first and second anchors compress the native valve leaflets between an outer pinch point such that the implantable device is secured to the native valve leaflets;
and a cover attached to at least one of the inner frame portion and the outer frame portion of the first and second anchors to provide a compressive force against the native valve leaflets in an area between the inner and outer pinch points when the implantable device is secured to the native valve leaflets.

6. The implantable device according to claim 5, wherein the cover creates a canopy that extends between the first and second anchors.

7. The implantable device according to claim 1, wherein each of the first and second paddle frames comprise:
a paddle frame that includes an inner frame portion and an outer frame portion, wherein the outer frame portion is configured to be moved between a narrowed position and expanded position; and
a flexible cover is configured to be in a taut state when the outer frame portion is in the narrowed position, and wherein the flexible cover is configured to stretch when the outer frame portion is in the expanded position.

8. The implantable device of claim 7 wherein the flexible cover comprises one or more stretchable portions that allow the cover to stretch when the paddle frame is in the expanded position.

9. The implantable device of claim 1 further comprising a cap operatively connected to the one or more anchors such that movement of the cap relative to the coaptation element by the one or more actuation elements of the delivery device causes the one or more anchors to move between the open and closed positions, wherein the cap comprises a distal opening that is in communication with a lumen of the coaptation element, and a distal cover element that is positioned to inhibit blood from moving through the distal opening of the cap and into an interior of the implantable device.

10. A valve repair system for repairing a native valve of a patient, the valve repair system comprising:
a delivery device;
an implantable device coupled to the delivery device, the implantable device comprising:
a coaptation element that defines a first area when viewed from above;
one or more anchors coupled to the coaptation element, the one or more anchors being movable between an open position and a closed position and configured to attach to one or more leaflets of a native heart valve, each of the one or more anchors comprising a paddle frame, wherein the paddle frame defines an outer portion of the implantable device when viewed from above and the one or more anchors are in the closed position, wherein the outer portion of the implantable device has a second area when viewed from above; and
wherein a ratio of the second area to the first area is greater than or equal to 2 to 1.

11. The valve repair system according to claim 10, wherein the ratio of the second area to the first area is greater than or equal to 3 to 1.

12. The valve repair system according to claim 10, wherein the coaptation element is injection molded.

13. The valve repair system according to claim 10, wherein the coaptation element comprises a polymer material.

14. The valve repair system of claim 10 wherein the delivery device has a width adjustment element that includes an external threaded portion;
wherein the implantable device includes a coupler for removably connecting the width adjustment element of the delivery device to the inner end of the one or more anchors;
wherein the coupler comprises one or more attachment projections that extend inward from a body of the coupler;
wherein the one or more attachment projections are configured to removably attach to the external threaded portion of the width adjustment element; and
wherein the width adjustment element is configured to move the paddle frame between a narrowed position and an expanded position.

15. The valve repair system according to claim 14, wherein the coupler comprises at least two arms that are movable between a normal position and an engaged position, wherein the arms are in the normal position when the coupler is disconnected from the implantable device, and wherein the arms are in the engaged position when the coupler is connected to the width adjustment element.

16. The valve repair system of claim 14, wherein the delivery device includes a receiver;
wherein the coupler is configured to be removably coupled to internal internal threads of the receiver to secure an inner end of the one or more anchors in a desired position relative to the receiver;
wherein an unattachable portion of the receiver prevents the coupler from connecting to the receiver when the coupler is disposed within the unattachable portion.

17. The valve repair system of claim 16, wherein the coupler comprises at least two arms that are movable between a normal position and an engaged position, each of the arms comprise a central portion, a first connection member that connects the central portion to a proximal end of the coupler, and a second connection member that connects the central portion to a distal end of the coupler, wherein the arms are configured to attach to internal threads of the lumen of the receiver when the arms are in the normal position.

18. An implantable device, comprising:
an anchor portion comprising a first anchor and a second anchor;
each of the first and second anchors comprising a paddle frame that includes an outer frame portion;
wherein the outer frame portion has a proximal end and a distal end;
wherein the outer frame portion is configured to be moved between a narrowed position and an expanded position;
wherein a proximal width of the proximal end of the outer frame portion is greater than a distal width of the distal end of the outer frame portion when the outer frame portion is in the expanded position; and
wherein the first and second anchors are configured to be moved to a closed position such that the implantable device is secured to the native valve.

19. The implantable device according to claim 18, wherein engagement between the paddle frame and the native valve when the implantable device is secured to the native valve causes a force on an annulus of the native valve that reshapes the annulus.

20. The implantable device according to claim 18, in a first phase, starting at the expanded position and moving to an intermediate position, the distal width narrows at a faster rate than the proximal width.
